SEQUENCE LISTING (78 sequences)

SEQ ID NO: 1 - protein, 24 aa, Mus sp.
NAASIPSGSHKVTLSSWYHDRGWA

SEQ ID NO: 2 - protein, 24 aa, Rattus sp.
NAADIPSGSHKVSLSSWYHDRGWA

SEQ ID NO: 3 - protein, 24 aa, Canis sp.
NATDIPSGSHKVSLSSWYHDRGWA

SEQ ID NO: 4 - protein, 24 aa, Homo sapiens
NATDIPSGSHKVSLSSWYHDRGWA

SEQ ID NO: 5 - protein, 12 aa, Mus sp.
HETSGSVPTDYL

SEQ ID NO: 6 - protein, 12 aa, Rattus sp.
HETSGSVPADYL

SEQ ID NO: 7 - protein, 12 aa, Canis sp.
HETSGDLATEYL

SEQ ID NO: 8 - protein, 12 aa, Homo sapiens
HETSGDLATEYL

SEQ ID NO: 9 - protein, 8 aa, Mus sp.
TSIKIPSS

SEQ ID NO: 10 - protein, 8 aa, Rattus sp.
TSIKIPSS

SEQ ID NO: 11 - protein, 8 aa, Canis sp.
TSIKIPSS

SEQ ID NO: 12 - protein, 8 aa, Homo sapiens
TSIKIPSS

SEQ ID NO: 13 - protein, 9 aa, Mus sp.
KNWSGNSEF

SEQ ID NO: 14 - protein, 9 aa, Rattus sp.
KNWSGNSEF

SEQ ID NO: 15 - protein, 9 aa, Canis sp.
KYWSGNSEF

SEQ ID NO: 16 - protein, 9 aa, Homo sapiens
KYWSGNSEF

SEQ ID NO: 17 - protein, 316 aa, Mus sp.
MRRASRDYGKYLRSSEEMGSGPGVPHEGPLHPAPSAPAPAPPPAASRSMFLALLGLGLGQVVCSIALFLYFRAQMDPNRISEDSTHCFYRILRLHENAGLQDSTLESEDTLPDSCRRMKQAFQGAVQKELQHIVGPQRFSGAPAMMEGSWLDVAQRGKPEAQPFAHLTINAASIPSGSHKVTLSSWYHDRGWAKISNMTLSNGKLRVNQDGFYYLYANICFRHHETSGSVPTDYLQLMVYVVKTSIKIPSSHNLMKGGSTKNWSGNSEFHFYSINVGGFFKLRAGEEISIQVSNPSLLDPDQDATYFGAFKVQDID

SEQ ID NO: 18 - protein, 24 aa, Artificial Sequence
  Description of Artificial Sequence Synthetic peptide
  MOD_RES (1): Asn, Arg, Asp, Gly, or Ser
  MOD_RES (2): Ala, Asp or Ser
  MOD_RES (3): Ala, Gln, Ile, Lys or Thr
  MOD_RES (4): Ser, Asn, Asp, Gln or Thr
  MOD_RES (5): Ile, Ala, Arg, Asn, Asp, Gln, Glu, Gly, Leu, Lys, Met, Phe, Pro, Ser, Thr or Val
  MOD_RES (6): Pro, Asn or Tyr
  MOD_RES (7): Ser or Thr
  MOD_RES (8): Gly, Asn or Gln
  MOD_RES (9): Ser, Glu or Thr
  MOD_RES (10): His, Arg, Cys, Glu or Pro
  MOD_RES (11): Lys, Arg or Asn
  MOD_RES (12): Val, Ala or His
  MOD_RES (13): Thr, Asn, Pro, Ser or Val
  MOD_RES (14): Leu, Thr or Val
  MOD_RES (15): Ser, His, Ile or Thr
  MOD_RES (16): Ser, Cys, Ile or Thr
  MOD_RES (18): Tyr, Ala, Asn, Asp or His
  MOD_RES (19): His, Asn, Gln or Phe
  MOD_RES (20): Asp, Arg or Lys
  MOD_RES (21): Arg, Glu, His, Lys or Ser
  MOD_RES (22): Gly or Phe
  MOD_RES (23): Trp, Gln, Gly or Phe
  MOD_RES (24): Ala or Cys
XXXXXXXXXXXXXXXXWXXXXXXX

SEQ ID NO: 19 - protein, 24 aa, Artificial Sequence
  Description of Artificial Sequence Synthetic peptide
  MOD_RES (3): Ala or Thr
  MOD_RES (4): Ser or Asp
  MOD_RES (13): Thr or Ser
NAXXIPSGSHKVXLSSWYHDRGWA

SEQ ID NO: 20 - protein, 12 aa, Artificial Sequence
  Description of Artificial Sequence Synthetic peptide
  MOD_RES (1): His, Pro or Trp
  MOD_RES (2): Glu, Ile, Leu or Ser
  MOD_RES (3): Thr, Leu, Pro or Val
  MOD_RES (4): Ser, Gln or Lys
  MOD_RES (5): Gly, Ala or Asn
  MOD_RES (6): Ser, Ala, Asn, Asp or Lys
  MOD_RES (7): Val, Asp, Glu or Leu
  MOD_RES (8): Pro, Ala, Gln, Ser, Thr or Tyr
  MOD_RES (9): Thr, Ala, Arg, Lys or Ser
  MOD_RES (10): Asp, Arg, Asn, Glu or Ser
  MOD_RES (11): Tyr, Ala, Asn, Gly or Val
  MOD_RES (12): Leu, Thr or His
XXXXXXXXXXXX

SEQ ID NO: 21 - protein, 12 aa, Artificial Sequence
  Description of Artificial Sequence Synthetic peptide
  MOD_RES (6): Ser or Asp
  MOD_RES (7): Val or Leu
  MOD_RES (8): Pro or Ala
  MOD_RES (9): Thr or Ala
  MOD_RES (10): Asp or Glu
HETSGXXXXXYL

SEQ ID NO: 22 - protein, 8 aa, Artificial Sequence
  Description of Artificial Sequence Synthetic peptide
  MOD_RES (1): Thr, Ala, Glu or Lys
  MOD_RES (2): Ser, Asn, Lys or Thr
  MOD_RES (3): Ile, Leu, Lys or Thr
  MOD_RES (4): Lys, Asn or Ser
  MOD_RES (5): Ile, Arg, Gln or Tyr
  MOD_RES (6): Pro, Arg, Asn or Leu
  MOD_RES (7): Ser, Arg, Asn or Pro
  MOD_RES (8): Ser, Lys, Pro or Tyr
XXXXXXXX

SEQ ID NO: 23 - protein, 8 aa, Artificial Sequence
  Description of Artificial Sequence Synthetic peptide
  MOD_RES (6): Pro, Arg or Lys
TSIKIXSS

SEQ ID NO: 24 - protein, 9 aa, Artificial Sequence
  Description of Artificial Sequence Synthetic peptide
  MOD_RES (1): Lys, Ala or Met
  MOD_RES (2): Asn, Arg, Cys, Ile or Tyr
  MOD_RES (4): Ser, Ala, Asn or Leu
  MOD_RES (5): Gly, Ala, Arg, Asn, Asp, Gln, Glu, Ile, Leu, Phe, Lys, Pro, Ser, Thr, Tyr or Val
  MOD_RES (6): Asn or Asp
  MOD_RES (7): Ser, Ala or Thr
  MOD_RES (8): Glu, Ser or Val
  MOD_RES (9): Phe or Tyr
XXWXXXXXX

SEQ ID NO: 25 - protein, 9 aa, Artificial Sequence
  Description of Artificial Sequence Synthetic peptide
  MOD_RES (2): Asn or Tyr
KXWSGNSEF

SEQ ID NO: 26 - protein, 11 aa, Artificial Sequence
  Description of Artificial Sequence Synthetic peptide
LTINAASIPSG

SEQ ID NO: 27 - protein, 16 aa, Artificial Sequence
  Description of Artificial Sequence Synthetic peptide
SHKVTLSSWYDHRGWG

SEQ ID NO: 28 - protein, 14 aa, Artificial Sequence
  Description of Artificial Sequence Synthetic peptide
SGSVPTDYLQLMVY

SEQ ID NO: 29 - protein, 12 aa, Artificial Sequence
  Description of Artificial Sequence Synthetic peptide
QVSNPSLLDPDQ

SEQ ID NO: 30 - protein, 12 aa, Artificial Sequence
  Description of Artificial Sequence Synthetic peptide
EVSNPSLLDPDQ

SEQ ID NO: 31 - protein, 14 aa, Artificial Sequence
  Description of Artificial Sequence Synthetic peptide
SGSLPTDYLQLMVY

SEQ ID NO: 32 - protein, 14 aa, Artificial Sequence
  Description of Artificial Sequence Synthetic peptide
SGDVPTEYLQLMVT

SEQ ID NO: 33 - protein, 14 aa, Artificial Sequence
  Description of Artificial Sequence Synthetic peptide
SGSVPAEYLQLMVY

SEQ ID NO: 34 - protein, 14 aa, Artificial Sequence
  Description of Artificial Sequence Synthetic peptide
SGSVATDYLQLMVY

SEQ ID NO: 35 - protein, 14 aa, Artificial Sequence
  Description of Artificial Sequence Synthetic peptide
SGDVATEYLQLMVT

SEQ ID NO: 36 - protein, 14 aa, Artificial Sequence
  Description of Artificial Sequence Synthetic peptide
SGSVAADYLQLMVY

SEQ ID NO: 37 - protein, 14 aa, Artificial Sequence
  Description of Artificial Sequence Synthetic peptide
SGSVAAEYLQLMVY

SEQ ID NO: 38 - protein, 14 aa, Artificial Sequence
  Description of Artificial Sequence Synthetic peptide
SGDLPTEYLQLMVT

SEQ ID NO: 39 - protein, 14 aa, Artificial Sequence
  Description of Artificial Sequence Synthetic peptide
SGSLPADYLQLMVY

SEQ ID NO: 40 - protein, 14 aa, Artificial Sequence
  Description of Artificial Sequence Synthetic peptide
SGSLPAEYLQLMVY

SEQ ID NO: 41 - protein, 14 aa, Artificial Sequence
  Description of Artificial Sequence Synthetic peptide
SGSLATDYLQLMVY

SEQ ID NO: 42 - protein, 14 aa, Artificial Sequence
  Description of Artificial Sequence Synthetic peptide
SGSLAADYLQLMVY

SEQ ID NO: 43 - protein, 14 aa, Artificial Sequence
  Description of Artificial Sequence Synthetic peptide
SGSLAAEYLQLMVY

SEQ ID NO: 44 - protein, 170 aa, Homo sapiens
MVDGSWLDLAKRSKLEAQPFAHLTINATDIPSGSHKVSLSSWYHDRGWGKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRD

SEQ ID NO: 45 - protein, 170 aa, Mus musculus
MMEGSWLDVAQRGKPEAQPFAHLTINAASIPSGSHKVTLSSWYHDRGWAKISNMTLSNGKLRVNQDGFYYLYANICFRHHETSGSVPTDYLQLMVYVVKTSIKIPSSHNLMKGGSTKNWSGNSEFHFYSINVGGFFKLRAGEEISIQVSNPSLLDPDQDATYFGAFKVQD

SEQ ID NO: 46 - protein, 170 aa, Rattus norvegicus
MMEGSWLDVARRGKPEAQPFAHLTINAADIPSGSHKVSLSSWYHDRGWAKISNMTLSNGKLRVNQDGFYYLYANICFRHHETSGSVPADYLQLMVYVVKTSIKIPSSHNLMKGGSTKNWSGNSEFHFYSINVGGFFKLRAGEEISVQVSNPSLLDPDQDATYFGAFKVQD

SEQ ID NO: 47 - protein, 170 aa, Pan troglodyte
MVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRD

SEQ ID NO: 48 - protein, 170 aa, Canis familiaris
MMEGSWLEMARRGKTHTQPFAHLTINATDIPSGSHKVSLSSWYHDRGWAKISNMTFSNGKLIVNQDGFYFLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVLD

SEQ ID NO: 49 - protein, 170 aa, Bos taurus
MVEGSWLDLARRNKPEIQPFAHLTINATDIPSGSHKVTLSCWYHDRGWAKISNMTFSNGKLIVNQDGFYYLYANICFRHHETSGNLSANYLQLMVYVTKTSIKIPSSHTLMKGGSTKYWLGNSEFHFYSINVGGFFKLRSGEKISIEVSNPSLLDPDQDATYFGAFKVRD

SEQ ID NO: 50 - protein, 169 aa, Gallus gallus
MEAIGMELYRRNKPEKQPFAHLIIDDKNIPTGTRKVNLTSWHHDKGQANLSNMTFSDGKLIVNQDGFYYLYANICFRHHETSGNLTKRGLQLMVYMTKTNLKIRRSDVLMKGGSTKYWSGNSEFHFYSVNIGGFLKLKTGDMISIQVSNPLLLDSSQEATYFGAFKVRD

SEQ ID NO: 51 - protein, 6 aa, Artificial Sequence
  Description of Artificial Sequence Synthetic peptide
QPFAHL

SEQ ID NO: 52 - protein, 11 aa, Mus musculus
LTINAASIPSG

SEQ ID NO: 53 - protein, 11 aa, Rattus norvegicus
LTINAADIPSG

SEQ ID NO: 54 - protein, 11 aa, Homo sapiens
LTINAADIPSG

SEQ ID NO: 55 - protein, 11 aa, Artificial Sequence
  Description of Artificial Sequence Synthetic peptide
LTINAADIPSG

SEQ ID NO: 56 - protein, 16 aa, Bos taurus
SHKVTLSSWYDHRGWG

SEQ ID NO: 57 - protein, 14 aa, Mus musculus
SGSVPTDYLQLMVY

SEQ ID NO: 58 - protein, 14 aa, Artificial Sequence
  Description of Artificial Sequence Synthetic peptide
SGSVPTDYLQLMVY

SEQ ID NO: 59 - protein, 14 aa, Rattus norvegicus
SGSVPTDYLQLMVY

SEQ ID NO: 60 - protein, 14 aa, Homo sapiens
SGSVPTDYLQLMVY

SEQ ID NO: 61 - protein, 12 aa, Mus musculus
QVSNPSLLDPDQ

SEQ ID NO: 62 - protein, 12 aa, Homo sapiens
EVSNPSLLDPDQ

SEQ ID NO: 63 - protein, 170 aa, Homo sapiens; identical to SEQ ID NO: 47

SEQ ID NO: 64 - protein, 172 aa, Mus musculus
MMEGSWLDVAQRGKPEAQPFAHLTINAASIPSGSHKVTLSSWYHDRGWAKISNMTLSNGKLRVNQDGFYYLYANICFRHHETSGSVPTDYLQLMVYVVKTSIKIPSSHNLMKGGSTKNWSGNSEFHFYSINVGGFFKLRAGEEISIQVSNPSLLDPDQDATYFGAFKVQDID

SEQ ID NO: 65 - protein, 172 aa, Mus musculus; identical to SEQ ID NO: 64

SEQ ID NO: 66 - protein, 172 aa, Mus musculus; identical to SEQ ID NO: 64

SEQ ID NO: 67 - protein, 172 aa, Rattus norvegicus
MMEGSWLDVARRGKPEAQPFAHLTINAADIPSGSHKVSLSSWYHDRGWAKISNMTLSNGKLRVNQDGFYYLYANICFRHHETSGSVPADYLQLMVYVVKTSIKIPSSHNLMKGGSTKNWSGNSEFHFYSINVGGFFKLRAGEEISVQVSNPSLLDPDQDATYFGAFKVQDID

SEQ ID NO: 68 - protein, 172 aa, Mus musculus; identical to SEQ ID NO: 64

SEQ ID NO: 69 - protein, 172 aa, Homo sapiens
MVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 70 - protein, 172 aa, Homo sapiens
MVDGSWLDLAKRSKLEAQPFAHLTINATDIPSGSHKVSLSSWYHDRGWGKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 71 - protein, 172 aa, Homo sapiens; identical to SEQ ID NO: 69

SEQ ID NO: 72 - protein, 172 aa, Canis familiaris
MMEGSWLEMARRGKTHTQPFAHLTINATDIPSGSHKVSLSSWYHDRGWAKISNMTFSNGKLIVNQDGFYFLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVLDID

SEQ ID NO: 73 - protein, 172 aa, Pan troglodytes; identical to SEQ ID NO: 69

SEQ ID NO: 74 - protein, 172 aa, Bos taurus
MVEGSWLDLARRNKPEIQPFAHLTINATDIPSGSHKVTLSCWYHDRGWAKISNMTFSNGKLIVNQDGFYYLYANICFRHHETSGNLSANYLQLMVYVTKTSIKIPSSHTLMKGGSTKYWLGNSEFHFYSINVGGFFKLRSGEKISIEVSNPSLLDPDQDATYFGAFKVRDID

SEQ ID NO: 75 - protein, 172 aa, Gallus gallus
AMEAIGMELYRRNKPEKQPFAHLIIDDKNIPTGTRKVNLTSWHHDKGQANLSNMTFSDGKLIVNQDGFYYLYANICFRHHETSGNLTKRGLQLMVYMTKTNLKIRRSDVLMKGGSTKYWSGNSEFHFYSVNIGGFLKLKTGDMISIQVSNPLLLDSSQEATYFGAFKVRDLD

SEQ ID NO: 76 - protein, 170 aa, Xenopus laevis
RTEARYGTRNNSWPVAHLTVGSITPSNSEQSKVNLTSWNFREGWANLRNIKYHNGKLEILQDGYYFVYANLCFRHHILKGKEYRDKALQLMIYICKATKNRRPYETLMKGGKTAIWSNNSVYHFYSVYQGGVFKLHAGDEIFIQASYLELLDPAQEATYFGAFKIHSLDV

SEQ ID NO: 77 - protein, 169 aa, Tetraodon nigroviridis
CKTLRESLPSAHLPIRAQQMYSKKGEPNAVTIIWDQDHGFCNRMNYQKGKLLVVESGFYYVYAKTCFRYYKYLPEDSVQAAVPTVDVSNTQLIQYILYESIKQNSKAVVLMKTGSTMRWNNTSYNMYCAQQGRGVRLETGDALFVNVSNAWMLDPEGEGTYFGAIKLGH

SEQ ID NO: 78 - protein, 185 aa, Danio rerio
RTHSYNTTGSKFMTTVMQRPSAHLTLSSASDNSRPQSDMHQPQFDLHQSCRHPVHTWANKSFGAHLYNMTLTNGRLRVPQDGRYYLYSQVYFRYPSPSDSDQSSVSHQLVQCIYKKTSYLNPIQLLKGVGTKCWAPDAEYALHSVYQGGLFELRAGDEVFVSVSSPTMVYGEDSSSYFGAFRLDL

(19) United States
(12) Patent Application Publication
(10) Pub. No.: US 2008/0107597 A1
(43) Pub. Date: May 8, 2008

(54) ISOLATION OF ANTIBODIES THAT CROSS-REACT AND NEUTRALIZE RANKL ORIGINATING FROM MULTIPLE SPECIES

(75) Inventor: William J. Boyle, Malibu, CA (US)

Correspondence Address: WILSON SONSINI GOODRICH & ROSATI, 650 PAGE MILL ROAD, PALO ALTO, CA, 94304-1050, US

(73) Assignee: ANAPTYS BIOSCIENCES, INC., La Jolla, CA (US)

(21) Appl. No.: 11/863,845

(22) Filed: Sep. 28, 2007

Related U.S. Application Data
(63) Continuation-in-part of application No. 11/622,928, filed on Jan. 12, 2007, now abandoned.
(60) Provisional application No. 60/758,319, filed on Jan. 12, 2006.

(51) Int. Cl. A61K 39/395; C07K 7/00; C07K 14/00; A61K 39/00; A61P 19/00; A61K 51/00; C07K 16/18

(52) U.S. Cl. 424/1.49; 530/325; 530/328; 530/326; 530/350; 530/387.7; 530/388.1; 530/389.1; 530/387.3; 424/138.1; 424/178.1; 424/183.1; 424/185.1

(57) ABSTRACT

The invention provides specific binding members (e.g., antibodies or antigen-binding fragments thereof) which bind to RANKL originating from multiple species. An epitope recognized by the specific binding members can be selected from surface exposed loop domains that bind to and activate its cognate receptor, RANK (Receptor Activator of NFkB), on the surface of osteoclast precursors and other cell types. The invention provides peptides for generating such anti-RANKL antibodies, including murine sequences, other non-human sequences and cross-reactive peptides. The specific binding members are useful in the diagnosis and treatment of lytic bone diseases, including osteoporosis, rheumatoid arthritis, bone metastasis and hypercalcemia of malignancy, glucocorticoid-induced bone loss, a periodontal disease or condition, a cancer and Juvenile Paget's Disease. The binding members can also be used in therapy in combination with chemotherapeutics or anti-cancer agents and/or with other antibodies or antigen-binding fragments thereof.

### RELATED APPLICATIONS

The present application is a continuation in part of U.S. patent application Ser. No. 11/622,928, entitled “ISOLATION OF ANTIBODIES THAT CROSS-REACT AND NEUTRALIZE RANKL ORIGINATING FROM MULTIPLE SPECIES,” filed on Jan. 12, 2007, which claims the benefit of U.S. Provisional application No. 60/758,319, entitled “ISOLATION OF ANTIBODIES THAT CROSS-REACT AND NEUTRALIZE RANKL ORIGINATING FROM MULTIPLE SPECIES,” filed on Jan. 12, 2006, each of which is specifically incorporated in its entirety by reference herein.

### FIELD OF THE INVENTION

The field of the present invention relates to molecular biology and immunology.

### BACKGROUND OF THE INVENTION

RANKL is a tumor necrosis factor (TNF)-related protein that binds to, and activates, the TNF receptor (TNFR)-related protein Receptor Activator of NF-kB (RANK). RANKL acts as the key cytokine that regulates osteoclast differentiation and activation during normal bone remodeling and during disease (Boyle et al, Nature. 15; 423(6937):337-42, 2003). RANKL inhibitors block osteoclast differentiation and activation in vitro and in vivo, and can block the pathological loss of bone in disease models that mimic osteoporosis, hypercalcemia of malignancy and bone metastasis, rheumatoid arthritis, steroid induced bone loss, and bone loss due to disuse and skeletal unloading. Recombinant RANKL inhibitors have been engineered from the ligand binding domains of Osteoprotegerin (OPG) and RANK, and from biologically active anti-RANKL neutralizing monoclonal antibodies.

Using the deduced crystal structure of mouse RANKL (Lam et al., United States Patent Application Publication Number 20030050223; Lam et al (2001) J Clin Invest 108(7):971-9) we have determined the amino acid sequences that are predicted to be crucial for RANKL binding and activation of RANK on the surface of osteoclast precursors and mature osteoclasts, and are required for inducing osteoclast differentiation and activation. The amino acid sequences correspond to ectodomains of the RANKL polypeptide that are formed by association of interdispersed β-pleated sheets, and extend from the core RANKL structure as peptide loops. The loop regions are located in precise regions of the primary amino acid sequence of RANKL, and can be directly mapped using the crystal structure coordinates. These sequences of the mouse polypeptide could serve as targets for antibodies and selective binding proteins that block or neutralize RANKL activity. A human monoclonal antibody to human RANKL has neutralizing activity in vivo, and can be administered to reduce bone resorption in post menopausal women (Bekker et al., J Bone Miner Res. July; 19(7):1059-66, 2004).

Thus, while the extant evidence of activity of anti-human RANKL antibodies is encouraging, the observed limitations on cross-reactivity to RANKL of other species and the absence of alternative efficacious, and potentially more effective or higher affinity, antibodies directed against distinct RANKL epitopes remain. Accordingly, it would be desirable to develop antibodies and like agents that demonstrate efficacy with RANKL of multiple species (e.g., that bind to both mouse and human RANKL polypeptides), and could be used in rodent disease models to predict efficacy in humans. It is toward the achievement of that objective that the present application is directed.

The citation of references herein shall not be construed as an admission that such is prior art to the present application.

### SUMMARY OF THE INVENTION

The present application provides an isolated specific binding member (e.g., an antibody or fragment thereof) which recognizes a mammalian RANKL epitope which is found in RANKL expressing cells such as osteoblasts, stromal cells, activated T cells, activated endothelium, and mammary epithelium. In a further aspect, the present application provides a specific binding member which recognizes a human RANKL epitope which is found in osteoblasts, stromal cells, activated T cells, activated endothelium, and mammary epithelium cells. In a still further aspect, the present application provides a specific binding member which recognizes a murine RANKL epitope which is found in osteoblasts, stromal cells, activated T cells, activated endothelium, and mammary epithelium cells. In one aspect, the murine and human epitopes are conserved, allowing these anti-RANKL antibodies to bind with the same affinity to mouse and human RANKL.

The specific binding member, which can be an antibody or a fragment thereof, such as an immunogenic fragment thereof, binds to both mouse and human RANKL and neutralizes the bioactivity of both mouse and human RANKL. In certain embodiments, the specific binding member has higher affinity for mouse RANKL over human RANKL. In alternate embodiments, the specific binding member has higher affinity for human RANKL over mouse RANKL.

In certain aspects, the specific binding member is an antibody, or antigen-binding fragment, that binds to a RANKL polypeptide consisting essentially of an amino acid sequence selected from the group consisting of SEQ ID NO: 18, SEQ ID NO: 19, SEQ ID NO: 20, SEQ ID NO: 22, SEQ ID NO: 21, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 and SEQ ID NO: 43, and wherein the antibody, or antigen-binding fragment, neutralizes the activity of RANKL. In certain other aspects, the specific binding member is an antibody that binds to a RANKL polypeptide consisting essentially of an amino acid sequence selected from the group consisting of SEQ ID NO: 18, SEQ ID NO: 19, SEQ ID NO: 20, SEQ ID NO: 22, SEQ ID NO: 21, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 and SEQ ID NO: 43, wherein the antibody is selected from among a polyclonal antibody, a monoclonal antibody, a chimeric antibody, and a humanized antibody. In one embodiment, the antibody is a monoclonal antibody. In another embodiment, the antibody is a chimeric antibody. In still another embodiment, the antibody is a humanized antibody.

In still other aspects, the specific binding member is an antigen binding fragment that binds to a RANKL polypeptide consisting essentially of an amino acid sequence selected from the group consisting of SEQ ID NO: 18, SEQ ID NO: 19, SEQ ID NO: 20, SEQ ID NO: 22, SEQ ID NO: 21, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 and SEQ ID NO: 43, wherein the antigen binding fragment is selected from among Fab, Fd, scFv, dAb, a F(ab′)2, a bi-specific Fab2, a multivalent antibody fragment, and a bi-specific scFv. In one embodiment, the antigen binding fragment is a Fab fragment. In another embodiment, the antigen binding fragment is an Fd fragment. In yet another embodiment, the antigen binding fragment is a scFv. In still another embodiment, the antigen binding fragment is a dAb. In yet still another embodiment, the antigen binding fragment is an F(ab′)₂. In another embodiment, the antigen binding fragment is a bi-specific F(ab′)₂. In still another embodiment, the antigen binding fragment is a multivalent antibody fragment. In still another embodiment, the antigen binding fragment is a bi-specific scFv.

In certain other embodiments, the specific binding members described herein which are based upon the CDR3 regions of the heavy or light chain, or both, of an antibody that can cross-react and neutralize RANKL from multiple species, can be useful specific binding members for in vivo therapy.

The present inventors have further discovered an approach to produce novel antibodies, exemplified herein by an antibody that can cross-react and neutralize RANKL from multiple species, which specifically recognize a conserved RANKL epitope or a cross-reactive RANKL epitope. For example, the antibodies of the present application recognize a conserved epitope which is found in osteoblasts, stromal cells, activated T cells, activated endothelium, and mammary epithelium cells, wherein the epitope is enhanced or evident upon induction of bone resorption. In certain embodiments, the antibodies of the present application are monoclonal antibodies. In other embodiments, the antibodies of the present application are monoclonal antibodies that specifically recognize RANKL originating from multiple species, including mouse and human.

In certain aspects, described herein is a method of preparing an antibody, which specifically binds a RANKL polypeptide consisting essentially of an amino acid sequence selected from the group consisting of SEQ ID NO: 18, SEQ ID NO: 19, SEQ ID NO: 20, SEQ ID NO: 22, SEQ ID NO: 21, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 and SEQ ID NO: 43, the method comprising the steps of: a) preparing a RANKL polypeptide consisting essentially of an amino acid sequence selected from the group consisting of SEQ ID NO: 18, SEQ ID NO: 19, SEQ ID NO: 20, SEQ ID NO: 22, SEQ ID NO: 21, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 and SEQ ID NO: 43; b) immunizing a rodent (e.g., a mouse) with a solution comprising the RANKL polypeptide of a) to generate an immune response in the mouse to the RANKL polypeptide; c) isolating one or more spleen cells from the rodent of step b) after a sufficient amount of time for the mouse to generate an immune response; d) fusing the isolated one or more spleen cells of step c) with a myeloma cell line to form a population of hybridoma cells; e) culturing the hybridoma cell in a cell culture media comprising HAT; f) selecting one or more cells within the population of hybridoma cells of e) which express a antibody that binds to the RANKL polypeptide of a); and g) clonally expanding the one or more hybridoma cells of f). In certain other embodiments, the methods can further comprise the step of: h) identifying the antibody sequence of the antibody expressed in the one or clonal populations of cells of g). In certain embodiments, the antibody produced by the methods described herein are monoclonal antibodies which specifically binds a RANKL polypeptide consisting essentially of an amino acid sequence selected from the group consisting of SEQ ID NO: 18, SEQ ID NO: 19, SEQ ID NO: 20, SEQ ID NO: 22, SEQ ID NO: 21, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 and SEQ ID NO: 43.

In certain other aspects, described herein is a method of generating a neutralizing antibody which specifically binds a RANKL polypeptide consisting essentially of an amino acid sequence selected from the group consisting of SEQ ID NO: 18, SEQ ID NO: 19, SEQ ID NO: 20, SEQ ID NO: 22, SEQ ID NO: 21, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 and SEQ ID NO: 43, the method comprising the steps of: a) preparing a RANKL polypeptide consisting essentially of an amino acid sequence selected from the group consisting of SEQ ID NO: 18, SEQ ID NO: 19, SEQ ID NO: 20, SEQ ID NO: 22, SEQ ID NO: 21, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 and SEQ ID NO: 43; b) immunizing a rodent (e.g., a mouse) with a solution comprising the RANKL polypeptide of a) to generate an immune response in the mouse to the RANKL polypeptide; c) isolating one or more spleen cells from the rodent of step b) after a sufficient amount of time for the mouse to generate an immune response; d) fusing the isolated one or more spleen cells of step c) with a myeloma cell line to form a population of hybridoma cells; e) culturing the population hybridoma cells in a cell culture media comprising HAT; f) selecting one or more cells within the population of hybridoma cells of e) which express a antibody that binds to the RANKL polypeptide of a); and g) clonally expanding the one or more hybridoma cells of f). In certain other aspects, the methods can further comprise the step of: h) identifying the antibody sequence of the antibody expressed in the one or clonal populations of cells of g). In certain embodiments, the neutralizing antibodies produced by the methods described herein are monoclonal antibodies which specifically binds a RANKL polypeptide consisting essentially of an amino acid sequence selected from the group consisting of SEQ ID NO: 18, SEQ ID NO: 19, SEQ ID NO: 20, SEQ ID NO: 22, SEQ ID NO: 21, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 and SEQ ID NO: 43.

In further aspects, the application provides an isolated nucleic acid which comprises a sequence encoding a specific binding member as defined above, and methods of preparing specific binding members of the application which comprise expressing said nucleic acids under conditions to bring about expression of said binding member, and recovering the binding member.

The antibodies of the present application can specifically categorize the nature of cells, by staining or otherwise recognizing those cells wherein RANKL is present. Further, the antibodies of the present application, as exemplified by antibodies that can cross-react and neutralize RANKL from multiple species, can demonstrate significant in vivo anti-RANKL activity, and can neutralize RANKL-induced osteoclastogenesis and osteoclast activation, thereby modulating bone resorption.

The unique specificity of antibodies that can cross-react and neutralize RANKL from multiple species, provides diagnostic and therapeutic uses to determine the endogenous levels of RANKL in various disease states associated with increased bone resorption rates, including osteoporosis, lytic bone metastasis, Rheumatoid Arthritis, and periodontal disease.

The antibody can be one which has the characteristics of the antibody which the inventors have described, recognizing RANKL originating from multiple species, including mouse and human, and neutralizing the bioactivity of both human and mouse protein. In one non-limiting example, the antibody is a monoclonal antibody that can cross-react and neutralize RANKL from multiple species, or active fragments thereof. In a further aspect, the antibody contains amino acid sequences encoding the V_{H} and V_{L} regions, respectively. In certain embodiments, the antibody binds with higher affinity to mouse RANKL over human RANKL. In other embodiments, the antibody binds with higher affinity to human RANKL over mouse RANKL.

In other embodiments, the present application provides an isolated RANKL polypeptide consisting essentially of an amino acid sequence such as, for example, SEQ ID NO: 18, SEQ ID NO: 19, SEQ ID NO: 20, SEQ ID NO: 22, SEQ ID NO: 21, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43. Polypeptides of the application have use in diagnostic assays or kits.

In certain other embodiments, the present application provides compositions comprising a RANKL polypeptide and an acceptable excipient, wherein said RANKL epitope polypeptide consists essentially of an amino acid sequence such as, for example, SEQ ID NO: 18, SEQ ID NO: 19, SEQ ID NO: 20, SEQ ID NO: 22, SEQ ID NO: 21, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43. In certain aspects, the acceptable excipient is, for example, a carrier, a buffer, a stabilizer, or any combination thereof, which can be formulated for oral or injection (e.g., intravenous) administration.

Yet a further aspect of the application are compositions of such binding proteins with additional binding proteins, such as binding proteins which bind to both human and mouse RANKL with equal affinity, which inhibit ligand binding thereto. Such compositions can be “one pot” cocktails, kits, and so forth, formulated for ease of administration.

Specific binding members can be used in a method of treatment or diagnosis of a patient (e.g., a human or animal body), such as a method of treatment of bone loss in a patient which comprises administering to said patient an effective amount of a specific binding member as described herein. Such bone loss conditions include, for example, osteoporosis, rheumatoid arthritis, bone metastasis and hypercalcemia of malignancy, glucocorticoid-induced bone loss, a periodontal disease or condition, a cancer and Juvenile Paget's Disease.

The present application also relates to a recombinant DNA molecule or cloned gene, or a degenerate variant thereof, which encodes an antibody as described herein. The full DNA sequence of the recombinant DNA molecule or cloned gene of the antibody that can cross-react and neutralize RANKL from multiple species can be operatively linked to an expression control sequence which can be introduced into an appropriate host for expression. The application accordingly extends to unicellular hosts transformed with the cloned gene or recombinant DNA molecule comprising a DNA sequence encoding the V_{H} and/or V_{L}, or portions thereof, of the antibody.

The present application contemplates several means for preparation of the antibodies and active fragments thereof including, as illustrated herein, known recombinant techniques, and the application is accordingly intended to cover such synthetic or chimeric antibody preparations within its scope. The isolation of the cDNA and amino acid sequences of the antibodies that can cross-react and neutralize RANKL from multiple species provided herein facilitates the reproduction of the antibodies by such recombinant techniques and, accordingly, the application extends to expression vectors prepared from the disclosed DNA sequences for expression in host systems by recombinant DNA techniques, and to the resulting transformed hosts.

The diagnostic utility of the present application extends to the use of the antibodies in assays to measure RANKL levels during lytic bone diseases, including in vitro and in vivo diagnostic assays. In an immunoassay, a control quantity of the antibodies, or the like, can be prepared and labeled with an enzyme, a specific binding partner and/or a radioactive element, and can then be introduced into a cellular sample. After the labeled material or its binding partner(s) has had an opportunity to react with sites within the sample, the resulting mass can be examined by known techniques, which can vary with the nature of the label attached.

Specific binding members can carry a detectable or functional label. The specific binding members can carry a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ²¹¹At, ¹⁹⁸Au, ⁶⁷Cu, ²²⁵Ac, ²¹³Bi, ⁹⁹Tc and ⁸⁶Re, or any others described herein. When radioactive labels are used, known currently available counting procedures can be utilized to identify and quantitate the specific binding members. In the instance where the label is an enzyme, detection can be accomplished by any of the presently utilized calorimetric, spectrophotometric, fluorospectrophotometric, amperometric or gasometric techniques known in the art. Other conventional means known for detection of labels are included herein.

The radiolabelled specific binding members (e.g., antibodies and fragments thereof) described herein are useful in in vitro diagnostics techniques and in in vivo radioimaging techniques. In a further aspect, immunoconjugates of radiolabelled specific binding members (i.e., radioimmunoconjugates), are useful in radioimmunotherapy, such as radiolabelled antibodies for cancer therapy. In a still further aspect, the radiolabelled specific binding members are useful in radioimmuno-guided surgery techniques, wherein they can identify and indicate the presence and/or location of RANKL expressing cells in a patient exhibiting symptoms or diagnoses as having a disease state characterized by excessive bone loss.

Immunoconjugates or antibody fusion proteins of the present application, wherein the specific binding members are conjugated or attached to other molecules or agents further include, but are not limited to binding members conjugated to a chemical ablation agent, a toxin, an immunomodulator, a cytokine, a cytotoxic agent, a chemotherapeutic agent or a drug.

The present application includes an assay system which can be prepared in the form of a test kit for the quantitative analysis of the extent of the presence of, for instance, RANKL. The system or test kit can comprise a labeled component prepared by one of the radioactive and/or enzymatic techniques discussed herein, coupling a label to the specific binding member, and one or more additional immunochemical reagents, at least one of which is a free or immobilized component to be determined or their binding partner(s).

In a further embodiment, the present application relates to certain therapeutic methods which would be based upon the activity of the binding member, antibody, or active fragments thereof, or upon agents or other drugs determined to possess the same activity. A therapeutic method can be associated with the prevention or treatment of bone loss associated with increased osteoclast activity, such as primary osteoporosis.

The binding members described herein, that can cross-react and neutralize RANKL from multiple species (e.g., antibodies or active fragments thereof, and chimeric (bispecific) or synthetic antibodies derived therefrom) can be prepared in compositions (e.g., pharmaceutical compositions), including a suitable vehicle, carrier or diluent, for administration in instances wherein therapy is appropriate, such as to treat osteoporosis. Such compositions can also include methods of modulating the half-life of the binding members, antibodies or fragments by methods known in the art such as pegylation. Such pharmaceutical compositions can further comprise additional antibodies or therapeutic agents.

In certain embodiments, the compositions comprise an antibody, or antigen-binding fragment, that binds to a RANKL polypeptide consisting essentially of an amino acid sequence such as, for example SEQ ID NO: 18, SEQ ID NO: 19, SEQ ID NO: 20, SEQ ID NO: 22, SEQ ID NO: 21, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43, and a pharmaceutically acceptable excipient. In one embodiment, the pharmaceutically acceptable excipient can be, for example, a carrier, a buffer, a stabilizer, or any combination thereof, which can be formulated for oral or injectable (e.g., intravenous) administration. In another embodiment, the composition can further comprise a detectable or functional label. In such one embodiment, the detectable label is a radioisotope. In another such embodiment, the functional label is a cytotoxic drug.

In still other embodiments, the compositions comprising a specific binding member are conjugated to a second pharmaceutically active agent. In one such embodiment, the pharmaceutically active agent includes, but is not limited to, chemical ablation agents, toxins, immunomodulators, cytokines, and chemotherapeutic agents.

In other aspects, the present application encompasses isolated polypeptides comprising an amino acid sequence of a RANKL peptide, including a murine or other non-human RANKL polypeptide, consisting essentially of an amino acid sequence of any of the RANKL polypeptide sequences set forth in SEQ ID NO: 18, SEQ ID NO: 19, SEQ ID NO: 20, SEQ ID NO: 21, SEQ ID NO: 22, SEQ ID NO: 23, SEQ ID NO: 24, and SEQ ID NO: 25. The isolated peptides, including combinations of one or more thereof, are suitable for use in generating antibodies which recognize RANKL and have anti-RANKL/RANK activity and are useful in modulating bone remodeling, osteoclast differentiation and bone disease. In certain other aspects, the present application is directed to an isolated RANKL polypeptide which consists essentially of the amino acid sequence set forth in any of SEQ ID NO: 18, SEQ ID NO: 19, SEQ ID NO: 20, SEQ ID NO: 22, SEQ ID NO: 21, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 and SEQ ID NO: 43.

In certain embodiments, the application provides isolated RANKL loop peptides, including novel loop peptides and peptides having amino acid sequences taken from one or more mammalian species. These RANKL loop peptides are capable of generating and raising anti-RANKL antibodies which recognize, bind to and neutralize RANKL from more than one mammalian species such as, for example, antibodies that bind mouse and human RANKL and neutralize the activity of the mouse and human RANKL polypeptides.

Exemplary A-A′ loop based peptides are provided herein. The application provides an isolated peptide having an amino acid sequence X1 X2 X3 X4 X5 X6 X7 X8 X9 X10 X11 X12 X13 X14 X15 X16 X17 X18 X19 X20 X21 X22 X23, wherein each residue can be independently selected as follows (SEQ ID NO: 18):
X1 is N, R, D, G or S;
X2 is A, D, or S;
X3 is A, Q, I, K or T;
X4 is S, N, D, Q or T;
X5 is I, A, R, N, D, Q, E, G, L, K, M, F, P, S, T or V;
X6 is P, N or Y;
X7 is S or T;
X8 is G, N or Q;
X9 is S, E or T;
X10 is H, R, C, E or P;
X11 is K, R, or N;
X12 is V, A or H;
X13 is T, N, P, S or V;
X14 is L, T or V;
X15 is S, H, I or T;
X16 is S, C, I or T;
X17 is Y, A, N, D or H;
X18 is H, N, Q or F;
X19 is D, R, or K;
X20 is R, E, H, K or S;
X21 is G or F;
X22 is W, Q, G or F;
X23 is A or C.

The application provides an isolated peptide having an amino acid sequence:

N A X1 X2 I P S G S H K V X3 L S S W Y H D R G W A, wherein each Xn residue is selected as follows (SEQ ID NO:19):
X1 is A or T;
X2 is S or D; and
X3 is T or S.

Exemplary C-D loop based peptides are provided herein. The application provides an isolated peptide having an amino acid sequence X1 X2 X3 X4 X5 X6 X7 X8 X9 X10 X11 X12, wherein each residue can be independently selected as follows (SEQ ID NO:20):
X1 is H, P or W;
X2 is E, I, L or S;
X3 is T, L, P or V;
X4 is S, Q or K;
X5 is G, A or N;
X6 is S, A, N, D or K;
X7 is V, D, E or L;
X8 is P, A, Q, S, T or Y;
X9 is T, A, R, K or S;
X10 is D, R, N, E or S;
X11 is Y, A, N, G or V; and
X12 is L, T or H.

The application provides an isolated peptide having an amino acid sequence H E T S G X1 X2 X3 X4 X5 Y L, wherein each Xn residue is selected as follows (SEQ ID NO:21):
X1 is S or D;
X2 is V or L;
X3 is P or A;
X4 is T or A; and
X5 is D or E.

Exemplary D-E loop based peptides are provided herein. The application provides an isolated peptide having an amino acid sequence X1 X2 X3 X4 X5 X6 X7 X8, wherein each residue can be independently selected as follows (SEQ ID NO:22):
X1 is T, A, E or K;
X2 is S, N, K or T;
X3 is I, L, K or T;
X4 is K, N or S;
X5 is I, R, Q or Y;
X6 is P, R, N or L;
X7 is S, R, N or P; and
X8 is S, K, P or Y.

The application provides an isolated peptide having an amino acid sequence T S I K I X1 S S, wherein X1 is selected from P, R or K (SEQ ID NO:23).

Exemplary E-F loop based peptides are provided herein. The application provides an isolated peptide having an amino acid sequence X1 X2 W X3 X4 X5 X6 X7 X8, wherein each residue can be independently selected as follows (SEQ ID NO:24):
X1 is K, A or M;
X2 is N, R, C, I or Y;
X3 is S, A, N or L;
X4 is G, A R, N, D, Q, E, I, L, F, K, P, S, T, Y or V;
X5 is N or D;
X6 is S, A or T;
X7 is E, S or V; and
X8 is F or Y.

The application provides an isolated peptide having an amino acid sequence K X1 W S G N S E F, wherein X1 is selected from N or Y (SEQ ID NO:25).

The application also provides isolated peptides having the amino acid sequences set forth in Table 1:

TABLE 1

| | |
|---|---|
| Amino Acid Sequence | SEQ ID NO: |

| | |
|---|---|
| LTINAASIPSG | SEQ ID NO:26 |
| SHKVTLSSWYDHRGWG | SEQ ID NO:27 |
| SGSVPTDYLQLMVY | SEQ ID NO:28 |
| QVSNPSLLDPDQ | SEQ ID NO:29 |
| EVSNPSLLDPDQ | SEQ ID NO:30 |
| SGSLPTDYLQLMVY | SEQ ID NO:31 |
| SGDVPTEYLQLMVT | SEQ ID NO:32 |
| SGSVPAEYLQLMVY | SEQ ID NO:33 |
| SGSVATDYLQLMVY | SEQ ID NO:34 |
| SGDVATEYLQLMVT | SEQ ID NO:35 |
| SGSVAADYLQLMVY | SEQ ID NO:36 |
| SGSVAAEYLQLMVY | SEQ ID NO:37 |
| SGDLPTEYLQLMVT | SEQ ID NO:38 |
| SGSLPADYLQLMVY | SEQ ID NO:39 |
| SGSLPAEYLQLMVY | SEQ ID NO:40 |
| SGSLATDYLQLMVY | SEQ ID NO:41 |
| SGSLAADYLQLMVY | SEQ ID NO:42 |
| SGSLAAEYLQLMVY | SEQ ID NO:43 |

The present application further provides an isolated nucleic acid encoding a RANKL polypeptide consisting essentially of an amino acid sequence such as, for example, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26 (LTINAASIPSG), SEQ ID NO: 27 (SHKVTLSSWYDHRGWG), SEQ ID NO: 28 (SGSVPTDYLQLMVY), SEQ ID NO: 29 (QVSNPSLLDPDQ), SEQ ID NO: 30 (EVSNPSLLDPDQ), SEQ ID NO: 31 (SGSLPTDYLQLMVY), SEQ ID NO: 32 (SGDVPTEYLQLMVT), SEQ ID NO: 33 (SGSVPAEYLQLMVY), SEQ ID NO: 34 (SGSVATDYLQLMVY), SEQ ID NO: 35 (SGDVATEYLQLMVT), SEQ ID NO: 36 (SGSVAADYLQLMVY), SEQ ID NO: 37 (SGSVAAEYLQLMVY), SEQ ID NO: 38 (SGDLPTEYLQLMVT), SEQ ID NO: 39 (SGSLPADYLQLMVY), SEQ ID NO: 40 (SGSLPAEYLQLMVY), SEQ ID NO: 41 (SGSLATDYLQLMVY), SEQ ID NO: 42 (SGSLAADYLQLMVY) or SEQ ID NO: 43 (SGSLAAEYLQLMVY).

The present application extends to an immunogenic RANKL peptide including, but not limited to, a peptide sequence consisting essentially of any of SEQ ID NOS: 18-43. The present application also extends to immunogenic RANKL peptides wherein such polypeptides comprise a combination of at least one immunogenic RANKL peptide, selected from a peptide sequence consisting essentially of any of SEQ ID NOS: 18-43.

The application provides a method for immunizing a mammal comprising administering a RANKL epitope peptide, whereby antibodies which are immunoreactive with RANKL, and which can neutralize RANKL and modulate RANKL/RANK signals are produced. The application further provides a method for immunizing a mammal comprising administering a RANKL polypeptide consisting essentially of an amino acid sequence of any of SEQ ID NOS: 18-43, whereby antibodies which are immunoreactive with RANKL from multiple species (e.g., human and mouse RANKL) are produced.

The application provides a method for immunizing a mammal comprising administering a RANKL receptor peptide which does not fully correspond to murine or human RANKL peptide sequence selected from a RANKL polypeptide consisting essentially of an amino acid sequence of any of SEQ ID NOS: 18-43, whereby antibodies which are immunoreactive with RANKL from multiple species (e.g., human and mouse RANKL) are produced.

In a further aspect, the present application extends to immunogenic compositions based on the RANKL peptides described herein. The present application provides an immunogenic composition consisting essentially of one or more RANKL peptide described herein and provided in the Figures, including those selected from a RANKL peptide of any of SEQ ID NOS: 18-43, and a pharmaceutically acceptable adjuvant. In one embodiment, the present application provides an immunogenic composition consisting essentially of one or more peptides of any of SEQ ID NOS: 18-43, and a pharmaceutically acceptable adjuvant.

In addition to cross-reactive binding sequences, provided herein are methods for the development of RANKL antibodies, and antigen-binding fragments thereof, using a cross-reactive binding sequence. Also included are uses of the antibodies, and antigen-binding fragments thereof, for in vitro and in vivo diagnostic, detection, monitoring, prophylaxis and treatment.

Other objects and advantages will become apparent to those skilled in the art from a review of the ensuing detailed description, which proceeds with reference to the following illustrative drawings, and the attendant claims.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

FIG. 1: Crystal structure of the mouse RANKL polypeptide as deduced by Lam et al. (2003). Left is the homotrimeric soluble extracellular domain. Right is the structure of an individual monomer, showing the position of the surface loops that make contact with its receptor, the TNF-related protein, RANK.

FIG. 2: Primary structure of the mouse RANKL polypeptide indicating the position of the surface loops (A-A′ loop, C-D loop, D-E loop, and E-F loop) (SEQ ID NO:17).

FIG. 3: Alignment of the mouse, rat, dog, and human RANKL peptide sequences corresponding to the surface loops (A-A′ loop, C-D loop, D-E loop, and E-F loop) (SEQ ID NOS: 1-16).

FIG. 4: A; Multiple sequence alignment of 15 independent RANKL polypeptide sequences detected using a profile obtained from the mouse, rat, dog, and human RANKL surface loops. B; Position specific matrix (PSSM) for the surface loop sequences generated from the multiple sequence alignment in FIG. 4A. This matrix indicates the amino acid variables for each position in surface loops A-A′ loop, C-D loop, D-E loop, and E-F loop, respectively.

FIG. 5: A; Example of the predicted peptide sequences for the surface loop A=A′ loop using the position specific matrix (PSSM) generated from the RANKL multiple sequence alignment, the mouse sequence is shown in bold at top. B; Similar example of the predicted peptide sequences for the surface loop=C-D using the position specific matrix (PSSM) generated from the RANKL multiple sequence alignment, the mouse sequence is shown in bold at top. C; Similar example of the predicted peptide sequences for the surface loop=D-E using the position specific matrix (PSSM) generated from the RANKL multiple sequence alignment, the mouse sequence is shown in bold at top. D; Similar example of the predicted peptide sequences for the surface loop=E-F using the position specific matrix (PSSM) generated from the RANKL multiple sequence alignment, the mouse sequence is shown in bold at top. Each matrix includes substitutions based on the position specific matrix shown in FIG. 4B. Substitutions at each position were given equal weighting independent of the relative calculated frequency shown in FIG. 4B.

FIG. 6. Predicted peptide sequences based on the mouse, rat, canine, and human RANKL loop sequences shown in FIG. 3. A; The A-A′ loop, mouse sequence is shown at top. B; The C-D loop, mouse sequence is shown at top. C; The E-F loop, mouse sequence is shown at top.

FIG. 7. Blast 2.0 using the substitution matrix generated in FIG. 4.

FIG. 8. Flow chart for the isolation of anti-mouse antibodies with neutralizing activity that also bind and neutralize human RANKL.

FIG. 9. Provides aligned RANKL sequences from the following species: Homo sapiens (SEQ ID NO: 44), Mus musculus (SEQ ID NO: 45), Rattus norvegicus (SEQ ID NO: 46), Pan troglodyte (SEQ ID NO: 47), Canis familiaris (SEQ ID NO: 48), Bos taurus (SEQ ID NO: 49) and G. gallus (SEQ ID NO: 50). The figure distills the available sequence and structure information, diagramming a multiple sequence alignment of terrestrial RANKL polypeptides with structurally homologous positions known to participate in receptor or antibody binding highlighted in various colors. The C-terminal 170 residues of RANKL form the extracellular domain that binds RANK (displayed on the surface of neighboring cells), and begin with the residues QPFAHL that form the first β-sheet. Residues colored in aqua and orange, mapped by structural homology, form the binding interface in the CD40 neutralizing antibody (aqua) or TRAIL and TNFβ receptor (orange) structures. Residues highlighted in yellow are positions that differ between mouse and human sequences. Likewise, positions highlighted in light blue, green or magenta detail that sequence differences of dog, cow and chicken with the human sequence. Beneath the multiple sequence alignment, red highlights positions of 100% sequence conservation, and dark blue that positions participating in the hydrophobic trimeric interface that is largely unavailable for ligand binding.

FIG. 10. FIGS. 10 A-D deconstruct RANKL binding surfaces into four continuous peptides, which are hereafter referred to as peptides A, B, C and D. FIGS. 10 E and 10 F illustrate a single RANKL domain, with FIG. 10 E displaying a ribbon diagram with the individual strands labeled for reference, and with the solvent-accessible surface positioned towards the viewer. FIG. 10 F shows an identical view with structurally equivalent positions, (i.e., those that are bound by receptors or antibodies in the CD40, TRAIL, and TNFβ complex structures), shown in blue, space filling formats. This contiguous binding surface is composed of three pairs of beta-strand pairs; a-a′, c-d, and g-h. FIG. 10 G illustrates the molecule of FIG. 10 F rotated 180 degrees around the Z-axis.

FIG. 11. FIGS. 11 A-C illustrate the electrostatic RANKL binding surfaces of three species: Rattus norvegicus, Mus musculus, and Homo sapiens, respectively. The Rattus norvegicus and Homo sapiens protein structures were constructed using the x-ray crystal structure of the mouse protein, with side chain substitutions and conformer side chain energy minimization performed using the program SwissPDBviewer. The Rattus norvegicus sequence, whose RANKL binding surface is highlighted by the dotted oval, represents a charge and binding surface intermediate to the mouse and human binding surfaces.

### DETAILED DESCRIPTION OF THE INVENTION

It is to be understood that this application is not limited to particular formulations or process parameters, as these may, of course, vary. It is also to be understood that the terminology used herein is for the purpose of describing particular embodiments only, and is not intended to be limiting. Further, it is understood that a number of methods and materials similar or equivalent to those described herein can be used in the practice of the present invention(s).

In accordance with the present application, there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984), each of which is specifically incorporated herein by reference in its entirety.

I. Overview

An isolated specific binding member, such as an antibody or fragment thereof, which recognizes a mammalian RANKL epitope found in RANKL expressing cells such as osteoblasts, stromal cells, activated T cells, activated endothelium, and mammary epithelium can be generated using the methods described herein and further used in the methods described herein. In one aspect, the specific binding member recognizes a human RANKL epitope which is found in osteoblasts, stromal cells, activated T cells, activated endothelium, and mammary epithelium cells. In another aspect, the specific binding member recognizes a murine RANKL epitope which is found in osteoblasts, stromal cells, activated T cells, activated endothelium, and mammary epithelium cells. In yet another aspect, the murine and human epitopes are conserved, allowing these anti-RANKL specific binding members to bind to mouse and human RANKL and neutralize the bioactivity of both mouse and human RANKL.

II. Terminology

As used herein and in the appended claims, the terms “a,” “an” and “the” can mean, for example, one or more, or at least one, of a unit unless the context clearly dictates otherwise. Thus, for example, reference to “an antibody” includes a plurality of such antibodies and reference to “a variable domain” includes reference to one or more variable domains and equivalents thereof known to those skilled in the art, and so forth. In the event that there is a plurality of definitions for a term herein, those in this section prevail unless stated otherwise.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges can independently be included or excluded in the range, and each range where either, neither or both limits are included in the smaller ranges is also encompassed, subject to any specifically excluded limit in the stated range. Where the stated range includes one or both of the limits, ranges excluding either or both of those included limits are also included.

The term “consisting essentially of” refers to a product such as a peptide sequence, of a defined number of residues which is not covalently attached to a larger product. In the case of the peptide referred to above, those of skill in the art can appreciate that minor modifications to the N- or C-terminal of the peptide can however be contemplated, such as the chemical modification of the terminal to add a protecting group or the like, e.g. the amidation of the C-terminus. The term “consisting essentially of” as used in reference to a peptide including one or more designated amino acid sequences also indicates that no more than 20 to 30 amino acids are added to the designated amino acid sequence(s) and, furthermore, that these additional amino acids do not substantially alter the function of the designated amino acid sequence(s). Consisting essentially of, as used in reference to a polynucleotide including one or more designated polynucleotides also indicates that no more than 20 to 30 nucleotides are added to the designated polynucleotides and, furthermore, that these additional nucleotides do not substantially alter the function of the designated polynucleotides.

The term “isolated” refers to the state in which polypeptides or polynucleotides will be in accord with the present application. Polypeptides or polynucleotides will be free or substantially free of material with which they are naturally associated such as other polypeptides or polynucleotides with which they are found in their natural environment, or the environment in which they are prepared (e.g. cell culture) when such preparation is by recombinant DNA technology practiced in vitro or in vivo. Polypeptides or polynucleotides can be formulated with diluents or adjuvants and still for practical purposes be isolated—for example, the polypeptides or polynucleotides can be mixed with gelatin or other carriers if used to coat microtitre plates for use in immunoassays, or will be mixed with pharmaceutically acceptable carriers or diluents when used in diagnosis or therapy. Polypeptides or polynucleotides can be glycosylated, either naturally or by systems of heterologous eukaryotic cells, or they can be (for example, if produced by expression in a prokaryotic cell) unglycosylated.

The term “selection” refers to the separation of one or more members, such as polynucleotides, proteins or cells from a library of such members. Selection can involve both detection and selection, for example where cells are selected by use of a fluorescence activated cell sorter (FACS) that detects a reporter gene and then sorts the cells accordingly.

As used herein, “pg” means picogram, “ng” means nanogram, “ug” or “μg” mean microgram, “mg” means milligram, “ul” or “μl” mean microliter, “ml” means milliliter, “l” means liter, “kb” means kilobases, “uM” or “μM” means micromolar, “nM” means nanomolar, “pM” means picomolar, “fM” means femtomolar and “M” means molar.

The phrase “pharmaceutically acceptable” refers to molecular entities and compositions that are physiologically tolerable and do not typically produce an allergic or similar untoward reaction, such as gastric upset, dizziness and the like, when administered to a human.

A. Antibody Terminology

The term “antibody” describes an immunoglobulin whether natural or partly or wholly synthetically produced. The term also covers any polypeptide or protein having a binding domain which is, or is homologous to, an antigen-binding domain. As used herein, CDR grafted antibodies are also contemplated by this term.

As used herein, “natural” or “naturally occurring” antibodies or antibody variable domains, refers to antibodies or antibody variable domains having a sequence of an antibody or antibody variable domain identified from a non-synthetic source, for example, from a germline sequence, or differentiated antigen-specific B cell obtained ex vivo, or its corresponding hybridoma cell line, or from the serum of an animal. These antibodies can include antibodies generated in any type of immune response, either natural or otherwise induced. Natural antibodies include the amino acid sequences, and the nucleotide sequences that constitute or encode these antibodies, for example, as identified in the Kabat database.

The term, “synthetic antibody” or “synthetic gene” means that the corresponding polynucleotide sequence, or amino acid sequence, is derived, in whole, or part, from a sequence that has been designed, or synthesized de novo, or modified, compared to an equivalent unmodified sequence. Synthetic genes can be prepared in whole or part, via chemical synthesis, or amplified via PCR, or similar enzymatic amplification systems. Synthetic genes are, in some embodiments, different from unmodified genes, either at the amino acid, or polynucleotide level, (or both) and are, for example, located within the context of synthetic expression control sequences. Synthetic gene sequences can include amino acid or polynucleotide sequences that have been changed, for example, by the replacement, deletion, or addition, of one or more, amino acids or nucleotides, thereby providing an amino acid sequence, or a polynucleotide coding sequence that is different from the source sequence. Synthetic gene polynucleotide sequences may not necessarily encode proteins with different amino acids, compared to the unmodified gene, for example, they can also encompass synthetic polynucleotide sequences that incorporate different codons or motifs, but which encode the same amino acid(s); i.e., the nucleotide changes can represent silent mutations at the amino acid level. Synthetic genes can be iteratively modified using the methods described herein and, in each successive iteration, a corresponding polynucleotide sequence or amino acid sequence, is derived in whole, or part, from a sequence that has been designed, or synthesized de novo, or modified, compared to an equivalent unmodified sequence.

“Native antibodies” and “native immunoglobulins” are usually heterotetrameric glycoproteins of about 150,000 daltons, composed of two identical light (L) chains and two identical heavy (H) chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“V_{H}”) followed by a number of constant domains (“C_{H}”). Each light chain has a variable domain at one end (“V_{L}”) and a constant domain (“C_{L}”) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The term “variable domain” refers to protein domains that differ extensively in sequence among family members (i.e. among different isoforms, or in different species). With respect to antibodies, the term “variable domain” refers to the variable domains of antibodies that are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the “framework region” or “FR”. The variable domains of unmodified heavy and light chains each comprise four FRs (FR1, FR2, FR3 and FR4, respectively), largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991), pages 647-669). The constant domains are not involved directly in binding an antibody to an antigen, but exhibit various effector functions, such as participation of the antibody in antibody-dependent cellular toxicity or complement-mediated cell lysis.

The term “hypervariable region” when used herein refers to the amino acid residues of an antibody which are responsible for antigen-binding. The hypervariable region comprises amino acid residues from three “complementarity determining regions” or “CDRs”, which directly bind, in a complementary manner, to an antigen and are known as CDR1, CDR2, and CDR3 respectively.

In the light chain variable domain, the CDRs typically correspond to approximately residues 24-34 (CDRL1), 50-56 (CDRL2) and 89-97 (CDRL3), and in the heavy chain variable domain the CDRs typically correspond to approximately residues 31-35 (CDRH1), 50-65 (CDRH2) and 95-102 (CDRH3); Kabat et al., Sequences of Proteins of Immunological Interest, 5th Ed. Public Health Service, National Institutes of Health, Bethesda, Md. (1991)) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901 917 (1987)).

As used herein, “variable framework region” or “VFR” refers to framework residues that form a part of the antigen binding pocket or groove and/or that may contact antigen. In some embodiments, the framework residues form a loop that is a part of the antigen binding pocket or groove. The amino acids residues in the loop may or may not contact the antigen. In an embodiment, the loop amino acids of a VFR are determined by inspection of the three-dimensional structure of an antibody, antibody heavy chain, or antibody light chain. The three-dimensional structure can be analyzed for solvent accessible amino acid positions as such positions are likely to form a loop and/or provide antigen contact in an antibody variable domain. Some of the solvent accessible positions can tolerate amino acid sequence diversity and others (e.g. structural positions) can be less diversified. The three dimensional structure of the antibody variable domain can be derived from a crystal structure or protein modeling. In some embodiments, the VFR comprises, consists essentially of, or consists of amino acid positions corresponding to amino acid positions 71 to 78 of the heavy chain variable domain, the positions defined according to Kabat et al., 1991. In some embodiments, VFR forms a portion of Framework Region 3 located between CDRH2 and CDRH3. The VFR can form a loop that is well positioned to make contact with a target antigen or form a part of the antigen binding pocket.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG1, IgG2, IgG3, IgG4, IgA, and IgA2. The heavy-chain constant domains (Fc) that correspond to the different classes of immunoglobulins are called α, δ, ε, γ, and μ, respectively. The subunit structures and three-dimensional configurations of different classes of immunoglobulins are well known.

The “light chains” of antibodies (immunoglobulins) from any vertebrate species can be assigned to one of two clearly distinct types, called kappa or (“κ”) and lambda or (“λ”), based on the amino acid sequences of their constant domains.

The terms “antigen-binding portion of an antibody,” “antigen-binding fragment”, “antigen-binding domain”, “antibody fragment” or a “functional fragment of an antibody” are used interchangeably to mean one or more fragments of an antibody that retain the ability to specifically bind to an antigen, (see generally, Holliger et al., Nature Biotech. 23 (9) 1126-1129 (2005)). Non-limiting examples of antibody fragments included within, but not limited to, the term “antigen-binding portion” of an antibody include (i) a Fab fragment, a monovalent fragment consisting of the V_{L}, V_{H}, C_{L} and C_{H}, domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544 546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423 426; and Huston et al. (1988) Proc. Natl. Acad. Sci. USA 85:5879 5883; and Osbourn et al. (1998) Nat. Biotechnol. 16:778). Such single chain antibodies are also intended to be encompassed within the term “antigen-binding portion” of an antibody. Any V_{H} and V_{L} sequences of specific scFv can be linked to human immunoglobulin constant region cDNA or genomic sequences, in order to generate expression vectors encoding complete IgG molecules or other isotypes. V_{H} and V_{L} can also be used in the generation of Fab, Fv or other fragments of immunoglobulins using either protein chemistry or recombinant DNA technology. Other forms of single chain antibodies, such as diabodies are also encompassed.

“F(ab′)₂” and “Fab′” moieties can be produced by treating immunoglobulin (monoclonal antibody) with a protease such as pepsin and papain, and includes an antibody fragment generated by digesting immunoglobulin near the disulfide bonds existing between the hinge regions in each of the two H chains. For example, papain cleaves IgG upstream of the disulfide bonds existing between the hinge regions in each of the two H chains to generate two homologous antibody fragments in which an L chain composed of V_{L} (L chain variable region) and C_{L} (L chain constant region), and an H chain fragment composed of V_{H} (H chain variable region) and C_{Hγ1} (γ1 region in the constant region of H chain) are connected at their C terminal regions through a disulfide bond. Each of these two homologous antibody fragments is called Fab′. Pepsin also cleaves IgG downstream of the disulfide bonds existing between the hinge regions in each of the two H chains to generate an antibody fragment slightly larger than the fragment in which the two above-mentioned Fab′ are connected at the hinge region. This antibody fragment is called F(ab′)₂.

The Fab fragment also contains the constant domain of the light chain and the first constant domain (C_{H}1) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

“Single-chain Fv” or “sFv” antibody fragments comprise the V_{H} and V_{L} domains of an antibody, wherein these domains are present in a single polypeptide chain. In some embodiments, the Fv polypeptide further comprises a polypeptide linker between the V_{H} and V_{L} domains which enables the sFv to form the desired structure for antigen binding. For a review of sFv see, e.g., Pluckthun in The Pharmacology of Monoclonal Antibodies, Vol. 113, Rosenburg and Moore eds. Springer-Verlag, New York, pp. 269 315 (1994).

The term “Avimer™” refers to a new class of therapeutic proteins that are from human origin, which are unrelated to antibodies and antibody fragments, and are composed of several modular and reusable binding domains, referred to as A-domains (also referred to as class A module, complement type repeat, or LDL-receptor class A domain). They were developed from human extracellular receptor domains by in vitro exon shuffling and phage display, (Silverman et al., 2005, Nat. Biotechnol. 23:1493-94; Silverman et al., 2006, Nat. Biotechnol. 24:220). The resulting proteins can comprise multiple independent binding domains that can exhibit improved affinity (in some cases sub-nanomolar) and specificity compared with single-epitope binding proteins. See, for example, U.S. Patent Application Publ. Nos. 2005/0221384, 2005/0164301, 2005/0053973 and 2005/0089932, 2005/0048512, and 2004/0175756, each of which is hereby incorporated by reference herein in its entirety.

Each of the known 217 human A-domains comprises ˜35 amino acids (4 kDa) and domains are separated by linkers that average five amino acids in length. Native A-domains fold quickly and efficiently to a uniform, stable structure mediated primarily by calcium binding and disulfide formation. A conserved scaffold motif of only 12 amino acids is required for this common structure. The end result is a single protein chain containing multiple domains, each of which represents a separate function. Each domain of the proteins binds independently and that the energetic contributions of each domain are additive. These proteins were called “Avimers™” from avidity multimers.

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444 6448 (1993).

Antibodies also include heavy chain dimers, such as antibodies from camelids and sharks. Camelid and shark antibodies comprise a homodimeric pair of two chains of V-like and C-like domains (neither has a light chain). Since the VH region of a heavy chain dimer IgG in a camelid does not have to make hydrophobic interactions with a light chain, the region in the heavy chain that normally contacts a light chain is changed to hydrophilic amino acid residues in a camelid. VH domains of heavy-chain dimer IgGs are called VHH domains. Shark Ig-NARs comprise a homodimer of one variable domain (termed a V-NAR domain) and five C-like constant domains (C-NAR domains).

In camelids, the diversity of antibody repertoire is determined by the complementary determining regions (CDR) 1, 2, and 3 in the VH or VHH regions. The CDR3 in the camel VHH region is characterized by its relatively long length averaging 16 amino acids (Muyldermans et al., 1994, Protein Engineering 7(9): 1129). This is in contrast to CDR3 regions of antibodies of many other species. For example, the CDR3 of mouse VH has an average of 9 amino acids.

Libraries of camelid-derived antibody variable regions, which maintain the in vivo diversity of the variable regions of a camelid, can be made by, for example, the methods disclosed in U.S. Patent Application Ser. No. 20050037421, published Feb. 17, 2005.

As used herein, an “intrabody or fragment thereof” refers to antibodies that are expressed and function intracellularly. Intrabodies, in some embodiments, lack disulfide bonds and are capable of modulating the expression or activity of target genes through their specific binding activity. Intrabodies include single domain fragments such as isolated V_{H} and V_{L} domains and scFvs. An intrabody can include sub-cellular trafficking signals attached to the N or C terminus of the intrabodies to allow them to be expressed at high concentrations in the sub-cellular compartments where a target protein is located. Upon interaction with the target gene, an intrabody modulates target protein function, and/or achieves phenotypic/functional knockout by mechanisms such as accelerating target protein degradation and sequestering the target protein in a non-physiological sub-cellular compartment. Other mechanisms of intrabody-mediated gene inactivation can depend on the epitope to which the intrabody is directed, such as binding to the catalytic site on a target protein or to epitopes that are involved in protein-protein, protein-DNA or protein-RNA interactions. In one embodiment, an intrabody is a scFv.

“Humanized” forms of non-human (e.g., murine) antibodies are chimeric antibodies which contain minimal sequence derived from non-human immunoglobulin. For the most part, humanized antibodies are human immunoglobulins (recipient antibody) in which hypervariable region residues of the recipient are replaced by hypervariable region residues from a non-human species (donor antibody) such as mouse, rat, rabbit or non-human primate having the desired specificity, affinity, and capacity. In some instances, framework region (FR) residues of the human immunoglobulin are replaced by corresponding non-human residues. Furthermore, humanized antibodies can comprise residues which are not found in the recipient antibody or in the donor antibody. These modifications are made to further refine antibody performance. A humanized antibody can comprise substantially all of at least one and, in some cases two, variable domains, in which all or substantially all of the hypervariable regions correspond to those of a non-human immunoglobulin and all, or substantially all, of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522 525 (1986); Reichmann et al., Nature 332:323 329 (1988); and Presta, Curr. Op. Struct. Biol. 2:593 596 (1992).

The term “neutralize” as used herein refers to the ability of an antibody, or antigen-binding fragment, to specifically bind a polypeptide (e.g. RANKL), or portion thereof, and inhibit the binding and/or interaction of the polypeptide (e.g. RANKL) with its cognate receptor (e.g. RANK). In certain embodiments, the antibodies, or antigen-binding fragments, described herein can neutralize the activity of RANKL wherein the in vivo or in vitro effects of the interaction between RANKL/RANK are reduced or eliminated.

The term “monoclonal antibody” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to conventional (polyclonal) antibody preparations, which can include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the embodiments described herein can be made by the hybridoma method first described by Kohler et al., Nature 256:495 (1975), or can be made by recombinant DNA methods (see, e.g., U.S. Pat. No. 4,816,567). In certain embodiments, the “monoclonal antibodies” can also be isolated from phage antibody libraries using the techniques described in Clackson et al., Nature 352:624 628 (1991) and Marks et al., J. Mol. Biol. 222:581 597 (1991), for example.

In other embodiments, monoclonal antibodies can be isolated and purified from the culture supernatant or ascites mentioned above by saturated ammonium sulfate precipitation, euglobulin precipitation method, caproic acid method, caprylic acid method, ion exchange chromatography (DEAE or DE52), or affinity chromatography using anti-immunoglobulin column, a protein A column, or any other conventionally or known technology.

A polyclonal antibody (antiserum) or monoclonal antibody can be produced by known methods. Namely, mammals, such as mice, rats, hamsters, guinea pigs, rabbits, cats, dogs, pigs, goats, horses, or cows are immunized, for example, with an antigen mentioned above with, if necessary, an adjuvant (e.g., Complete or Incomplete Freund's). The polyclonal antibody can be obtained from the serum obtained from the animal so immunized. The monoclonal antibodies are produced as follows. Hybridomas are produced by fusing the antibody-producing cells obtained from the animal so immunized and myeloma cells incapable of producing auto-antibodies. Then the hybridomas are cloned, and clones producing the monoclonal antibodies showing the specific affinity to the antigen used for immunizing the mammal are screened.

An “isolated specific binding member” is one which has been identified and separated and/or recovered from a component of its natural environment. Contaminant components of its natural environment are materials which would interfere with diagnostic or therapeutic uses for the specific binding member and include, for example, enzymes, hormones, and other proteinaceous or nonproteinaceous solutes. In certain embodiments, the specific binding member can be purified (1) to greater than 95% by weight as determined by the Lowry or comparable assay method (e.g., more than 99% by weight), (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain. Isolated specific binding members include those in situ within recombinant cells since at least one component of the specific binding member's natural environment will not be present. Ordinarily, however, isolated specific binding members can be prepared by at least one purification step (e.g., Protein A, G or L purification columns or other conventional techniques known in the art).

The “cell producing an antibody reactive to a protein or a fragment thereof” means any cell producing any of the antibodies or antigen-binding fragments described herein.

The term “germline gene segments” refers to the genes from the germline (the haploid gametes and those diploid cells from which they are formed). The germline DNA contain multiple gene segments that encode a single immunoglobin heavy or light chain. These gene segments are carried in the germ cells but cannot be transcribed and translated into heavy and light chains until they are arranged into functional genes. During B-cell differentiation in the bone marrow, these gene segments are randomly shuffled by a dynamic genetic system capable of generating more than 108 specificities. Most of these gene segments are published and collected by the germline database.

As used herein, “library” refers to a plurality of polynucleotides, proteins, or cells comprising a collection of two or two or more non-identical, but related members. A “synthetic library” refers to a plurality of synthetic polynucleotides, or a population of cells that comprise said plurality of synthetic polynucleotides. A “semi-synthetic library” refers to a plurality of semi-synthetic polynucleotides, or a population of cells that comprise said plurality of semi-synthetic polynucleotides.

The terms “synthetic polynucleotide”, “synthetic gene” or “synthetic polypeptide,” as used herein, mean that the corresponding polynucleotide sequence or portion thereof, or amino acid sequence or portion thereof, is derived, from a sequence that has been designed, or synthesized de-novo, or modified, compared to the equivalent naturally occurring sequence. Synthetic polynucleotides or synthetic genes can be prepared by methods known in the art, including but not limited to, the chemical synthesis of nucleic acid or amino acid sequences. Synthetic genes are typically different from naturally occurring genes, either at the amino acid, or polynucleotide level, (or both) and are typically located within the context of synthetic expression control sequences. For example, synthetic gene sequences can include amino acid, or polynucleotide, sequences that have been changed, for example, by the replacement, deletion, or addition, of one or more, amino acids, or nucleotides, thereby providing an antibody amino acid sequence, or a polynucleotide coding sequence that is different from the source sequence. Synthetic gene polynucleotide sequences, may not necessarily encode proteins with different amino acids, compared to the natural gene, for example, they can also encompass synthetic polynucleotide sequences that incorporate different codons but which encode the same amino acid; i.e. the nucleotide changes represent silent mutations at the amino acid level

The terms “semi-synthetic polynucleotide” or “semi-synthetic gene,” as used herein, refer to a polynucleotide sequences that consist in part of a nucleic acid sequence that has been obtained via polymerase chain reaction (PCR) or other similar enzymatic amplification system which utilizes a natural donor (i.e., peripheral blood monocytes) as the starting material for the amplification reaction. The remaining “synthetic” polynucleotides, i.e., those portions of semi-synthetic polynucleotide not obtained via PCR or other similar enzymatic amplification system, can be synthesized de-novo using methods known in the art including, but not limited to, the chemical synthesis of nucleic acid sequences.

As used herein, the term “antigen” refers to substances that are capable, under appropriate conditions, of inducing an immune response to the substance and of reacting with the products of the immune response. For example, an antigen can be recognized by antibodies (humoral immune response) or sensitized T-lymphocytes (T helper or cell-mediated immune response), or both. Antigens can be soluble substances, such as toxins and foreign proteins, or particulates, such as bacteria and tissue cells; however, only the portion of the protein or polysaccharide molecule known as the antigenic determinant (epitopes) combines with the antibody or a specific receptor on a lymphocyte. More broadly, the term “antigen” refers to any substance to which an antibody binds, or for which antibodies are desired, regardless of whether the substance is immunogenic. For such antigens, antibodies can be identified by recombinant methods, independently of any immune response.

As used herein, the term “affinity” refers to the equilibrium constant for the reversible binding of two agents and is expressed as Kd. Affinity of a binding protein to a ligand such as affinity of an antibody for an epitope can be, for example, from about 100 nanomolar (nM) to about 0.1 nM, from about 100 nM to about 1 picomolar (pM), or from about 100 nM to about 1 femtomolar (fM). As used herein, the term “avidity” refers to the resistance of a complex of two or more agents to dissociation after dilution.

“Epitope” refers to that portion of an antigen or other macromolecule capable of forming a binding interaction that interacts with the variable region binding pocket of a binding protein (e.g., an antibody). Such binding interaction can be manifested as an intermolecular contact with one or more amino acid residues of a CDR. Antigen binding can involve a CDR3 or a CDR3 pair. An epitope can be a linear peptide sequence (i.e., “continuous”) or can be composed of noncontiguous amino acid sequences (i.e., “conformational” or “discontinuous”). A binding protein can recognize one or more amino acid sequences; therefore an epitope can define more than one distinct amino acid sequence. Epitopes recognized by binding protein can be determined by peptide mapping and sequence analysis techniques well known to one of skill in the art. Typically, such binding interaction is manifested as an intermolecular contact with one or more amino acid residues of a CDR. Often, the antigen binding involves a CDR3 or a CDR3 pair.

A “cryptic epitope” or a “cryptic binding site” is an epitope or binding site of a protein sequence that is not exposed or substantially protected from recognition within an unmodified polypeptide, but is capable of being recognized by a binding protein of a denatured or proteolyzed polypeptide. Amino acid sequences that are not exposed, or are only partially exposed, in the unmodified polypeptide structure are potential cryptic epitopes. If an epitope is not exposed, or only partially exposed, then it is likely that it is buried within the interior of the polypeptide. Candidate cryptic epitopes can be identified, for example, by examining the three-dimensional structure of an unmodified polypeptide.

The term “specific” refers to a situation in which one member of a specific binding pair will not show any significant binding to molecules other than its specific binding partner(s). The term is also applicable where e.g. an antigen binding domain is specific for a particular epitope which is cross-reactive a number of antigens, in which case the specific binding member carrying the antigen binding domain will be able to bind to the various antigens carrying the cross-reactive epitope.

The term “binding” refers to a direct association between two molecules, due to, for example, covalent, electrostatic, hydrophobic, and ionic and/or hydrogen-bond interactions under physiological conditions, and includes interactions such as salt bridges and water bridges, as well as any other conventional means of binding.

The term “specific binding member” describes a member of a pair of molecules which have binding specificity for one another. The members of a specific binding pair can be naturally derived or wholly or partially synthetically produced. One member of the pair of molecules has an area on its surface, or a cavity, which specifically binds to and is therefore complementary to a particular spatial and polar organization of the other member of the pair of molecules. Thus, the members of the pair have the property of binding specifically to each other. Examples of types of specific binding pairs include antigen-antibody, Avimer™-substrate, biotin-avidin, hormone-hormone receptor, receptor-ligand, protein-protein, and enzyme-substrate.

“Adjuvant” refers to a compound or mixture that enhances the immune response. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Often, a primary challenge with an antigen alone, in the absence of an adjuvant, will fail to elicit a humoral or cellular immune response. Previously known and utilized adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvant such as BCG (Bacille Calmette-Guerin) and Corynebacterium parvum. Mineral salt adjuvants include but are not limited to: aluminum hydroxide, aluminum phosphate, calcium phosphate, zinc hydroxide and calcium hydroxide. The adjuvant composition can further comprise a lipid of fat emulsion comprising about 10% (by weight) vegetable oil and about 1-2% (by weight) phospholipids. In one non-limiting example, the adjuvant composition further optionally comprises an emulsion form having oily particles dispersed in a continuous aqueous phase, having an emulsion forming polyol in an amount of from about 0.2% (by weight) to about 49% (by weight), optionally a metabolizable oil in an emulsion-forming amount of up to 15% (by weight), and optionally a glycol ether-based surfactant in an emulsion-stabilizing amount of up to about 5% (by weight).

As used herein, the term “immunomodulator” refers to an agent which is able to modulate an immune response. An example of such modulation is an enhancement of antibody production. Another example of such modulation is an enhancement of a T cell response.

An “immunological response” to a composition or vaccine comprised of an antigen is the development in the host of a cellular- and/or antibody-mediated immune response to the composition or vaccine of interest. Usually, such a response consists of the subject producing antibodies, B cells, helper T cells, suppressor T cells, and/or cytotoxic T cells directed specifically to an antigen or antigens included in the composition or vaccine of interest.

B. Molecular Biological Terminology

The term “nucleotide” as used herein refers to a monomeric unit of a polynucleotide that consists of a heterocyclic base, a sugar, and one or more phosphate groups. The naturally occurring bases, (guanine, (G), adenine, (A), cytosine, (C), thymine, (T), and uracil (U)) are typically derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleic acids are linked via phosphate bonds to form nucleic acids, or polynucleotides, though many other linkages are known in the art (such as, though not limited to phosphorothioates, boranophosphates and the like).

The terms “nucleic acid” and “polynucleotide” as used herein refer to a polymeric form of nucleotides of any length, either ribonucleotides (RNA) or deoxyribonucleotides (DNA). These terms refer to the primary structure of the molecules and, thus, include double- and single-stranded DNA, and double- and single-stranded RNA. These terms include, as equivalents, analogs of either RNA or DNA made from nucleotide analogs and modified polynucleotides such as, though not limited to, methylated and/or capped polynucleotides.

A “DNA molecule” refers to the polymeric form of deoxyribonucleotides (adenine, guanine, thymine, or cytosine) in its either single stranded form or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA).

An “origin of replication” refers to those DNA sequences that participate in DNA synthesis.

A DNA “coding sequence” or “coding region” is a double-stranded DNA sequence which is transcribed and translated into a polypeptide in vivo when placed under the control of appropriate expression control sequences. The boundaries of the coding sequence (the “open reading frame” or “ORF”) are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and synthetic DNA sequences. A polyadenylation signal and transcription termination sequence will usually be located 3′ to the coding sequence. The term “non-coding sequence” or “non-coding region” refers to regions of a polynucleotide sequence that are not translated into amino acids (e.g. 5′ and 3′ un-translated regions).

The term “reading frame” refers to one of the six possible reading frames, three in each direction, of the double stranded DNA molecule. The reading frame that is used determines which codons are used to encode amino acids within the coding sequence of a DNA molecule.

As used herein, an “antisense” nucleic acid molecule comprises a nucleotide sequence which is complementary to a “sense” nucleic acid encoding a protein, e.g., complementary to the coding strand of a double-stranded cDNA molecule, complementary to an mRNA sequence or complementary to the coding strand of a gene. Accordingly, an antisense nucleic acid molecule can hydrogen bond to a sense nucleic acid molecule.

The term “base pair” or (“bp”): a partnership of adenine (A) with thymine (T), or of cytosine (C) with guanine (G) in a double stranded DNA molecule. In RNA, uracil (U) is substituted for thymine.

As used herein a “codon” refers to the three nucleotides which, when transcribed and translated, encode a single amino acid residue; or in the case of UUA, UGA or UAG encode a termination signal. Codons encoding amino acids are well known in the art and are provided for convenience herein in Table 2.

TABLE 2
Codon Usage Table

| | | | | | | | |
|---|---|---|---|---|---|---|---|
| Codon | Amino acid | AA | Abbr. | Codon | Amino acid | AA | Abbr. |

| | | | | | | | |
|---|---|---|---|---|---|---|---|
| UUU | Phenylalanine | Phe | F | UCU | Serine | Ser | S |
| UUC | Phenylalanine | Phe | F | UCC | Serine | Ser | S |
| UUA | Leucine | Leu | L | UCA | Serine | Ser | S |
| UUG | Leucine | Leu | L | UCG | Serine | Ser | S |
| CUU | Leucine | Leu | L | CCU | Proline | Pro | P |
| CUC | Leucine | Leu | L | CCC | Proline | Pro | P |
| CUA | Leucine | Leu | L | CCA | Proline | Pro | P |
| CUG | Leucine | Leu | L | CCG | Proline | Pro | P |
| AUU | Isoleucine | Ile | I | ACU | Threonine | Thr | T |
| AUC | Isoleucine | Ile | I | ACC | Threonine | Thr | T |
| AUA | Isoleucine | Ile | I | ACA | Threonine | Thr | T |
| AUG | Methionine | Met | M | ACH | Threonine | Thr | T |
| GUU | Valine | Val | V | GCU | Alanine | Ala | A |
| GUC | Valine | Val | V | GCC | Alanine | Ala | A |
| GUA | Valine | Val | V | GCA | Alanine | Ala | A |
| GUG | Valine | Val | V | GCG | Alanine | Ala | A |
| UAU | Tyrosine | Tyr | Y | UGU | Cysteine | Cys | C |
| UAC | Tyrosine | Tyr | Y | UGC | Cysteine | Cys | C |
| UUA | | Stop | | UGA | | Stop | |
| UAG | | Stop | | UGG | Tryptophan | Trp | W |
| CAU | Histidine | His | H | CGU | Arginine | Arg | R |
| CAC | Histidine | His | H | CGC | Arginine | Arg | R |
| CAA | Glutamine | Gln | Q | CGA | Arginine | Arg | R |
| CAG | Glutamine | Gln | Q | CGG | Arginine | Arg | R |
| AAU | Asparagine | Asn | N | AGU | Serine | Ser | S |
| AAC | Asparagine | Asn | N | AGC | Serine | Ser | S |
| AAA | Lysine | Lys | K | AGA | Arginine | Arg | R |
| AAG | Lysine | Lys | K | AGG | Arginine | Arg | R |
| GAU | Aspartate | Asp | D | GGU | Glycine | Gly | G |
| GAC | Aspartate | Asp | D | GGC | Glycine | Gly | G |
| GAA | Glutamate | Glu | E | GGA | Glycine | Gly | G |
| GAG | Glutamate | Glu | E | GGG | Glycine | Gly | G |

In keeping with standard polypeptide nomenclature, J. Biol. Chem., 243:3552-59 (1969), abbreviations for amino acid residues are shown above. AA: amino acid; Abbr: abbreviation. It should be understood that the codons specified above are for RNA sequences. The corresponding codons for DNA have a T substituted for U. Optimal codon usage is indicated by codon usage frequencies for expressed genes, for example, as shown in the codon usage chart from the program “Human—High.cod” from the Wisconson Sequence Analysis Package, Version 8.1, Genetics Computer Group, Madison, Wis. Codon usage is also described in, for example, R. Nussinov, “Eukaryotic Dinucleotide Preference Rules and Their Implications for Degenerate Codon Usage,” J. Mol. Biol. 149: 125-131 (1981). The codons which are most frequently used in highly expressed human genes are presumptively the optimal codons for expression in human host cells and, thus, form the bases for constructing a synthetic coding sequence.

As used herein, a “wobble position” refers to the third position of a codon. Mutations in a DNA molecule within the wobble position of a codon, in some embodiments, result in silent or conservative mutations at the amino acid level. For example, there are four codons that encode Glycine, i.e., GGU, GGC, GGA and GGG, thus mutation of any wobble position nucleotide, to any other nucleotide, does not result in a change at the amino acid level of the encoded protein and, therefore, is a silent substitution.

Accordingly a “silent substitution” or “silent mutation” is one in which a nucleotide within a codon is modified, but does not result in a change in the amino acid residue encoded by the codon. Examples include mutations in the third position of a codon, as well in the first position of certain codons, such as in the codon “CGG”, which when mutated to AGG, still encodes the amino acid Arginine (Arg, or R).

The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids can be defined where amino acids within a group exchange preferentially with each other, and therefore resemble each other most in their impact on the overall protein structure (Schulz, G. E. and R. H. Schirmer, Principles of Protein Structure, Springer-Verlag).

Examples of amino acid groups defined in this manner include: a “charged/polar group”, consisting of Glu, Asp, Asn, Gln, Lys, Arg and H is; an “aromatic, or cyclic group,” consisting of Pro, Phe, Tyr and Trp; and an “aliphatic group” consisting of Gly, Ala, Val, Leu, Ile, Met, Ser, Thr and Cys.

Within each group, subgroups can also be identified, for example, the group of charged/polar amino acids can be sub-divided into the sub-groups consisting of the “positively-charged sub-group,” consisting of Lys, Arg and His; the negatively-charged sub-group”, consisting of Glu and Asp, and the “polar sub-group” consisting of Asn and Gln.

The aromatic or cyclic group can be sub-divided into the sub-groups consisting of the “nitrogen ring sub-group,” consisting of Pro, His and Trp; and the “phenyl sub-group” consisting of Phe and Tyr.

The aliphatic group can be sub-divided into the sub-groups consisting of the “large aliphatic non-polar sub-group”, consisting of Val, Leu and Ile; the “aliphatic slightly-polar sub-group,” consisting of Met, Ser, Thr and Cys; and the “small-residue sub-group,” consisting of Gly and Ala.

Examples of conservative mutations include amino acid substitutions of amino acids within the sub-groups above, for example, Lys for Arg and vice versa such that a positive charge can be maintained; Glu for Asp and vice versa such that a negative charge can be maintained; Ser for Thr such that a free —OH can be maintained; and Gln for Asn such that a free —NH2 can be maintained.

“Semi-conservative mutations” include amino acid substitutions of amino acids with the same groups listed above, that do not share the same sub-group. For example, the mutation of Asp for Asn, or Asn for Lys all involve amino acids within the same group, but different sub-groups.

“Non-conservative mutations” involve amino acid substitutions between different groups, for example Lys for Leu, or Phe for Ser, etc.

The term “amino acid residue” refers to the radical derived from the corresponding alpha-amino acid by eliminating the OH portion of the carboxyl group and the H-portion of the alpha amino group. For the most part, the amino acids used in the application are those naturally occurring amino acids found in proteins, or the naturally occurring anabolic or catabolic products of such amino acids which contain amino and carboxyl groups. Alternatively, un-natural amino acids can be incorporated into proteins to facilitate the chemical conjugation to other proteins, toxins, small organic compounds or anti-cancer agents (Datta et al., J Am Chem. Soc., 2002; 124 (20):5652-3). The abbreviations used herein for designating the amino acids and the protective groups are based on recommendations of the IUPAC-IUB Commission on Biochemical Nomenclature (see Biochemistry (1972) 11: 1726-1732). The term “amino acid residue” also includes analogs, derivatives and congeners of any specific amino acid referred to herein, as well as C-terminal or N-terminal protected amino acid derivatives (e.g., modified with an N-terminal or C-terminal protecting group). For example, the present application contemplates the use of amino acid analogs wherein a side chain is lengthened or shorted while still providing a carboxyl, amino or other reactive precursor functional group for cyclization, as well as amino acid analogs having variant side chains with appropriate functional groups).

The term “amino acid side chain” is that part of an amino acid exclusive of the —CH—(NH2)COOH portion, as defined by K. D. Kopple, “Peptides and Amino Acids,” W. A. Benjamin Inc., New York and Amsterdam, 1996, pages 2 and 33; examples of such side chains of the common amino acids are —CH2CH2SCH3 (the side chain of methionine), —CH2(CH3)-CH2CH3 (the side chain of isoleucine), —CH2CH(CH3)₂ (the side chain of leucine) or H—(the side chain of glycine).

The amino acid residues described herein are preferred to be in the “L” isomeric form. However, residues in the “D” isomeric form can be substituted for any L-amino acid residue, as long as the desired functional property of antibody (immunoglobulin)-binding is retained by the polypeptide. NH2 refers to the free amino group present at the amino terminus of a polypeptide. COOH refers to the free carboxy group present at the carboxy terminus of a polypeptide.

An “amino acid motif” is a sequence of amino acids, optionally a generic set of conserved amino acids, associated with a particular functional activity.

As used herein, the terms “protein,” “peptide” and “polypeptide” are used interchangeably to refer to polymers of amino acid residues of any length connected to one another by peptide bonds between the alpha-amino group and carboxy group of contiguous amino acid residues. Polypeptides, proteins and peptides can exist as linear polymers, branched polymers or in circular form. These terms also include forms that are post-translationally modified in vivo, or chemically modified during synthesis. Polypeptides displaying substantially equivalent or altered activity are likewise contemplated. Such modifications can be deliberate, for example, such as modifications obtained through site-directed mutagenesis, or can be accidental, such as those obtained through mutations in hosts that are producers of the complex or its named subunits. Polypeptides are intended to include within their scope proteins specifically recited herein as well as all substantially homologous analogs and allelic variations.

It should be noted that all amino acid residue sequences are represented herein by formulae whose left and right orientation is in the conventional direction of amino-terminus to carboxy terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

The terms “gene,” “recombinant gene” and “gene construct” as used herein, refer to a DNA molecule, or portion of a DNA molecule, that encodes a protein or a portion thereof. The DNA molecule can contain an open reading frame encoding the protein (as exon sequences) and can further include intron sequences. The term “intron” as used herein, refers to a DNA sequence present in a given gene which is not translated into protein and is found in some, but not all cases, between exons. It can be desirable for the gene to be operably linked to, (or it can comprise), one or more promoters, enhancers, repressors and/or other regulatory sequences to modulate the activity or expression of the gene, as is well known in the art.

As used herein, a “complementary DNA” or “cDNA” includes recombinant polynucleotides synthesized by reverse transcription of mRNA and from which intervening sequences (introns) have been removed.

The term “operably linked” as used herein, describes the relationship between two polynucleotide regions such that they are functionally related or coupled to each other. For example, a promoter (or other expression control sequence) is operably linked to a coding sequence if it controls (and is capable of effecting) the transcription of the coding sequence. Although an operably linked promoter is generally located upstream of the coding sequence, it is not necessarily contiguous with it.

“Expression control sequences” are DNA regulatory sequences, such as promoters, enhancers, polyadenylation signals, terminators, internal ribosome entry sites (IRES) and the like, that provide for the expression of a coding sequence in a host cell. Expression control sequences can contribute to control and regulation of the transcription and translation of another DNA sequence. A coding sequence is “under the control” of transcriptional and translational control sequences in a cell when RNA polymerase transcribes the coding sequence into mRNA, which is then translated into the protein encoded by the coding sequence. Exemplary expression control sequences are described in Goeddel; Gene Expression Technology: Methods in Enzymology 185, Academic Press, San Diego, Calif. (1990).

A “promoter” is a DNA regulatory region capable of binding RNA polymerase in a cell and initiating transcription of a downstream (3′ direction) coding sequence. As used herein, the promoter sequence is bounded at its 3′ terminus by the transcription initiation site and extends upstream (5′ direction) to include the minimum number of bases or elements necessary to initiate transcription at levels detectable above background. A transcription initiation site (conveniently defined by mapping with nuclease S1) can be found within a promoter sequence, as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

A large number of promoters, including constitutive, inducible and repressible promoters, from a variety of different sources are well known in the art. Representative sources include for example, viral, mammalian, insect, plant, yeast, and bacterial cell types), and suitable promoters from these sources are readily available, or can be made synthetically, based on sequences publicly available on line or, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araa) bacterial expression system, the cytomegalovirus (CMV) promoter, the SV40 promoter, the RSV promoter. Inducible promoters include the Tet system, (U.S. Pat. Nos. 5,464,758 and 5,814,618), the Ecdysone inducible system (No et al., Proc. Natl. Acad. Sci. (1996) 93 (8) 3346-3351; the T-RE_{X™ system (Invitrogen Carlsbad, Calif.), LacSwitch® (Stratagene, (San Diego, Calif.) and the Cre-ER}^{T} tamoxifen inducible recombinase system (Indra et al. Nuc. Acid. Res. (1999) 27 (22)4324-4327; Nuc. Acid. Res. (2000) 28 (23) e99; U.S. Pat. No. 7,112,715). See generally, Kramer & Fussenegger Methods Mol. Biol. (2005) 308: 123-144) or any promoter known in the art suitable for expression in the desired cells.

As used herein, a “minimal promoter” refers to a partial promoter sequence which defines the transcription start site but which by itself is not capable, if at all, of initiating transcription efficiently. The activity of such minimal promoters depends on the binding of activators such as a tetracycline-controlled transactivator to operably linked binding sites.

The terms “IRES” or “internal ribosome entry site” refer to a polynucleotide element that acts to enhance the translation of a coding sequence encoded with a polycistronic messenger RNA. IRES elements, mediate the initiation of translation by directly recruiting and binding ribosomes to a messenger RNA (mRNA) molecule, bypassing the 7-methyl guanosine-cap involved in typical ribosome scanning. The presence of an IRES sequence can increase the level of cap-independent translation of a desired protein. Early publications descriptively refer to IRES sequences as “translation enhancers.” For example, cardioviral RNA “translation enhancers” are described in U.S. Pat. No. 4,937,190 to Palmenberg et al. and U.S. Pat. No. 5,770,428 to Boris-Lawrie.

The term “enhancer” as used herein, refers to a DNA sequence that increases transcription of, for example, a gene or coding sequence to which it is operably linked. Enhancers can be located many kilobases away from the coding sequence and can mediate the binding of regulatory factors, patterns of DNA methylation or changes in DNA structure. A large number of enhancers, from a variety of different sources are well known in the art and available as or within cloned polynucleotides (from, e.g., depositories such as the ATCC as well as other commercial or individual sources). A number of polynucleotides comprising promoters (such as the commonly-used CMV promoter) also comprise enhancer sequences. Operably linked enhancers can be located upstream, within, or downstream of coding sequences. The term “Ig enhancers” refers to enhancer elements derived from enhancer regions mapped within the Ig locus (such enhancers include for example, the heavy chain (mu) 5′ enhancers, light chain (kappa) 5′ enhancers, kappa and mu intronic enhancers, and 3′ enhancers, (see, e.g., Paul W E (ed) Fundamental Immunology, 3rd Edition, Raven Press, New York (1993) pages 353-363; U.S. Pat. No. 5,885,827).

“Terminator sequences” are those that result in termination of transcription. Termination sequences are known in the art and include, but are not limited to, poly A (e.g., Bgh Poly A and SV40 Poly A) terminators. A transcriptional termination signal will typically include a region of 3′ untranslated region (or “3′ ut”), an optional intron (also referred to as intervening sequence or “IVS”) and one or more poly adenylation signals (“p(A)” or “pA”. Terminator sequences can also be referred to as “IVS−pA”, “IVS+p(A)”, “3′ ut+p(A)” or “3′ ut/p(A)”. Natural or synthetic terminators can be used as a terminator region.

The terms “polyadenylation”, “polyadenylation sequence,” “polyadenylation signal,” “Poly A,” “p(A)” or “pA” refer to a nucleic acid sequence present in a RNA transcript that allows for the transcript, when in the presence of the polyadenyl transferase enzyme, to be polyadenylated. Many polyadenylation signals are known in the art. Non-limiting examples include the human variant growth hormone polyadenylation signal, the SV40 late polyadenylation signal and the bovine growth hormone polyadenylation signal.

The term “splice site” as used herein refers to polynucleotides that are capable of being recognized by the spicing machinery of a eukaryotic cell as suitable for being cut and/or ligated to a corresponding splice site. Splice sites allow for the excision of introns present in a pre-mRNA transcript. In one example, the 5′ portion of the splice site is referred to as the splice donor and the 3′ corresponding splice site is referred to as the acceptor splice site. The term splice site includes, for example, naturally occurring splice sites, engineered splice sites, for example, synthetic splice sites, canonical or consensus splice sites, and/or non-canonical splice sites, for example, cryptic splice sites.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

“Post-translational modification” can encompass any one of or a combination of modification(s), including covalent modification(s), which a protein undergoes after translation is complete and after being released from the ribosome or on the nascent polypeptide co-translationally. Post-translational modification includes but is not limited to phosphorylation, myristylation, ubiquitination, glycosylation, coenzyme attachment, methylation, S-nitrosylation and acetylation. Post-translational modification can modulate or influence the activity of a protein, its intracellular or extracellular destination, its stability or half-life, and/or its recognition by ligands, receptors or other proteins. Post-translational modification can occur in cell organelles, in the nucleus or cytoplasm or extracellularly.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer can be either single-stranded or double-stranded and must be sufficiently long to prime the synthesis of the desired extension product in the presence of the inducing agent. The exact length of the primer can depend upon many factors, including temperature, source of primer and use of the method. For example, for diagnostic applications, depending on the complexity of the target sequence, an oligonucleotide primer can contain about 15 to about 25 or more nucleotides, although it can contain fewer nucleotides. The polynucleotide primers can be prepared using any suitable method, such as, for example, the phosphotriester on phosphodiester methods see Narang et al., Meth. Enzymol., 68:90, (1979); U.S. Pat. No. 4,356,270; and Brown et al., Meth. Enzymol., 68:109, (1979).

The primers herein are selected to be “substantially” complementary to different strands of a particular target polynucleotide sequence. This means that the primers must be sufficiently complementary to hybridize with their respective strands. Therefore, the primer sequence need not reflect the exact sequence of the template. For example, a non-complementary nucleotide fragment can be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

The term “multiple cloning site” as used herein, refers to a segment of a vector polynucleotide which can recognize one or more different restriction enzymes.

A “replicon” is any genetic element (e.g., plasmid, episome, chromosome, yeast artificial chromosome, YAC, virus) that functions as an autonomous unit of DNA replication in vivo; i.e., capable of replication under its own control, and containing autonomous replicating sequences.

A “vector” or “cloning vector” is a replicon, such as plasmid, phage or cosmid, into which another polynucleotide segment can be introduced so as to bring about the replication of the inserted segment. Vectors typically exist as circular, double stranded DNA, and range in size form a few kilobases (kb) to hundreds of kb. Cloning vectors can be modified from naturally occurring plasmids to facilitate the cloning and recombinant manipulation of polynucleotide sequences. Many such vectors are well known in the art; see for example, by Sambrook (In. “Molecular Cloning: A Laboratory Manual”, second edition, edited by Sambrook, Fritsch, & Maniatis, Cold Spring Harbor Laboratory, (1989)), Maniatis, In: Cell Biology: A Comprehensive Treatise, Vol. 3, Gene Sequence Expression, Academic Press, NY, pp. 563-608 (1980).

The term “expression vector” as used herein, refers to a vector used for expressing certain polynucleotides within a host cell or in-vitro expression system. The term includes plasmids, episomes, cosmids retroviruses or phages; the expression vector can be used to express a DNA sequence encoding a desired protein and in one aspect includes a transcriptional unit comprising an assembly of expression control sequences. The choice of promoter and other regulatory elements can vary according to the intended host cell, or in-vitro expression system.

An “episomal expression vector” is able to replicate in the host cell, and persists as an extrachromosomal segment of DNA within the host cell in the presence of appropriate selective pressure. (See for example, Conese et al., Gene Therapy 11 1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7 from Invitrogen, pcDNA3.1 from Invitrogen, and pBK-CMV from Stratagene represent non-limiting examples of an episomal vector that uses T-antigen and the SV40 origin of replication in lieu of EBNA1 and orip.

An “integrating expression vector” can randomly integrate into the host cell's DNA, or can include a recombination site to enable the specific recombination between the expression vector and the host cells chromosome. Such integrating expression vectors can utilize the endogenous expression control sequences of the host cell's chromosomes to effect expression of the desired protein. Examples of vectors that integrate in a site specific manner include, for example, components of the flp-in system from Invitrogen (e.g., pcDNA™5/FRT), or the cre-lox system, such as can be found in the pExchange-6 Core Vectors from Stratagene. Examples of vectors that integrate into host cell chromosomes in a random fashion include, for example, pcDNA3.1 (when introduced in the absence of T-antigen) from Invitrogen, pCI or pFN10A (ACT) Flexi® from Promega.

Representative commercially available viral expression vectors include, but are not limited to, the adenovirus-based Per.C6 system available from Crucell, Inc., the lentiviral-based pLP1 from Invitrogen, and the Retroviral Vectors pFB-ERV plus pCFB-EGSH from Stratagene.

Alternatively, the expression vector can be used to introduce and integrate a strong promoter or enhancer sequences into a locus in the cell so as to modulate the expression of an endogenous gene of interest (Capecchi M R. Nat Rev Genet. (2005); 6 (6):507-12; Schindehutte et al., Stem Cells (2005); 23 (1):10-5). This approach can also be used to insert an inducible promoter, such as the Tet-On promoter (U.S. Pat. Nos. 5,464,758 and 5,814,618), in to the genomic DNA of the cell so as to provide inducible expression of an endogenous gene of interest. The activating construct can also include targeting sequence(s) to enable homologous or non-homologous recombination of the activating sequence into a desired locus specific for the gene of interest (see for example, Garcia-Otin & Guillou, Front Biosci. (2006) 11:1108-36). Alternatively, an inducible recombinase system, such as the Cre-ER system, can be used to activate a transgene in the presence of 4-hydroxytamoxifen. (Indra et al. Nuc. Acid. Res. (1999) 27 (22) 4324-4327; Nuc. Acid. Res. (2000) 28 (23) e99; U.S. Pat. No. 7,112,715).

Expression vectors can also include anti-sense, ribozymes or siRNA polynucleotides to reduce the expression of target sequences. (See generally, Sioud M, & Iversen, Curr. Drug Targets (2005) 6 (6):647-53; Sandy et al., Biotechniques (2005) 39 (2):215-24).

Transcriptional and translational control sequences are DNA regulatory sequences, such as promoters, enhancers, polyadenylation signals, terminators, and the like, that provide for the expression of a coding sequence in a host cell.

The term “oligonucleotide,” as used herein, refers to a molecule comprised of two or more ribonucleotides, preferably more than three. Its exact size will depend upon many factors which, in turn, depend upon the ultimate function and use of the oligonucleotide.

As used herein, a “recombination system” refers to one which allows for recombination between a vector of the present application and a chromosome for incorporation of a gene of interest. Recombination systems are known in the art and include, for example, Cre/Lox systems and FLP-IN systems.

As used herein an “in-vitro expression system” refers to cell free systems that enable the transcription, or coupled transcription and translation of DNA templates. Such systems include for example the classical rabbit reticulocyte system, as well as novel cell free synthesis systems, (J. Biotechnol. (2004) 110 (3) 257-63; Biotechnol Annu. Rev. (2004) 10 1-30).

As used herein, a “Cre/Lox” system refers to one such as described by Abremski et al., Cell, 32: 1301-1311 (1983) for a site-specific recombination system of bacteriophage P1. Methods of using Cre-Lox systems are known in the art; see, for example, U.S. Pat. No. 4,959,317, which is hereby incorporated in its entirety by reference. The system consists of a recombination site designated loxp and a recombinase designated Cre. In methods for producing site-specific recombination of DNA in eukaryotic cells, DNA sequences having first and second lox sites are typically introduced into eukaryotic cells and contacted with Cre, thereby producing recombination at the lox sites.

As used here, “FLP-IN” recombination refers to systems in which a polynucleotide activation/inactivation and site-specific integration system has been developed for mammalian cells. The system is based on the recombination of transfected sequences by FLP, a recombinase derived from Saccharomyces. In several cell lines, FLP has been shown to rapidly and precisely recombine copies of its specific target sequence. FLP-IN systems have been described in, for example, U.S. Pat. Nos. 5,654,182 and 5,677,177).

The term “transfection,” “transformation,” or “transduction” as used herein, refers to the introduction of one or more exogenous polynucleotides into a host cell by using one or physical or chemical methods. A cell has been transformed by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA can be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations. Many transfection techniques are known to those of ordinary skill in the art including but not limited to calcium phosphate DNA co-precipitation (see Methods in Molecular Biology, Vol. 7, Gene Transfer and Expression Protocols, Ed. E. J. Murray, Humana Press (1991)); DEAE-dextran; electroporation; cationic liposome-mediated transfection; tungsten particle-facilitated microparticle bombardment (Johnston, S. A., Nature 346: 776-777 (1990)); and strontium phosphate DNA co-precipitation (Brash D. E. et al. Molec. Cell. Biol. 7: 2031-2034 (1987). Phage, or retroviral vectors can be introduced into host cells, after growth of infectious particles in packaging cells that are commercially available.

The terms “cells”, “cell cultures”, “cell line”, “recombinant host cells”, “recipient cells” and “host cells” are often used interchangeably and will be clear from the context in which they are used. These terms include the primary subject cells and any progeny thereof, without regard to the number of transfers. It should be understood that not all progeny are exactly identical to the parental cell (due to deliberate or inadvertent mutations or differences in environment), however, such altered progeny are included in these terms, so long as the progeny retain the same functionality as that of the originally transformed cell. For example, though not limited to, such a characteristic might be the ability to produce a particular recombinant protein. A “clone” is a population of cells derived from a single cell or common ancestor by mitosis.

A “reporter gene” refers to a polynucleotide that confers the ability to be specifically detected (or detected and selected), when expressed with a cell of interest. Numerous reporter gene systems are known in the art and include, for example, alkaline phosphatase (Berger, J., et al., Gene 66 1-10 (1988); Kain, S R., Methods Mol. Biol. 63 49-60 (1997)), beta-galactosidase (U.S. Pat. No. 5,070,012), chloramphenicol acetyltransferase (Gorman et al., Mol. Cell. Biol. 2: 1044-51 (1982)), beta glucuronidase, peroxidase, beta lactamase (U.S. Pat. Nos. 5,741,657, 5,955,604), catalytic antibodies, luciferases (U.S. Pat. Nos. 5,221,623; 5,683,888; 5,674,713; 5,650,289; 5,843,746) and naturally fluorescent proteins (Tsien, R Y, Annu. Rev. Biochem. 67 509-544 (1998)). The term “reporter gene”, also includes any peptide which can be specifically detected based on the use of one or more, antibodies, epitopes, binding partners, substrates, modifying enzymes, receptors, or ligands that are capable of, or desired to (or desired not to), interact with the peptide of interest to create a detectable signal. Reporter genes also include genes that can modulate cellular phenotype.

The term “selectable marker gene” as used herein, refers to polynucleotides that allow cells carrying the polynucleotide to be specifically selected for or against, in the presence of a corresponding selective agent. Selectable markers can be positive, negative or bifunctional. Positive selectable markers allow selection for cells carrying the marker, whereas negative selectable markers allow cells carrying the marker to be selectively eliminated. The selectable marker polynucleotide can either be directly linked to the polynucleotides to be expressed, or introduced into the same cell by co-transfection. A variety of such marker polynucleotides have been described, including bifunctional (i.e., positive/negative) markers (see, e.g., WO 92/08796, published May 29, 1992, and WO 94/28143, published Dec. 8, 1994), hereby incorporated in their entirety by reference herein. Specific examples of selectable markers of drug-resistance genes include, but are not limited to, ampicillin, tetracycline, blasticidin, puromycin, hygromycin, ouabain or kanamycin. Specific examples of selectable markers are those, for example, that encode proteins that confer resistance to cytostatic or cytocidal drugs, such as the DHFR protein, which confers resistance to methotrexate (Wigler et al., Proc. Natl. Acad. Sci. USA, 77:3567 (1980); O'Hare et al., Proc. Natl. Acad. Sci. USA, 78:1527 (1981)); the GPF protein, which confers resistance to mycophenolic acid (Mulligan & Berg, Proc. Natl. Acad. Sci. USA, 78:2072 (1981)), the neomycin resistance marker, which confers resistance to the aminoglycoside G-418 (Colberre-Garapin et al., J. Mol. Biol., 150:1 (1981)); the hygromycin protein, which confers resistance to hygromycin (Santerre et al., Gene, 30:147 (1984)); murine Na+, K+-ATPase alpha subunit, which confers resistance to ouabain (Kent et al., Science, 237:901-903 (1987); and the Zeocin™ resistance marker (available commercially from Invitrogen). In addition, the herpes simplex virus thymidine kinase (Wigler et al., Cell, 11:223 (1977)), hypoxanthine-guanine phosphoribosyltransferase (Szybalska & Szybalski, Proc. Natl. Acad. Sci. USA, 48:2026 (1962)), and adenine phosphoribosyltransferase (Lowy et al., Cell, 22:817 (1980)) can be employed in tk-, hgprt- or aprt-cells, respectively. Glutamine synthetase permits the growth of cells in glutamine(GS)-free media (see, e.g., U.S. Pat. Nos. 5,122,464; 5,770,359; and 5,827,739). Other selectable markers encode, for example, puromycin N-acetyl transferase or adenosine deaminase.

“Homology” or “identity” or “similarity” refers to sequence similarity between two peptides or between two nucleic acid molecules. Homology and identity can each be determined by comparing a position in each sequence which can be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, less than 35% identity, less than 30% identity, or less than 25% identity with a sequence described herein. In comparing two sequences, the absence of residues (amino acids or nucleic acids) or presence of extra residues also decreases the identity and homology/similarity.

The term “homology” describes a mathematically based comparison of sequence similarities which is used to identify genes or proteins with similar functions or motifs. The nucleic acid and protein sequences described herein can be used as a “query sequence” to perform a search against public databases to, for example, identify other family members, related sequences or homologs. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules described herein. BLAST protein searches can be performed with the XBLAST program, score=50, wordlength=3 to obtain amino acid sequences homologous to protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used (See www.ncbi.nlm.nih.gov).

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm can also be used to determine identity.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

Two DNA sequences are “substantially homologous” when at least about 75%, at least about 80%, or at least about 90 or 95%, of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra and Nucleic Acid Hybridization, supra. The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also considered in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined melting temperature (Tm) with washes of higher stringency, if desired.

It should be appreciated that also within the scope of the embodiments presented herein are DNA sequences encoding specific binding members (antibodies) which code for e.g. an antibody having the same amino acid sequence, but which are degenerate. By “degenerate to” is meant that a different three-letter codon is used to specify a particular amino acid. It is well known in the art that the following codons can be used interchangeably to code for each specific amino acid:

| | |
|---|---|
| Phenylalanine (Phe or F) | UUU or UUC |
| Leucine (Leu or L) | UUA or UUG or CUU or CUC |
| | or CUA or CUG |
| Isoleucine (Ile or I) | AUU or AUC or AUA |
| Methionine (Met or M) | AUG |
| Valine (Val or V) | GUU or GUC of GUA or GUG |
| Serine (Ser or S) | UCU or UCC or UCA or UCG |
| | or AGU or AGC |
| Proline (Pro or P) | CCU or CCC or CCA or CCG |
| Threonine (Thr or T) | ACU or ACC or ACA or ACG |
| Alanine (Ala or A) | GCU or GCG or GCA or GCG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | CGU or CGC or CGA or CGG |
| | or AGA or AGG |
| Glycine (Gly or G) | GGU or GGC or GGA or GGG |
| Tryptophan (Trp or W) | UGG |
| Termination codon | UAA (ochre) or UAG (amber) |
| | or UGA (opal) |

It should be understood that the codons specified above are for RNA sequences. The corresponding codons for DNA have a T substituted for U.

Mutations can be made such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A nonconservative change is more likely to alter the structure, activity or function of the resulting protein. Polynucleotides should be considered to include sequences containing conservative changes which do not significantly alter the activity or binding characteristics of the resulting protein.

The following represents one example of various groupings of amino acids:
(a) Amino acids with non-polar R groups: Alanine, Valine, Leucine, Isoleucine, Proline, Phenylalanine, Tryptophan and Methionine.
(b) Amino acids with uncharged polar R groups: Glycine, Serine, Threonine, Cysteine, Tyrosine, Asparagine and Glutamine.
(c) Amino acids with charged polar R groups (negatively charged at Ph 6.0): Aspartic acid, Glutamic acid.
(d) Basic amino acids (positively charged at pH 6.0): Lysine, Arginine and Histidine (at pH 6.0).

Another grouping can be those amino acids with phenyl groups such as, Phenylalanine, Tryptophan and Tyrosine.

Another grouping can be according to molecular weight (i.e., size of R groups):

| | |
|---|---|
| Glycine | 75 |
| Alanine | 89 |
| Serine | 105 |
| Proline | 115 |
| Valine | 117 |
| Threonine | 119 |
| Cysteine | 121 |
| Leucine | 131 |
| Isoleucine | 131 |
| Asparagine | 132 |
| Aspartic acid | 133 |
| Glutamine | 146 |
| Lysine | 146 |
| Glutamic acid | 147 |
| Methionine | 149 |
| Histidine | 155 (at pH 6.0) |
| Phenylalanine | 165 |
| Arginine | 174 |
| Tyrosine | 181 |
| Tryptophan | 204 |

Substitutions include, for example:

(a) Lys for Arg and vice versa such that a positive charge can be maintained;

(b) Glu for Asp and vice versa such that a negative charge can be maintained;

(c) Ser for Thr such that a free —OH can be maintained; and

(d) Gln for Asn such that a free NH₂ can be maintained.

Amino acid substitutions can also be introduced to substitute an amino acid with a particular property. For example, a Cys can be introduced a potential site for disulfide bridges with another Cys. A His can be introduced as a “catalytic” site (i.e., His can act as an acid or base and is the most common amino acid in biochemical catalysis). Pro can be introduced because of its planar structure, which induces β-turns in the protein's structure. Alternatively, un-natural amino acids can be incorporated into proteins to facilitate the chemical conjugation to other proteins, toxins, small organic compounds or anti-cancer agents (Datta et al., J Am Chem. Soc. 2002 May 22; 124(20):5652-3).

Two amino acid sequences are “substantially homologous” when at least about 70%, at least about 80%, or at least about 90 or 95%, of the amino acid residues are identical, or represent conservative substitutions.

C. Phage Display Terminology

“Phage display” is a technique by which variant polypeptides are displayed as fusion proteins to at least a portion of a coat protein on the surface of phage, e.g., filamentous phage, particles. A utility of phage display lies in the fact that large libraries of randomized protein variants can be rapidly and efficiently sorted for those sequences that bind to a target molecule with high affinity. Display of peptide and protein libraries on phage has been used for screening millions of polypeptides for ones with specific binding properties. Polyvalent phage display methods have been used for displaying small random peptides and small proteins through fusions to either gene III or gene VIII of filamentous phage (Wells and Lowman, Curr. Opin. Struct. Biol., 3:355-362 (1992), and references cited therein). In monovalent phage display, a protein or peptide library is fused to a gene III or a portion thereof, and expressed at low levels in the presence of wild type gene III protein so that phage particles display one copy or none of the fusion proteins. Avidity effects are reduced relative to polyvalent phage so that sorting is on the basis of intrinsic ligand affinity, and phagemid vectors are used, which simplify DNA manipulations. Lowman and Wells, Methods: A companion to Methods in Enzymology, 3:205-0216 (1991).

A “phagemid” is a plasmid vector having a bacterial origin of replication, e.g., Co1E1, and a copy of an intergenic region of a bacteriophage. Phagemids can be used on any known bacteriophage, including filamentous bacteriophage and lambdoid bacteriophage. Generally, the plasmid will also contain a selectable marker for antibiotic resistance. Segments of DNA cloned into these vectors can be propagated as plasmids. When cells harboring these vectors are provided with all genes necessary for the production of phage particles, the mode of replication of the plasmid changes to rolling circle replication to generate copies of one strand of the plasmid DNA and package phage particles. The phagemid can form infectious or non-infectious phage particles. This term includes phagemids, which contain a phage coat protein gene or fragment thereof linked to a heterologous polypeptide gene as a gene fusion such that the heterologous polypeptide is displayed on the surface of the phage particle.

The term “phage vector” means a double stranded replicative form of a bacteriophage containing a heterologous gene and capable of replication. The phage vector has a phage origin of replication allowing phage replication and phage particle formation. The phage can be a filamentous bacteriophage, such as an M13, fl, fd, Pf3 phage or a derivative thereof, or a lambdoidphage, such as lambda, 21, phi80, phi81, 82, 424, 434, etc., or a derivative thereof.

The term “coat protein” means a protein, at least a portion of which is present on the surface of the virus particle. From a functional perspective, a coat protein is any protein, which associates with a virus particle during the viral assembly process in a host cell, and remains associated with the assembled virus until it infects another host cell. The coat protein can be a major coat protein or a minor coat protein. A “major” coat protein is generally a coat protein which is present in the viral coat at, at least about 5, at least about 7, at least about 10 copies of the protein or more. A major coat protein can be present in tens, hundreds or even thousands of copies per virion. One non-limiting example of a major coat protein is the p8 protein of filamentous phage.

A “fusion protein” and a “fusion polypeptide” refer to a polypeptide having two portions covalently linked together, where each of the portions is a polypeptide having a different property. The property can be a biological property, such as activity in vitro or in vivo. The property can also be a simple chemical or physical property, such as binding to a target molecule, catalysis of a reaction, etc. The two portions can be linked directly by a single peptide bond or through a peptide linker containing one or more amino acid residues. Generally, the two portions and the linker will be in reading frame with each other.

III. Compounds and Compositions and Related Methods

A. Methods of Identifying Cross-Reactive Polypeptides

Tumor necrosis factor receptor (TNFR)-related protein Receptor Activator of NF-κB (RANK) and RANK Ligand (RANKL) function as a cytokine/receptor pair that interact to initiate osteoclast differentiation and activation during bone remodeling, in both normal and disease processes (Boyle et al. Nature, 423: 337-342, 2003).

The deduced crystal structure of mouse RANKL (Lam et al., United States Patent Application Publication Number 20030050223; Lam et al (2001) J Clin Invest 108(7):971-9) has been determined, and the amino acid sequences involved in RANKL binding and activation of RANK on the surface of osteoclast precursors and mature osteoclasts, and which induce osteoclast differentiation and activation have been identified. The amino acid sequences correspond to ectodomains of the RANKL polypeptide that are formed by association of interdispersed β-pleated sheets, and extend from the core RANKL structure as peptide loops. The loop regions are located in precise regions of the primary amino acid sequence of RANKL, and can be directly mapped using the crystal structure coordinates. These sequences of the mouse polypeptide could serve as targets for antibodies and selective binding proteins that block or neutralize RANKL activity. A human monoclonal antibody to human RANKL has neutralizing activity in vivo and can be administered to reduce bone resorption in post menopausal women (Bekker et al., J. Bone Miner. Res. July; 19(7):1059-66, 2004). Certain anti-RANKL antibodies isolated using human RANKL polypeptide can bind selectively to human, but not murine RANKL (Desphande et al., US Patent Application Publication Number 20030103978).

RANKL inhibitors block osteoclast differentiation and activation in vitro and in vivo, and can block the pathological loss of bone in disease models that mimic osteoporosis, hypercalcemia of malignancy and bone metastasis, rheumatoid arthritis, steroid induced bone loss, and bone loss due to disuse and skeletal unloading. Recombinant RANKL inhibitors can be engineered from the ligand binding domains of Osteoprotegerin (OPG) and RANK, and from biologically active anti-RANKL neutralizing monoclonal antibodies.

Inhibitors of RANKL, including AMG162 (Denosumab) currently in clinical development, block osteoclast maturation and are thought to have desired therapeutic properties in disease areas such as osteoporosis, hypercalcemia associated with bone metastasis, rheumatoid arthritis, and other disorders associated with bone remodeling. Denosumab, a human anti-RANKL antibody, has been shown impact bone resorption in early clinical trails assessing its efficacy in treating osteoporosis (Schwartz and Ritchlin, (2007) Arthritis Res. Ther. 9(Supp 1): S7). Denosumab has been shown to bind and neutralizes human RANKL, but not mouse RANKL.

Provided herein is a method of identifying species cross-reactive binding sequences, (e.g., to two or more different species of RANKL). Further, provided herein are neutralizing antibodies, antigen-binding fragments and binding proteins that have use in prevention, and/or treatment, of animal diseases associated with cross-reactive binding sequence (e.g., a RANKL cross-reactive binding sequence) binding and/or function.

Provided herein is the identification of one or more binding surfaces of RANKL and determinants of binding specificity across one or more species. An understanding of this interaction leads directly to peptide and protein epitopes which, when used in the selection of antibodies, antigen-binding fragments, and binding proteins can be used to generate inter-species antibodies, antigen-binding fragments, and binding proteins and/or neutralizing antibodies, antigen-binding fragments, and binding proteins.

RANKL belongs to a family of TNF (tumor necrosis factor) receptor/ligand pairs, which function in numerous physiologic processes and whose receptor and ligand sequences share similarity to one another. The x-ray crystal structure of mouse extracellular domain of RANKL has been solved (as a physiologic homotrimer), as have other TNF/TNF ligand complexes, including the TNFβ/TNFR1 complex, the TRAIL-DR5 complex, and the CD40 neutralizing antibody complex (Banner et al. Cell., 73(3): 431-445 (1993), Hymowitz et al. Mol. Cell., 4(4): 563-571 (1999), Karpusas et al. Structure, 9(4):321-329 (2001).

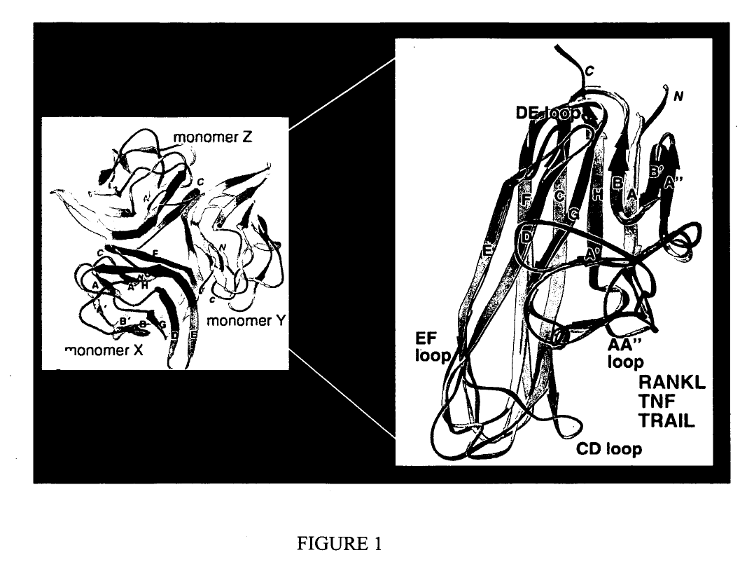

Within the RANKL family, seven (7) RANKL proteins have been sequenced in various organisms (see, e.g., FIG. 1). Together, these data provide insights into those portions of the RANKL sequence that mediate binding to RANK and which, are involved in the identification of epitopes/cross-reactive binding sequences and binding regions for neutralizing antibodies, antigen-binding fragments, and binding proteins.

RANKL cross-reactive binding sequences can be obtained by rational design for in vitro and in vivo uses such as, for example, diagnosis, detection, monitoring and/or therapy. In one non-limiting rational design procedure, the three-dimensional structure of a RANKL polypeptide can be analyzed by, for example, by analyzing the nucleic acid sequence encoding the polypeptide or the amino acid sequence making up the RANKL polypeptide. The three-dimensional structure and/or sequences can then be used to predict structures of inter-species and/or intra-species cross-reactive binding sequences by, for example, art-recognized computer modeling. The predicted structures can then be produced by, for example, chemical synthesis or recombinant DNA technology. In one aspect, RANKL cross-species reactive binding sequences can be modeled on an unconstrained polypeptide chain. Where the structure of cross-reactive binding sequences is unconstrained, the desired structure can be modeled without respect to NMR or X-ray crystallography, and any of a number of art-recognized programs for modeling short polypeptide chains can be employed as described elsewhere herein and known in the art.

RANKL cross-reactive binding sequences can be evaluated for their ability to be bound by a specific binding member. Provided herein are cross-reactive binding sequences (linear or conformational) found across one or more species (i.e., inter-species) of RANKL which can be used to induce an immune response, diagnose, detect, monitor, and/or treat a condition, disease or disorder associated with RANKL. The cross-reactive binding sequences can be linear, conformational, surface, cryptic and/or cyclic. Also provided herein are methods for synthesizing cross-reactive RANKL binding sequences. In one aspect, cross-reactive binding sequences can be used as immunogens to induce an immune response. RANKL cross-reactive binding sequences can be, for example, used to generate monoclonal antibodies, which monoclonal antibodies can be administered to a subject in need thereof. Species cross-reactive binding sequences can also be administered to a subject to elicit an immune response thereto. Immune responses include, but are not limited to, generation of antibodies, initiation of T helper responses and/or initiation of a cytotoxic T lymphocyte reaction. In some instances, the cross-reactive binding sequence is linked to another moiety (e.g., keyhole limpet hemocyanin (KLH)) to render it more visible the immune system (i.e., more immunogenic).

Species cross-reactive binding sequences can be synthesized using techniques that are known to those skilled in the art, including polypeptide and recombinant DNA techniques. Synthetic chemistry techniques, such as a solid-phase Merrifield-type synthesis, can be advantageous since it produces products having high purity, antigenic specificity, freedom from undesired side products, ease of production and the like. Summaries of the some techniques available can be found in, e.g., Steward et al., “Solid Phase Peptide Synthesis,” W. H. Freeman Co., San Francisco, 1969; Bodanszky, et al., “Peptide Synthesis,” John Wiley & Sons, Second Edition, 1976; J. Meienhofer, “Hormonal Proteins and Peptides,” Vol. 2, p. 46, Academic Press (New York), 1983; Merrifield, Adv. Enzymol. 1969, 32:221-96; Fields et al., Int. J. Peptide Protein Res. 1990, 35:161-214; U.S. Pat. No. 4,244,946 for solid phase peptide synthesis; and Schroder et al., “The Peptides,” Vol. 1, Academic Press (New York), 1965 (for classical solution synthesis). Such synthesis can utilize appropriate protective groups which are described in J. F. W. McOmie, “Protective Groups in Organic Chemistry,” Plenum Press, New York, 1973.

In addition, a species cross-reactive binding sequence useful in the methods described herein can be prepared without including a free ionic salt in which the charged acid or base groups present in the amino acid residue side groups (e.g., Arg, Asp, and the like) associate and neutralize each other to form an “inner salt” compound.

Solid-phase synthesis methods generally comprise the sequential addition of one or more amino acid residues or suitably protected amino acid residues to a growing peptide chain. Normally, either the amino or carboxyl group of the first amino acid residue is protected by a suitable, selectively removable protecting group. For amino acids containing a reactive side group (e.g., lysine), a different, selectively removable protecting group is utilized.

In solid phase synthesis, the protected or derivatized amino acid is attached to an inert solid support through its unprotected carboxyl or amino group. The protecting group of the amino or carboxyl group is then selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group is then removed from this newly added amino acid residue, and the next suitably protected amino acid is then added, and so forth. After all the desired amino acids have been linked in the proper sequence, any remaining terminal and side group protecting groups (and solid support) are removed sequentially or concurrently, to afford the final linear polypeptide.

Linear polypeptides can be reacted to form their corresponding cyclic peptides. A method for preparing a cyclic peptide is described by Zimmer et al., Peptides 1992, pp. 393-394, ESCOM Science Publishers, B.V., 1993. Typically, tertbutoxycarbonyl protected peptide methyl ester is dissolved in methanol, sodium hydroxide solution is added, and the admixture is reacted at 20° C. to hydrolytically remove the methyl ester protecting group. After evaporating the solvent, the tertbutoxycarbonyl protected peptide is extracted with ethyl acetate from acidified aqueous solvent. The tertbutoxycarbonyl protecting group is then removed under mildly acidic conditions in dioxane cosolvent. The unprotected linear peptide with free amino and carboxy termini so obtained is converted to its corresponding cyclic peptide by reacting a dilute solution of the linear peptide, in a mixture of dichloromethane and dimethylformamide, with dicyclohexylcarbodiimide in the presence of 1-hydroxybenzotriazole and N-methylmorpholine. The resultant cyclic peptide is then purified by chromatography. Cyclic peptide synthesis can be achieved by alternative methods as described by Gurrath et al., Eur. J. Biochem. 1992, 210:911-921.

Polypeptides described herein, for the most part, can be made in transformed host cells using art-recognized recombinant DNA techniques. Briefly, a recombinant DNA molecule coding for the polypeptide is prepared. Methods of preparing such DNA molecules are well known in the art. Reference works on the general principles of recombinant DNA Technology include Watson et al., Molecular Biology of the Gene, Volumes I and II, The Benjamin/Cummings Publishing Company, Inc., Menlo Park, Calif. (1987); Darnell et al., Molecular Cell Biology, Scientific American Books, Inc., New York, N.Y. (1986); Lewin, Genes II, John Wiley & Sons, New York, N.Y. (1985); Old, et al., Principles of Gene Manipulation: An Introduction to Genetic Engineering, 2 k edition, University of California Press, Berkeley, Calif. (1981); Sambrook et al., Molecular Cloning, A Laboratory Manual, Cold Spring Harbor Laboratory Press, Cold Spring Harbor, N.Y. (1989); and Ausubel et al, Current Protocols in Molecular Biology, Wiley Interscience, New York, (1987, 1992). These references are herein entirely incorporated by reference as are the references cited therein. For example, polynucleotide sequences coding for polypeptides can be excised from DNA using suitable restriction enzymes. Alternatively, the DNA molecule can be synthesized using chemical synthesis techniques, such as the phosphoramidate method. Also, a combination of these techniques can be used.

Provided herein are compositions of a RANKL cross-reactive binding sequence and an acceptable carrier or excipient. In one aspect, the present application also provides for an acceptable salt of any of the RANKL species cross-reactive binding sequence provided herein.

Provided herein are cross-reactive binding sequences which are epitopes present in RANKL polypeptides found in one or more species. In another aspect, a cross-reactive binding sequence can be an epitope that is found, for example, in two or more (e.g., human and mouse) species of RANKL. In one non-limiting example of an inter-species cross-reactive binding sequence, the RANKL cross-reactive binding sequence can be an epitope that is found in two or more species such as, for example, two or more of dog, cat, rat, horse, cow, primate (e.g., gorilla, ape, chimpanzee, monkey), fish, shellfish, frog, sheep, poultry (e.g., chickens, turkeys, etc), etc.

B. RANKL Cross-Reactive Binding Sequences

RANKL cross-reactive binding sequences can, optionally, further be conjugated to a therapeutic moiety, a detectable moiety, an affinity tag (e.g., a purification tag) and/or can be a fusion protein as described elsewhere herein.

RANKL cross-reactive binding sequences can be obtained by rational design for in vitro and in vivo uses such as, for example, diagnosis, detection, monitoring and/or therapy. In one non-limiting rational design procedure, the three-dimensional structure of a RANKL can be analyzed by, for example, by analyzing the nucleic acid sequence encoding the polypeptide or the amino acid sequence making up the polypeptide. The three-dimensional structure and/or sequences can then be used to predict structures of inter-species cross-reactive binding sequences by, for example, art-recognized computer modeling. The predicted structures can then be produced by, for example, chemical synthesis or recombinant DNA technology. In one aspect, RANKL cross-reactive binding sequences can be modeled on an unconstrained polypeptide chain. Where the structure of cross-reactive binding sequences is unconstrained, the desired structure can be modeled without respect to NMR or X-ray crystallography, and any of a number of art-recognized programs for modeling short polypeptide chains can be employed.

RANKL cross-reactive binding sequences can be evaluated for their ability to be bound by an antibody or antigen-binding fragment thereof. Provided herein are RANKL cross-reactive binding sequences (linear or conformational) found across one or more species (i.e., intra-species and/or inter-species), of RANKL which can be used to induce an immune response, diagnose, detect, monitor, and/or treat a condition, disease or disorder associated with RANKL.

Provided herein is an isolated polypeptide consisting essentially of the epitope comprising residues 119-318 of mouse RANKL forms another aspect of the present invention. An isolated polypeptide consisting essentially of the epitope comprising residues 119-318 of non-human and non-mouse mammalian RANKL forms another aspect of the present invention, including the sequence of a rat, a canine or a cross-reactive sequence.

In one aspect, encompassed herein are isolated polypeptides comprising an amino acid sequence of a RANKL peptide, including a murine or other non-human RANKL peptide, consisting essentially of an amino acid sequence of any of SEQ ID NOS: 1, 2, 3, 5, 6, 7, 9, 10, 11, 13, 14, 15 or 17 or a RANKL peptide of any of SEQ ID NOS: 18-43. The isolated peptides, including combinations of one or more thereof, are suitable for use in generating antibodies which recognize RANKL and have anti-RANKL/RANK activity and are useful in modulating bone remodeling, osteoclast differentiation and bone disease.

The present application is directed to an isolated receptor polypeptide which consist essentially of the amino acid sequence of any of SEQ ID NOS: 1, 2, 3, 5, 6, 7, 9, 10, 11, 13, 14, 15, 17 or any one of SEQ ID NOS: 18-43. In one aspect, the application is directed to an isolated receptor peptide which consists essentially of a non-murine and non-human RANKL amino acid sequence of any of SEQ ID NOS: 2, 3, 6, 7, 10, 11, 14, 15 or any of SEQ ID NOS: 18-43.

The application provides isolated RANKL loop peptides, including novel cross-reactive loop peptides and peptides having amino acid sequences taken from one or more mammalian species. These RANKL loop peptides are capable of generating and raising anti-RANKL antibodies which recognize, bind to and neutralize RANKL from more than one mammalian species (e.g., mouse and human RANKL).

Exemplary A-A′ loop based peptides are provided herein. The application provides an isolated peptide having an amino acid sequence X1 X2 X3 X4 X5 X6 X7 X8 X9 X10 X11 X12 X13 X14 X15 X16 W X17 X18 X19 X20 X21 X22 X23, wherein each residue can be independently selected as follows (SEQ ID NO: 18):

X1 is N, R, D, G or S;

X2 is A, D, or S;

X3 is A, Q, I, K or T;

X4 is S, N, D, Q or T;

X5 is I, A, R, N, D, Q, E, G, L, K, M, F, P, S, T or V;

X6 is P, N or Y;

X7 is S or T;

X8 is G, N or Q;

X9 is S, E or T;

X10 is H, R, C, E or P;

X11 is K, R, or N;

X12 is V, A or H;

X13 is T, N, P, S or V;

X14 is L, T or V;

X15 is S, H, I or T;

X16 is S, C, I or T;

X17 is Y, A, N, D or H;

X18 is H, N, Q or F;

X19 is D, R, or K;

X20 is R, E, H, K or S;

X21 is G or F;

X22 is W, Q, G or F;

X23 is A or C.

The application provides an isolated peptide having an amino acid sequence N A X1 X2 I P S G S H K V X3 L S S W Y H D R G W A, wherein each Xn residue is selected as follows (SEQ ID NO: 19):

X1 is A or T;

X2 is S or D; and

X3 is T or S.

Exemplary C-D loop based peptides are provided herein. The application provides an isolated peptide having an amino acid sequence X1 X2 X3 X4 X5 X6 X7 X8 X9 X10 X11 X12, wherein each residue can be independently selected as follows (SEQ ID NO:20):

X1 is H, P or W;

X2 is E, I, L or S;

X3 is T, L, P or V;

X4 is S, Q or K;

X5 is G, A or N;

X6 is S, A, N, D or K;

X7 is V, D, E or L;

X8 is P, A, Q, S, T or Y;

X9 is T, A, R, K or S;

X10 is D, R, N, E or S;

X11 is Y, A, N, G or V; and

X12 is L, T or H.

The application provides an isolated peptide having an amino acid sequence H E T S G X1 X2 X3 X4 X5 Y L, wherein each Xn residue is selected as follows (SEQ ID NO:21):

X1 is S or D;

X2 is V or L;

X3 is P or A;

X4 is T or A; and

X5 is D or E.

Exemplary D-E loop based peptides are provided herein. The application provides an isolated peptide having an amino acid sequence X1 X2 X3 X4 X5 X6 X7 X8, wherein each residue can be independently selected as follows (SEQ ID NO:22):

X1 is T, A, E or K;

X2 is S, N, K or T;

X3 is I, L, K or T;

X4 is K, N or S;

X5 is I, R, Q or Y;

X6 is P, R, N or L;

X7 is S, R, N or P; and

X8 is S, K, P or Y.

The application provides an isolated peptide having an amino acid sequence T S I K I X1 S S, wherein X1 is selected from P, R or K (SEQ ID NO:23).

Exemplary E-F loop based peptides are provided herein. The application provides an isolated peptide having an amino acid sequence X1 X2 W X3 X4 X5 X6 X7 X8, wherein each residue can be independently selected as follows (SEQ ID NO:24):

X1 is K, A or M;

X2 is N, R, C, I or Y;

X3 is S, A, N or L;

X4 is G, A R, N, D, Q, E, I, L, F, K, P, S, T, Y or V;

X5 is Nor D;

X6 is S, A or T;

X7 is E, S or V; and

X8 is F or Y.

The application provides an isolated peptide having an amino acid sequence K X1 W S G N S E F, wherein X1 is selected from N or Y (SEQ ID NO:25).

The application also provides isolated peptides having the amino acid sequences as set forth above in Table 1 and provided herein as follows:

TABLE 1

| | |
|---|---|
| Amino Acid Sequence | SEQ ID NO: |

| | |
|---|---|
| LTINAASIPSG | SEQ ID NO:26 |
| SHKVTLSSWYDHRGWG | SEQ ID NO:27 |
| SGSVPTDYLQLMVY | SEQ ID NO:28 |
| QVSNPSLLDPDQ | SEQ ID NO:29 |
| EVSNPSLLDPDQ | SEQ ID NO:30 |
| SGSLPTDYLQLMVY | SEQ ID NO:31 |
| SGDVPTEYLQLMVT | SEQ ID NO:32 |
| SGSVPAEYLQLMVY | SEQ ID NO:33 |
| SGSVATDYLQLMVY | SEQ ID NO:34 |
| SGDVATEYLQLMVT | SEQ ID NO:35 |
| SGSVAADYLQLMVY | SEQ ID NO:36 |
| SGSVAAEYLQLMVY | SEQ ID NO:37 |
| SGDLPTEYLQLMVT | SEQ ID NO:38 |
| SGSLPADYLQLMVY | SEQ ID NO:39 |
| SGSLPAEYLQLMVY | SEQ ID NO:40 |
| SGSLATDYLQLMVY | SEQ ID NO:41 |
| SGSLAADYLQLMVY | SEQ ID NO:42 |
| SGSLAAEYLQLMVY | SEQ ID NO:43 |

The present application further provides an isolated nucleic acid encoding a RANKL polypeptide consisting essentially of an amino acid sequence such as, for example, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26 (LTINAASIPSG), SEQ ID NO: 27 (SHKVTLSSWYDHRGWG), SEQ ID NO: 28 (SGSVPTDYLQLMVY), SEQ ID NO: 29 (QVSNPSLLDPDQ), SEQ ID NO: 30 (EVSNPSLLDPDQ), SEQ ID NO: 31 (SGSLPTDYLQLMVY), SEQ ID NO: 32 (SGDVPTEYLQLMVT), SEQ ID NO: 33 (SGSVPAEYLQLMVY), SEQ ID NO: 34 (SGSVATDYLQLMVY), SEQ ID NO: 35 (SGDVATEYLQLMVT), SEQ ID NO: 36 (SGSVAADYLQLMVY), SEQ ID NO: 37 (SGSVAAEYLQLMVY), SEQ ID NO: 38 (SGDLPTEYLQLMVT), SEQ ID NO: 39 (SGSLPADYLQLMVY), SEQ ID NO: 40 (SGSLPAEYLQLMVY), SEQ ID NO: 41 (SGSLATDYLQLMVY), SEQ ID NO: 42 (SGSLAADYLQLMVY) or SEQ ID NO: 43 (SGSLAAEYLQLMVY).

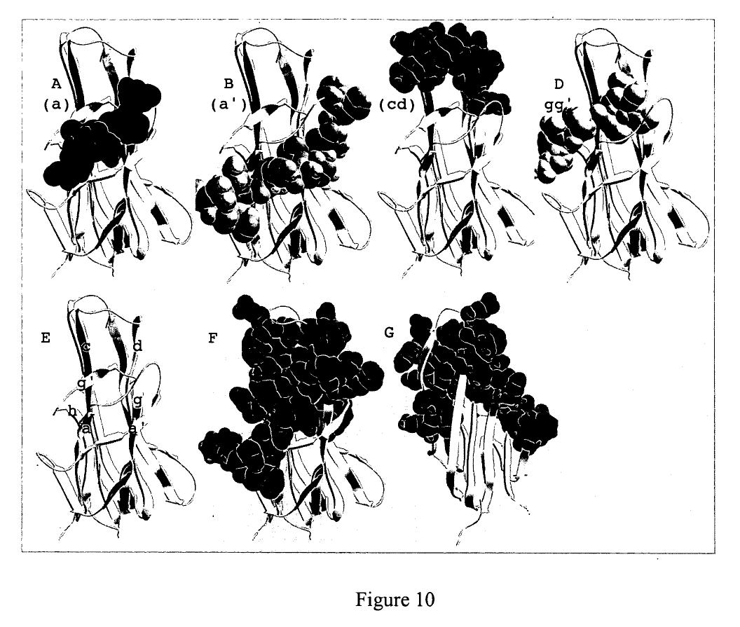

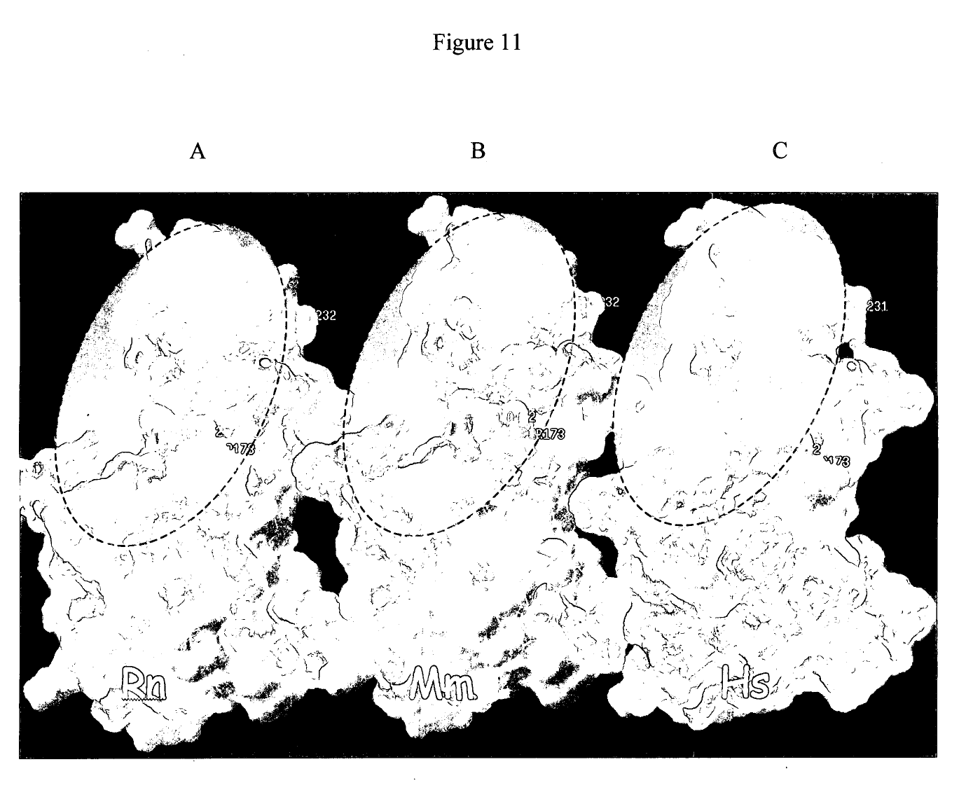

In one non-limiting aspect, a RANKL cross-reactive binding sequence is, for example, a cross-reactive binding sequence as illustrated in FIGS. 10A-G. In another non-limiting aspect, a RANKL cross-reactive binding sequence is, for example, a binding sequence as set forth in Table 3.

TABLE 3

| | | |
|---|---|---|
| | | Residue |
| Species | Sequence | Nos. |

Family A

| | | |
|---|---|---|
| Mus musculus | LTINAASIPSG | 6-16 |
| Rattus norvegicus | LTINAADIPSG | 6-16 |
| Homo sapiens | LTINAADIPSG | 6-16 |
| Cross-reactive | LTINAADIPSG | 6-16 |
| binding sequence | | |
| Family B | | |
| Bos taurus | SHKVTLSSWYDHRGWG | 17-32 |
| Family C | | |
| Mus musculus | SGSVPTDYLQLMVY | 66-79 |
| Cross-reactive | SGSVPTDYLQLMVY | (1) 66-79 |
| binding sequence | | |
| Rattus norvegicus | SGSVPTDYLQLMVY | 66-79 |
| Cross-reactive | SGSVPAEYLQLMVY | (2) 66-79 |
| binding sequence | | |
| Cross-reactive | SGSVATDYLQLMVY | (3) 66-79 |
| sequence | | |
| Cross-reactive | SGDVATEYLQLMVT | (4) 66-79 |
| binding sequence | | |
| Cross-reactive | SGSVAADYLQLMVY | (5) 66-79 |
| binding sequence | | |
| Cross-reactive | SGSVAAEYLQLMVY | (6) 66-79 |
| binding sequence | | |
| Cross-reactive | SGSLPTDYLQLMVY | (7) 66-79 |
| binding sequence | | |
| Cross-reactive | SGDLPTEYLQLMVT | (8) 66-79 |
| binding sequence | | |
| Cross-reactive | SGSLPADYLQLMVY | (9) 66-79 |
| binding sequence | | |
| Cross-reactive | SGSLPAEYLQLMVY | (10) 66-79 |
| binding sequence | | |
| Cross-reactive | SGSLATDVLQLMVY | (11)66-79 |
| binding sequence | | |
| Homo sapiens | SGSVPTDYLQLMVY | 66-79 |
| Cross-reactive | SGSLAADYLQLMVY | (12) 66-79 |
| binding sequence | | |
| Cross-reactive | SGSLAAEYLQLMVY | (13) 66-79 |
| binding sequence | | |
| Family D | | |
| Mus musculus | QVSNPSLLDPDQ | 130-141 |
| Homo sapiens | EVSNPSLLDPDQ | 130-141 |

Families A-D reference the peptide family of origin, see FIG. 10; the peptide sequence is given, with binding residues in bold. The set of parentheses (#) with respect to the residue numbers refer to the cross-reactive binding sequence variant for that family of peptides; the residue numbers indicated describe the sequence range of the peptide numbered from the first residue of the extracellular domains first beta-strand.

In one aspect, an isolated RANKL cross-reactive binding sequence comprises an amino acid sequence set forth as, for example, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43, wherein said peptide is about 10 to 60, from about 10 to about 55, from about 10 to about 50, from about 10 to about 45, from about 10 to about 40, from about 10 to about 35, from about 10 to about 30, from about 10 to about 25, from about 10 to about 20, or from 10 to about 15, amino acid residues in length.

In one embodiment, the RANKL cross-reactive binding sequence is a human and mouse RANKL cross-reactive binding sequence such as, but not limited to, an amino acid sequence set forth in amino acid residues 2-12 of SEQ ID NO: 29 or 30.

In one aspect, a RANKL cross-reactive binding sequence consists essentially of, or consisting of, a peptide having an amino acid sequence set forth as, for example, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43.

In one embodiment, the RANKL cross-reactive binding sequence is a human and mouse RANKL cross-reactive binding sequence such as, but not limited to, an amino acid sequence set forth in amino acid residues 2-12 of SEQ ID NO: 29 and 30.

A RANKL cross-reactive binding sequence can further comprise a detectable moiety. A RANKL cross-reactive binding sequence can further comprise an affinity tag.

As used herein, a polypeptide or peptide consisting essentially of, or consisting of, a RANKL cross-reactive binding sequence contains the sequence identified and can, optionally, include any one of 1, 2, 3, 4, 5, 6, 7, 8, 9, 10, or up to 30 additional RANKL amino acid residues on either one or both of the 5′ and 3′ ends of the polypeptide or peptide, capping, etc. Amino acid sequences for RANKL polypeptides have been described elsewhere herein.

In other embodiments, the present application provides an isolated cross-reactive RANKL polypeptide consisting essentially of, or consisting of, an amino acid sequence set forth as, for example, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43. In certain embodiments, the polypeptides of the application are useful in diagnostic assays or kits.

The application provides RANKL epitopes which can be utilized in generating antibodies which modulate bone remodeling, osteoclast differentiation and/or bone disease. The epitopes include loops that are exposed on the protein surface and form receptor contacts. These loop epitopes are capable of generating antibodies which bind or interact at the RANK/RANKL interface and neutralize RANKL, thereby modulating, including preventing or inhibiting RANK activity, receptor activation and signaling. The application provides RANKL epitopes including, but not limited to, RANKL loop epitopes, which can be utilized in generating antibodies which are potent inhibitors of RANKL activity in vivo. The anti-RANKL antibodies so generated are immunoreactive with RANKL from multiple species, e.g., with human and mouse RANKL.

In accordance with the present application, RANKL peptides are provided which are capable of generating antibodies, such as monoclonal antibodies, when injected into a subject, which antibodies can, for example, inhibit RANKL activity and, modulate bone remodeling, osteoclast differentiation and bone disease.

C. Binding Members

Provided herein are antibodies, antigen-binding fragments and binding proteins that bind to a RANKL cross-reactive binding sequence. As used herein, “antibody,” “antigen-binding fragment,” and “binding protein” are used interchangeably. Antibodies, antigen-binding fragments and binding proteins that bind to a RANKL cross-reactive binding sequence and can inhibit (partially or fully) or manage/treat (partially or fully) symptoms associated with a bone loss disease or disorder. The RANKL cross-reactive binding sequence polypeptide can contain linear and/or conformational cross-reactive binding sequences. The cross-reactive binding sequences can be located on surface exposed domains or can be cryptic cross-reactive binding sequences. In one aspect, an antibody, antigen-binding fragment or binding protein binds to a cross-reactive binding sequence on native RANKL. In another aspect, the antibody, antigen-binding fragment or binding protein binds to a portion of RANKL containing a cross-reactive binding sequence. The application also describes cell lines which produce the antibodies, methods for producing the cell lines, methods for producing monoclonal antibodies, and methods for producing antigen-binding fragments and binding proteins.

As used herein, “immunoreactive” refers to binding agents (members), antibodies or fragments thereof that are specific to a sequence of amino acid residues (“binding site” or “epitope”); and, if cross-reactive to other peptides/proteins, are not toxic at the levels at which they are formulated for administration to human use. As used herein, “polyspecific” refers to binding agents, antibodies or fragments thereof that are specific to a sequence of amino acid residues comprising a “composite” or “hybrid” and which are cross-reactive among one or more species or one or more family members of a polypeptide. The terms “preferentially binds” or “specifically binds” mean that the binding agents, antibodies or fragments thereof bind to the binding site with greater affinity than it binds unrelated amino acid sequences. In one aspect, such affinity is at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the antibody or fragment thereof for unrelated amino acid sequences. The terms “immunoreactive,” “binds,” “preferentially binds” and “specifically binds” are used interchangeably herein.

The term “antibody” also includes molecules which have been engineered through the use of molecular biological technique to include only portions of the native molecule as long as those molecules have the ability to bind a particular antigen or sequence of amino acids. Such alternative antibody molecules include classically known portions of the antibody molecules, single chain antibodies and single chain binding molecules.

An “antibody combining site” is that structural portion of an antibody molecule comprised of heavy and light chain variable and hypervariable regions that specifically binds antigen. Antibody combining sites include both framework and complementarity determining regions (CDRs) on heavy and light chain variable region polypeptides.

Exemplary antibodies for use in the compositions and methods described herein are intact immunoglobulin molecules, such as, for example, a polyclonal antibody, a monoclonal antibody, a chimeric antibody, a humanized antibody, a human antibody, an engineered antibody, a Camel immunoglobulin, an NAR immunoglobulin, an IgG antibody, a minibody, a diabody, a triabody and a tetrabody, as well as substantially intact immunoglobulin molecules and those portions of an immunoglobulin molecule that contain the antigen binding site (i.e., paratope), including those portions known in the art as Fab, Fab′, F(ab)′, F(ab′)₂, Fd, scFv, a variable heavy domain, a variable light domain, a variable NAR domain, bi-specific scFv, a bi-specific Fab₂, a tri-specific Fab₃ and a single chain binding polypeptides and others also referred to as antigen-binding fragments.

In one exemplary embodiment, the application contemplates a single chain binding polypeptide having a heavy chain variable region, and/or a light chain variable region and, optionally, an immunoglobulin Fc region. Such a molecule is a single chain variable fragment optionally having effector function through the presence of the immunoglobulin Fc region. Methods of preparing single chain binding polypeptides are known in the art (e.g., US. Patent Application 2005/0238646).

In another exemplary embodiment, the application contemplates a truncated immunoglobulin molecule comprising a Fab fragment derived from a monoclonal antibody. The Fab fragment, lacking Fc receptor, is soluble, and affords therapeutic advantages in serum half life, and diagnostic advantages in modes of using the soluble Fab fragment. The preparation of a soluble Fab fragment is generally known in the immunological arts and can be accomplished by a variety of methods.

For example, Fab and F(ab′)₂ portions (fragments) of antibodies are prepared by the proteolytic reaction of papain and pepsin, respectively, on substantially intact antibodies by methods that are well known. See for example, U.S. Pat. No. 4,342,566 by Theofilopolous and Dixon. Fab′ antibody portions also are well known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact immunoglobulin molecules are utilized as illustrative herein.

In one aspect, antibodies, antigen-binding fragments and binding proteins bind to cross-reactive binding sequences of RANKL. Such antibodies include a polyclonal antibody, a monoclonal antibody, a chimeric antibody, a humanized antibody, a human antibody, an engineered antibody, a Camel immunoglobulin, an NAR immunoglobulin, an IgG antibody, a minibody, a diabody, a triabody and a tetrabody and the like as provided above. Also provided herein are functional fragments of such antibodies including, but not limited to, Fab, Fab′, F(ab)′, F(ab′)₂, Fd, scFv, a variable heavy domain, a variable light domain, a variable NAR domain, bi-specific scFv, a bi-specific Fab₂, a tri-specific Fab₃ a single chain binding polypeptide and the like. Also provided herein are binding proteins such as Avimers™. One of ordinary skill in the art would recognize that antibodies, antigen-binding fragments and binding proteins generated against cross-reactive binding sequences could be tested using the assays provided herein for the ability to bind to isolated cross-reactive RANKL binding sequences.

Provided herein are antibodies, antigen-binding fragments, and binding proteins that bind to a RANKL cross-reactive binding sequence across one or more species. The RANKL cross-reactive binding sequence recognized by the antibodies, antigen-binding fragments, and binding proteins can be, for example, a surface exposed (e.g., loop) domain or a cryptic epitope. These antibodies, antigen-binding fragments, and binding proteins can be useful in diagnosis, detecting, monitoring and/or treatment of any condition, disease or disorder associated with the RANKL polypeptides (e.g., bone loss diseases and disorders such as, for example, lytic bone diseases, including osteoporosis, rheumatoid arthritis, hypercalcemia of malignancy, bone metastasis, bone loss associated with steroid administration, and periodontal disease). The antibodies, antigen-binding fragments, and binding proteins can also be used in therapy in combination with one or more other therapeutic procedures, agents or moieties. In one non-limiting example, the one or more other therapeutic procedures can be, for example, in the case of metastatic cancers extending to bone, one or more chemotherapeutic agents, radiation, surgery, anti-CD20 antibodies, etc.

Provided herein are antibodies, antigen-binding fragments, and binding proteins that bind to a RANKL cross-reactive binding sequence. RANKL cross-reactive binding sequences to which the antibodies, antigen-binding fragments, and binding proteins bind are epitopes present in RANKL found across one or more species. In one embodiment, an antibody, antigen-binding fragment or binding protein can bind a cross-reactive binding sequence found in two or more species such as, for example, two or more of dog, cat, rat, horse, cow, primate (e.g., gorilla, ape, chimpanzee, monkey), fish, shellfish, frog, sheep, poultry (e.g., chickens, turkeys, etc), etc.

In certain embodiments, the present application provides an isolated specific binding member, such as an antibody or functional (antigen-binding) fragment thereof, which recognizes a mammalian RANKL epitope which is found in RANKL expressing cells such as osteoblasts, stromal cells, activated T cells, activated endothelium, and mammary epithelium. In further aspects, the present application provides a specific binding member which recognizes a human RANKL epitope which is found in osteoblasts, stromal cells, activated T cells, activated endothelium, and mammary epithelium cells. In a still further aspect, the present application provides a specific binding member which recognizes a murine RANKL epitope which is found in osteoblasts, stromal cells, activated T cells, activated endothelium, and mammary epithelium cells. In one aspect, the murine and human epitopes are conserved, allowing these anti-RANKL antibodies to bind with the same affinity to mouse and human RANKL.

The specific binding member, which can be an antibody or a fragment thereof, such as an immunogenic fragment thereof, binds to both mouse and human RANKL and neutralizes the bioactivity of both mouse and human RANKL.

The present application provides a specific binding member which recognizes an epitope which is found in the mammalian RANKL polypeptide wherein the epitope is conserved in the mouse and human RANKL polypeptide, and the novel specific binding member neutralizes both mouse and human RANKL activity. The application provides a novel specific binding member including immunogenic fragments, which recognizes an epitope which is found in the mouse RANKL polypeptide wherein the epitope is conserved in the human RANKL polypeptide, and the novel specific binding member neutralizes both mouse and human RANKL activity. The specific binding member, such as the antibody or fragment thereof, binds to both human and mouse RANKL sequences.

In one aspect, and as stated above, the present inventors have developed monoclonal antibodies, exemplified herein by a monoclonal antibody that can cross-react and neutralize RANKL from multiple species, which specifically recognize both mouse and human RANKL. In one such aspect, the monoclonal antibody is generated using RANKL peptides or nucleic acids encoding peptides which do not correspond precisely in sequence to either mouse or human RANKL sequences, but wherein the antibodies so generated recognize all of the immunogen, mouse and human RANKL. Additionally, monoclonal antibodies can bind to the extracellular domain of mouse and human RANKL polypeptide immobilized on the surface of ELISA plates, indicating a conformational epitope with a polypeptide aspect. The monoclonal antibodies bind to membrane bound RANKL, and can block cell associated RANKL activity in mouse and human cells.

In one aspect, the antibody is one which has the characteristics of the antibody which the inventors have identified and characterized, e.g., recognizing both mouse and human RANKL with equal affinity and can block the biological activity of RANKL originating from multiple species including mouse and humans. In one aspect, the antibody is a monoclonal antibody (or active fragments thereof) that can cross-react and neutralize RANKL from multiple species. In a further aspect the antibody comprises the V_{H} and V_{L} amino acid sequences of a monoclonal antibody (or active fragments thereof) that can cross-react and neutralize RANKL from multiple species.

In certain aspects, the specific binding member is an antibody, or antigen-binding fragment, that binds to a cross-reactive RANKL polypeptide consisting essentially of, or consisting of, an amino acid sequence set forth as, for example, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43, and wherein the antibody, or antigen-binding fragment, neutralizes the activity of RANKL. In certain other aspects, the specific binding member is an antibody that binds to a cross-reactive RANKL polypeptide consisting essentially of, or consisting of an amino acid sequence set forth as, for example, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43, wherein the antibody can be, for example, a polyclonal antibody, a monoclonal antibody, a chimeric antibody, and a humanized antibody. In one embodiment, the antibody is a monoclonal antibody. In another embodiment, the antibody is a chimeric antibody. In still another embodiment, the antibody is a humanized antibody.

In still other aspects, the specific binding member is an antigen binding fragment that binds to a cross-reactive RANKL polypeptide consisting essentially of, or consisting of, an amino acid sequence set forth as, for example, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43, wherein the antigen binding fragment is selected from among Fab, Fd, scFv, dAb, a F(ab′)₂, a bi-specific Fab₂, a multivalent antibody fragment, and a bi-specific scFv. In one embodiment, the antigen binding fragment is a Fab fragment. In another embodiment, the antigen binding fragment is a Fd fragment. In yet another embodiment, the antigen binding fragment is a scFv. In still another embodiment, the antigen binding fragment is a dAb. In yet still another embodiment, the antigen binding fragment is a F(ab′)₂. In another embodiment, the antigen binding fragment is a bi-specific Fab₂. In still another embodiment, the antigen binding fragment is a multivalent antibody fragment. In still another embodiment, the antigen binding fragment is a bi-specific scFv.

In one aspect, a specific binding member binds to a mouse and human RANKL binding sequence, wherein said mouse and human RANKL binding sequence is an amino acid sequence set forth in amino acid residues 2-12 of SEQ ID NO: 29 or 30.

In another aspect, the antibody, antigen-binding fragment, or binding protein, binds to a RANKL cross-reactive binding sequence comprising an amino acid sequence set forth as, for example, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43, wherein said peptide is about 10 to 60, from about 10 to about 55, from about 10 to about 50, from about 10 to about 45, from about 10 to about 40, from about 10 to about 35, from about 10 to about 30, from about 10 to about 25, from about 10 to about 20, or from 10 to about 15 amino acid residues in length, and said antibody, or antigen-binding fragment, or binding protein neutralizes the activity of RANKL.

In certain other embodiments, the specific binding members described herein which are based upon the CDR3 regions of the heavy or light chain, or both, of an antibody that can cross-react and neutralize RANKL from multiple species, can be useful specific binding members for in vivo therapy.

In one non-limiting embodiment, the epitope recognized by anti-RANKL binding members is located within the extracellular domain of RANKL polypeptide comprising amino acid residues 119-318 of the mature normal or wild type RANKL sequence. In one such aspect, the epitope recognized by anti-RANKL binding members is located within the extracellular domain of murine RANKL polypeptide comprising amino acid residues 119-318 of the mature normal or wild type RANKL sequence. Therefore, also provided are specific binding proteins (such as antibodies) which bind to this region, which is involved in active RANKL polypeptide function. The epitope can be determined by any conventional epitope mapping techniques known to the person skilled in the art. Alternatively, the DNA sequence encoding amino acid residues 119-318 can be digested, and the resultant fragments expressed in a suitable host. In a further approach, nucleic acid encoding RANKL loop, such as a cross-reactive RANKL extracellular domain, portions thereof, or RANKL extracellular domain which does not correspond precisely to murine or human RANKL extracellular domain in sequence can be used. Antibody binding could be determined as mentioned above.

In one aspect, the antibody is one which has the characteristics of the antibody described herein which recognizes RANKL originating from multiple species, including mouse and human, and neutralizing the bioactivity of both the human and mouse proteins. In one embodiment, the antibody comprises the VH and VL polypeptides, respectively.

In another aspect, the present application provides an antibody capable of competing with the anti-RANKL antibody, under conditions in which at least 10% of an antibody having the VH and VL sequences of the antibody is blocked from binding to both mouse and human RANKL by competition with such an antibody in an ELISA assay. In one such aspect, anti-idiotype antibodies are contemplated.

In yet another aspect, binding of an antibody to its target epitope is mediated through the complementarity determining regions (CDRs) of its heavy and light chains, with the role of CDR3 being of further consideration. Accordingly, specific binding members based on the CDR3 regions of the heavy or light chain, or both, of anti-RANKL are useful specific binding members for in vivo therapy.

The present invention provides drugs or other entities, including antibodies (e.g., anti-idiotype antibodies), that are capable of binding to the antibody thereby modulating, inhibiting or potentiating the antibody activity. Thus, anti-idiotype antibodies to anti-RANKL antibodies are provided and exemplified herein. Such anti-idiotype antibodies are useful in the development of drugs that would specifically bind the antibodies such as an anti-RANKL antibody or its epitope or that would potentiate its activity.

In one embodiment, the epitope specifically bound by the specific binding member or antibody is located within the region comprising residues 119-318 of the mature normal or wild type mammalian RANKL protein sequence and does not correspond to either mouse or human RANKL sequence. In a further aspect the epitope of the specific binding member or antibody is located within the region comprising residues 119-318 of the mature normal or wild type murine RANKL protein sequence. Therefore, also provided are specific binding proteins, such as antibodies, which bind to an epitope located within the region comprising residues 119-318 murine RANKL protein sequence. The epitope can be determined by any conventional epitope mapping techniques known to the person skilled in the art. Alternatively, the DNA sequence encoding residues 119-318 can be digested, and the resultant fragments expressed in a suitable host. Antibody binding can be determined as mentioned above.

A binding member can bind to an epitope comprising residues 119-318 of the mature normal or wild type RANKL. However other antibodies which show the same or a substantially similar pattern of reactivity also form an aspect of the invention. This can be determined by comparing such members with an antibody comprising the V_{H} and V_{L} domains, respectively. The comparison can typically be made using a Western blot in which binding members are bound to duplicate blots prepared from a nuclear preparation of cells so that the pattern of binding can be directly compared.

In another aspect, the present application provides an antibody capable of competing with an anti-RANK antibody or binding member, under conditions in which at least 10% of an antibody having the V_{H} and V_{L} sequences of the anti-RANKL antibody is blocked from binding to both mouse and human RANKL polypeptide by competition with such an antibody in an ELISA assay. As set forth above, anti-idiotype antibodies are contemplated.

Binding of an antibody, antigen-binding fragment, or binding protein to a RANKL cross-reactive binding sequence can partially (e.g., 5%, 10%, 20%, 30%, 40%, 50%, 60%, 70%, 80%, 90%, 95%, 98%, 99% or any number therein) or completely neutralize the activity of the polypeptide. Such neutralization can be determined using an in vitro assay and/or in vivo using art-recognized assays. The neutralizing activity of an antibody, or antigen-binding fragment thereof, can be determined using art recognized assays.

Provided herein are neutralizing antibodies, antigen-binding fragments or binding proteins that bind to a cross-reactive binding sequence on a polypeptide and neutralize the activity of RANKL. The neutralizing antibody can for example, prevent or inhibit the binding of RANKL to RANK.

Percentage (%) of inhibition of binding of a ligand to a cognate protein/receptor (or vice versa) by an antibody, or antigen, binding fragment thereof, which recognizes a cross-reactive binding sequence on the ligand can be, for example, at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, or greater than negative controls is indicative of an antibody, or antigen, binding fragment thereof, that inhibits binding of the ligand to its cognate protein/receptor. Percentage of inhibition of binding of the ligand to its cognate protein/receptor by such an antibody, or antigen, binding fragment thereof, of less than 2-fold greater than negative controls is indicative of an antibody, or antigen, binding fragment thereof, that does not inhibit binding of the ligand to its cognate protein/receptor.

In one non-limiting example, percentage of inhibition of binding of RANKL to RANK by an antibody, or antigen, binding fragment thereof, which recognizes a RANKL cross-reactive binding sequence can be, for example, at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, or greater than negative controls is indicative of an antibody, or antigen, binding fragment thereof, that inhibits binding of RANKL to RANK. Percentage (%) of inhibition of binding of RANKL to RANK by such an antibody, or antigen, binding fragment thereof, of less than 2-fold greater than negative controls is indicative of an antibody, or antigen, binding fragment thereof, that does not inhibit binding of RANKL to RANK.

In one aspect, the antibody is one which has the characteristics of the antibody which the inventors have described recognizing RANKL originating from multiple species, including mouse and human, and neutralizing the bioactivity of both human and mouse protein. In one non-limiting example, the antibody is a monoclonal antibody that can cross-react and neutralize RANKL from multiple species, or active fragments thereof. In a further aspect, the antibody comprises the V_{H} and V_{L} amino acid, respectively.

In certain embodiments, the application provides pharmaceutical compositions comprising a monoclonal antibody that can cross-react and neutralize RANKL from multiple species immunoreactive with one or more of polypeptides consisting essentially of, or consisting of, an amino acid sequence set forth as, for example, any of SEQ ID NOS: 1, 2, 3, 5, 6, 7, 9, 10, 11, 13, 14, 15, 17 or 18-25, and a pharmaceutically acceptable carrier. The application provides pharmaceutical compositions comprising a monoclonal antibody that can cross-react and neutralize RANKL from multiple species immunoreactive with one or more of polypeptides consisting essentially of, or consisting of, an amino acid sequence set forth as, for example, any of SEQ ID NOS: 26-43, and a pharmaceutically acceptable carrier.

The present application contemplates several means for preparation of the antibodies and active fragments thereof, including as illustrated herein known recombinant techniques, and is accordingly intended to cover such synthetic or chimeric antibody preparations within its scope. The isolation of the cDNA and amino acid sequences of the antibodies provided herein facilitates the reproduction of the antibody of the present invention by such recombinant techniques, and accordingly, the application extends to expression vectors prepared from the disclosed DNA sequences for expression in host systems by recombinant DNA techniques, and to the resulting transformed hosts.

In further aspects, the present application provides an approach to produce antibodies, exemplified herein by antibodies that can cross-react and neutralize RANKL from multiple species, which specifically recognize a conserved RANKL epitope or a cross-reactive RANKL epitope. Antibodies can recognize a conserved epitope which is found in osteoblasts, stromal cells, activated T cells, activated endothelium, and mammary epithelium cells, wherein the epitope is enhanced or evident upon induction of bone resorption. In certain embodiments, the antibodies are monoclonal antibodies. In other embodiments, the antibodies are monoclonal antibodies that specifically recognize RANKL originating from multiple species, including mouse and human.

In certain aspects, the application described herein provides a method of preparing an antibody, which specifically binds a cross-reactive RANKL polypeptide consisting essentially of, or consisting of, an amino acid sequence set forth as, for example, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43, the method comprising the steps of: a) preparing a cross-reactive RANKL polypeptide consisting essentially of, or consisting of, an amino acid sequence set forth as, for example, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43; b) immunizing a rodent (e.g., a mouse) with a solution comprising the RANKL polypeptide of a) to generate an immune response in the mouse to the RANKL polypeptide; c) isolating one or more spleen cells from the rodent of step b) after a sufficient amount of time for the mouse to generate an immune response; d) fusing the isolated one or more spleen cells of step c) with a myeloma cell line to form a population of hybridoma cells; e) culturing the hybridoma cell in a cell culture media comprising HAT; f) selecting one or more cells within the population of hybridoma cells of e) which express a antibody that binds to the RANKL polypeptide of a); and g) clonally expanding the one or more hybridoma cells of f). In certain other embodiments, the methods can further comprise the step of: h) identifying the antibody sequence of the antibody expressed in the one or clonal populations of cells of g). In certain embodiments, the antibody produced by the methods described herein are monoclonal antibodies which specifically binds a cross-reactive RANKL polypeptide consisting essentially of, or consisting of, an amino acid sequence set forth as, for example, SEQ ID NO: 18, SEQ ID NO: 19, SEQ ID NO: 20, SEQ ID NO: 21, SEQ ID NO: 22, SEQ ID NO: 23, SEQ ID NO: 24 or SEQ ID NO: 25. In certain embodiments, the antibody produced by the methods described herein are monoclonal antibodies which specifically binds a cross-reactive RANKL polypeptide consisting essentially of, or consisting of, an amino acid sequence set forth as, for example, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43.

In certain other aspects, the application described herein provides a method of generating a neutralizing antibody which specifically binds a cross-reactive RANKL polypeptide consisting essentially of, or consisting of, an amino acid sequence set forth as, for example, SEQ ID NO: 18, SEQ ID NO: 19, SEQ ID NO: 20, SEQ ID NO: 21, SEQ ID NO: 22, SEQ ID NO: 23, SEQ ID NO: 24 or SEQ ID NO: 25. In certain other aspects, the application described herein provides a method of generating a neutralizing antibody which specifically binds a cross-reactive RANKL polypeptide consisting essentially of, or consisting of, an amino acid sequence set forth as, for example, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43. The method comprising: a) preparing a cross-reactive RANKL polypeptide consisting essentially of, or consisting of, an amino acid sequence set forth as, for example, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43; b) immunizing a rodent (e.g., a mouse) with a solution comprising the RANKL polypeptide of a) to generate an immune response in the mouse to the RANKL polypeptide; c) isolating one or more spleen cells from the rodent of step b) after a sufficient amount of time for the mouse to generate an immune response; d) fusing the isolated one or more spleen cells of step c) with a myeloma cell line to form a population of hybridoma cells; e) culturing the population hybridoma cells in a cell culture media comprising HAT; f) selecting one or more cells within the population of hybridoma cells of e) which express a antibody that binds to the RANKL polypeptide of a); and g) clonally expanding the one or more hybridoma cells of f). In certain other aspects, the methods can further comprise the step of: h) identifying the antibody sequence of the antibody expressed in the one or clonal populations of cells of g). In certain embodiments, the neutralizing antibodies produced by the methods described herein are monoclonal antibodies which specifically binds a cross-reactive RANKL polypeptide consisting essentially of, or consisting of, an amino acid sequence set forth as, for example, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43.

“Monoclonal antibody” in its various grammatical forms refers to a population of antibody molecules that contain only one species of antibody combining site capable of immunoreacting with an epitope. A population of monoclonal antibodies is typically composed of antibodies produced by clones of a single cell called a hybridoma that secretes (produces) antibodies having the same binding specificity molecule. Methods of making monoclonal antibodies are described below.

The general methodology for making monoclonal antibodies by hybridomas is well known. Immortal, antibody-producing cell lines can also be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies And T-cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; 4,493,890, each of which is incorporated by reference herein in its entirety.

Panels of monoclonal antibodies produced against non-human (e.g. murine) or non-human and non-mouse RANKL extracellular domain can be screened for various properties; i.e., isotype, epitope, affinity, etc. Of interest are monoclonal antibodies that mimic the activity of OPG or its subunits. Such monoclonals can be readily identified in specific binding member activity assays. High affinity antibodies are also useful when immunoaffinity purification of native or recombinant specific binding member is desirable.

Methods for producing polyclonal antibodies are well-known in the art. See U.S. Pat. No. 4,493,795, incorporated by reference herein in its entirety, to Nestor et al. A monoclonal antibody, typically containing Fab and/or F(ab′)₂ portions of useful antibody molecules, can be prepared using the hybridoma technology described in Antibodies—A Laboratory Manual, Harlow and Lane, eds., Cold Spring Harbor Laboratory, New York (1988), which is incorporated herein by reference. Briefly, to form the hybridoma from which the monoclonal antibody composition is produced, a myeloma or other self-perpetuating cell line is fused with lymphocytes obtained from the spleen of a mammal hyperimmunized with an appropriate antigen.

Splenocytes are typically fused with myeloma cells using polyethylene glycol (PEG) 6000. Fused hybrids are selected by their sensitivity to HAT. Hybridomas producing a monoclonal antibody are identified by their ability to immunoreact with the present antibody or binding member and their ability to inhibit specified tumorigenic or hyperproliferative activity in target cells.

A monoclonal antibody can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques.

Media useful for the preparation of these compositions are both well-known in the art and commercially available and include synthetic culture media, inbred mice and the like. An exemplary synthetic medium is Dulbecco's minimal essential medium (DMEM; Dulbecco et al., Virol. 8:396 (1959)) supplemented with 4.5 gm/l glucose, 20 mm glutamine, and 20% fetal calf serum. An exemplary inbred mouse strain is the BALB/c.

Methods for producing monoclonal antibodies are also well-known in the art; see Niman et al., Proc. Natl. Acad. Sci. USA, 80:4949-4953 (1983), which is incorporated herein by reference. Typically, a peptide analog is used either alone or conjugated to an immunogenic carrier, as the immunogen in the before described procedure for producing the monoclonal antibodies. The hybridomas are screened for the ability to produce an antibody that immunoreacts with mouse and/or human RANKL present on osteoblastic cells.

Alternatively, the mouse DNA sequences, or DNA sequences encoding any other non-human or non-mouse RANKL sequence or cross-reactive sequence(s), can be used to screen for monoclonal antibodies in a yeast-based two-hybrid genetic screen (Hua et al., Gene 215(1): 143-152, 1998), which is incorporated herein by reference. Using this approach, one could insert the precise DNA sequences which encode the region to be targeted, such as the loop sequences identified form the murine RANKL crystal structure (Lam et al., United States Patent Application Publication Number 20030050223; Lam et al (2001) J Clin Invest 108(7):971-9). Cross reactive antibodies are identified by using a cDNA antibody library screened against all, or limited subfragments of, of the murine RANKL extracellular domain, or any other non-human or non-mouse extracellular domain or a cross-reactive extracellular domain.

Other methods of producing a monoclonal antibody, a hybridoma cell, or a hybridoma cell culture also are well known. See, for example, the methods of isolating monoclonal antibodies from an immunological repertoire as described by Sastry et al. (1989) Proc. Natl. Acad. Sci. USA, 86:5728-5732; and Huse et al. (1989) Science, 246:1275-1281.

Also contemplated herein is a hybridoma cell and cultures containing hybridoma cells that produce monoclonal antibodies provided herein.

It also is possible to determine if a monoclonal antibody has an equivalent specificity (immunoreaction characteristics) as a monoclonal antibody by ascertaining whether the former prevents the latter from binding to a pre-selected target molecule (e.g., a cross-reactive binding sequence). If the monoclonal antibody being tested competes with a monoclonal antibody described herein, as shown by a decrease in binding by the monoclonal antibody of the invention in standard competition assays for binding to the target molecule when present in the solid phase, then it is likely that the two monoclonal antibodies bind to the same, or a closely related, cross-reactive binding sequence or binding site.

An additional way to determine whether a monoclonal antibody has the specificity of a monoclonal antibody described herein is to identify the sequence of amino acid residues of the CDR regions of the antibodies in question. Antibody molecules having identical, or functionally equivalent, amino acid residues in their CDR regions are likely to have the same binding specificity. Methods for sequencing polypeptides are well known in the art.

The immunospecificity of an antibody, its binding capacity and the attendant affinity the antibody exhibits for a cross-reactive binding sequence, are determined by a cross-reactive binding sequence with which the antibody immunoreacts (binds). The cross-reactive binding sequence specificity can be defined, at least in part, by the amino acid residues of the variable region of the heavy chain of the immunoglobulin the antibody and, in part, by the light chain variable region amino acid residue sequence.

Use of the term “having the binding specificity of” indicates that equivalent monoclonal antibodies exhibit the same or similar immunoreaction (binding) characteristics and compete for binding to a pre-selected target binding sequence.

Humanized monoclonal antibodies or human monoclonal antibodies offer some advantages over murine monoclonal antibodies insofar as they can be used therapeutically in humans.

Specifically, human antibodies are generally not cleared from circulation as rapidly as “foreign” antigens, and do not activate the immune system in the same manner as foreign antigens and foreign antibodies. Methods of preparing “humanized” antibodies are generally well known in the art and can readily be applied to the antibodies provided herein.

In one embodiment, a monoclonal antibody can be humanized by grafting mouse CDRs into a human antibody framework without substantially interfering with the ability of the antibody to bind antigen.

An antibody can also be a fully human antibody (i.e., “humanized”) such as those generated, for example, by selection from an antibody phage display library displaying human single chain or double chain antibodies such as those described in de Haard, H. J. et al. (1999) J. Biol. Chem. 274:18218-30 and in Winter, G. et al. (1994) Annu. Rev. Immunol. 12:433-455.

Recombinant, chimeric and bispecific binding members and functional fragments thereof can be made using conventional techniques in the art.

In general, the CDR3 regions, comprising amino acid sequences substantially as set out as the CDR3 regions of the monoclonal antibodies described herein will be carried in a structure which allows for binding of the CDR3 regions to mouse RANKL protein. In the case of the CDR3 region of a monoclonal antibody that can cross-react and neutralize RANKL from multiple species, this can be carried by the V_{L} region of antibody.

The structure for carrying the CDR3s will generally be of an antibody heavy or light chain sequence or substantial portion thereof in which the CDR3 regions are located at locations corresponding to the CDR3 region of naturally occurring V_{H} and V_{L} antibody variable domains encoded by rearranged immunoglobulin genes. The structures and locations of immunoglobulin variable domains can be determined by reference to Kabat, E. A. et al, Sequences of Proteins of Immunological Interest. 4th Edition. US Department of Health and Human Services. 1987, and updates thereof, now available on the Internet (immuno.bme.nwu.edu).

The amino acid sequence of the CDR3 of the antibody can be carried as the CDR3 in a human heavy chain variable domain or a substantial portion thereof, and the amino acid sequences of the antibody are carried as the CDRs 1-3 respectively in a human light chain variable domain or a substantial portion thereof.

The variable domains can be derived from any germline or rearranged human variable domain, or can be a synthetic variable domain based on consensus sequences of known human variable domains. The CDR3-derived sequences, as defined in the preceding paragraph, can be introduced into a repertoire of variable domains lacking CDR3 regions, using recombinant DNA technology.

For example, Marks et al (Bio/Technology, 1992, 10:779-783) describe methods of producing repertoires of antibody variable domains in which consensus primers directed at or adjacent to the 5′ end of the variable domain area are used in conjunction with consensus primers to the third framework region of human V_{H} genes to provide a repertoire of V_{H} variable domains lacking a CDR3. Marks et al further describe how this repertoire can be combined with a CDR3 of a particular antibody. Using analogous techniques, the CDR3-derived sequences can be shuffled with repertoires of V_{H} or V_{L} domains lacking a CDR3, and the shuffled complete V_{H} or V_{L} domains combined with a cognate V_{L} or V_{H} domain to provide specific binding members described herein. The repertoire can then be displayed in a suitable host system such as the phage display system of WO92/01047, incorporated herein by reference herein in its entirety, so that suitable specific binding members can be selected. As described herein, a repertoire can consist of from anything from 10⁴ individual members upwards, for example from 10⁶ to 10⁸ or 10¹⁰ members.

A substantial portion of an immunoglobulin variable domain will comprise at least the three CDR regions, together with their intervening framework regions. The portion can also include at least about 50% of either or both of the first and fourth framework regions, the 50% being the C-terminal 50% of the first framework region and the N-terminal 50% of the fourth framework region. Additional residues at the N-terminal or C-terminal end of the substantial part of the variable domain can be those not normally associated with naturally occurring variable domain regions. For example, construction of specific binding members described herein made by recombinant DNA techniques can result in the introduction of N- or C-terminal residues encoded by linkers introduced to facilitate cloning or other manipulation steps. Other manipulation steps include the introduction of linkers to join variable domains to further protein sequences including immunoglobulin heavy chains, other variable domains (for example in the production of diabodies) or protein labels as discussed in more detail below.

Although in one aspect, specific binding members comprising a pair of binding domains based on sequences substantially set out in the V_{H} and V_{L} sequences of a monoclonal antibody that can cross-react and neutralize RANKL from multiple species, single binding domains based on either of these sequences form further aspects of the application. In the case of the binding domains based on the sequence substantially set out in the V_{H} sequences a monoclonal antibody that can cross-react and neutralize RANKL from multiple species, such binding domains can be used as targeting agents for tumor antigens since it is known that immunoglobulin V_{H} domains are capable of binding target antigens in a specific manner.

In the case of either of the single chain specific binding domains, these domains can be used to screen for complementary domains capable of forming a two-domain specific binding member which has in vivo properties as good as or equal to the mAb antibody disclosed herein.

This can be achieved by phage display screening methods using the so-called hierarchical dual combinatorial approach as disclosed in U.S. Pat. No. 5,969,108, which is incorporated herein by reference in its entirety, in which an individual colony containing either an H or L chain clone is used to infect a complete library of clones encoding the other chain (L or H) and the resulting two-chain specific binding member is selected in accordance with phage display techniques such as those described in that reference. This technique is also disclosed in Marks et al., id.

Specific binding members can further comprise antibody constant regions or parts thereof. For example, specific binding members based on a monoclonal antibody that can cross-react and neutralize RANKL from multiple specie can be attached at their C-terminal end to antibody light chain constant domains including human Cκ or Cλ chains. Similarly, specific binding members based on a monoclonal antibody that can cross-react and neutralize RANKL from multiple species can be attached at their C-terminal end to all or part of an immunoglobulin heavy chain derived from any antibody isotype, e.g., IgG, IgA, IgE, IgD and IgM and any of the isotype sub-classes, such as, for example, IgG1, IgG2b, and IgG4.

The advent of monoclonal antibody (mAb) technology 25 years ago has provide an enormous repertoire of useful research reagents and created the opportunity to use antibodies as approved pharmaceutical reagents in cancer therapy, autoimmune disorders, transplant rejection, antiviral prophylaxis and as anti-thrombotics (Glennie and Johnson (2000) Immunol Today 21(8):403-10). The application of molecular engineering to convert murine mAbs into chimeric mAbs (mouse V-region, human C-region) and humanized reagents where only the mAb complementarity-determining regions (CDR) are of murine origin are considered with respect to the clinical success of mAb therapy. The engineered mAbs have markedly reduced or absent immunogenicity, increased serum half-life and the human Fc portion of the mAb increases the potential to recruit the immune effectors of complement and cytotoxic cells (Clark M (2000) Immunol Today 21(8):397-402). Investigations into the biodistribution, pharmacokinetics and induction of an immune response to clinically administered mAbs requires the development of analyses to discriminate between the pharmaceutical and endogenous proteins.

Bi- and tri-specific multimers can be formed by association of different scFv molecules and have been designed as cross-linking reagents for T-cell recruitment into tumors (immunotherapy), viral retargeting (gene therapy) and as red blood cell agglutination reagents (immunodiagnostics), see e.g. Todorovska et al., J Immunol Methods. 2001 Feb. 1; 248(1-2):47-66; Tomlinson et al., Methods Enzymol. 2000; 326:461-79; McCall et al., J. Immunol. 2001 May 15; 166(10):6112-7, each of which is which is incorporated herein by reference in its entirety.

Fully human antibodies can be prepared by immunizing transgenic mice carrying large portions of the human immunoglobulin heavy and light chains. These mice, examples of such mice are the Xenomouse™ (Abgenix, Inc.) (U.S. Pat. Nos. 6,075,181 and 6,150,584, each of which is which is incorporated herein by reference in its entirety), the HuMAb-Mouse™ (Medarex, Inc./GenPharm) (U.S. Pat. Nos. 5,545,806 and 5,569,825, each of which is which is incorporated herein by reference in its entirety), the TransChromo Mouse™ (Kirin) and the KM Mouse™ (Medarex/Kirin), are well known within the art. Antibodies can then be prepared by, e.g. standard hybridoma technique or by phage display. These antibodies will then contain only fully human amino acid sequences.

Fully human antibodies can also be generated using phage display from human libraries. Phage display can be performed using methods well known to the skilled artisan, as in Hoogenboom et al and Marks et al (Hoogenboom H R and Winter G. (1992) J Mol. Biol. 227(2):381-8; Marks J D et al (1991) J Mol. Biol. 222(3):581-97; and also U.S. Pat. Nos. 5,885,793 and 5,969,108), each of which is which is incorporated herein by reference in its entirety.

Chimeric antibodies of the monoclonal antibodies that can cross-react and neutralize RANKL from multiple species can also be generated. Chimeric antibodies are a class of molecules in which heavy and light chain variable regions of for instance, a mouse, rat or other species are joined onto human heavy and light chain regions. Chimeric antibodies are produced recombinantly. One advantage of chimeric antibodies is that they can reduce xenoantigenic effects, the inherent immunogenicity of non-human antibodies (for instance, mouse, rat or other species). In addition, recombinantly prepared chimeric antibodies can often be produced in large quantities when utilizing high level expression vectors.

Antibodies can be sequenced using conventional techniques known in the art and the amino acid sequences of the complementarity determining regions (CDRs) determined. In one aspect, the amino acid sequences of one or more of the CDRs is inserted into a synthetic sequence of, for example, a human antibody (or antigen-binding fragment thereof) framework to create a human antibody that could limit adverse side reactions of immunizing a human patient with a non-human antibody. The amino acid sequences of one or more of the CDRs can also be inserted into a synthetic sequence of, for example, into a binding protein such as an avimer to create a construct for administration to a human patient. Such techniques can be modified depending on the species of animal to be immunized. For example, for veterinary uses, an antibody, antigen-binding fragment or binding protein can be synthesized for administration of a primate, a cow, a horse, etc.

In another aspect, using art-recognized techniques such as those provided and incorporated herein, nucleotides encoding amino acid sequences of one or more of the CDRs can inserted, for example, by recombinant techniques in restriction endonuclease sites of an existing polynucleotide that encodes an antibody, antigen-binding fragment or binding protein.

Antibodies, antigen-binding fragments, and binding proteins generated using such methods can be tested for one or more of their binding affinity, avidity, and neutralizing capabilities. Useful antibodies, antigen-binding fragments, and binding proteins can be used to administer a patient to prevent, inhibit, manage or treat a condition disease or disorder associated with RANKL.

For high level production, the most widely used mammalian expression system is one which utilizes the gene amplification procedure offered by dehydrofolate reductase deficient (“dhfr-”) Chinese hamster ovary cells. The system is well known to the skilled artisan. The system is based upon the dehydrofolate reductase “dhfr” gene, which encodes the DHFR enzyme, which catalyzes conversion of dehydrofolate to tetrahydrofolate. In order to achieve high production, dhfr-CHO cells are transfected with an expression vector containing a functional DHFR gene, together with a gene that encodes a desired protein. In this case, the desired protein is recombinant antibody heavy chain and/or light chain.

By increasing the amount of the competitive DHFR inhibitor methotrexate (MTX), the recombinant cells develop resistance by amplifying the dhfr gene. In standard cases, the amplification unit employed is much larger than the size of the dhfr gene, and as a result the antibody heavy chain is co-amplified.

When large scale production of the protein, such as the antibody chain, is desired, both the expression level and the stability of the cells being employed are taken into account. In long term culture, recombinant CHO cell populations lose homogeneity with respect to their specific antibody productivity during amplification, even though they derive from a single, parental clone.

In one aspect, specific binding members can be evaluated for their ability to neutralize the activity of RANKL. Measurement binding affinity, association rates, disassociation rates and avidity can be accomplished using art-recognized assays including, but not limited to, an enzyme-linked-immunosorbent assay (ELISA), Scatchard Analysis, BIACORE analysis, etc., as well as other assays commonly used and well-known to those of ordinary skill in the art.

Measurement of binding of specific binding members to a cross-reactive binding sequence and/or the ability of the specific binding members to, for example, neutralize the activity of RANKL containing the cross-reactive binding sequence, prevent binding of the polypeptide containing the cross-reactive binding sequence to a receptor or ligand, etc., can be determined using, for example, an enzyme-linked-immunosorbent assay (ELISA), a competitive binding assay, an ELISOPT assay, or any other useful assay known in the art. These assays are commonly used and well-known to those of ordinary skill in the art. Another technique for measuring apparent binding affinity familiar to those of skill in the art is a surface plasmon resonance technique (analyzed on a BIACORE 2000 system) (Liljeblad, et al., Glyco. J. 2000, 17:323-329). Standard measurements and traditional binding assays are described by Heeley, R. P. (Endocr. Res. 2002, 28:217-229).

In one non-limiting embodiment, an ELISA assay can be used to measure the neutralizing capability of specific binding members that binds to a cross-reactive binding sequence of RANKL, to prevent binding of RANKL to RANK.

Assays, such as an ELISA, also can be used to identify specific binding members which exhibit increased specificity for one or more cross-reactive binding sequences in comparison to other cross-reactive binding sequences. Assays, such as an ELISA, also can be used to identify specific binding members with bind to epitopes across one or more species (e.g., cross-reactive binding sequences). The specificity assay can be conducted by running parallel ELISAs in which a test specific binding members is screened concurrently in separate assay chambers for the ability to bind one or more epitopes on different species of the polypeptide containing the epitopes to identify specific binding members that bind to a cross-reactive binding sequence. Another technique for measuring apparent binding affinity familiar to those of skill in the art is a surface plasmon resonance technique (analyzed on a BIACORE 2000 system) (Liljeblad, et al., Glyco. J. 2000, 17:323-329). Standard measurements and traditional binding assays are described by Heeley, R. P., Endocr. Res. 2002, 28:217-229.

Specific binding members can also be identified by their ability to compete for binding with an antibody known to bind to an epitope specific to a particular species. For example, antibodies can be screened by monitoring their effect on the affinity of a known anti-epitope, such as anti-RANKL antibodies, described, e.g., in Niman et al. (PNAS USA, 80: 4949-4953 (1983)), the subject matter of which is incorporated herein in its entirety by reference.

Specific binding members also can be assayed for their ability to influence disease progression, e.g., bone loss. Any suitable assay known to one of skill in the art can be used to monitor such effects. Several such techniques are described herein.

D. Fusion Proteins and Labels

In general, fusion proteins can be produced using recombinant DNA methods known and described in the art, in which the subject polypeptide is expressed as a fusion with a second carrier protein such as a glutathione sulfhydryl transferase (GST) or other well-known carrier. Methods for preparing fusion proteins by chemical conjugation or recombinant engineering are well-known in the art. Providing a binding member with another moiety can be accomplished by administering both components at the same time in separate compositions, combining both components in the same composition, or linking the components. Methods of covalently and non-covalently linking components are well-known in the art.

Compounds can also be synthesized and expressed as fusion proteins with one or more additional domains linked thereto for producing a more immunogenic polypeptide, to more readily isolate a recombinantly synthesized polypeptide, or to identify and isolate antibodies or antibody-expressing B cells. Domains facilitating detection and purification include, for example, metal chelating peptides such as polyhistidine tracts and histidine-tryptophan modules that allow purification on immobilized metals; protein A domains that allow purification on immobilized immunoglobulin; and the domain utilized in the FLAGS® extension/affinity purification system (Immunex Corp, Seattle, Wash.). The inclusion of a cleavable linker sequence, such as Factor Xa or enterokinase (Invitrogen, San Diego Calif.), between a purification domain/tag and a polypeptide can be used to facilitate polypeptide purification. For example, an expression vector can include a peptide-encoding polynucleotide linked to six histidine residues followed by a thioredoxin and an enterokinase cleavage site (see e.g., Williams (1995) Biochemistry 34:1787 1797; Dobeli (1998) Protein Expr. Purif. 12:404-414). The histidine residues facilitate detection and purification of the fusion protein while the enterokinase cleavage site provides a means for purifying the polypeptide from the remainder of the fusion protein. Technology pertaining to vectors encoding fusion proteins and application of fusion proteins is known in the art (see e.g., Kroll (1993) DNA Cell. Biol. 12: 441-453).

Compounds can also be conjugated or recombinantly fused to any cellular toxin, bacterial or other, e.g. pseudomonas exotoxin, ricin, or diphtheria toxin. The part of the toxin used can be the whole toxin, or any particular domain of the toxin. Such antibody-toxin molecules have successfully been used for targeting and therapy of different kinds of cancers, see e.g. Pastan, Biochim Biophys Acta. 1997 Oct. 24; 1333(2):C1-6; Kreitman et al., N Engl J. Med. 2001 Jul. 26; 345(4):241-7; Schnell et al., Leukemia. 2000 January; 14(1):129-35; Ghetie et al., Mol. Biotechnol. 2001 July; 18(3):251-68, each of which is which is incorporated herein by reference in its entirety.

An antibody, antigen-binding fragment or binding protein can be conjugated to a therapeutic moiety or be a fusion protein containing a therapeutic moiety. An antibody, antigen-binding fragment or binding protein can be conjugated to a detectable moiety or be a fusion protein containing a detectable moiety. In one embodiment, the antibody, antigen-binding fragment or binding protein can be conjugated to both a therapeutic moiety and a detectable moiety. An antibody, antigen-binding fragment or binding protein can be conjugated to, or recombinantly engineered with, an affinity tag (e.g., a purification tag).

It may be necessary, in some instances, to introduce an unstructured polypeptide linker region between a label or a moiety and one or more portion of the antibodies, antigen-binding fragments or binding proteins described herein. A linker can facilitate enhanced flexibility, and/or reduce steric hindrance between any two fragments. The linker can also facilitate the appropriate folding of each fragment to occur. The linker can be of natural origin, such as a sequence determined to exist in random coil between two domains of a protein. One linker sequence is the linker found between the C-terminal and N-terminal domains of the RNA polymerase a subunit. Other examples of naturally occurring linkers include linkers found in the 1CI and LexA proteins.

Within a linker, an amino acid sequence can be varied based on the characteristics of the linker as determined empirically or as revealed by modeling. Considerations in choosing a linker include flexibility of the linker, charge of the linker, and presence of some amino acids of the linker in the naturally-occurring subunits. The linker can also be designed such that residues in the linker contact deoxyribose nucleic acid (DNA), thereby influencing binding affinity or specificity, or to interact with other proteins. In some cases, such as when it is necessary to span a longer distance between subunits or when the domains must be held in a particular configuration, the linker can, optionally, contain an additional folded domain. In some embodiments, the design of a linker can involve an arrangement of domains which requires the linker to span a relatively short distance, e.g., less than about 10 Angstroms (Å). However, in certain embodiments, linkers span a distance of up to about 50 Angstroms.

It may be necessary, in some instances, to introduce an unstructured polypeptide linker region between a label and a compound and/or between a compound and a fusion partner. As used herein, “linker” refers to an unstructured polypeptide linker region between a label and portions of an antibody, antigen-binding fragment or binding protein. The linker can facilitate enhanced flexibility, and/or reduce steric hindrance between any two fragments. The linker can also facilitate the appropriate folding of each fragment to occur. The linker can be of natural origin, such as a sequence determined to exist in random coil between two domains of a protein. An exemplary linker sequence is the linker found between the C-terminal and N-terminal domains of the RNA polymerase a subunit. Other examples of naturally occurring linkers include linkers found in the 1CI and LexA proteins. The linker can facilitate enhanced flexibility, and/or reduce steric hindrance between any two fragments. The linker can also facilitate the appropriate folding of each fragment to occur. The linker can be of natural origin, such as a sequence determined to exist in random coil between two domains of a protein. An exemplary linker sequence is the linker found between the C-terminal and N-terminal domains of the RNA polymerase a subunit. Other examples of naturally occurring linkers include linkers found in the 1CI and LexA proteins.

Within the linker, the amino acid sequence can be varied based on the characteristics of the linker as determined empirically or as revealed by modeling. Considerations in choosing a linker include flexibility of the linker, charge of the linker, and presence of some amino acids of the linker in the naturally-occurring subunits. The linker can also be designed such that residues in the linker contact DNA, thereby influencing binding affinity or specificity, or to interact with other proteins. In some cases, when it is necessary to span a longer distance between subunits or when the domains must be held in a particular configuration, the linker can optionally contain an additional folded domain.

In some embodiments, the design of a linker can involve an arrangement of domains which requires the linker to span a relatively short distance (e.g., less than about 10 Angstroms (Å)). However, in certain embodiments, linkers can span a distance of up to about 50 Å to span a longer distance.

Antibodies, antigen-binding fragments or binding proteins provided herein are such that they can be conjugated or linked to a therapeutic moiety and/or an imaging or a detectable moiety and/or an affinity tag. Methods for conjugating or linking polypeptides are well known in the art. Associations (binding) between compounds and labels include any means known in the art including, but not limited to, covalent and non-covalent interactions, chemical conjugation as well as recombinant techniques.

Methods for coupling polypeptides (free or cell-bound) to beads are known in the art. Methods for selecting coupled polypeptides or cells displaying a polypeptide are also known in the art. Briefly, paramagnetic polystyrene microparticles are commercially available (Spherotech, Inc., Libertyville, Ill.; Invitrogen, Carlsbad, Calif.) that couple peptides to microparticle surfaces that have been modified with functional groups or coated with various antibodies or ligands such as, for example, avidin, streptavidin or biotin.

The paramagnetic property of microparticles allows them to be separated from solution using a magnet. The microparticles can be easily re-suspended when removed from the magnet.

Polypeptides can be coupled to paramagnetic polystyrene microparticles coated with a polyurethane layer in a tube. The hydroxy groups on the microparticle surface are activated by reaction with p-toluensulphonyl chloride (Nilsson K and Mosbach K. “p-Toluenesulfonyl chloride as an activating agent of agarose for the preparation of immobilized affinity ligands and proteins.” Eur. J. Biochem. 1980:112: 397-402). The resulting sulphonyl ester can subsequently react covalently with peptide amino or sulfhydryl groups. The polypeptides are quickly absorbed onto the surface of the activated microparticles followed by the formation of covalent amine bonds with further incubation. The microparticles (209 microparticles/milliliter) are washed two times by placing the tube containing 1 milliliter (mL) of microparticles on a magnet, allowing the microparticles to migrate to the magnet side of the tube, removing the supernatant, and re-suspending the microparticles in 1 mL of 100 millimolar (mM) borate buffer, pH 9.5. After washing, the microparticles are re-suspended in 100 mM borate buffer, pH 9.5 at a concentration of 10⁹ microparticles/mL. Eleven nanomoles of peptide are added to the microparticles and the microparticle/polypeptide mixture is vortexed for 1 minute to mix. The microparticles are incubated with polypeptides at room temperature for at least 48 hours with slow tilt rotation. To ensure an optimal orientation of the polypeptide on the microparticles, bovine serum albumin (BSA) is added to the microparticle/polypeptide mixture to a final concentration of 0.1% (weight/volume) after incubation has proceeded for 10 minutes. After incubation, the tube containing the microparticle/polypeptide mixture is placed on the magnet until the microparticles migrate to the magnet side of the tube. The supernatant is removed and the microparticles are washed four times with 1 mL phosphate buffered saline solution (PBS), pH 7.2 containing 1% (weight/volume) BSA. Finally, the microparticles are re-suspended in 1 mL PBS solution, pH 7.2 containing 1% (weight/volume) BSA.

Alternatively, paramagnetic polystyrene microparticles containing surface carboxylic acid can be activated with a carbodiimide followed by coupling to a polypeptide, resulting in a stable amide bond between a primary amino group of the polypeptide and the carboxylic acid groups on the surface of the microparticles (Nakajima N and Ikade Y, Mechanism of amide formation by carbodiimide for bioconjugation in aqueous media, Bioconjugate Chem. 1995, 6(1), 123-130; Gilles M A, Hudson A Q and Borders C L Jr, Stability of water-soluble carbodiimides in aqueous solution, Anal Biochem. 1990 Feb. 1; 184(2):244-248; Sehgal D and Vijay I K, a method for the high efficiency of water-soluble carbodiimide-mediated amidation, Anal Biochem. 1994 April; 218(1):87-91; Szajani B et al, Effects of carbodiimide structure on the immobilization of enzymes, Appl Biochem Biotechnol. 1991 August; 30(2):225-231). The microparticles (209 microparticles/milliliter) are washed twice with 1 mL of 25 mM 2-[N-morpholino]ethane sulfonic acid, pH 5 for 10 minutes with slow tilt rotation at room temperature. The washed microparticles are re-suspended in 700 microliters (μL) 25 mM 2-[N-morpholino]ethane sulfonic acid, pH 5 followed by the addition of 21 nanomoles of peptide re-suspended in 25 mM 2-[N-morpholino]ethane sulfonic acid, pH 5 to the microparticle solution. The microparticle/polypeptide mixture is mixed by vortexing and incubated with slow tilt rotation for 30 minutes at room temperature. After this first incubation, 300 μL of ice-cold 100 milligram (mg)/mL 1-ethyl-3-(3-dimethylaminopropyl) carbodiimide hydrochloride re-suspended in 25 mM 2-[N-morpholino]ethane sulfonic acid, pH 5 is added to the polypeptide/microparticle mixture and incubated overnight at 4° C. with slow tilt rotation. The polypeptide-coupled microparticles are washed four times with 1 mL 50 mM Tris pH 7.4/0.1% BSA for 15 minutes at room temperature with slow tilt rotation. After washing, the polypeptide-coupled microparticles are re-suspended at a concentration of 10⁹ microparticles/mL in PBS solution, pH 7.2 containing 1% (weight/volume) BSA.

Another option is to couple biotinylated polypeptides to paramagnetic polystyrene microparticles whose surfaces have been covalently linked with a monolayer of streptavidin. Briefly, one mL of the streptavidin microparticles are transferred to a microcentrifuge tube and washed four times by placing the tube on a magnet and allowing the microparticles to collect on the magnet side of the tube. The solution is then removed and the microparticles are gently re-suspended in 1 mL of PBS solution, pH 7.2 containing 1% (weight/volume) BSA. After the final wash, the microparticles are re-suspended in 1 mL of PBS solution, pH 7.2 containing 1% (weight/volume) BSA; and 33 picomoles of biotinylated polypeptide are added to the microparticle solution. The microparticle/polypeptide solution is incubated for 30 minutes at room temperature with slow tilt rotation. After coupling, the unbound biotinylated polypeptide is removed from the microparticles by washing four times with PBS solution, pH 7.2 containing 1% (weight/volume) BSA. After the final wash, the microparticle/polypeptide mixture is re-suspended to a final bead concentration of 10⁹ microparticles/mL. (Argarana C E, Kuntz I D, Birken S, Axel R, Cantor C R. Molecular cloning and nucleotide sequence of the streptavidin gene. Nucleic Acids Res. 1986; 14(4):1871-82; Pahler A, Hendrickson W A, Gawinowicz Kolks M A, Aragana C E, Cantor C R. Characterization and crystallization of core streptavidin. J Biol Chem 1987:262(29): 13933-7).

Polypeptides can be conjugated to a wide variety of fluorescent dyes, quenchers and haptens such as fluorescein, R-phycoerythrin, and biotin. Conjugation can occur either during polypeptide synthesis or after the polypeptide has been synthesized and purified.

Biotin is a small (244 kilodaltons) vitamin that binds with high affinity to avidin and streptavidin proteins and can be conjugated to most peptides without altering their biological activities. Biotin-labeled polypeptides are easily purified from unlabeled polypeptides using immobilized streptavidin and avidin affinity gels, and streptavidin or avidin-conjugated probes can be used to detect biotinylated polypeptides in, for example, ELISA, dot blot or Western blot applications.

N-hydroxysuccinimide esters of biotin are the most commonly used type of biotinylation agent. N-hydroxysuccinimide-activated biotins react efficiently with primary amino groups in physiological buffers to form stable amide bonds. Polypeptides have primary amines at the N-terminus and can also have several primary amines in the side chain of lysine residues that are available as targets for labeling with N-hydroxysuccinimide-activated biotin reagents. Several different N-hydroxysuccinimide esters of biotin are available, with varying properties and spacer arm length (Pierce, Rockford, Ill.). The sulfo-N-hydroxysuccinimide ester reagents are water soluble, enabling reactions to be performed in the absence of organic solvents.

The sulfosuccinimidyl-6-(biotinamido)-6-hexanamidohexanoate reagent (Pierce product number 21338, Pierce, Rockford, Ill.) is equilibrated to room temperature before opening the vial. The peptide (1 milligram) is dissolved in 0.5 ml phosphate buffered saline (PBS) solution, pH 7.2. Immediately before use, a 10 millimolar solution of the biotin reagent is prepared. A 20-fold molar excess of biotin is added to the polypeptide to be labeled and incubated on ice for 2 hours. The biotinylated polypeptide can be separated from excess non-reacted and hydrolyzed biotin reagent by desalting or dialysis.

The mole-to-mole ratio of biotin to polypeptide can be estimated using a 2-(4′-Hydroxyazobenzene-2-carboxylic acid) assay using art-recognized techniques (Green, N M, (1975) “Avidin. In Adv. in Protein Chemistry.” Academic Press, New York. 29, 85-133; Green, N M, (1971) “The use of bifunctional biotinyl compounds to determine the arrangement of subunits in avidin.” Biochem J. 125, 781-791; Green, N M., (1965) “A spectrophotometric assay for avidin and biotin based on binding of dyes by avidin.” Biochem. J. 94, 23c-24c).

Several biotin molecules can be conjugated to a polypeptide and each biotin molecule can bind one molecule of avidin. The biotin-avidin bond formation is very rapid and stable in organic solvents, extreme pH and denaturing reagents. To quantitate biotinylation, a solution containing the biotinylated polypeptide is added to a mixture of 2-(4′-Hydroxyazobenzene-2-carboxylic acid) and avidin. Because biotin has a higher affinity for avidin, it displaces the 2-(4′-Hydroxyazobenzene-2-carboxylic acid) and the absorbance at 500 nanometers decreases proportionately. The amount of biotin in a solution can be quantitated in a single cuvette by measuring the absorbance of the 2-(4′-Hydroxyazobenzene-2-carboxylic acid)-avidin solution before and after addition of the biotin-containing peptide. The change in absorbance relates to the amount of biotin in the sample by the extinction coefficient of the 2-(4′-Hydroxyazobenzene-2-carboxylic acid)-avidin complex.

Alternatively, an antibody, antigen-binding fragment or binding protein can be conjugated with R-Phycoerythrin, a red fluorescent protein. R-Phycoerythrin is a phycobiliprotein isolated from marine algae. There are several properties that make R-Phycoerythrin ideal for labeling peptides, including an absorbance spectra that includes a wide range of potential excitation wavelengths, solubility in aqueous buffers and low nonspecific binding. R-Phycoerythrin also has a high fluorescence quantum yield (0.82 at 578 nanometers) that is temperature and pH independent over a broad range. Conjugating peptides with R-Phycoerythrin can be accomplished using art-recognized techniques described in, for example, Glazer, A N and Stryer L. (1984). Phycofluor probes. Trends Biochem. Sci. 9:423-7; Kronick, M N and Grossman, P D (1983) Immunoassay techniques with fluorescent phycobiliprotein conjugates. Clin. Chem. 29:1582-6; Lanier, L L and Loken, M R (1984) Human lymphocyte subpopulations identified by using three-color immunofluorescence and flow cytometry analysis: Correlation of Leu-2, Leu-3, Leu-7, and Leu-11 cell surface antigen expression. J. Immunol., 132:151-156; Parks, D R et al. (1984) Three-color immunofluorescence analysis of mouse B-lymphocyte subpopulations. Cytometry 5:159-68; Hardy, R R et al. (1983) demonstration of B-cell maturation in X-linked immunodeficient mice by simultaneous three-color immunofluorescence. Nature 306:270-2; Hardy R R et al. (1984) J. Exp. Med. 159:1169-88; and Kronick, M N (1986) The use of phycobiliproteins as fluorescent labels in immunoassay. J. Immuno. Meth. 92:1-13.

A number of cross-linkers can be used to produce phycobiliprotein conjugates including, but not limited to, N-Succinimidyl 3-[2-pyridyldithio]-propionamido, (Succinimidyl 6-(3-[2-pyridyldithio]-propionamido)hexanoate, or (Sulfosuccinimidyl 6-(3-[pyridyldithio]-propianamido)hexanoate. Such cross-linkers react with surface-exposed primary amines of the phycobiliprotein and create pyridyldisulfide group(s) that can be reacted with peptides that contain either free sulfhydryl groups or primary amines.

The reaction involves adding 300 micrograms of (Sulfosuccinimidyl 6-(3-[pyridyldithio]-propianamido)hexanoate to a 1 milligram/milliliter solution of phycobiliprotein in 1 milliliter of borate buffered saline solution, pH 8.5 and incubating for 30 minutes at room temperature. The non-reacted cross-linker can be removed by applying the reacted phycobiliprotein to a desalting column equilibrated with phosphate buffered saline solution, pH 7.2. Phosphate buffered saline solution, pH 7.2 is then added to the column and 1 milliliter fractions are collected in tubes. The pyridyldisulfide-activated phycobiliprotein will be the first peak to emerge from the column as measured by the absorbance at 280 nanometers. This pyridyldisulfide-activated phycobiliprotein can now be reacted with polypeptide containing either free sulfhydryl groups or primary amines. To conjugate the pyridyldisulfide-activated phycobiliprotein to primary amines in a polypeptide, 25 microliters of a 20 millimolar (Sulfosuccinimidyl 6-(3-[pyridyldithio]-propianamido)hexanoate solution are added to 2 milligrams polypeptide dissolved in 1 milliliter of phosphate buffered saline solution containing 1 millimolar ethylenediaminetetraacetic acid, pH 7.5 (PBS-EDTA) and incubated for 30 minutes at room temperature. A desalting column is equilibrated with PBS-EDTA, and the (Sulfosuccinimidyl 6-(3-[pyridyldithio]-propianamido)hexanoate-modified polypeptide is applied to the column to remove the reaction byproducts and excess (Sulfosuccinimidyl 6-(3-[pyridyldithio]-propianamido)hexanoate reagent. PBS-EDTA is then added to the column and 1 milliliter fractions are collected in tubes. The (Sulfosuccinimidyl 6-(3-[pyridyldithio]-propianamido)hexanoate-modified peptide will be the first peak to emerge from the column as measured by the absorbance at 280 nanometers. Approximately 1-3 moles pyridyldisulfide-activated phycobiliprotein are added to the (Sulfosuccinimidyl 6-(3-[pyridyldithio]-propianamido)hexanoate-modified polypeptide and the reaction is incubated overnight at room temperature to complete the conjugation. A Pyridine-2-Thione assay can be used to determine the level of (Sulfosuccinimidyl 6-(3-[pyridyldithio]-propianamido)hexanoate-modification. First, one hundred (100) microliters of (Sulfosuccinimidyl 6-(3-[pyridyldithio]-propianamido)hexanoate-modified and desalted polypeptide is diluted to 1 milliliter with phosphate buffered saline solution, pH 7.2. The absorbance at 343 nanometers of the peptide sample is compared to a PBS-EDTA blank in triplicate. To the 1 milliliter (Sulfosuccinimidyl 6-(3-[pyridyldithio]-propianamido)hexanoate-modified peptide, 10 microliters of a 15 milligram/milliliter dithiothreitol (DTT) solution are added and mixed. After exactly 15 minutes, the absorbance at 343 nanometers of the reduced sample is recorded. The change in absorbance and the molar ratio of (Sulfosuccinimidyl 6-(3-[pyridyldithio]-propianamido)hexanoate to polypeptide are calculated using the following equation: (change in absorbance/8080)ₓ((molecular weight of peptide)/(milligrams/milliliter of protein))=moles of (Sulfosuccinimidyl 6-(3-[pyridyldithio]-propianamido)hexanoate per mole of polypeptide where the value 8080 is the extinction coefficient for pyridine-2-thione at 343 nanometers.

Another option is to label polypeptide with fluorescein isothiocyanate (molecular weight 389). The isothiocyanate group on the fluorescein will cross-link with amino, sulfhydryl, imidazoyl, tyrosyl or carbonyl groups on peptides, but generally only derivatives of primary and secondary amines yield stable products. Fluorescein isothiocyanate has an excitation and emission wavelengths at 494 and 520 nanometers respectively and a molar extinction coefficient of 72,0000 M-1 cm-1 in an aqueous buffer at pH 8 (Der-Balian G, Kameda, N and Rowley, G. (1988) Fluorescein labeling of Fab while preserving single thiol. Anal. Biochem. 173:59-63).

To conjugate fluorescein isothiocyanate to a polypeptide, 1 milligram of polypeptide is dissolved in 1 milliliter of 50 millimolar borate, pH 8.5. Transfer 100 microliters of the polypeptide solution to a tube and add a 24 molar excess of fluorescein isothiocyanate (10 milligrams/milliliter in dimethylformamide). Mix well and incubate at room temperature for 1 hour. Remove the excess fluorescent dye by dialysis against phosphate buffered saline solution, pH 7.2.

In one non-limiting embodiment, an antibody, antigen-binding fragment or binding protein can be associated with (conjugated to) a toxin, a radionuclide, an iron-related compound, a dye, an imaging reagent, a fluorescent label, a chemical ablation agent, an immunomodulator, a cytokine, a cytotoxic agent, a chemotherapeutic agent, a drug or a chemotherapeutic agent which could be toxic when delivered to a cell.

Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. Enzyme labels include, but are not limited to, peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043, each of which is which is incorporated herein by reference in its entirety, are referred to by way of example for their disclosure of alternate labeling material and methods.

Alternatively, the compounds can be associated with detectable label, such as a radionuclide, iron-related compound, a dye, an imaging agent or a fluorescent agent for immunodetection of target antigens.

Non-limiting examples of radiolabels include, for example, ³²P, ³³P, ⁴³K, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/⁸¹MKr, ⁸⁷MSr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi. Radiolabels can be attached to compounds using conventional chemistry known in the art of antibody imaging. Radiolabeled compounds are useful in in vitro diagnostics techniques and in in vivo radioimaging techniques and in radioimmunotherapy. For example, in the instance of in vivo imaging, the specific binding members can be conjugated to an imaging agent rather than a radioisotope(s), including but not limited to a magnetic resonance image enhancing agent, wherein for instance an antibody molecule is loaded with a large number of paramagnetic ions through chelating groups. Examples of chelating groups include EDTA, porphyrins, polyamines crown ethers and polyoximes. Examples of paramagnetic ions include gadolinium, iron, manganese, rhenium, europium, lanthanium, holmium and ferbium. In a further aspect, radiolabelled specific binding members or radioimmunoconjugates, are useful in radioimmunotherapy for cancer therapy. In a still further aspect, the radiolabelled specific binding members are useful in radioimmuno-guided surgery techniques, wherein they can identify and indicate the presence and/or location of cancer cells, precancerous cells, tumor cells, and hyperproliferative cells, prior to, during or following surgery to remove such cells.

Functional labels include substances which are designed to be targeted to the site of a tumor to cause destruction of tumor tissue. Such functional labels include, but are not limited to, toxins or cytotoxic drugs such as 5-fluorouracil or ricin and enzymes such as bacterial carboxypeptidase or nitroreductase, which are capable of converting prodrugs into active drugs at the site of a tumor.

Non-limiting examples of toxins include, for example, ricin A chain (ricin toxin), Pseudomonas exotoxin (PE), diphtheria toxin (DT), Clostridium perfringens phospholipase C (PLC), bovine pancreatic ribonuclease (BPR), antiviral protein (PAP), abrin, abrin A chain (abrin toxin), cobra venom factor (CVF), gelonin (GEL), saporin (SAP) and viscumin.

Non-limiting examples of iron-related compounds include, for example, magnetic iron-oxide particles, ferric or ferrous particles, Fe²⁰³ and Fe³⁰⁴. Iron-related compounds and methods of labeling polypeptides, proteins and peptides can be found, for example, in U.S. Pat. Nos. 4,101,435 and 4,452,773, and U.S. published applications 20020064502 and 20020136693, each of which are hereby incorporated by reference in their entirety with respect to these compounds.

In certain embodiments, the subject antibodies, antigen-binding fragments and binding proteins can be covalently or non-covalently coupled to a cytotoxin or other cell proliferation inhibiting compound, in order to localize delivery of that agent to a tumor cell. For instance, the agent can be selected from the group consisting agents, enzyme inhibitors, proliferation inhibitors, lytic agents, DNA or RNA synthesis inhibitors, membrane permeability modifiers, DNA metabolites, dichloroethylsulfide derivatives, protein production inhibitors, ribosome inhibitors, inducers of apoptosis, and neurotoxins.

In certain embodiments, the subject antibodies, antigen-binding fragments and binding proteins can be coupled with an agent useful in imaging in vitro, in vivo and ex vivo. Such agents include: metals; metal chelators; lanthanides; lanthanide chelators; radiometals; radiometal chelators; positron-emitting nuclei; microbubbles (for ultrasound); liposomes; molecules microencapsulated in liposomes or nanosphere; monocrystalline iron oxide nanocompounds; magnetic resonance imaging contrast agents; light absorbing, reflecting and/or scattering agents; colloidal particles; fluorophores, such as near-infrared fluorophores. In many embodiments, such secondary functionality/moiety will be relatively large, e.g., at least 25 amu in size, and in many instances can be at least 50, 100 or 250 amu in size.

In certain embodiments, the secondary functionality is a chelate moiety for chelating a metal, e.g., a chelator for a radiometal or paramagnetic ion. In embodiments, it is a chelator for a radionuclide useful for radiotherapy or imaging procedures.

Radionuclides include gamma-emitters, positron-emitters, Auger electron-emitters, X-ray emitters and fluorescence-emitters, with beta- or alpha-emitters preferred for therapeutic use. Examples of radionuclides useful as toxins in radiation therapy include, for example, ³²P, ³³P, ⁴³K, ⁵²Fe, ⁵⁷Co, ⁶⁴Cu, ⁶⁷Ga ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/⁸¹MKr, ⁸⁷MSr, 90Y, ⁹⁷Ru, ⁹⁹Tc, ¹⁰⁰Pd, ¹⁰¹Rh, ¹⁰³Pb, 105Rh, ¹⁰⁹Pd, ¹¹¹Ag, ¹¹¹In, ¹¹³In, ¹¹⁹Sb, ¹²¹Sn, ¹²³I, ¹²⁵I, ¹²⁷Cs, ¹²⁸Ba, ¹²⁹Cs, 131I, ¹³¹Cs, ¹⁴³Pr, 153Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi. Preferred therapeutic radionuclides include ¹⁸⁸Re, ¹⁸⁶Re, ²⁰³Pb, ²¹²Pb, ²¹²Bi, ¹⁰⁹Pd, ⁶⁴Cu, ⁶⁷Cu, ⁹⁰Y, ¹²⁵I, ¹³¹I, ⁷⁷Br, ²¹¹At, ⁹⁷Ru, ¹⁰⁵Rh, ¹⁹⁸Au and ¹⁹⁹Ag, ¹⁶⁶Ho or ¹⁷⁷Lu. Conditions under which a chelator will coordinate a metal are described, for example, by Gasnow et al. U.S. Pat. Nos. 4,831,175, 4,454,106 and 4,472,509, which are incorporated with respect to radionuclides by reference herein. Within the present application, “radionuclide” and “radiolabel” are interchangeable.

⁹⁹Tc is an attractive radioisotope for diagnostic applications, as it is readily available to all nuclear medicine departments, is inexpensive, gives minimal patient radiation doses, and has ideal nuclear imaging properties. It has a half-life of six hours which means that rapid targeting of a technetium-labeled antibody is desirable. Accordingly, in certain embodiments, the modified antibodies include a chelating agent for technium.

In still other embodiments, the secondary functionality can be a radiosensitizing agent, e.g., a moiety that increases the sensitivity of cells to radiation. Examples of radiosensitizing agents include nitroimidazoles, metronidazole and misonidazole (see: DeVita, V. T. in Harrison's Principles of Internal Medicine, p. 68, McGraw-Hill Book Co., NY, 1983, which is incorporated herein by reference with respect to these agents). The modified antibodies, antigen-binding fragments and binding proteins that comprise a radiosensitizing agent as the active moiety can be administered and localize at the target cell. Upon exposure of the individual to radiation, the radiosensitizing agent is “excited” and causes cell death.

There are a wide range of moieties which can serve as chelators and which can be derivatized to the compounds of the present application. For instance, the chelator can be a derivative of 1,4,7,10-tetraazacyclododecanetetraacetic acid (DOTA), ethylenediaminetetraacetic acid (EDTA), diethylenetriaminepentaacetic acid (DTPA) and 1-p-Isothiocyanato-benzyl-methyl-diethylenetriaminepentaacetic acid (ITC-MX). These chelators typically have groups on the side chain by which the chelator can be used for attachment to subject compounds. Such groups include, e.g., benzylisothiocyanate, by which the DOTA, DTPA or EDTA can be coupled to, e.g., an amine group.

In one embodiment, the chelate moiety is an “NxSy” chelate moiety. As defined herein, the “NxSy chelates” include bifunctional chelators that are capable of coordinately binding a metal or radiometal and can have N₂S₂ or N₃S cores. Exemplary NxSy chelates are described, e.g., in Fritzberg et al. (1998) PNAS 85: 4024-29; and Weber et al. (1990) Chem. 1: 431-37; and in the references cited therein.

The Jacobsen et al. PCT application WO 98/12156 (incorporated by reference herein with respect to binding moieties) provides methods and compositions, i.e., synthetic libraries of binding moieties, for identifying compounds which bind to a metal atom. The approach described in the Jacobsen publication can be used to identify binding moieties which can subsequently be added to the compounds to derive a modified compound.

One issue that may be encountered with the use of conjugated proteins in radiodiagnostic applications is a potentially dangerous accumulation of the radiolabeled moiety fragments in the kidney. When the conjugate is formed using an acid- or base-labile linker, cleavage of the radioactive chelate from the protein can advantageously occur. If the chelate is of relatively low molecular weight, as most of the subject compounds are expected to be, it is not retained in the kidney and is excreted in the urine, thereby reducing the exposure of the kidney to radioactivity. However, in certain instances, it can be advantageous to utilize acid- or base-labile in the subject ligands for the same reasons they have been used in labeled proteins.

Accordingly, certain of the labeled/modified compounds can be synthesized, by standard methods in the art, to provide reactive functional groups which can form acid-labile linkages with, e.g., a carbonyl group of the ligand. Examples of suitable acid-labile linkages include hydrazone and thiosemicarbazone functions. These are formed by reacting oxidized carbohydrate with chelates bearing hydrazide, thiosemicarbazide, and functions, respectively.

Alternatively, base-cleavable which have been used for the enhanced clearance of the radiolabel from the kidneys, can be used. See, for example, Weber et al. 1990 Bioconjg. Chem. 1:431. The coupling of a bifunctional chelate to a compound via a hydrazide linkage can incorporate base-sensitive ester moieties in a linker spacer arm. Such an ester-containing linker unit is exemplified by ethylene glycolbis(succinimidyl succinate), (EGS, available from Pierce Chemical Co., Rockford, Ill.), which has two terminal N-hydroxysuccinimide (NHS) ester derivatives of two 1,4-dibutyric acid units, each of which are linked to a single ethylene glycol moiety by two alkyl esters. One NHS ester can be replaced with a suitable amine-containing BFC (for example 2-aminobenzyl DTPA), while the other NHS ester is reacted with a limiting amount of hydrazine. The resulting hyrazide is used for coupling to the antibodies, forming a ligand-BFC linkage containing two alkyl ester functions. Such a conjugate is stable at physiological pH, but readily cleaved at basic pH.

Antibodies, antigen-binding fragments or binding proteins labeled these methods can be subject to radiation-induced scission of the chelator and to loss of radioisotope by dissociation of the coordination complex. In some instances, metal dissociated from the complex can be re-complexed, providing more rapid clearance of non-specifically localized isotope and therefore less toxicity to non-target tissues. For example, chelator compounds such as EDTA or DTPA can be infused into patients to provide a pool of chelator to bind released radiometal and facilitate excretion of free radioisotope in the urine.

In still other embodiments, the compounds can be coupled to a Boron addend, such as a carborane. For example, carboranes can be prepared with carboxyl functions on pendant side chains, as is well known in the art. Attachment of such carboranes to amine peptides, can be achieved by activation of the carboxyl groups of the carboranes and condensation with the amine group to produce the conjugate. Such modified compounds can be used for neutron capture therapy.

The present application also contemplates the modification of the subject compounds with dyes, for example, useful in therapy, and used in conjunction with appropriate non-ionizing radiation. The use of light and porphyrins in methods of the present invention is also contemplated and their use in cancer therapy has been reviewed by van den Bergh, Chemistry in Britain, 22: 430-437 (1986), which is incorporated by reference herein with respect to the use of light and porphyrins.

One embodiment includes compounds labeled with a fluorescent label. Common fluorescent labels include, for example, FITC, PE, Texas Red, cytochrome c, etc. Techniques for labeling polypeptides and fragments thereof, such as those provided herein, are well-known in the art.

Chemotherapeutics useful as active moieties which, when conjugated to compounds, can be specifically delivered to cells and are, typically, small chemical entities produced by chemical synthesis. Chemotherapeutics include cytotoxic and cytostatic drugs. Chemotherapeutics can include those which have other effects on cells such as reversal of the transformed state to a differentiated state or those which inhibit cell replication. Examples of known cytotoxic agents are listed, for example, in Goodman et al., “The Pharmacological Basis of Therapeutics,” Sixth Edition, A. B. Gilman et al., eds./Macmillan Publishing Co. New York, 1980. These include, but are not limited to, taxanes, such as paclitaxel and docetaxel; nitrogen such as mechlorethamine, melphalan, uracil mustard and chlorambucil; ethylenimine derivatives, such as thiotepa; alkyl sulfonates, such as busulfan; nitrosoureas, such as lomustine, semustine and streptozocin; triazenes, such as dacarbazine; folic acid analogs, such as methotrexate; pyrimidine analogs, such as fluorouracil, cytarabine and azaribine; purine analogs, such as mercaptopurine and thioguanine; vinca alkaloids, such as vinblastine and vincristine; antibiotics, such as dactinomycin, daunorubicin, doxorubicin, and mitomycin; enzymes, such as platinum coordination complexes, such as cisplatin and carboplatinin; substituted urea, such as hydroxyurea; methyl hydrazine derivatives, such as procarbazine; adrenocortical suppressants, such as mitotane; hormones and antagonists, such as adrenocortisteroids (prednisone), progestins (hydroxyprogesterone caproate, acetate and megestrol acetate), estrogens (diethylstilbestrol and ethinyl estradiol), and androgens (testosterone propionate and fluoxymesterone).

Drugs that interfere with protein synthesis can also be used; such drugs are known to those skilled in the art and include, for example, puromycin, cycloheximide, and ribonuclease.

Most of the chemotherapeutic agents currently in use possess functional groups that are amenable to chemical cross-linking directly with an amine or carboxyl group of a compound. For example, free amino groups are available on methotrexate, doxorubicin, daunorubicin, cytosinarabinoside, bleomycin, fludarabine, and cladribine while free carboxylic acid groups are available on methotrexate, melphalan, and chlorambucil. These functional groups, that is free amino and carboxylic acids, are targets for a variety of homobifunctional and heterobifunctional chemical cross-linking agents which can crosslink these drugs directly to a free amino group of a compound.

Chemotherapeutic agents contemplated also include other chemotherapeutic drugs that are commercially available. Merely to illustrate, the chemotherapeutic can be an inhibitor of chromatin function, a inhibitor, a inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), a DNA repair inhibitor.

Chemotherapeutic agents can be categorized by their mechanism of action into, for example, the following groups: anti-metabolites/anti-cancer agents, such as pyrimidine analogs floxuridine, capecitabine, and cytarabine) and purine analogs, folate antagonists and related inhibitors antiproliferative/antimitotic agents including natural products such as vinca alkaloid (vinblastine, vincristine, and microtubule such as taxane (paclitaxel, docetaxel), vinblastin, nocodazole, epothilones and navelbine, epidipodophyllotoxins (etoposide, teniposide), DNA damaging agents (actinomycin, amsacrine, busulfan, carboplatin, chlorambucil, cisplatin, cyclophosphamide, cytoxan, dactinomycin, daunorubicin, doxorubicin, epirubicin, iphosphamide, melphalan, merchlorethamine, mitomycin, mitoxantrone, nitrosourea, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards cyclophosphamide and analogs, melphalan, chlorambucil), and (hexamethylmelamine and thiotepa), alkyl nitrosoureas (BCNU) and analogs, streptozocin), trazenes-dacarbazinine (DTIC); antiproliferative/antimitotic antimetabolites such as folic acid analogs (methotrexate); platinum coordination complexes (cisplatin, oxiloplatinim, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel; antimigratory agents; antisecretory agents (breveldin); immunosuppressives tacrolimus sirolimus azathioprine, mycophenolate; compounds (TNP-470, genistein) and growth factor inhibitors (vascular endothelial growth factor inhibitors, fibroblast growth factor inhibitors); angiotensin receptor blocker, nitric oxide donors; anti-sense oligonucleotides; antibodies (trastuzumab, rituximab); cell cycle inhibitors and differentiation inducers (tretinoin); inhibitors, topoisomerase inhibitors (doxorubicin (adriamycin), daunorubicin, dactinomycin, eniposide, epirubicin, etoposide, idarubicin, irinotecan and mitoxantrone, topotecan, irinotecan), corticosteroids (cortisone, dexamethasone, hydrocortisone, methylpednisolone, prednisone, and prenisolone); growth factor signal transduction kinase inhibitors; dysfunction inducers, toxins such as Cholera toxin, ricin, Pseudomonas exotoxin, Bordetella pertussis adenylate cyclase toxin, or diphtheria toxin, and caspase activators; and chromatin. Dosages of the chemotherapeutic agents to be considered are consistent with currently prescribed dosages.

Additionally, other labels, such as biotin followed by streptavidin-alkaline phosphatase (AP), horseradish peroxidase are contemplated herein.

As used herein, the terms “nucleic acid damaging treatment” and “nucleic acid damaging agent” refer to any treatment regimen that directly or indirectly damages nucleic acid (e.g., DNA, cDNA, genomic DNA, mRNA, tRNA or rRNA). Specific examples of such agents include alkylating agents, nitrosoureas, anti-metabolites, plant alkaloids, plant extracts and radioisotopes. Specific examples of agents also include nucleic acid damaging drugs, for example, 5-fluorouracil (5-FU), capecitabine, S-1 (Tegafur, 5-chloro-2,4-dihydroxypyridine and oxonic acid), 5-ethynyluracil, arabinosyl cytosine (ara-C), 5-azacytidine (5-AC), 2′,2′-difluoro-2′-deoxycytidine (dFdC), purine antimetabolites (mercaptopurine, azathiopurine, thioguanine), gemcitabine hydrochloride (Gemzar), pentostatin, allopurinol, 2-fluoro-arabinosyl-adenine (2F-ara-A), hydroxyurea, sulfur mustard (bischloroetyhylsulfide), mechlorethamine, melphalan, chlorambucil, cyclophosphamide, ifosfamide, thiotepa, AZQ, mitomycin C, dianhydrogalactitol, dibromoducitol, alkyl sulfonate (busulfan), nitrosoureas (BCNU, CCNU, 4-methyl CCNU or ACNU), procarbazine, decarbazine, rebeccamycin, anthracyclins such as doxorubicin (adriamycin; ADR), daunorubibcin (Cerubicine), idarubicin (Idamycin) and epirubicin (Ellence), anthracyclin analogues such as mitoxantrone, actinomycin D, non intercalating topoisomerase inhibitors such as epipodophyllotoxins (etoposide=VP16, teniposide=VM-26), podophylotoxin, bleomycin (Bleo), pepleomycin, compounds that form adducts with nucleic acid including platinum derivatives (e.g., cisplatin (CDDP), trans analogue of cisplatin, carboplatin, iproplatin, tetraplatin and oxaliplatin), camptothecin, topotecan, irinotecan (CPT-11), and SN-38. Specific examples of nucleic acid damaging treatments include radiation (e.g., focused microwaves, ultraviolet (UV), infrared (IR), or alpha-, beta- or gamma-radiation) and environmental shock (e.g., hyperthemmia).

As used herein, the terms “anti-proliferative treatment” and “anti-proliferative agent” refer to any treatment regimen that directly or indirectly inhibits proliferation of a cell, virus, bacteria or other unicellular or multicellular organism regardless of whether or not the treatment or agent damages nucleic acid. Exemplary examples of anti-proliferative agents are anti-tumor and anti-viral drugs, which inhibit cell proliferation or virus proliferation or replication. Specific examples include, inter alia, cyclophosphamide, azathioprine, cyclosporin A, prednisolone, melphalan, chlorambucil, mechlorethamine, busulphan, methotrexate, 6-mercaptopurine, thioguanine, cytosine arabinoside, taxol, vinblastine, vincristine, doxorubicin, actinomycin D, mithramycin, carmustine, lomustine, semustine, streptozotocin, hydroxyurea, cisplatin, mitotane, procarbazine, dacarbazine and dibromomannitol. Anti-proliferative agents that cause nucleic acid replication errors or inhibit nucleic acid replication are those such as nucleoside and nucleotide analogues (e.g., AZT or 5-AZC).

Methods for labeling polypeptides (antibodies, antigen-binding fragments or binding proteins) include, but are not limited to, those provided herein and which are well known in the art. Compounds can be labeled with a radiolabel or toxin, and the compounds can be prepared as compositions for therapeutic treatment of patients or the like where the compositions are administered to the patient in an effective amount. When the compounds are labeled with a label that can be visualized, the compounds can be prepared as compositions useful for diagnosis of patients where the compositions are administered to the patient in an effective amount for in vivo imaging or where the compositions are tested in an in vitro assay.

Flow cytometry and the related flow sorting (also known as fluorescence activated cell sorting, or FACS) are methods by which individual cells can be quantitatively assayed for the presence of a specific component or component variant based upon staining with a fluorescent reporter. Briefly, cells are stained with the specific reporter and then hydrodynamically focused into a single cell steam for interrogation with a laser which excites the fluorescent moiety. Fluorescent emission is detected through a wavelength restricted optical pathway and converted to numeric data correlated to an individual cell. In the case of flow sorting, predefined subsets of emission criteria can be met and the cells of interest diverted into a collection receptacle for further use by electrostatic repulsion or mechanical action (Herzenberg L A, Sweet R G, Herzenberg L A: Fluorescence activated cell sorting, Sci Amer 234(3):108, March 1976).

Fluorescent moieties to be detected include, but are not limited to, compounds such as fluorescein (commonly called FITC), phycobiliproteins such as phycoerythrin (PE) and allophycocyanin (APC) (Kronick, M. N. J. Imm. Meth. 92: 1-13 (1986)), fluorescent semiconductor nanocrystals such as Quantum dot (QDot) bioconjugates for ultrasensitive nonisotopic detection (Chan W C, Nie S. Science 281, 2016-8 (1998)), coumarin derivatives such as Fluorescent Acylating Agents derived from 7-Hydroxycoumarin (Baker W, Collis C B. 12 (1949)), rhodamine, auramine, Texas Red, AMCA blue and Lucifer Yellow. One example of a detecting material is anti-rabbit antibody prepared in goats and conjugated with fluorescein through an isothiocyanate.

Fluorescence can be reported from fluorescent proteins such as Teal Fluorescent Protein (TFP), from chemical stains of cellular components such as DAPI bound to DNA, from fluorescent moieties covalently conjugated to antibodies that recognize cellular products, from fluorescent moieties covalently conjugated to ligands of cellular receptors, and from fluorescent moieties covalently conjugated to substrates of cellular enzymes.

Cells stained with generally membrane impermeant reporters, such as antibodies, can be sorted for subsequent processing to recover components such as genes, episomes, or proteins of interest. Cells stained for surface expression components or stained with cell membrane permeant reporters can also be sorted intact for propagation.

E. Nucleic Acids, Vectors and Host Cells

The present application provides an isolated polynucleotide (nucleic acid) encoding a polypeptide such as, for example, a cross-reactive binding sequence or a specific binding member, vectors containing such polynucleotides, and host cells and expression systems for transcribing and translating such polynucleotides into polypeptides.

The present application also provides constructs in the form of plasmids, vectors, transcription or expression cassettes which comprise at least one polynucleotide as above.

The present application also provides a recombinant host cell which comprises one or more constructs as above. A nucleic acid encoding any specific binding member as provided itself forms an aspect of the present application, as does a method of production of the specific binding member which method comprises expression from encoding nucleic acid therefrom. Expression can conveniently be achieved by culturing under appropriate conditions recombinant host cells containing the nucleic acid. Following production by expression a specific binding member can be isolated and/or purified using any suitable technique, then used as appropriate.

Specific binding members and encoding nucleic acid molecules and vectors described herein can be provided isolated and/or purified, e.g. from their natural environment, in substantially pure or homogeneous form, or, in the case of nucleic acid, free or substantially free of nucleic acid or genes origin other than the sequence encoding a polypeptide with the required function. Nucleic acid can comprise DNA or RNA and can be wholly or partially synthetic.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common bacterial host is E. coli.

The expression of antibodies and antibody fragments in prokaryotic cells such as E. coli is well established in the art. For a review, see for example Plückthun, A. Bio/Technology 9: 545-551 (1991). Expression in eukaryotic cells in culture is also available to those skilled in the art as an option for production of a specific binding member, see for recent reviews, for example Raff, M. E. (1993) Curr. Opinion Biotech. 4: 573-576; Trill J. J. et al. (1995) Curr. Opinion Biotech 6: 553-560, each of which is which is incorporated herein by reference in its entirety.

Suitable vectors can be chosen or constructed, containing appropriate regulatory sequences, including promoter sequences, terminator sequences, polyadenylation sequences, enhancer sequences, marker genes and other sequences as appropriate. Vectors can be plasmids, viral e.g. ‘phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Short Protocols in Molecular Biology, Second Edition, Ausubel et al. eds., John Wiley & Sons, 1992. The disclosures of Sambrook et al. and Ausubel et al. are incorporated herein by reference in their entirety.

Thus, a further aspect provides a host cell containing nucleic acid as disclosed herein. A still further aspect provides a method comprising introducing such nucleic acid into a host cell. The introduction can employ any available technique. For eukaryotic cells, suitable techniques can include, for example, calcium phosphate transfection, DEAE Dextran, electroporation, liposome-mediated transfection and transduction using retrovirus or other virus, e.g. vaccinia or, for insect cells, baculovirus. For bacterial cells, suitable techniques can include, for example, calcium chloride transformation, electroporation and transfection using bacteriophage.

The introduction of the nucleic acids described herein into a host cell can be followed by causing or allowing expression from the nucleic acid, e.g. by culturing host cells under conditions for expression of the gene.

In one embodiment, the nucleic acid is integrated into the genome (e.g. chromosome) of the host cell. Integration can be promoted by inclusion of sequences which promote recombination with the genome, in accordance with standard techniques.

The present application also provides a method which comprises using a construct as stated above in an expression system in order to express a specific binding member or polypeptide as above.

The present application further provides an isolated nucleic acid encoding a specific binding member described herein.

The present application also relates to isolated nucleic acids, such as recombinant DNA molecules or cloned genes, or degenerate variants thereof, mutants, analogs, or fragments thereof, which encode a isolated RANKL cross-reactive binding sequence described herein. In one aspect, the present invention provides a nucleic acid which codes for a polypeptide of the invention as defined above, including a polypeptide A-A′ loop, C-D loop, D-E loop, or E-F loop, such as set out in any of the sequences provided herein and in the Figures, including as any of SEQ ID NOS: 1, 2, 3, 5, 6, 7, 9, 10, 11, 13, 14, 15, 17 and 18-25.

In one aspect, the present application provides a nucleic acid which codes for a polypeptide as defined above, including a polypeptide A-A′ loop, C-D loop, D-E loop, or E-F loop, such as set out in any of the sequences provided herein and in the Figures, including as any of SEQ ID NOS: 26-43. In one embodiment, the RANKL cross-reactive binding sequence is a human and mouse RANKL cross-reactive binding sequence such as, but not limited to, an amino acid sequence set forth in amino acid residues 2-12 of SEQ ID NO: 29 or 30. In one embodiment, the RANKL cross-reactive binding sequence is a human and mouse RANKL cross-reactive binding sequence such as, but not limited to, an amino acid sequence set forth in amino acid residues 2-12 of SEQ ID NO: 29 or 30. The polynucleotide encoding a RANKL cross-reactive binding sequence peptide can further encode a detectable moiety and/or an affinity tag as described elsewhere herein.

In one embodiment, the present application relates to isolated nucleic acids, such as recombinant DNA molecules or cloned genes, or degenerate variants thereof, mutants, analogs, or fragments thereof, which encode a RANKL cross-reactive binding sequence consisting essentially of, or consisting of, an amino acid sequence set forth as, for example, any of SEQ ID NOS: 1, 2, 3, 5, 6, 7, 9, 10, 11, 13, 14, 15, 17 or 18-25.

In one embodiment, the present application relates to isolated nucleic acids, such as recombinant DNA molecules or cloned genes, or degenerate variants thereof, mutants, analogs, or fragments thereof, which encode a RANKL cross-reactive binding sequence consisting essentially of, or consisting of, an amino acid sequence set forth as, for example, any of SEQ ID NOS: 26-43.

In a further embodiment, the DNA sequence of the recombinant DNA molecule or cloned gene can be operatively linked to an expression control sequence which can be introduced into an appropriate host. The application accordingly extends to unicellular hosts transformed with the recombinant DNA molecule comprising a DNA sequence encoding a RANKL cross-reactive binding sequence. The application accordingly extends to unicellular hosts transformed with the recombinant DNA molecule comprising a DNA sequence encoding a RANKL cross-reactive binding sequence consisting essentially, or consisting of, of an amino acid sequence set forth as, for example, any of SEQ ID NOS: 1, 2, 3, 5, 6, 7, 9, 10, 11, 13, 14, 15, 17 or 18-25. The application accordingly extends to unicellular hosts transformed with the recombinant DNA molecule comprising a DNA sequence encoding a RANKL cross-reactive binding sequence consisting essentially, or consisting of, of an amino acid sequence set forth as, for example, any of SEQ ID NOS: 26-43.

In certain embodiments, the present application provides nucleic acids capable of encoding a RANKL cross-reactive binding sequence set forth as, for example, any of SEQ ID NOS: 1, 2, 3, 5, 6, 7, 9, 10, 11, 13, 14, 15, 17 or 18-25 which is a recombinant DNA molecule. In other embodiments, nucleic acids capable of encoding a RANKL cross-reactive binding sequence set forth as, for example, any of SEQ ID NOS: 2, 3, 6, 7, 10, 11, 14, 15 or 18-25 which is a recombinant DNA molecule are provided. Such recombinant DNA molecules wherein the DNA molecules are operatively linked to an expression control sequence are also provided herein.

In certain embodiments, the present application provides nucleic acids capable of encoding a RANKL cross-reactive binding sequence set forth as, for example, any of SEQ ID NOS: 26-43 which is a recombinant DNA molecule. In other embodiments, nucleic acids capable of encoding a RANKL cross-reactive binding sequence set forth as, for example, any of SEQ ID NOS: 26-43 which is a recombinant DNA molecule are provided. Such recombinant DNA molecules wherein the DNA molecules are operatively linked to an expression control sequence are also provided herein.

The present application further provides a vector which comprises the nucleic acid capable of encoding a RANKL cross-reactive binding sequence set forth as, for example, any of SEQ ID NOS: 1, 2, 3, 5, 6, 7, 9, 10, 11, 13, 14, 15, 17 or 18-25 and a promoter. The present application provides a vector which comprises the nucleic acid capable of encoding a non-murine and non-human RANKL cross-reactive binding sequence set forth as, for example, any of SEQ ID NOS: 2, 3, 6, 7, 10, 11, 14, 15 or 18-25 and a promoter. The present application further provides a vector which comprises the nucleic acid capable of encoding a RANKL cross-reactive binding sequence set forth as, for example, any of SEQ ID NOS: 26-43 and a promoter. The present application provides a vector which comprises the nucleic acid capable of encoding a non-murine and non-human RANKL cross-reactive binding sequence set forth as, for example, any of SEQ ID NOS: 26-43 and a promoter. A promoter can comprises a bacterial, yeast, insect or mammalian promoter. Vectors include, but are not limited to, a plasmid, cosmid, yeast artificial chromosome (YAC), bacteriophage or eukaryotic viral DNA.

In other embodiments, a host vector system can be used for the production of a polypeptide which comprises the vector capable of encoding a RANKL cross-reactive binding sequence set forth as, for example, any of SEQ ID NOS: 1, 2, 3, 5, 6, 7, 9, 10, 11, 13, 14, 15, 17 or 18-25 in a suitable host cell. The present application provides a host vector system for the production of a polypeptide which comprises the vector capable of encoding a non-murine and non-human RANKL cross-reactive binding sequence set forth as, for example, any of SEQ ID NOS: 2, 3, 6, 7, 10, 11, 14, 15 or 18-25 in a suitable host cell. A host vector system is provided wherein the suitable host cell comprises a prokaryotic or eukaryotic cell. A unicellular host transformed with a recombinant DNA molecule or vector capable of encoding a RANKL cross-reactive binding sequence set forth as, for example, any of SEQ ID NOS: 1, 2, 3, 5, 6, 7, 9, 10, 11, 13, 14, 15, 17 or 18-25 is thereby provided.

In other embodiments, provided herein is a host vector system for the production of a polypeptide which comprises the vector capable of encoding a RANKL cross-reactive binding sequence set forth as, for example, any of SEQ ID NOS: 26-43 in a suitable host cell. Also provided herein is a host vector system for the production of a polypeptide which comprises the vector capable of encoding a non-murine and non-human RANKL cross-reactive binding sequence set forth as, for example, any of SEQ ID NOS: 26-43 in a suitable host cell. A host vector system is provided wherein the suitable host cell comprises a prokaryotic or eukaryotic cell. A unicellular host transformed with a recombinant DNA molecule or vector capable of encoding a RANKL cross-reactive binding sequence set forth as, for example, any of SEQ ID NOS: 26-43 is thereby provided.

As stated above, the present application also relates to a recombinant DNA molecule or cloned gene, or a degenerate variant thereof, which encodes a polypeptide antigen, including a RANKL loop peptide, or encoding an anti-RANKL antibody or a fragment thereof that binds to a RANKL loop peptide, wherein the RANKL loop peptide consists essentially of an amino acid sequence set forth in the polypeptide sequences described herein and in the Figures, including as set out in any of SEQ ID NOS:1, 2, 3, 5, 6, 7, 9, 10, 11, 13, 14, 15, 17 and 18-25.

As stated above, the present application also relates to a recombinant DNA molecule or cloned gene, or a degenerate variant thereof, which encodes a polypeptide antigen, including a RANKL loop peptide, or encoding an anti-RANKL antibody or a fragment thereof that binds to a RANKL loop peptide, wherein the RANKL loop peptide consists essentially of an amino acid sequence set forth in the polypeptide sequences described herein and in the Figures, including as set out in any of SEQ ID NOS: 26-43.

Also provided herein is an isolated polynucleotide encoding a RANKL cross-reactive binding sequence or a peptide containing a RANKL cross-reactive binding sequence such as those described herein. In one non-limiting aspect, a polynucleotide codes for a polypeptide as defined above including, but not limited to, a polypeptide containing any of the following of RANKL: A-A′ loop, C-D loop, D-E loop, or E-F loop, such as set out in any of the sequences provided herein and in the Figures, including as any of SEQ ID NOS: 1, 2, 3, 5, 6, 7, 9, 10, 11, 13, 14, 15, 17 and 18-25. In another non-limiting aspect, a polynucleotide codes for a polypeptide as defined above including, but not limited to, a polypeptide containing any of the following of RANKL: A-A′ loop, C-D loop, D-E loop, or E-F loop, such as set out in any of the sequences provided herein and in the Figures, including as any of SEQ ID NOS: 26-43.

Provided herein is an isolated polynucleotide encoding a binding member such as those described herein which binds to any of the RANKL cross-reactive binding sequences provided herein. Also provided herein are binding members that bind to RANKL and which can neutralize RANKL. Thus, in one aspect, provided herein is an isolated polynucleotide which comprises a sequence encoding a specific binding member as defined above, and methods of preparing specific binding members of the invention which comprise expressing said polynucleotides under conditions to bring about expression of said binding member, and recovering the binding member.

Provided herein are isolated polynucleotides encoding a binding member that specifically binds to a RANKL cross-reactive binding sequence such as those described herein.

The present application also relates to a recombinant DNA molecule or cloned gene, or a degenerate variant thereof, which encodes, for example, an antibody described herein. In another embodiment, the present application also relates to a recombinant DNA molecule or cloned gene, or a degenerate variant thereof (e.g., a nucleic acid molecule, a recombinant DNA molecule or cloned gene), encoding the antibody.

In a further embodiment, the full DNA sequence of the recombinant DNA molecule or cloned gene of an antibody described herein can be operatively linked to an expression control sequence which can be introduced into an appropriate host. The application accordingly extends to unicellular hosts transformed with the cloned gene or recombinant DNA molecule comprising a DNA sequence encoding the V_{H} and/or V_{L}, or portions thereof, of the antibody.

Another feature is the expression of the DNA sequences disclosed herein. As is well known in the art, DNA sequences can be expressed by operatively linking them to an expression control sequence in an appropriate expression vector and employing that expression vector to transform an appropriate unicellular host.

Such operative linking of a DNA sequence to an expression control sequence, of course, includes, if not already part of the DNA sequence, the provision of an initiation codon, ATG, in the correct reading frame upstream of the DNA sequence.

Polynucleotides and vectors can be provided in an isolated and/or a purified form (e.g., free or substantially free of polynucleotides of origin other than the polynucleotide encoding a polypeptide with the required function). As used herein, “substantially pure” and “substantially free,” refer to a solution or suspension containing less than, for example, 20% or less extraneous material, 10% or less extraneous material, 5% or less extraneous material, 4% or less extraneous material, 3% or less extraneous material, 2% or less extraneous material, or 1% or less extraneous material.

A wide variety of host/expression vector combinations can be employed in expressing the DNA sequences of this invention. Useful expression vectors, for example, can consist of segments of chromosomal, non-chromosomal and synthetic DNA sequences. Suitable vectors include derivatives of SV40 and known bacterial plasmids, e.g., E. coli plasmids col E1, Pcr1, Pbr322, Pmb9 and their derivatives, plasmids such as RP4; phage DNAs, e.g., the numerous derivatives of phage λ, e.g., NM989, and other phage DNA, e.g., M13 and filamentous single stranded phage DNA; yeast plasmids such as the 2u plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Also provided herein is a recombinant host cell which comprises one or more polynucleotide constructs. A polynucleotide encoding an antibody, antigen-binding fragment or binding protein as provided herein forms an aspect of the present application, as does a method of production of the antibody, antigen-binding fragment or binding protein which method comprises expression from the polynucleotide. Expression can be achieved, for example, by culturing under appropriate conditions recombinant host cells containing the polynucleotide. An antibody, antigen-binding fragment or binding protein can then be isolated and/or purified using any suitable technique, and used as appropriate.

Any of a wide variety of expression control sequences—sequences that control the expression of a DNA sequence operatively linked to it—can be used in these vectors to express the DNA sequences. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage X, the control regions of fd coat protein, the promoter for 3-phosphoglycerate kinase or other glycolytic enzymes, the promoters of acid phosphatase (e.g., PhoS), the promoters of the yeast-mating factors, and other sequences known to control the expression of genes of prokaryotic or eukaryotic cells or their viruses, and various combinations thereof.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary (CHO) cells, HeLa cells, baby hamster kidney cells, NSO mouse melanoma cells and many others. A common, bacterial host can be, for example, E. coli.

The expression of antibodies, antibody fragments and binding proteins in prokaryotic cells, such as E. coli, is well established in the art. For a review, see for example Plückthun, A. Bio/Technology 9: 545-551 (1991). Expression in eukaryotic cells in culture is also available to those skilled in the art (Raff, M. E. (1993) Curr. Opinion Biotech. 4: 573-576; Trill J. J. et al. (1995) Curr. Opinion Biotech 6: 553-560).

A wide variety of unicellular host cells are also useful in expressing the DNA sequences. These hosts include well-known eukaryotic and prokaryotic hosts, such as strains of E. coli, Pseudomonas, Bacillus, Streptomyces, fungi such as yeasts, and animal cells, such as CHO, YB/20, NSO, SP2/0, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

It will be understood that not all vectors, expression control sequences and hosts will function equally well to express the DNA sequences. Neither will all hosts function equally well with the same expression system. However, one skilled in the art will be able to select the proper vectors, expression control sequences, and hosts without undue experimentation to accomplish the desired expression without departing from the scope of this application. For example, in selecting a vector, the host must be considered because the vector must function in it. The vector's copy number, the ability to control that copy number, and the expression of any other proteins encoded by the vector, such as antibiotic markers, will also be considered. One of ordinary skill in the art can select the proper vectors, expression control sequences, and hosts to accomplish the desired expression without departing from the scope of this application. For example, in selecting a vector, the host is considered because the vector functions in it. The vector's copy number, the ability to control that copy number, and the expression of any other proteins encoded by the vector, such as antibiotic markers, can also be considered.

The present application also provides constructs in the form of plasmids, vectors, transcription or expression cassettes as described elsewhere herein which comprise at least one polynucleotide as above. Suitable vectors can be chosen or constructed, containing appropriate regulatory sequences, including promoter sequences, terminator sequences, polyadenylation sequences, enhancer sequences, selectable markers and other sequences as appropriate. Vectors can be plasmids, viral e.g., phage, phagemid, etc., as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Short Protocols in Molecular Biology, Second Edition, Ausubel et al. eds., John Wiley & Sons, 1992. The disclosures of Sambrook et al. and Ausubel et al. are incorporated herein by reference.

In selecting an expression control sequence, a variety of factors will normally be considered. These include, for example, the relative strength of the system, its controllability, and its compatibility with the particular DNA sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products.

A further aspect provides a host cell containing one or more polynucleotides as disclosed herein. Yet a further aspect provides a method of introducing such one or more polynucleotides into a host cell, any available technique. For eukaryotic cells, suitable techniques can include, for example, calcium phosphate transfection, DEAEDextran, electroporation, liposome-mediated transfection and transduction using retrovirus or other virus (e.g. vaccinia) or, for insect cells, baculovirus. For bacterial cells, suitable techniques can include, for example calcium chloride transformation, electroporation and transfection using bacteriophages.

The introduction can be followed by causing or allowing expression from the one or more polynucleotides, e.g. by culturing host cells under conditions for expression of one or more polypeptides from one or more polynucleotides. Inducible systems can be used and expression induced by addition of an activator.

In one embodiment, the polynucleotides can be integrated into the genome (e.g., chromosome) of the host cell. Integration can be promoted by inclusion of sequences which promote recombination with the genome, in accordance with standard techniques. In another embodiment, the nucleic acid is maintained on an episomal vector in the host cell.

Methods are provided herein which include using a construct as stated above in an expression system in order to express a specific polypeptide.

Considering these and other factors, a person skilled in the art will be able to construct a variety of vector/expression control sequence/host combinations that will express the DNA sequences on fermentation or in large scale animal culture.

A polynucleotide encoding an antibody, antigen-binding fragment, or a binding protein can be prepared recombinantly/synthetically in addition to, or rather than, cloned. The polynucleotide can be designed with the appropriate codons for the antibody, antigen-binding fragment, or a binding protein. In general, one will select preferred codons for an intended host if the sequence will be used for expression. The complete polynucleotide can be assembled from overlapping oligonucleotides prepared by standard methods and assembled into a complete coding sequence. See, e.g., Edge, Nature, 292:756 (1981); Nambair et al., Science, 223:1299 (1984); Jay et al., J. Biol. Chem., 259:6311 (1984).

Synthetic polynucleotides allow convenient construction of genes which will express antibody, or antigen-binding fragment thereof, or “muteins.” Alternatively, polynucleotides encoding muteins can be made by site-directed mutagenesis of native antibody, or antigen-binding fragment thereof, genes or cDNAs, and muteins can be made directly using conventional polypeptide synthesis.

A general method for site-specific incorporation of unnatural amino acids into proteins is described in Christopher J. Noren, Spencer J. Anthony-Cahill, Michael C. Griffith, Peter G. Schultz, Science, 244:182-188 (April 1989). This method can be used to create analogs with unnatural amino acids.

Thus, it is further intended that specific binding member analogs can be prepared from nucleotide sequences of the protein complex/subunit derived within the scope of the present application. Analogs, such as fragments, can be produced, for example, by pepsin digestion of specific binding member material. Other analogs, such as muteins, can be produced by standard site-directed mutagenesis of specific binding member coding sequences. Analogs exhibiting “specific binding member activity” such as small molecules, whether functioning as promoters or inhibitors, can be identified by known in vivo and/or in vitro assays.

As mentioned above, a DNA sequence encoding a specific binding member can be prepared synthetically rather than cloned. The DNA sequence can be designed with the appropriate codons for the specific binding member amino acid sequence. In general, one will select preferred codons for the intended host if the sequence will be used for expression. The complete sequence is assembled from overlapping oligonucleotides prepared by standard methods and assembled into a complete coding sequence. See, e.g., Edge, Nature, 292:756 (1981); Nambair et al., Science, 223:1299 (1984); Jay et al., J. Biol. Chem., 259:6311 (1984), each of which is which is incorporated herein by reference in its entirety.

Synthetic DNA sequences allow convenient construction of genes which will express specific binding member analogs or “muteins”. Alternatively, DNA encoding muteins can be made by site-directed mutagenesis of native specific binding member genes or cDNAs, and muteins can be made directly using conventional polypeptide synthesis.

A general method for site-specific incorporation of unnatural amino acids into proteins is described in Christopher J. Noren, Spencer J. Anthony-Cahill, Michael C. Griffith, Peter G. Schultz, Science, 244:182-188 (April 1989). This method can be used to create analogs with unnatural amino acids.

F. Pharmaceutical and Therapeutic Compositions

Each of the compounds set forth as antibodies, antigen-binding fragments or binding proteins as described herein can be used as a composition when combined with an acceptable carrier or excipient. Such compositions are useful for in vitro analysis or for administration to a subject in vivo or ex vivo, and for diagnosis, detection, monitoring and/or treating a subject with the disclosed compounds, for example.

Thus pharmaceutical compositions can comprise, in addition to active ingredient, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration.

Pharmaceutical formulations comprising a protein of interest, e.g., an antibody, identified by the methods described herein can be prepared for storage by mixing the protein having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

The formulation described herein can also contain more than one active compound as necessary for the particular indication being treated. Such molecules are suitably present in combination in amounts that are effective for the purpose intended.

In one embodiment, the pharmaceutical formulations can comprise an antibody identified by the methods described herein. In certain embodiments, the pharmaceutical formulation can be in a microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

In still other embodiments, sustained-release preparations can be prepared. Suitable examples of sustained-release preparations include semi-permeable matrices of solid hydrophobic polymers containing the protein, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and .gamma. ethyl-L-glutamate, non-degradable ethylene-vinyl acetate, degradable lactic acid-glycolic acid copolymers such as the Lupron Depot® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated proteins remain in the body for a long time, they can denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

Acceptable carriers are physiologically acceptable to the administered patient and retain the therapeutic properties of the compounds with/in which it is administered. Acceptable carriers and their formulations are and generally described in, for example, Remington' pharmaceutical Sciences (18th Edition, ed. A. Gennaro, Mack Publishing Co., Easton, Pa. 1990). One exemplary carrier is physiological saline. The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Each carrier must be “acceptable” in the sense of being compatible with the other ingredients of the formulation and not injurious to a subject to whom it is administered. Nor should an acceptable carrier alter the specific activity of the subject compounds. Exemplary carriers and excipients have been provided elsewhere herein.

In one aspect, provided herein are pharmaceutically acceptable or physiologically acceptable compositions including solvents (aqueous or non-aqueous), solutions, emulsions, dispersion media, coatings, isotonic and absorption promoting or delaying agents, compatible with pharmaceutical administration. Pharmaceutical compositions or pharmaceutical formulations therefore refer to a composition suitable for pharmaceutical use in a subject. The pharmaceutical compositions and formulations include an amount of a compound described herein, for example, an effective amount of a cross-reactive binding sequence polypeptide or a binding member (e.g., an antibody, antigen-binding fragment, or binding protein) described herein, and a pharmaceutically or physiologically acceptable carrier.

Compositions can be formulated to be compatible with a particular route of administration, systemic or local. Thus, compositions include carriers, diluents, or excipients suitable for administration by various routes. Pharmaceutical compositions for oral administration can be in tablet, capsule, powder or liquid form. A tablet can comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can, if needed, be included.

In a further embodiment, the compositions can further comprise, if needed, an acceptable additive in order to improve the stability of the compounds in composition and/or to control the release rate of the composition. Acceptable additives do not alter the specific activity of the subject antibodies, antigen-binding fragments or cross-reactive binding sequences. Exemplary acceptable additives include, but are not limited to, a sugar such as mannitol, sorbitol, glucose, xylitol, trehalose, sorbose, sucrose, galactose, dextran, dextrose, fructose, lactose and mixtures thereof. Acceptable additives can be combined with acceptable carriers and/or excipients such as dextrose. Alternatively, exemplary acceptable additives include, but are not limited to, a surfactant such as polysorbate 20 or polysorbate 80 to increase stability of the peptide and decrease gelling of the solution. The surfactant can be added to the composition in an amount of 0.01% to 5% of the solution. Addition of such acceptable additives increases the stability and half-life of the composition in storage.

Formulations or enteral (oral) administration can be contained in a tablet (coated or uncoated), capsule (hard or soft), microsphere, emulsion, powder, granule, crystal, suspension, syrup or elixir. Conventional nontoxic solid carriers which include, for example, pharmaceutical grades of mannitol, lactose, starch, magnesium stearate, sodium saccharin, talcum, cellulose, glucose, sucrose, magnesium carbonate, can be used to prepare solid formulations. Supplementary active compounds (e.g., preservatives, antibacterial, antiviral and antifungal agents) can also be incorporated into the formulations. A liquid formulation can also be used for enteral administration. The carrier can be selected from various oils including petroleum, animal, vegetable or synthetic, for example, peanut oil, soybean oil, mineral oil, sesame oil. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

Compositions for enteral, parenteral, or transmucosal delivery include, for example, water, saline, phosphate buffered saline, Hank's solution, Ringer's solution, dextrose/saline, and glucose solutions. The formulations can contain auxiliary substances to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. Additional parenteral formulations and methods are described in Bai (1997) J. Neuroimmunol. 80:65 75; Warren (1997) J. Neurol. Sci. 152:31 38; and Tonegawa (1997) J. Exp. Med. 186:507 515. The parenteral preparation can be enclosed in ampules, disposable syringes or multiple dose vials made of glass or plastic.

Compositions for intradermal or subcutaneous administration can include a sterile diluent, such as water, saline solution, fixed oils, polyethylene glycols, glycerine, propylene glycol or other synthetic solvents; antibacterial agents such as benzyl alcohol or methyl parabens; antioxidants such as ascorbic acid, glutathione or sodium bisulfite; chelating agents such as ethylenediaminetetraacetic acid; buffers such as acetates, citrates or phosphates and agents for the adjustment of tonicity such as sodium chloride or dextrose.

Compositions for injection include aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyetheylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as manitol, sorbitol, and sodium chloride can, if needed, be included in the composition. The resulting solutions can be packaged for use as is, or lyophilized; the lyophilized preparation can later be combined with a sterile solution prior to administration.

Compositions can be conventionally administered intravenously, such as by injection of a unit dose, for example. For injection, an active ingredient can be in the form of a parenterally acceptable aqueous solution which is substantially pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as needed.

The term “unit dose” when used in reference to a therapeutic composition refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, and degree of RANKL binding capacity desired. Precise amounts of active ingredient required to be administered depend on the judgment of the practitioner and are peculiar to each individual. However, suitable dosages range from about 0.1 to about 20, about 0.5 to about 10, or about one to about several, milligrams of active ingredient per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated.

In one embodiment, the composition is lyophilized. When the compositions are considered for medicaments, or use in any of the methods provided herein, it is contemplated that the composition can be substantially free of pyrogens such that the composition will not cause an inflammatory reaction or an unsafe allergic reaction.

Acceptable carriers can contain a compound that stabilizes, increases or delays absorption or clearance. Such compounds include, for example, carbohydrates, such as glucose, sucrose, or dextrans; low molecular weight proteins; compositions that reduce the clearance or hydrolysis of peptides; or excipients or other stabilizers and/or buffers. Agents that delay absorption include, for example, aluminum monostearate and gelatin. Detergents can also be used to stabilize or to increase or decrease the absorption of the pharmaceutical composition, including liposomal carriers. To protect from digestion the compound can be complexed with a composition to render it resistant to acidic and enzymatic hydrolysis, or the compound can be complexed in an appropriately resistant carrier such as a liposome. Means of protecting compounds from digestion are known in the art (see, e.g., Fix (1996) Pharm Res. 13:1760 1764; Samanen (1996) J. Pharm. Pharmacol. 48:119 135; and U.S. Pat. No. 5,391,377, describing lipid compositions for oral delivery of therapeutic agents).

For intravenous, injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as needed.

The preparation of compositions (e.g., pharmaceutical or therapeutic compositions) which contain compounds (e.g., polypeptides, antibodies or active fragments) as active ingredients is well understood in the art. Typically, such compositions are prepared as injectables, either as liquid solutions or suspensions. However, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof.

In addition, if desired, the composition can contain minor amounts of auxiliary substances such as wetting or emulsifying agents, pH buffering agents which enhance the effectiveness of the active ingredient.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be through nasal sprays or suppositories (see, e.g., Sayani (1996) “Systemic delivery of peptides and proteins across absorptive mucosae” Crit. Rev. Ther. Drug Carrier Syst. 13:85 184). For transdermal administration, the active compound can be formulated into ointments, salves, gels, or creams as generally known in the art. Transdermal delivery systems can also be achieved using patches.

For inhalation delivery, the formulation can be administered in the form of an aerosol or mist. For aerosol administration, the formulation can be supplied in finely divided form along with a surfactant and propellant. In another embodiment, the device for delivering the formulation to respiratory tissue is in which the formulation vaporizes. Other delivery systems known in the art include dry powder aerosols, liquid delivery systems, inhalers, air jet nebulizers and propellant systems (see, e.g., Patton (1998) Biotechniques 16:141 143; Dura Pharmaceuticals, San Diego, Calif.; Aradigm, Hayward, Calif.; Aerogen, Santa Clara, Calif.; and Inhale Therapeutic Systems, San Carlos, Calif.).

Biodegradable, biocompatible polymers can be used, such as ethylene vinyl acetate, polyanhydrides, polyglycolic acid, collagen, polyorthoesters, and polylactic acid. Methods for preparation of such formulations are known to those skilled in the art. The materials can also be obtained commercially from Alza Corporation and Nova Pharmaceuticals, Inc. Liposomal suspensions (including liposomes targeted to cells or tissues using antibodies or viral coat proteins) can also be used as acceptable carriers. These can be prepared according to methods known in the art, for example, as described in U.S. Pat. Nos. 4,235,871; 4,501,728; 4,522,811; 4,837,028; 6,110,490; 6,096,716; 5,283,185; 5,279,833; Akimaru (1995) Cytokines Mol. Ther. 1:197 210; Alving (1995) Immunol. Rev. 145: 5 31; and Szoka (1980) Ann. Rev. Biophys. Bioeng. 9:467). Biodegradable microspheres or capsules or other biodegradable polymer configurations capable of sustained delivery of small molecules including peptides are known in the art (see, e.g., Putney (1998) Nat. Biotechnol. 16:153 157). Compounds can be incorporated within micelles (see, e.g., Suntres (1994) J. Pharm. Pharmacol. 46:23 28; Woodle (1992) Pharm. Res. 9:260 265). Antibodies can be attached to the surface of the lipid monolayer or bilayer. For example, antibodies can be attached to hydrazide-PEG-(distearoylphosphatidy-1) ethanolamine-containing liposomes (see, e.g., Zalipsky (1995) Bioconjug. Chem. 6: 705 708). Alternatively, any form of lipid membrane, such as a planar lipid membrane or the cell membrane of an intact cell, e.g., a red blood cell, can be used. Liposomal and lipid-containing formulations can be delivered by any means, including, for example, intravenous, transdermal (see, e.g., Vutla (1996) J. Pharma Sci. 85:5 8), transmucosal, or oral administration.

Compositions (an antibody, antigen-binding fragment or binding protein) can be combined with one or more other therapeutic moieties or detectable/imaging/diagnostic moieties as provided herein. Therapeutic moieties and/or detectable/imaging/diagnostic moieties can be provided as separate compositions, or as conjugated moieties. Linkers can be included for conjugated moieties as needed and have been described elsewhere herein. Compositions can be purified using a cleavable or non-cleavable purification tag.

A composition (an antibody, antigen-binding fragment or binding protein) can be administered alone or in combination with other treatments, therapeutics or agents, either simultaneously or sequentially dependent upon the condition to be treated. In addition, the present application contemplates and includes compositions comprising a binding member (e.g., an antibody or antigen-binding fragment thereof) herein described and other agents or therapeutics such as anti-resorptive agents or therapeutics, hormones, anti-RANKL agents or antibodies, or immune modulators. The present application also contemplates and includes compositions comprising two or more binding members (e.g., an antibody or antigen-binding fragment thereof) herein described and, optionally, including other agents or therapeutics such as anti-resorptive agents or therapeutics, hormones, anti-RANKL agents or antibodies, or immune modulators. Other treatments or therapeutics can, if needed, include the administration of suitable doses of pain relief drugs such as non-steroidal anti-inflammatory drugs (e.g. aspirin, paracetamol, ibuprofen or ketoprofen) or opiates such as morphine, or antiemetics. The composition can be administered in combination (either sequentially (i.e. before or after) or simultaneously) with cytokine inhibitors that block the function of TNF alpha, IL-1 and CD40L. In addition, the composition can, if needed, be administered with hormones such as dexamethasone, immune modulators, such as interleukins, tumor necrosis factor (TNF) or other growth factors or cytokines which stimulate the immune response and reduction or elimination of cancer cells or tumors. An immune modulator such as TNF can, in some instances, be combined together with a binding member described herein in, for example, the form of a bispecific antibody recognizing mouse and human RANKL.

The present application further contemplates therapeutic compositions useful in practicing the therapeutic methods described herein. A subject therapeutic composition includes, in admixture, a pharmaceutically acceptable excipient (carrier) and one or more compounds (e.g., a specific binding member such as an antibody or an antigen-binding fragment thereof), as described herein as an active ingredient. In a further embodiment, the composition comprises an antigen or ligand capable of modulating the specific binding of the present binding member/antibody with a target cell or with RANK/RANKL.

Compositions provided herein can further include an adjuvant. The term “adjuvant” refers to a compound or mixture that enhances an immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Often, a primary challenge with an antigen alone, in the absence of an adjuvant, will fail to elicit a humoral or cellular immune response and a secondary challenge is administered. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and useful human adjuvant such as BCG (bacille Calmette-Guerin) and Corynebacterium parvum. Mineral salt adjuvants include but are not limited to: aluminum hydroxide, aluminum phosphate, calcium phosphate, zinc hydroxide and calcium hydroxide. The adjuvant composition can further include a lipid of fat emulsion comprising about 10% (by weight) vegetable oil and about 1-2% (by weight) phospholipids. The adjuvant composition can further optionally include an emulsion form having oily particles dispersed in a continuous aqueous phase, having an emulsion forming polyol in an amount of from about 0.2% (by weight) to about 49% (by weight), optionally a metabolizable oil in an emulsion-forming amount of up to 15% (by weight), and optionally a glycol ether-based surfactant in an emulsion-stabilizing amount of up to about 5% (by weight). Compositions provided herein can, optionally, be lyophilized.

As used herein, the term “immunomodulator” refers to a compound which is able to modulate an immune response. An example of such modulation is neutralization of RANKL binding to RANK. The term “effective amount” of an immunomodulator refers to an amount of an immunomodulator sufficient to bind to RANKL and inhibit binding of RANKL to RANK. An effective amount of an immunomodulator can be an amount capable of eliciting a demonstrable response. The appropriate amount of antigen to be used is dependent on the specific antigen and is known in the art or can be determined empirically. The exact effective amount necessary will vary from subject to subject, depending on the species, age and general condition of the subject, the severity of the condition being treated, the mode of administration, etc.

Compositions have been described above. In one embodiment, the compositions are formulated to be free of pyrogens such that they are acceptable for administration to human and/or veterinary patients. Testing compositions for pyrogens and preparing compositions free of pyrogens are well understood to one of ordinary skill in the art.

Provided herein is a composition of one or more of a cross-reactive binding sequence or of an antibody, an antigen-binding fragment, or a binding proteins that binds to a cross-reactive binding sequence. Exemplary compositions include those such as, for example, that contain one or more of any of the compositions described herein.

The phrase “pharmaceutically acceptable” refers to molecular entities and compositions that are physiologically tolerable and do not typically produce an allergic or similar untoward reaction, such as gastric upset, dizziness and the like, when administered to a human.

The phrase “therapeutically effective amount” is used herein to mean an amount sufficient to reduce by at least about 5 percent, at least about 10 percent, at least about 20 percent, at least about 30 percent, at least about 40 percent, at least about 50 percent, at least about 60 percent, at least about 70 percent, at least about 80 percent, at least about 90 percent, or more, a clinically significant change in bone resorption as measured by, for example, surrogate marker analysis. In one non-limiting example, the degree of activity in bone resorption can be measured, for example, by a reduction in urinary N-telopeptide (NTx) levels.

One embodiment contemplates the use of the compositions described herein to make a medicament for treating a condition, disease or disorder associated with bone loss such as, for example, primary osteoporosis, rheumatoid arthritis, bone metastasis and hypercalcemia of malignancy, glucocorticoid-induced bone loss and periodontal disease; or bone overgrowth. Medicaments can be formulated based on the physical characteristics of the patient/subject needing treatment, and can be formulated in single or multiple formulations based on the stage of the condition, disease or disorder. Medicaments can be packaged in a suitable package with appropriate labels for the distribution to hospitals and clinics wherein the label is for the indication of treating a condition, disease or disorder associated with bone loss or bone resorption in a subject. Medicaments can be packaged as a single or multiple units. Instructions for the dosage and administration of the compositions can be included with the packages as described below.

In still other embodiments, the pharmaceutical compositions containing a compound described herein (e.g., an antibody or antigen-binding fragment) are conjugated to a second pharmaceutically active agent. In one such embodiment, the pharmaceutically active agent is selected from among chemical ablation agents, toxins, immunomodulators, cytokines, and chemotherapeutic agents.

Thus, a composition can be formulated for administration alone or in combination with other treatments, therapeutics or agents, either simultaneously or sequentially dependent upon the condition to be treated. In addition, the present application contemplates and includes compositions comprising a binding member, (e.g., an antibody or antigen-binding fragment thereof), herein described and other agents or therapeutics such as therapeutics for bone disease, anti-cancer agents or immune modulators. More generally, these therapeutics for treatment of bone disease include one or more anti-resorptive or anabolic compound for treatment of bone disease, including but not limited to a bisphosphonate, a Vav3 or Vav3 pathway inhibitor, a calcitonin, a calcitriol, an estrogen, selective estrogen receptor modulators (SERM's) and a calcium source, a supplemental bone formation agent parathyroid hormone (PTH) or its derivative or fragments thereof, PTH related protein (PTHrp), a bone morphogenetic protein, osteogenin, NaF, PGE2 agonists, a statin, and a RANK ligand (RANKL), including an osteogenic form of RANKL such as GST-RANKL or other oligomerized form of RANKL.

It is a still further object of the present application to provide pharmaceutical compositions for use in therapeutic methods for modulation of bone disease which comprise a monoclonal antibody that can cross-react and neutralize RANKL from multiple species. In a further aspect, the application provides pharmaceutical compositions for use in therapeutic methods for modulation of bone disease which comprise an anti-RANKL antibody inhibitor and one or more anti-resorptive or anabolic compound for treatment of bone disease, including but not limited to a bisphosphonate, a Vav3 or Vav3 pathway inhibitor, a calcitonin, a calcitriol, an estrogen, selective estrogen receptor modulators (SERM's) and a calcium source, a supplemental bone formation agent parathyroid hormone (PTH) or its derivative or fragments thereof, PTH related protein (PTHrp), a bone morphogenetic protein, osteogenin, NaF, PGE2 agonists, a statin, and a RANK ligand (RANKL), including an osteogenic form of RANKL such as GST-RANKL or other oligomerized form of RANKL.

Yet, in a further aspect are compositions of binding proteins with additional binding proteins, such as binding proteins which bind to both human and mouse RANKL with equal affinity, inhibiting ligand binding thereto. Such compositions can be “one pot” cocktails, kits, and so forth, formulated for ease of administration.

In one non-limiting example, specific binding members can be used in a method of treatment or diagnosis of the human or animal body, such as a method of treatment of bone loss in a human patient which comprises administering to said patient an effective amount of a specific binding member described herein.

In certain aspects, a pharmaceutically acceptable excipient is, for example, a carrier, a buffer, a stabilizer, or any combination thereof. In one embodiment, the pharmaceutical composition is for oral administration. In another embodiment, the pharmaceutical composition is for intravenous administration.

In one aspect, the binding members (e.g., antibodies, antigen-binding fragments and binding proteins, or active fragments thereof, and chimeric (bispecific) or synthetic antibodies derived therefrom) and that can cross-react and neutralize RANKL from multiple species can be prepared in pharmaceutical compositions, including a suitable vehicle, carrier or diluent, for administration in instances wherein therapy is appropriate, such as to treat osteoporosis. Such pharmaceutical compositions can also include methods of modulating the half-life of the binding members, antibodies or fragments by methods known in the art such as pegylation. Such pharmaceutical compositions can further comprise additional antibodies or therapeutic agents.

In certain embodiments, the application provides pharmaceutical compositions comprising an antibody, or antigen-binding fragment, that binds to a cross-reactive RANKL polypeptide consisting essentially of an amino acid sequence set forth as, for example, any one of SEQ ID NOS: 18-25, and a pharmaceutically acceptable excipient. In certain embodiments, the pharmaceutical compositions comprise an antibody, or antigen-binding fragment, that binds to a cross-reactive RANKL polypeptide consisting essentially of an amino acid sequence set forth as, for example, any one of SEQ ID NOS: 26-43, and a pharmaceutically acceptable excipient.

In certain other embodiments, the application provides pharmaceutical compositions comprising an antibody, or antigen-binding fragment, that binds to a cross-reactive RANKL loop peptide and a pharmaceutically acceptable carrier. The application provides pharmaceutical compositions comprising a monoclonal antibody that can cross-react and neutralize RANKL from multiple species immunoreactive with one or more of polypeptides consisting essentially of, or consisting of, an amino acid sequence set forth as, for example, any of SEQ ID NOS: 1, 2, 3, 5, 6, 7, 9, 10, 11, 13, 14, 15, 17 or 18-25, and a pharmaceutically acceptable carrier. The application provides pharmaceutical compositions comprising a monoclonal antibody that can cross-react and neutralize RANKL from multiple species immunoreactive with one or more of polypeptides consisting essentially of, or consisting of, an amino acid sequence set forth as, for example, any of SEQ ID NOS: 26-43, and a pharmaceutically acceptable carrier.

In one embodiment, the pharmaceutically acceptable excipient is, for example, a carrier, a buffer, a stabilizer, or any combination thereof. In one embodiment, the pharmaceutical composition is for oral administration. In another embodiment, the pharmaceutical composition is for intravenous administration. In still other embodiments, the pharmaceutical composition can further comprise a detectable or functional label. In such one embodiment, the detectable label is a radioisotope. In another such embodiment, the functional label is a cytotoxic drug.

G. Packages and Kits

Provided herein is a package or kit useful for the methods provided herein. One embodiment of such packages or kits includes preparations (compositions) of one or more of any of the cross-reactive binding sequences, or binding members as provided herein.

In a further embodiment, commercial test kits suitable for use by a medical specialist can be prepared to determine the presence or absence of aberrant expression of including but not limited to amplified and/or an mutation, in suspected target cells. In accordance with the testing techniques discussed above, one class of such kits will contain at least the labeled or its binding partner, for instance an antibody specific thereto, and directions, of course, depending upon the method selected, e.g., “competitive,” “sandwich,” “DASP” and the like. The kits can also contain peripheral reagents such as buffers, stabilizers, etc.

Accordingly, a test kit can be prepared for the demonstration of the presence or capability of cells for aberrant expression or post-translational modification of RANKL, comprising: (a) a predetermined amount of at least one labeled immunochemically reactive component obtained by the direct or indirect attachment of the present specific binding member or a specific binding partner thereto, to a detectable label; (b) other reagents; and (c) directions for use of said kit.

More specifically, the diagnostic test kit can comprise: (a) a known amount of one or more of the specific binding members as described above (or a binding partner) generally bound to a solid phase to form an immunosorbent, or in the alternative, bound to a suitable tag, or plural such end products, etc. (or their binding partners) one of each; (b) if necessary, other reagents; and (c) directions for use of said test kit.

The present application includes an assay system which can be prepared in the form of a test kit for the quantitative analysis of the extent of the presence of, for instance, RANKL. The system or test kit can comprise a labeled component prepared by one of the radioactive and/or enzymatic techniques discussed herein, coupling a label to the antibody, and one or more additional immunochemical reagents, at least one of which is a free or immobilized component to be determined or their binding partner(s).

In a further variation, the test kit can be prepared and used for the purposes stated above, which operates according to a predetermined protocol (e.g. “competitive,” “sandwich,” “double antibody,” etc.), and comprises: (a) a labeled component which has been obtained by coupling the specific binding member to a detectable label; (b) one or more additional immunochemical reagents of which at least one reagent is a ligand or an immobilized ligand, which ligand is selected from the group consisting of: (i) a ligand capable of binding with the labeled component (a); (ii) a ligand capable of binding with a binding partner of the labeled component (a); (iii) a ligand capable of binding with at least one of the component(s) to be determined; and (iv) a ligand capable of binding with at least one of the binding partners of at least one of the component(s) to be determined; and (c) directions for the performance of a protocol for the detection and/or determination of one or more components of an immunochemical reaction between the EFGR, the specific binding member, and a specific binding partner thereto.

In accordance with the above, an assay system for screening potential drugs effective to modulate the activity of RANKL, the aberrant expression or post-translational modification of RANKL, and/or the activity or binding of the specific binding member can be prepared. The receptor or the binding member can be introduced into a test system, and the prospective drug can also be introduced into the resulting cell culture, and the culture thereafter examined to observe any changes in the S-phase activity of the cells, due either to the addition of the prospective drug alone, or due to the effect of added quantities of the known agent(s).

Packages and kits can additionally include a buffering agent, a preservative and/or a stabilizing agent in a pharmaceutical formulation. Each component of the kit can be enclosed within an individual container and all of the various containers can be within a single package. Invention kits can be designed for cold storage.

Additionally, the preparations can contain stabilizers to increase the shelf-life of the kits and include, for example, bovine serum albumin (BSA). Where the compositions are lyophilized, the kit can contain further preparations of solutions to reconstitute the lyophilized preparations. Acceptable reconstitution solutions are well known in the art and include, for example, pharmaceutically acceptable phosphate buffered saline (PBS).

Additionally, the packages or kits provided herein can further include any of the other moieties provided herein such as, for example, one or more therapeutic moieties/agents and/or one or more detectable moieties/agents.

Packages and kits can further include one or more components for an assay, such as, for example, an ELISA assay, Scatchard analysis, BIACORE analysis, FACS analysis, etc. Samples to be tested in this application include, for example, blood, plasma, and tissue sections and secretions, urine, lymph, and products thereof. Alternatively, preparations of the kits are used in immunoassays, such as immunohistochemistry to test patient tissue biopsy sections. Packages and kits can further include one or more components for collection of a sample (e.g., a syringe, a cup, a swab, etc.).

Packages and kits can further include a label specifying, for example, a product description, mode of administration and/or indication of treatment. Packages provided herein can include any of the compositions as described herein. The package can further include a label for preventing, inhibiting, treating or managing pain.

The term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions. Kits, therefore, can additionally include labels or instructions for using the kit components in any method of the invention. A kit can include a compound in a pack, or dispenser together with instructions for administering the compound in a method of the invention.

Instructions can include instructions for practicing any of the methods described herein including treatment, prophylaxis, detection, management, monitoring or diagnostic methods. Instructions can additionally include indications of a satisfactory clinical endpoint or any adverse symptoms that can occur, or additional information required by regulatory agencies such as the Food and Drug Administration for use on a human subject.

The instructions can be on “printed matter,” e.g., on paper or cardboard within or affixed to the kit, or on a label affixed to the kit or packaging material, or attached to a vial or tube containing a component of the kit. Instructions can additionally be included on a computer readable medium, such as a disk (floppy diskette or hard disk), optical CD such as CD- or DVD-ROM/RAM, magnetic tape, electrical storage media such as RAM and ROM, IC tip and hybrids of these such as magnetic/optical storage media.

The compositions of the kits can be formulated in single or multiple units for either a single test or multiple tests.

In one aspect, the preparations of the kit are substantially free of pyrogens. Methods for testing for the presence of and/or specific levels of pyrogens are routine in the art and kits are commercially available for such purpose.

Provided herein is a package of one or more of any of compositions described herein. A package containing a composition can, optionally, further include an acceptable carrier or excipient if the composition is lyophilized. In one aspect, the package can be labeled for the diagnosis, detection, monitoring, inhibition, management, prevention or treatment of a condition, disease or disorder associated with bone loss mediated by RANKL. A package can further include one or more components for administration of the composition, such as, for example, syringes for injection, a nebulizer or inhaler for bronchial administration, a patch for transdermal application, etc. The package can also include one or more components for administration.

Provided herein is a kit of one or more of any of compositions described herein. A kit containing a composition can, optionally, further include an acceptable carrier or excipient if the composition is lyophilized. A kit can further contain instructions for use. A kit can, optionally, contain one or more assay components and/or reagents. Exemplary assay components include, but are not limited to one or more components and/or reagents for use in, for example, an ELISA assay, FACS analysis, BIACORE analysis, magnetic bead binding or affinity selection. A kit can also contain one or more detectable moieties, one or more therapeutic moieties, one or more acceptable excipients, one or more acceptable carriers, etc.

IV. In Vitro Expression Systems

A. Prokaryotic Expression Systems

Useful microbial hosts include bacteria from the genera Bacillus, Escherichia (such as E. coli), Pseudomonas, Streptomyces, Salmonella, Erwinia, Bacillus subtilis, Bacillus brevis, the various strains of Escherichia coli (e.g., HB101, (ATCC NO. 33694) DH5a, DH10, and MC1061 (ATCC NO. 53338)).

B. Eukaryotic Expression Systems

i. Yeast

Many strains of yeast cells known to those skilled in the art are also available as host cells for the expression of polypeptides including those from the genera Hansenula, Kluyveromyces, Pichia, Rhino-sporidium, Saccharomyces, and Schizosaccharomyces, and other fingi. Specific yeast cells include, for example, Saccharomyces cerivisae and Pichia pastoris.

ii. Insect Cells

Additionally, where desired, insect cell systems can be utilized in the methods described herein. Such systems are described, for example, by Kitts et al., Biotechniques, 14:810-817 (1993); Lucklow, Curr. Opin. Biotechnol., 4:564-572 (1993); and Lucklow et al. (J. Virol., 67:4566-4579 (1993). Specific insect cells include, for example, Sf-9 and HI5 (Invitrogen, Carlsbad, Calif.).

iii. Mammalian Expression Systems

A number of suitable mammalian host cells are also known in the art and many are available from the American Type Culture Collection (ATCC), 10801 University Boulevard, Manassas, Va. 20110-2209. Examples include, but are not limited to, mammalian cells, such as Chinese hamster ovary cells (CHO) (ATCC No. CCL61) CHO DHFR-cells (Urlaub et al., Proc. Natl. Acad. Sci. USA, 97:4216-4220 (1980)), human embryonic kidney (HEK) 293 or 293T cells (ATCC No. CRL1573), Per.C6 (Crucell, Inc), or 3T3 cells (ATCC No. CCL92). The selection of suitable mammalian host cells and methods for transformation, culture, amplification, screening and product production and purification are known in the art. Other suitable mammalian cell lines are the monkey COS-1 (ATCC No. CRL1650) and COS-7 cell lines (ATCC No. CRL1651), and the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants, are also suitable. Candidate cells can be genotypically deficient in the selection gene, or can contain a dominantly acting selection gene. Other suitable mammalian cell lines include, but are not limited to, mouse neuroblastoma N2A cells, HeLa, mouse L-929 cells, 3T3 lines derived from Swiss, BALB/c or NIH mice, BHK or HaK hamster cell lines, which are available from the ATCC. Each of these cell lines is known by and available for protein expression.

Representative commercially available viral expression vectors include, but are not limited to, the adenovirus-based Per.C6 system available from Crucell, Inc., the lentiviral-based pLP1 from Invitrogen, and the Retroviral Vectors pFB-ERV plus pCFB-EGSH from Stratagene.

An episomal expression vector suitable for the expression of cDNA libraries is able to replicate in the host cell, and persists as an extrachromosomal episome within the host cell in the presence of appropriate selective pressure. (See for example, Conese et al., Gene Therapy 11 1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP), specific examples include the vectors pREP4, pCEP4, pREP7 from Invitrogen. The vectors pcDNA3.1 from Invitrogen, and pBK-CMV from Stratagene represent non-limiting examples of an episomal vector that uses T-antigen and the SV40 origin of replication in lieu of EBNA1 and orip.

An integrating expression vector suitable for expression can randomly integrate into the host cell's DNA, or can include a recombination site to enable the specific recombination between the expression vector and the host cells chromosome. Such integrating expression vectors can utilize the endogenous expression control sequences of the host cell's chromosomes to effect expression of the desired protein. Examples of vectors that integrate in a site specific manner include, for example, components of the flp-in system from Invitrogen (e.g., pcDNA™5/FRT), or the cre-lox system, such as can be found in the pExchange-6 Core Vectors from Stratagene. Examples of vectors that integrate into host cell chromosomes in a random fashion include, for example, pcDNA3.1 (when introduced in the absence of T-antigen) from Invitrogen, pCI or pFN10A (ACT) Flexi® from Promega.

Alternatively, the expression vector can be used to introduce and integrate a strong promoter or enhancer sequences into a locus in the cell so as to modulate the expression of an endogenous gene of interest (Capecchi M R. Nat Rev Genet. (2005); 6 (6):507-12; Schindehutte et al., Stem Cells (2005); 23 (1):10-5). This approach can also be used to insert an inducible promoter, such as the Tet-On promoter (U.S. Pat. Nos. 5,464,758 and 5,814,618), in to the genomic DNA of the cell so as to provide inducible expression of an endogenous gene of interest. The activating construct can also include targeting sequence(s) to enable homologous or non-homologous recombination of the activating sequence into a desired locus specific for the gene of interest (see for example, Garcia-Otin & Guillou, Front Biosci. (2006) 11:1108-36). Alternatively an inducible recombinase system, such as the Cre-ER system can be used to activate a transgene in the presence of 4-hydroxytamoxifen. (Indra et al. Nuc. Acid. Res. (1999) 27 (22) 4324-4327; Nuc. Acid. Res. (2000) 28 (23) e99; U.S. Pat. No. 7,112,715).

Elements to be included in an expression vector for use compounds and methods described herein are well known in the art, and any existing vector can be readily modified for use, for example, through the insertion or replacement of one or more polynucleotide sequences with synthetic polynucleotide sequences as described above.

V. Screening and Enrichment Systems

Polypeptides generated by the expression of cDNA libraries can be screened using a variety of standard physiological, pharmacological and biochemical procedures. Such assays include for example, biochemical assays such as binding assays, fluorescence polarization assays, solubility assays, folding assays, thermostability assays, proteolytic stability assays, and enzyme activity assays (see generally Glickman et al., J. Biomolecular Screening, 7 No. 13-10 (2002); Salazar et al., Methods. Mol. Biol. 230 85-97 (2003)), as well as a range of cell based assays including signal transduction, motility, whole cell binding, flow cytometry and fluorescent activated cell sorting (FACS) based assays. Cells expressing an antibody of interest can be enriched any art-recognized assay including, but not limited to, methods of coupling peptides to microparticles.

Many FACS and high throughput screening systems are commercially available (see, e.g., Zymark Corp., Hopkinton, Mass.; Air Technical Industries, Mentor, Ohio; Beckman Instruments Inc., Fullerton, Calif.; Precision Systems, Inc., Natick, Mass.) that enable these assays to be run in a high throughput mode. These systems typically automate entire procedures, including all sample and reagent pipetting, liquid dispensing timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, for example, Zymark Corp. provides technical bulletins describing screening systems for detecting the modulation of gene transcription, ligand binding, and the like.

A. Selection and Enrichment Strategies

i. Flow Cytometry and FACS

Flow cytometry and the related flow sorting (also known as fluorescence activated cell sorting, or FACS) are methods by which individual cells can be quantitatively assayed for the presence of a specific component or component variant based upon staining with a fluorescent reporter. Flow cytometry provides quantitative, real time analysis of living cells, and can achieve efficient cell sorting rates of 50,000 cells/second, and is capable of selecting individual cells or defined populations. Many commercial FACS systems are available, for example BD Biosciences (CA), Cytopeia (Seattle, Wash.) Dako Cytomation (Australia).

A FACS can be equipped with a variety of lasers, which can produce a wide range of available wavelengths for multiple parameter analysis, and for use with different fluorophores. Classically the water cooled ion lasers using argon, krypton, or a mix of both can produce several specific lines; 408 nm, 568 nm, and 647 nm for example are major emission lines for Krypton; 488 nm, 457 nm, and others are argon lines. These lasers require high voltage multiphase power and cooling water, but can produce high power outputs. Additionally tunable and non tunable diode lasers exist, for example a 408 nm line can be stably created via a light emitting diode (LED) and this can be easily added to a sorter. Additionally dye lasers can be used to further extend the range of available wavelengths available for FACS analysis.

During FACS analysis, cells are stained with the specific reporter and then hydrodynamically focused into a single cell steam for interrogation with a laser which excites the fluorescent moiety. Fluorescent emission is detected through a wavelength restricted optical pathway and converted to numeric data correlated to an individual cell. In the case of flow sorting, predefined subsets of emission criteria can be met and the cells of interest diverted into a collection receptacle for further use by electrostatic repulsion or mechanical action (Herzenberg L A, Sweet R G, Herzenberg L A (1976): Fluorescence activated cell sorting, Sci. Amer. 234(3):108).

FACS based approaches are compatible with wide variety of assays and can be efficiently applied to most mammalian, yeast and bacterial cells, as well as fluorescently tagged beads.

Fluorescent moieties to be detected include, but are not limited to, compounds such as fluorescein (commonly called FITC), phycobiliproteins such as phycoerythrin (PE) and allophycocyanin (APC) (Kronick, M. N. J. Imm. Meth. (1986) 92:1-13), fluorescent semiconductor nanocrystals such as Quantum dot (QDot) bioconjugates for ultrasensitive nonisotopic detection (Chan W C, Nie S., Science (1998) 281: 2016-8), labeled microspheres (Luminex) which can be distinguished either by variations in size or intrinsic fluorescence, (Kettman Cytometry (1998) 33 (2) 234-243) and coumarin derivatives such as Fluorescent Acylating Agents derived from 7-Hydroxycoumarin.

Fluorescence can also reported from fluorescent proteins such as Teal Fluorescent Protein (TFP), from chemical stains of cellular components such as DAPI bound to DNA, from fluorescent moieties covalently conjugated to antibodies that recognize cellular products, from fluorescent moieties covalently conjugated to ligands of cellular receptors, and from fluorescent moieties covalently conjugated to substrates of cellular enzymes.

Cells stained with membrane impermeant reporters, such as antibodies, can be sorted for subsequent processing to recover components such as genes, episomes, or proteins of interest. Cells stained for surface expression components or stained with cell membrane permeant reporters can also be sorted intact for propagation.

ii. Affinity Separation

Affinity separation based on the use microparticles enables the separation of surface displayed proteins based on affinity to a specific compound or sequence of interest. This approach is rapid, can easily be scaled up, and can be used iteratively with living cells.

Paramagnetic polystyrene microparticles are commercially available (Spherotech, Inc., Libertyville, Ill.; Invitrogen, Carlsbad, Calif.) that couple compounds or peptides to microparticle surfaces that have been modified with functional groups or coated with various antibodies or ligands such as, for example, avidin, streptavidin or biotin.

In one aspect paramagnetic beads can be used in which the paramagnetic property of microparticles allows them to be separated from solution using a magnet. The microparticles can be easily re-suspended when removed from the magnet thereby enabling the selective separation of cells that find to the attached probe.

In one embodiment, peptides can be coupled to paramagnetic polystyrene microparticles coated with a polyurethane layer in a tube. The hydroxyl groups on the microparticle surface are activated by reaction with p-toluensulphonyl chloride (Nilsson K and Mosbach K. “p-Toluenesulfonyl chloride as an activating agent of agarose for the preparation of immobilized affinity ligands and proteins.” Eur. J. Biochem. 1980:112: 397-402). The resulting sulphonyl ester can subsequently react covalently with peptide amino or sulfhydryl groups. The peptides are quickly absorbed onto the surface of the activated microparticles followed by the formation of covalent amine bonds with further incubation. The microparticles (2⁰⁹ microparticles/milliliter) are washed two times by placing the tube containing 1 milliliter (ml) of microparticles on a magnet, allowing the microparticles to migrate to the magnet side of the tube, removing the supernatant, and re-suspending the microparticles in 1 ml of 100 millimolar (mM) borate buffer, pH 9.5. After washing, the microparticles are re-suspended in 100 mM borate buffer, pH 9.5 at a concentration of 1⁰⁹ microparticles/ml. Eleven nanomoles of peptide are added to the microparticles and the microparticle/peptide mixture is vortexed for 1 minute to mix. The microparticles are incubated with peptides at room temperature for at least 48 hours with slow tilt rotation. To ensure an optimal orientation of the peptide on the microparticles, bovine serum albumin (BSA) is added to the microparticle/peptide mixture to a final concentration of 0.1% (weight/volume) after incubation has proceeded for 10 minutes. After incubation, the tube containing the microparticle/peptide mixture is placed on the magnet until the microparticles migrate to the magnet side of the tube. The supernatant is removed and the microparticles are washed four times with 1 ml phosphate buffered saline solution (PBS), pH 7.2 containing 1% (weight/volume) BSA. Finally, the microparticles are re-suspended in 1 ml PBS solution, pH 7.2 containing 1% (weight/volume) BSA.

Alternatively, paramagnetic polystyrene microparticles containing surface carboxylic acid can be activated with a carbodiimide followed by coupling to a peptide, resulting in a stable amide bond between a primary amino group of the peptide and the carboxylic acid groups on the surface of the microparticles (Nakajima N and Ikade Y, Mechanism of amide formation by carbodiimide for bioconjugation in aqueous media, Bioconjugate Chem. 1995, 6(1), 123-130; Gilles M A, Hudson A Q and Borders C L Jr, Stability of water-soluble carbodiimides in aqueous solution, Anal Biochem. 1990 Feb. 1; 184(2):244-248; Sehgal D and Vijay I K, a method for the high efficiency of water-soluble carbodiimide-mediated amidation, Anal Biochem. 1994 April; 218(1):87-91; Szajani B et al, Effects of carbodiimide structure on the immobilization of enzymes, Appl Biochem Biotechnol. 1991 August; 30(2):225-231). The microparticles (29 microparticles/milliliter) are washed twice with 1 ml of 25 mM 2-[N-morpholino]ethane sulfonic acid, pH 5 for 10 minutes with slow tilt rotation at room temperature. The washed microparticles are re-suspended in 700 microliters (μL) 25 mM 2-[N-morpholino]ethane sulfonic acid, pH 5 followed by the addition of 21 nanomoles of peptide re-suspended in 25 mM 2-[N-morpholino]ethane sulfonic acid, pH 5 to the microparticle solution. The microparticle/peptide mixture is mixed by vortexing and incubated with slow tilt rotation for 30 minutes at room temperature. After this first incubation, 300 μL of ice-cold 100 milligram (mg)/mL 1-ethyl-3-(3-dimethylaminopropyl)carbodiimide hydrochloride re-suspended in 25 mM 2-[N-morpholino]ethane sulfonic acid, pH 5 is added to the peptide/microparticle mixture and incubated overnight at 4° Celsius with slow tilt rotation. The peptide-coupled microparticles are washed four times with 1 ml 50 mM Tris pH 7.4/0.1% BSA for 15 minutes at room temperature with slow tilt rotation. After washing, the peptide-coupled microparticles are re-suspended at a concentration of 1⁹ microparticles/mil in PBS solution, pH 7.2 containing 1% (weight/volume) BSA.

Another option is to couple biotinylated peptides to paramagnetic polystyrene microparticles whose surfaces have been covalently linked with a monolayer of streptavidin. Briefly, one ml of the streptavidin microparticles are transferred to a microcentrifuge tube and washed four times by placing the tube on a magnet and allowing the microparticles to collect on the magnet side of the tube. The solution is then removed and the microparticles are gently re-suspended in 1 ml of PBS solution, pH 7.2 containing 1% (weight/volume) BSA. After the final wash, the microparticles are re-suspended in 1 ml of PBS solution, pH 7.2 containing 1% (weight/volume) BSA; and 33 picomoles of biotinylated peptide are added to the microparticle solution. The microparticle/peptide solution is incubated for 30 minutes at room temperature with slow tilt rotation. After coupling, the unbound biotinylated peptide is removed from the microparticles by washing four times with PBS solution, pH 7.2 containing 1% (weight/volume) BSA. After the final wash, the microparticle/peptide mixture is re-suspended to a final bead concentration of 1⁹ microparticles/ml. (Argarana C E, Kuntz I D, Birken S, Axel R, Cantor C R. Molecular cloning and nucleotide sequence of the streptavidin gene. Nucleic Acids Res. 1986; 14(4):1871-82; Pahler A, Hendrickson W A, Gawinowicz Kolks M A, Aragana C E, Cantor C R. Characterization and crystallization of core streptavidin. J Biol Chem 1987:262(29):13933-7)

VI. Diagnostic and Therapeutic Uses

The proteins of interest, e.g., antibodies, identified by the methods described herein can be used non-therapeutic agents, for example, as affinity purification agents. In such an embodiment, a protein of interest is immobilized on a solid phase such a Sephadex resin or filter paper, using methods well known in the art. The immobilized protein is contacted with a sample containing the target of interest (or fragment thereof) to be purified, and thereafter the support is washed with a suitable solvent that will remove substantially all the material in the sample except the target protein, which is bound to the immobilized antibody. Finally, the support is washed with another suitable solvent, such as glycine buffer, pH 5.0, which will release the target protein.

Compositions can be administered in a therapeutically effective amount which are effective for producing some desired therapeutic effect by inhibiting bone loss, inhibiting bone resorption, and preventing or treating a condition or disease associated with bone loss such as, for example, osteoporosis, rheumatoid arthritis, bone metastasis and hypercalcemia of malignancy, glucocorticoid-induced bone loss, a periodontal disease or condition, a cancer and Juvenile Paget's Disease, at a reasonable benefit/risk ratio applicable to any medical treatment. For the administration of the present compositions to human patients, the compositions can be formulated by methodology known by one of ordinary skill in the art.

An effective immune response is achieved when the patient experiences partial or total alleviation, or reduction of signs or symptoms of illness, and specifically includes, without limitation, prolongation of survival. The expected progression-free survival times can be measured in months to years, depending on prognostic factors including the number of relapses, stage of disease, and other factors. Prolonging survival includes without limitation times of at least 1 month (mo), about at least 2 months (mos.), about at least 3 mos., about at least 4 mos., about at least 6 mos., about at least 1 year, about at least 2 years, about at least 3 years, etc. Overall survival can also be measured in months to years. The patient's symptoms may remain static, and the bone loss may not increase.

A physician or veterinarian having ordinary skill in the art can readily determine and prescribe the effective amount (ED50) of the composition required. For example, the physician or veterinarian could start doses of the compounds employed in the composition at levels lower than that required in order to achieve the desired therapeutic effect and gradually increase the dosage until the desired effect is achieved.

Compositions can be administered to a patient by any convenient route, including, for example, subcutaneous, intravenous, intra-arterial, intraperitoneal, or intramuscular injection. Regardless of the route of administration selected, the compounds of the present invention, which can be used in a suitable hydrated form, and/or the compositions, are formulated into acceptable dosage forms such as described below or by other conventional methods known to those of skill in the art.

Actual dosage levels of the active ingredients in the compositions can be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound employed, the route of administration, the time of administration, the rate of excretion of the particular compound being employed, the duration of the treatment, other drugs, compounds and/or materials used in combination with the particular composition employed, the age, sex, weight, condition, general health and prior medical history of the patient being treated, and like factors well known in the medical arts.

RANKL cross-reactive binding sequences and/or binding members can be combined with a therapeutic moiety or to a detectable (imaging) moiety using methods known in the art such as, for example, chemical conjugation, covalent or non-covalent bonds or recombinant techniques to create conjugates or fusion proteins. Alternatively, cross-reactive binding sequences and/or binding members can be combined in separate compositions for simultaneous or sequential administration.

The unique specificity of the specific binding members, whereby the binding member(s) recognize an epitope which is found in both mouse and human RANKL and neutralize RANKL biological activity, provides diagnostic and therapeutic uses to in diseases characterized by excessive bone loss, including but not limited to primary osteoporosis, rheumatoid arthritis, bone metastasis and hypercalcemia of malignancy, glucocorticoid-induced bone loss and periodontal disease.

Also, antibodies including both polyclonal and monoclonal antibodies, and drugs that modulate the production or activity of the specific binding members, antibodies and/or their subunits can be used for diagnostic applications and can, for example, be utilized for the purpose of detecting and/or measuring conditions such as pathological bone loss. For example, the specific binding members, antibodies or their subunits can be used to produce both polyclonal and monoclonal antibodies to themselves in a variety of cellular media, by known techniques such as the hybridoma technique utilizing, for example, fused mouse spleen lymphocytes and myeloma cells. Likewise, small molecules that mimic or antagonize one or more activities of the specific binding members can be discovered or synthesized, and can be used in diagnostic and/or therapeutic protocols.

A. Diagnostic Uses

Provided herein are a variety of diagnostic applications, including methods for detecting the presence of cross-reactive binding sequences by reference to their ability to be recognized by an antibody, antigen-binding fragment or binding protein such as those as described herein. An anti-cross-reactive binding sequence antibody can be used to produce antibodies to itself by a variety of known techniques (anti-idiotype), and such antibodies can then be isolated and utilized in tests for the presence of particular activity in suspect target cells.

Diagnostic applications of the antibodies, antigen-binding fragments or binding proteins include in vitro and in vivo applications well known and standard to one or ordinary skill in the art and based on the present application. Diagnostic assays and kits for in vitro assessment and evaluation of bone resorption status, such as with regard to aberrant expression of RANKL, can be utilized to diagnose, evaluate and monitor patient samples including those known to have or suspected of having osteoporosis, for example. The assessment and evaluation of RANKL status can also be useful in determining the suitability of a patient for a clinical trial of a drug or for the administration of a therapeutic agent or an antibody, antigen-binding fragment or binding protein, including combinations thereof. In vivo applications include imaging of bones or assessing osteoporosis status of individuals such as, for example, radioimaging.

Provided herein are methods of detecting, diagnosing and/or monitoring one or more conditions, diseases or disorders by administering any one of the compositions provided herein to a subject. In one non-limiting aspect, the composition to be administered further comprises a detectable moiety and/or a therapeutic moiety.

In the detection, diagnosis or monitoring of bone loss, the subject patient is administered an antibody, antigen-binding fragment, or binding protein that binds to a cross-reactive RANKL polypeptide, which antibody, antigen-binding fragment or binding protein is bound to a detectable moiety. The detectable moiety can be visualized using art-recognized methods such as, but not limited to, magnetic resonance imaging (MRI), fluorescence, radioimaging, light sources supplied by endoscopes, laparoscopes, or intravascular catheter (i.e., via detection of photoactive agents), photoscanning, positron emission tomography (PET) scanning, whole body nuclear magnetic resonance (NMR), radioscintography, single photon emission computed tomography (SPECT), targeted near infrared region (NIR) scanning, etc. such as described, for example, in U.S. Pat. No. 6,096,289, U.S. Pat. No. 7,115,716, U.S. Pat. No. 7,112,412, U.S. Patent Application No. 20030003048 and U.S. Patent Application No. 20060147379, each of which is incorporated herein in its entirety by reference. Labels for detecting compounds using such methods are also known in the art and described in such patents and applications and are incorporated herein by reference. Visualization of the detectable moiety can allow for detection, diagnosis, and/or monitoring of a condition or disease associated with the RANKL cross-reactive binding sequences.

Proteins identified by the methods described herein can also be useful in diagnostic assays for the targeted protein, e.g., detecting its expression in specific cells, tissues, or serum. Such diagnostic methods can be useful in cancer diagnosis.

For diagnostic applications, the proteins will typically be labeled with a detectable moiety. In certain embodiments, the detectable moiety can be selected from the following categories: (a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Coligen et al., Ed. Wiley-Interscience, New York, N.Y., Pubs. (1991) for example and radioactivity can be measured using scintillation counting; (b) Fluorescent labels such as rare earth chelates (europium chelates) or fluorescein and its derivatives, rhodamine and its derivatives, dansyl, Lissamine, phycoerythrin and Texas Red are available; (c) enzyme-substrate labels.

Various enzyme substrate labels are known in the art and U.S. Pat. No. 4,275,149 provides a review of some of these. The enzyme generally catalyzes a chemical alteration of the chromogenic substrate which can be measured using various techniques. For example, the enzyme can catalyze a color change in a substrate, which can be measured spectrophotometrically. Alternatively, the enzyme can alter the fluorescence or chemiluminescence of the substrate. Techniques for quantifying a change in fluorescence are described above. The chemiluminescent substrate becomes electronically excited by a chemical reaction and can then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzymol. (ed J. Langone & H. Van Vunakis), Academic press, New York, 73:147 166 (1981).

In certain embodiments, enzyme-substrate combinations can include, for example: (i) Horseradish peroxidase (HRPO) with hydrogen peroxidase as a substrate, wherein the hydrogen peroxidase oxidizes a dye precursor (e.g., orthophenylene diamine (OPD)); (ii) alkaline phosphatase (AP) with para-Nitrophenyl phosphate as chromogenic substrate; and (iii) β-D-galactosidase (β-D-Gal) with a chromogenic substrate (e.g., p-nitrophenyl-β-D-galactosidase) or fluorogenic substrate 4-methylumbelliferyl-β-D-galactosidase.

Numerous other enzyme-substrate combinations are available to those skilled in the art. For a general review of these, see U.S. Pat. Nos. 4,275,149 and 4,318,980.

The proteins identified by the methods described herein can be employed in any known assay method, such as competitive binding assays, direct and indirect sandwich assays, and immunoprecipitation assays. Zola, Monoclonal Antibodies: A Manual of Techniques, pp. 147 158 (CRC Press, Inc. 1987).

The antibodies can also be used for in vivo diagnostic assays as described in more detail below. Generally, the antibody is labeled with a radio nuclide (such as ¹¹¹In, ¹⁴C, ¹³¹I, ¹²⁵I, ³H, ³²P or ³⁵S) so that a tumor can be localized using immunoscintiography.

A diagnostic method can include examining/assaying a sample by means of an assay including an effective amount of an antibody, antigen-binding fragment or binding protein that binds to RANKL. Such antibodies, antigen-binding fragments and binding protein have been described elsewhere herein. As previously discussed, patients capable of benefiting from this method include those suffering from bone loss. Approaches for making and isolating antibodies, antigen-binding fragments and binding proteins are provided herein and for methods for determining and optimizing the ability of antibodies, antigen-binding fragments and binding proteins to assist in the examination of the target cells are all well-known in the art.

The antibodies can thus specifically categorize the nature of osteoblasts, stromal cells, activated T cells, activated endothelium, and mammary epithelium cells, by staining or otherwise recognizing those cells wherein RANKL is present on the cell surface or released as a soluble polypeptide.

Non-limiting conditions, diseases and disorders to be considered for these methods include, but are not limited to, those associated with bone loss such as, for example, primary osteoporosis, rheumatoid arthritis, bone metastasis and hypercalcemia of malignancy, glucocorticoid-induced bone loss and periodontal disease. In the detection, diagnosis or monitoring of such bone loss diseases, the subject patient is administered a composition of an antibody, antigen-binding fragment, or binding protein that binds to a cross-reactive binding sequence of, for example, RANKL, which antibody, antigen-binding fragment, or binding protein is conjugated to a detectable moiety. The moiety can be visualized using art-recognized methods such as those described above. Visualization of the detectable moiety can allow for detection, diagnosis, and/or monitoring of a condition or disease associated with bone loss.

In accordance with the above, an assay system for screening drugs effective to modulate the activity of RANKL, the aberrant expression or post-translational modification of RANKL, and/or the activity or binding of the antibody, antigen-binding fragment or binding protein can be prepared. The receptor or the antibody can be introduced into a test system, and the prospective drug can also be introduced into the resulting cell culture, and the culture thereafter examined to observe any changes in the S-phase activity of the cells, due either to the addition of the prospective drug alone, or due to the effect of added quantities of the known agent(s).

There are multiple osteoclast culture systems or methods and bone formation assays that can be used successfully to screen potential an anti-RANKL antibody described herein. See, e.g., U.S. Pat. No. 6,080,779, incorporated herein by reference in its entirety.

One osteoclast culture for use in screening is a neonatal mouse calvaria assay. Briefly, four days after birth, the front and parietal bones of neonatal mouse pups (e.g., ICR Swiss white mice) are removed by microdissection and split along the sagittal suture. The bones are then incubated in a specified medium, wherein the medium contains either test or control compounds. Following the incubation, the bones are removed from the media, and fixed in 10% buffered formalin, decalcified in EDTA, processed through graded alcohols, and embedded in paraffin wax. Sections of the calvaria are prepared and assessed using histomorphometric analysis of bone formation and bone resorption. Bone changes are measured on sections. Osteoblasts and osteoclasts are identified by their distinctive morphology.

In addition to this assay, the effect of compounds on murine calvarial bone growth can also be tested in vivo. In one such example of this screening assay, young male mice (e.g., ICR Swiss white mice), aged 4-6 weeks are employed, using 4-5 mice per group. Briefly, the test compound or the appropriate control is injected into subcutaneous tissue over the right calvaria of normal mice. The mice are sacrificed (after allowing for bone growth or loss to occur, e.g. on day 14), and net bone growth is measured by histomorphometric means. Bone samples are cleaned from adjacent tissues and fixed in 10% buffered formalin, decalcified, processed through graded alcohols, and embedded in paraffin wax. Sections of the calvaria are prepared, and representative sections are selected for histomorphometric assessment of the effects of bone formation and bone resorption. In one embodiment, sections are measured by using a camera lucida attachment to trace directly the microscopic image onto a digitizing plate. Bone changes are measured on sections over adjacent 1×1 mm fields on both the injected and noninjected sides of calvaria. New bone can be identified by those skilled in the art by its characteristic tinctorial features, and osteoclasts and osteoblasts can be identified by their distinctive morphology or other suitable marker recognized by the skilled artisan. Histomorphometry software (OsteoMeasure, Osteometrix, Inc., Atlanta) can be used to process digitized input to determine cell counts and measure areas or perimeters.

Additional exemplary in vivo assays include dosing assays in intact animals, including dosing assays in acute ovariectomized (OVX) animals and assays in chronic OVX animals. Prototypical dosing in intact animals can be accomplished by subcutaneous, intraperitoneal or oral administration, and can be performed by injection, sustained release or other delivery techniques. The time period for administration of test compound can vary (for instance, 14 days, 28 days, as well as 35 days or longer).

As an example, in vivo oral or subcutaneous dosing assay can be performed as described: In a typical study, 70 three-month-old female Sprague-Dawley rats are weight-matched and divided into treatment groups, with at least several animals in each group. This includes a baseline control group of animals sacrificed at the initiation of the study; a control group administered vehicle only; a PBS or saline-treated control group; and a positive group administered a compound known to enhance net bone formation. Three dosage levels of the test compound are administered to the remaining groups. Test compound, saline, and vehicle are administered (e.g. once per day) for a number of days (for instance at least 14 days, 28 days, or 35 days—wherein an effect is expected in the positive group). All animals are injected calcein nine days and two days before sacrifice (to ensure proper labeling of newly formed bone). Weekly body weights are determined. At the end of the period of compound administration, the animals are weighed and bled by orbital or cardiac puncture. Serum calcium, phosphate, osteocalcin, and CBCs are determined. Both leg bones (femur and tibia) and lumbar vertebrae are removed, cleaned of adhering soft tissue, and stored in 70% ethanol or 10% formalin for evaluation, for instance as performed by peripheral quantitative computed tomography (pQCT; Ferretti, J, Bone, 17: 353S-364S, 1995), dual energy X-ray absorptiometry (DEXA; Laval-Jeantet A. et al., Calcif Tissue Intl, 56:14-18, 1995, and Casez J. et al., Bone and Mineral, 26:61-68, 1994) and/or histomorphometry. The effect of test compounds on bone remodeling or net bone formation, including bone loss and osteoclast function can thus be evaluated.

Test compounds can also be assayed in acute ovariectomized animals. Such assays can also include an estrogen-treated group as a control. An example of the test in these animals is briefly described: In a typical study, 80 three-month-old female Sprague-Dawley rats are weight-matched and divided into treatment groups, with at least several animals in each group. This includes a baseline control group of animals sacrificed at the initiation of the study; three control groups (sham OVX and vehicle only, OVX and vehicle only, and OVX and PBS only); and a control OVX group that is administered a compound known to block or reduce bone resorption or enhance bone formation (including an anti-resorptive or anabolic compound). Different dosage levels of the test compound are administered to remaining groups of OVX animals.

Since ovariectomy induces hyperphagia, all OVX animals are pair-fed with sham OVX animals throughout the study. Test compound, positive control compound, PBS or saline or vehicle alone is administered orally or subcutaneously (e.g., once per day) for the treatment period. As an alternative, test compounds can be formulated in implantable pellets that are implanted, or can be administered orally, such as by gastric gavage. All animals are injected with calcein nine days and two days before sacrifice. Weekly body weights are determined. At the end of the treatment cycle, the animal's blood and tissues are processed as described above.

Test compounds can also be assayed in chronic OVX animals. Briefly, six month old female, Sprague-Dawley rats are subjected to sham surgery (sham OVX), or ovariectomy (OVX) at the beginning of the experiment, and animals are sacrificed at the same time to serve as baseline controls. Body weights are monitored weekly. After approximately six weeks or more of bone depletion, sham OVX and OVX rats are randomly selected for sacrifice as depletion period controls. Of the remaining animals, 10 sham OVX and 10 OVX rats are used as placebo-treated controls. The remaining animals are treated with 3 to 5 doses of test compound for a period of 35 days. As a positive control, a group of OVX rats can be treated with a known anabolic or anti-resorptive agent in this model, such as bisphosphonate, a calcitonin, a calcitriol, an estrogen, selective estrogen receptor modulators (SERM's) and a calcium source, a supplemental bone formation agent parathyroid hormone (PTH) or its derivative (Kimmel et al., Endocrinology, 132: 1577-1584, 1993), PTHRP, a bone morphogenetic protein, osteogenin, NaF, PGE2 agonists, a statin, and a RANK ligand (RANKL), including an osteogenic form of RANKL such as GST-RANKL or other oligomerized form of RANKL. At the end of the experiment, the animals are sacrificed and femurs, tibiae, and lumbar vertebral to 4 are excised and collected. The proximal left and right tibiae are used for pQCT measurements, cancellous bone mineral density (BMD), and histology, while the midshaft of each tibiae is subjected to cortical BMD or histology. The femurs are prepared for pQCT scanning of the midshaft prior to biomechanical testing. With respect to lumbar vertebrae (LV), LV2 are processed for BMD (pQCT can also be performed), LV3 are prepared for undecalcified bone histology, and LV4 are processed for mechanical testing.

In addition, osteoclast cultures, containing macrophages, osteoclast precursors and osteoclasts, can be generated from bone marrow precursors (e.g., bone marrow macrophages) and utilized in assessment of compounds for osteoclast modulating activity. Bone marrow macrophages are cultured in 48- or 96-well cell culture dishes in the presence of M-CSF (10 ng/ml), RANKL (100 ng/ml), with or without addition of compound(s) or control(s), and medium changed (e.g., on day 3). Osteoclast-like cells are characterized by staining for tartrate-resistant acid phosphatase (TRAP) activity. In assessing bone resorption, for instance using a pit assay, osteoclasts are generated on whale dentin slices from bone marrow macrophages. After three days of culture to generate osteoclasts, compound(s) or control(s) are added to the culture for two days. At the end of the experiment, cells are TRAP stained and photographed to document cell number. Cells are then removed from the dentin slices with 0.5M ammonium hydroxide and mechanical agitation. Maximum resorption lacunae depth is measured using a confocal microscope (Microradiance, Bio-Rad Laboratories, Hercules, Calif.). For evaluation of pit number and resorbed area, dentin slices are stained with Coumassie brilliant blue and analyzed with light microscopy using Osteomeasure software (Osteometrics, Decatur, Ga.) for quantitation.

In a further method, osteoclast modulating ability of an antibody is tested in an in vitro assay utilizing osteoclasts, osteoclast precursor cells or osteoclast-like cells. General protocols for treatment of osteoclasts with a compound are well established and known in the art. For instance, bone marrow macrophages can be utilized to generate osteoclasts in vitro as described herein. It is to be noted that the conditions used will vary according to the cell lines and compound used, their respective amounts, and additional factors such as plating conditions and media composition. Such adjustments are readily determined by one skilled in this art.

The present application also relates to a variety of diagnostic applications, including methods for detecting the presence of RANKL or RANK, by reference to their ability to be recognized by the present specific binding member, or a monoclonal antibody that can cross-react and neutralize RANKL from multiple species.

Diagnostic applications of the specific binding members (e.g., antibodies and fragments thereof) include in vitro and in vivo applications well known and standard to the skilled artisan and based on the present description. Diagnostic assays and kits for in vitro assessment and evaluation of bone resorption status, such as with regard to aberrant expression of RANKL, can be utilized to diagnose, evaluate and monitor patient samples including those known to have or suspected of having osteoporosis, for example. The assessment and evaluation of RANKL status can also be useful in determining the suitability of a patient for a clinical trial of a drug or for the administration of a particular therapeutic agent Or Specific binding member of the present invention, including combinations thereof, versus a different agent or binding member. This type of diagnostic monitoring and assessment is already in practice utilizing antibodies against the HER2 protein in breast cancer (Hercep Test, Dako Corporation), where the assay is also used to evaluate patients for antibody therapy using Herceptin. In vivo applications include imaging of bones or assessing osteoporosis or cancer status of individuals, including radioimaging.

As suggested earlier, a diagnostic method can comprise examining a cellular sample or medium by means of an assay including an effective amount of an antagonist to RANKL, such as an anti-RANKL antibody (e.g., an affinity-purified polyclonal antibody or a mAb. In addition, the anti-RANKL antibody molecules used herein can be in the form of Fab, Fab′, F(ab′)₂ or F(v) portions (or any other functional portion described herein) or whole antibody molecules. As previously discussed, patients capable of benefiting from this method include those suffering from excessive bone loss osteoporosis and in certain cancers. Approaches for isolating and inducing antibodies are provided herein and for methods for determining and optimizing the ability of antibodies to assist in the examination of the target cells are all well-known in the art.

A, anti-RANKL antibody used in diagnostic methods can be an affinity purified polyclonal antibody or a monoclonal antibody (mAb). In addition, the anti-RANKL antibody molecules used herein can be in the form of Fab, Fab′, F(ab′)₂ or F(v) portions (or any other functional portion described herein) of whole antibody molecules.

As described in detail above, antibody(ies) to mouse RANKL sequences, or other non-human RANKL sequences or cross-reactive sequences, can be produced and isolated by standard methods including the well known hybridoma techniques. For convenience, the antibody(ies) to mouse/other species RANKL will be referred to herein as Ab1 and antibody(ies) raised in another species as Ab2.

In one non-limiting example, the presence of RANKL in cells can be ascertained by the usual in vitro or in vivo immunological procedures applicable to such determinations. A number of useful procedures are known. Three such procedures which are especially useful utilize either mouse or human RANKL labeled with a detectable label, antibody Ab1 labeled with a detectable label, or antibody Ab2 labeled with a detectable label. The procedures can be summarized by the following equations wherein the asterisk indicates that the particle is labeled, and “R” stands for RANKL or the unknown sample:

A. R*+Ab1=R*Ab1

B. R+Ab*=RAb1*

C. R+Ab1+Ab2*=RAb1Ab2*.

The procedures and their application are all familiar to those skilled in the art and accordingly can be utilized within the scope of the present application. The “competitive” procedure, Procedure A, is described in U.S. Pat. Nos. 3,654,090 and 3,850,752, each of which is which is incorporated herein by reference in its entirety. Procedure C, the “sandwich” procedure, is described in U.S. Pat. Nos. RE 31,006 and 4,016,043, each of which is which is incorporated herein by reference in its entirety. Still other procedures are known such as the “double antibody” or “DASP” procedure.

In each instance above, mouse or human RANKL forms complexes with one or more antibody(ies) or binding partners and one member of the complex is labeled with a detectable label. The fact that a complex has formed and, if desired, the amount thereof, can be determined by known methods applicable to the detection of labels.

It will be seen from the above, that a characteristic property of Ab2 is that it will react with Ab1. This is because Ab1 raised in one mammalian species has been used in another species as an antigen to raise the antibody Ab2. For example, Ab2 can be raised in goats using rabbit antibodies as antigens. Ab2 therefore would be anti-rabbit antibody raised in goats. For purposes of this description and claims, Ab1 will be referred to as a primary or anti-RANKL antibody, and Ab2 will be referred to as a secondary or anti-Ab1 antibody. For purposes herein, Ab1 refers to as a primary or anti-RANKL antibody, and Ab2 refers to as a secondary or anti-Ab I antibody. An Ab3 antibody refers to an antibody that binds to an Ab2 antibody (i.e., an anti-anti-idiotypic antibody). While the foregoing is exemplary, it illustrates the manner in which Ab1, Ab2 and Ab3 antibodies can be generated.

The diagnostic utility extends to the use of the binding members in assays to measure RANKL levels during lytic bone diseases, including in vitro and in vivo diagnostic assays. In an immunoassay, a control quantity of the antibodies, or the like can be prepared and labeled with an enzyme, a specific binding partner and/or a radioactive element, and may then be introduced into a cellular sample. After the labeled material or its binding partner(s) has had an opportunity to react with sites within the sample, the resulting mass may be examined by known techniques, which can vary with the nature of the label attached.

Specific binding members can carry a detectable or functional label. The specific binding members can carry a radioactive label, such as the isotopes ³H, ¹⁴C, ³²P, ³⁵S, ³⁶Cl, ⁵Cr, ⁵⁷Co, ⁵⁸Co, ⁵⁹Fe, ⁹⁰Y, ¹²¹I, ¹²⁴I, ¹³¹I, ¹¹¹In, ²¹¹At, ¹⁹⁸Au, ⁶⁷Cu, ²²⁵Ac, ²¹³Bi, ⁹⁹Tc and ¹⁸⁶Re. When radioactive labels are used, known currently available counting procedures can be utilized to identify and quantitate the specific binding members. In the instance where the label is an enzyme, detection can be accomplished by any of the presently utilized colorimetric, spectrophotometric, fluorospectrophotometric, amperometric or gasometric techniques known in the art.

Radiolabeled specific binding members (e.g., antibodies and antigen-binding fragments thereof) are useful in in vitro diagnostics techniques and in in vivo radioimaging techniques. In a further aspect, radiolabeled specific binding members (e.g., antibodies and fragments thereof) or radioimmunoconjugates are useful in radioimmunotherapy for treatment of cancer. In a still further aspect, the radiolabeled specific binding members are useful in radioimmuno-guided surgery techniques, wherein they can identify and indicate the presence and/or location of RANKL expressing cells during disease states characterized by excessive bone loss.

The labels most commonly employed for these studies are radioactive elements, enzymes, chemicals which fluoresce when exposed to ultraviolet light, and others as described else where herein.

One assay system that can be advantageously utilized in accordance with the present application is known as a receptor assay. In a receptor assay, the material to be assayed (such as the specific binding member) is appropriately labeled and then certain cellular test colonies are inoculated with a quantity of both the labeled and unlabeled material after which binding studies are conducted to determine the extent to which the labeled material binds to the cell receptors. In this way, differences in affinity between materials can be ascertained.

Accordingly, a purified quantity of the specific binding member can be radiolabeled and combined, for example, with antibodies or other inhibitors thereto, after which binding studies would be carried out. Solutions would then be prepared that contain various quantities of labeled and unlabeled uncombined specific binding member, and cell samples would then be inoculated and thereafter incubated. The resulting cell monolayers are then washed, solubilized and then counted in a gamma counter for a length of time sufficient to yield a standard error of <5%. These data are then subjected to Scatchard analysis after which observations and conclusions regarding material activity can be drawn. While the foregoing is exemplary, it illustrates the manner in which a receptor assay can be performed and utilized, in the instance where the cellular binding ability of the assayed material can serve as a distinguishing characteristic.

Another assay useful and contemplated is known as a “cis/trans” assay. Briefly, this assay employs two genetic constructs, one of which is typically a plasmid that continually expresses a particular receptor of interest when transfected into an appropriate cell line, and the second of which is a plasmid that expresses a reporter such as luciferase, under the control of a receptor/ligand complex. Thus, for example, if it is desired to evaluate a compound as a ligand for a particular receptor, one of the plasmids would be a construct that results in expression of the receptor in the chosen cell line, while the second plasmid would possess a promoter linked to the luciferase gene in which the response element to the particular receptor is inserted. If the compound under test is an agonist for the receptor, the ligand will complex with the receptor, and the resulting complex will bind the response element and initiate transcription of the luciferase gene. The resulting chemiluninescence is then measured photometrically, and dose response curves are obtained and compared to those of known ligands. The foregoing protocol is described in detail in U.S. Pat. No. 4,981,784 and PCT International Publication No. WO 88/03168, each of which is which is incorporated herein by reference in its entirety, for which purpose the artisan is referred.

B. Therapeutic Uses

Normal bone remodeling is a process in which new bone deposition by osteoblasts is balanced through bone resorption by osteoclasts (see Gowen, M., Every, J G, and Kumar S. Emerging therapies for osteoporosis. Emerging Drugs, 2000 5(1): p. 1-43). In several disease states, the balance between bone deposition and bone resorption is perturbed. In osteoporosis, for example, excess bone resorption leads to brittle bones and frequent fractures of the wrist, vertebrae, and hip. In rheumatoid arthritis, increased bone resorption leads to malformations of the bones within arthritic joints. Re-establishing normal bone remodeling in these and other disorders can be achieved by decreasing or increasing the number and activity of osteoclasts (See Rodan, G A, and Martin T J. Science 2000 289: p. 1508-1514.)

Several proteins modulate the bone remodeling orchestrated by osteoblasts and osteoclasts. (See US 2003/0013651 and WO 02/080955). Three key proteins are the cell-surface receptor RANK (Receptor Activator of NF-κB), the soluble decoy receptor OPG (osteoprotegerin), and the soluble and transmembrane forms of RANKL (RANK ligand, also known as RANKL, TNF-related activation induced cytokine (TRANCE), osteoclast differentiation factor (ODF), and osteoprotegerin ligand (OPGL). RANK is activated by the binding of its ligand, RANKL, which leads to the differentiation, survival, and fusion of pre-osteoclasts to form active bone resorbing osteoclasts (see Lacey et al. 1998 Cell 93: p. 165-176.). RANKL is a trimeric TNF family member that binds to the trimeric RANK receptor.

The RANKL/OPG/RANK biochemical axis has been successfully targeted to treat osteoporosis, rheumatoid arthritis, prosthesis-induced osteolysis, cancer-induced bone destruction, metastasis, hypercalcemia, and pain (see Hofbauer, L C, Neubauer, A, and Heufelder A E. Receptor activator of nuclear factor-κB ligand and osteoprotegrin. 2001 Cancer 92(3): p. 460-470; Takahashi N, Udagawa N, and Suda T., A new member of the tumor necrosis factor ligand family, ODF/OPGL/TRANCE/RANKL, regulates osteoclast differentiation and function. 1999 Biochem. Biophys. Res. Comm. 256: p. 449-455). Therapies utilizing OPG (see Honore et al., 2000 Nature Medicine 6(5):521-528) or the soluble RANK-Fc protein (See Oyajobi et al., 2001 Cancer Res 61(6): p. 2572-8; Childs et al. J. of Bone and Mineral Res. 17: p. 192-199) are also in development. OPG and soluble RANK-Fc protein constructs bind to RANKL, thereby decreasing amount of RANKL that is available for RANK receptor activation.

1. Bone Loss Disorders

RANK and RANKL are involved in a variety of diseases and disorders; therefore, the compounds and compositions as described herein have applicability to the treatment of such diseases and disorders.

Osteoporosis and Bone Reconstruction

RANKL is a ligand for osteoprotegerin (OPG) and RANK and functions as a key factor for osteoclast differentiation and activation. This protein has been shown to be a dendritic cell survival factor and is involved in the regulation of T cell-dependent immune responses. T cell activation induces expression of the RANKL gene and leads to an increase in osteoclastogenesis and bone loss.

Bone resorbing activity and apoptosis of osteoclasts are regulated by the RANKL-RANK pathways. Soluble RANKL treatment both extends the survival of mature osteoclasts, and stimulates their bone-resorbing activity in vitro and in vivo, while application of OPG induces apoptotic cell death of mature osteoclasts in vivo. The results from these in vitro and in vivo experiments revealed that the interactions between RANKL, RANK, and OPG regulate normal osteoclast development and are, therefore, involved in healthy bone growth. (Burgess T L et al. 1999, J. Cell Biol., 145:527-538; Lacey D L et al., 2000, Am J. Pathol., 157:435-448; Fuller K. et al., 1998, J Exp Med., 188:997-1001).

In osteoporosis the normal balance between bone formation and bone resorption is impaired. The primary cause of postmenopausal osteoporosis in women is estrogen deficiency, and bone destruction takes place at a faster rate after menopause. RANKL transcripts are highly expressed in bone tissues of ovariectomized rats (Xu J et al., 2000, J Bone Miner Res., 15:2178-2186). Riggs et al. demonstrated that postmenopausal women express higher levels of RANKL on marrow stromal cells and lymphocytes than premenopausal women or postmenopausal women taking estrogen therapy. RANKL expression per cell correlated well with the bone resorption markers serum C-terminal telopeptide of type I collagen and inversely with serum 17b-estradiol level (Eghbali-Fatourechi G et al., 2003, J Clin Invest, 111:1221-1230). A recent clinical trial suggests that in postmenopausal women with low bone mass, a fully human anti-RANKL antibody (Denosumab, AMG 162) increased bone mineral density and decreased bone resorption.

Daily administration of RANKL to mice and rats induces rapid bone resorption and elevation of serum calcium levels (Lacey D. L. et al., 1998, Cell, 93:165-176). RANKL-deficient mice show severe osteopetrosis, with no osteoclasts, marrow spaces, or tooth eruption, and exhibit profound growth retardation at several skeletal sites, including the limbs, skull, and vertebrae. These mice have marked chondrodysplasia, with thick, irregular growth plates and a relative increase in hypertrophic chondrocytes. RANKL-deficient mice exhibited defects in early differentiation of T and B lymphocytes, lack lymph nodes, and failed to form lobulo-alveolar mammary structures during pregnancy. Transplantation and RANKL rescue experiments in RANKL−/− and RANK−/− pregnant females showed that RANKL acts directly on RANK-expressing mammary epithelial cells. Overexpression of soluble RANKL in transgenic mice results in a skeletal phenotypes with many similarities to postmenopausal osteoporosis, including reduced BMD, increased bone resorption, cortical porosity and skeletal fragility along with a 50% reduction in the biomechanical strength of the femoral diaphysis (Mizuno A et al., 2002, J Bone Miner Metab, 20:337-344).

Additional RANKL responsive disorders include, but are not limited to, prosthesis fitting, prevention of prosthesis loosening, prosthesis-induced osteolysis, craniofacial reconstruction and in the treatment of other fractures, for example, hip, spine and long bone fractures, loss due to weightlessness, malnutrition, disuse osteopenia; bone alcohol use, spinal cord injury, periodontal disease, periodontal reconstruction, bone fractures, osteoporosis (postmenopausal) caused by elevated RANKL through oophorectomy; osteoclastoma associated with elevated RANKL; bone loss in hyperparathyroidism caused by elevated RANKL through parathyroid hormone (PTH); and osteoporosis (glucocorticoid-induced) caused by elevated RANKL through glucocorticoids among others.

Rheumatoid Arthritis

Rheumatoid arthritis (RA) is a chronic systemic inflammatory disorder with an unknown cause characterized by invasive synovial hyperplasia leading to progressive joint destruction. Bone erosion begins in the early stages of the disease and results in severe deformity of the affected joints which impairs the normal activity and quality of life of patients. Rheumatoid arthritis can be associated with elevated RANKL in T-cells, synovial fibroblasts, and bone marrow stroma;

Osteoclasts are primarily involved in the bone destruction in RA. Bone erosion usually beings at the interface of the cartilage and the proliferating synovium, and bone-resorbing osteoclasts are observed at the erosive synovium/bone interfaces. Histological analysis of the bone-pannus interface of RA joints by light microscopy revealed the presence of acid phosphatase-positive multi-nucleated cells with morphologic features of osteoclasts along the surface of mineralized subchondral bone and mineralized cartilage (Bromley M, Woolley D E, 1984, Arthritis Rheum., 27:968-975).

There's evidence supporting a link between the expression of RANKL messenger RNA and development of synovial lesions in RA. Patients with RA have elevated serum levels of soluble RANKL, which return to normal levels after TNF-α treatment. RANKL mRNA is not expressed in normal synovium, but it is expressed in the synovial lining layer in patients with RA, and RANKL as well as RANK are also detected in the synovium of rats with collagen-induced arthritis.

BXD2 mice develop arthritis with bone erosions, synovial hyperplasia with mononuclear cell infiltration, and joint deformation. These mice also have high levels of rheumatoid factor and anti-DNA auto-antibodies. In this model, inhibition of RANKL had no effect on inflammation, but completely prevented bone loss and partially protected against cartilage loss (Wu Y. et al., 2005, Arthritis Rheum, 52:3257-3268).

This result is at least partially supported by studies in adjuvant- or collagen-induced arthritis models in Lewis rats, in a type II collagen-induced arthritis model in mice, in an antibody-mediated arthritis (K/B×N mouse serum transfer model) mouse model, and in a TNF-α transgenic arthritic mouse model. (Kong Y Y et al., 1999, Nature 402:304-309; Mori H et al., 2002, Histochem Cell Biol, 117:283-292; Campagnuolo G. et al., 2002, Arthritis Rheum, 46:1926-1936; Bolon B et al., 2002, Cell Mol Life Sci, 59:1569-1576; and Pettit A R et al., 2001, 159:1689-1699.)

Periodontal Diseases

Periodontal diseases are chronic infectious inflammatory diseases characterized by increased leukocyte infiltration into the periodontal lesions. This infiltration results in the secretion of a number of cytokines, which leads to the destruction of periodontal tissues including alveolar bone (Taubman M A et al., 2001, Crit. Rev Oral Biol Med., 12:125-135).

RANKL expressed by either osteoblasts or infiltrating T cells in response to bacterial infection is involved in alveolar bone destruction in periodontal diseases. RANKL messenger RNA is upregulated in gums from patients with severe periodontitis. In an animal model of periodontitis, oral challenge with A. acinomycetemcomitans (a Gram negative bacterium that causes human periodontitis) of NOD/SCID mice in which peripheral blood lymphocytes (PBLs) from patients with localized juvenile periodontitis had been transplanted, led to localized bone resorption in alveolar bone surrounding the teeth. When CD4+ T cells isolated from these mice were stimulated in vitro with antigens from A. actinomycetemcomitans, RANLK expression increased. Another bacterium implicated in human periodontitis, Porphyromonas gingivalis, can also induce RANKL expression in lymphocytes which resulted in increased osteoclast formation in spleen cell cultures (Jiang Y et al., 2002, Infect Immun., 70:3143-3148.

In an animal model of periodontitis, peripheral blood leukocytes from patients with localized juvenile periodontitis were transplanted into NOD/SCID mice.

Periprosthetic bone loss leading to aseptic loosening of implants is one of the most challenging complications of joint replacement surgeries. Osteoclast-like multinucleated cells are observed in the bone-implant interface of the loosened joints and the fibroblastic cells in the perioprosthetic tissues have been shown to induce the differentiation of normal human peripheral blood mononuclear cells into mature osteoclasts by a mechanism that involves both RANKL and TNF-α (Sabokbar A. et al., 2005, J Orthop Res., 23:511-519).

Cancers

Hypercalcemia is a late stage complication of cancer, disrupting the body's ability to maintain normal levels of calcium, resulting in calcium deposit in the kidneys, heart conditions and neural dysfunction and occurs most frequently in patients with cancers of the lung and breast. Hypercalcemia also occurs in patients with multiple myeloma, cancers of the head and neck, sarcoma, cancers of unknown primary origin, lymphoma, leukemia, melanoma, renal cancer, and gastrointestinal cancers (e.g. esophageal, stomach, intestinal, colon and rectal cancers). RANK and RANKL play a role in bone loss associated with cancers. When RANKL+ myeloma cells are injected into C57BL mice, the mice develop bone disease characterized by a marked decrease in cancellous bone volume in the tibial and femoral metaphyses, increased osteoclast formation, and radiologic evidence of osteolytic bone lesions.

Other cancer indications, which the anti-RANKL compounds described herein can treat include, but are not limited to: hematologic neoplasisas and neoplastic-like conditions for example, Hodgkin's lymphoma; non-Hodgkin's lymphomas (Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymphoma, hairy cell leukemia and lymphoplasmacytic leukemia), tumors of lymphocyte precursor cells, including B-cell acute lymphoblastic leukemia/lymphoma, and T-cell acute lymphoblastic leukemia/lymphoma, thymoma, tumors of the mature T and NK cells, including peripheral T-cell leukemias, adult T-cell leukemia/T-cell lymphomas and large granular lymphocytic leukemia, Langerhans cell histocytosis, myeloid neoplasias such as acute myelogenous leukemias, including AML with maturation, AML without differentiation, acute promyelocytic leukemia, acute myelomonocytic leukemia, and acute monocytic leukemias, myelodysplastic syndromes, and chronic myeloproliferative disorders, including chronic myelogenous leukemia.

Additional cancers which can be treated by the anti-RANKL compounds described herein, include but are not limited to cancers which rarely or never metastasize to bone and in which hypercalcemia generally does not occur, for example: tumors of the central nervous system, e.g., brain tumors (glioma, neuroblastoma, astrocytoma, medulloblastoma, ependymoma, and retinoblastoma), solid tumors (nasopharyngeal cancer, basal cell carcinoma, pancreatic cancer, cancer of the bile duct, Kaposi's sarcoma, testicular cancer, uterine, vaginal or cervical cancers, ovarian cancer, primary liver cancer or endometrial cancer, and tumors of the vascular system (angiosarcoma and hemagiopericytoma), among others.

Some primary tumors and metastatic malignant tumors, such as breast cancer and lung cancer, invade bone tissues. Osteoclasts are primarily responsible for the osteolysis observed in these patients and there's evidence that in patients with severe osteolysis, the RANKL/OPG ratio is increased (Wittrant Y et al., Biochim Biophys Acta, 2004, 1704:49-57; Greimaud E et al., 2003, Am J Pathol., 163:2021-2031).

Among various bone tumors, the molecular mechanism of bone destruction in multiple myeloma is one of the most studied. Multiple myeloma is a plasma cell cancer that develops in bone marrow and causes skeletal destruction. Increased bone resorption caused by osteoclast activation and osteoblast inhibition is a major characteristic of multiple myeloma. Myeloma cells stimulate osteoclast differentiation and activation by increasing RANKL and decreasing OPG expression within the marrow microenvironment. The myeloma cells also secrete factors that act on osteoblasts to suppress their function, inhibit their differentiation, and induce apoptosis. The bone destruction that results releases several cytokines that promote myeloma cell growth; therefore, blocking the osteolytic process can also have an anti-tumor effect (Sezer O et al., 2003, Blood, 101:2094-2098; Roux S. et al., 2002, Br J. Haematol., 117:86-92; Farrugia A N et al, 2003, Cancer Res., 63:5438-5445; Croucher P I et al., 2001, Blood, 98:3534-3540; Guiliani N. et al., 2001, Blood, 98:3527-3533).

The RANKL/RANK/OPG system has also been reported to be involved in bone destruction in breast cancer cells, prostate cancer cells, and other metastatic bone tumors (Kitazawa S. et al., 2002, J. Pathol., 198:228-236; Park H R et al., 2003, J Korean Med Sci, 18:541-546; Zhang J et al., 2001, J Clin Invest., 107:1235-1244; Keller E T et al., 2001, Cancer Metastasis Rev., 20:333-349).

Specific blockade of RANKL prevents the skeletal complications in various animal models of myeloma and suppressed bone resorption in patients with myeloma bone disease. Treatment of myelomatous SCID-human mice with a RANK-Fc fusion protein reduced myeloma-induced bone resorption and resulted in a greater than 80% reduction in paraprotein. Treatment resulted in a reduced number of osteoclasts, but had no effect on the apoptosis and proliferation of myeloma cells, suggesting that the anti-myeloma effect of RANKL inhibitors is associated with inhibition of osteoclast activity (Yaccodby et al., 2002, Br. J. Haematol., 116:278-290).

Juvenile Paget's Disease

Some patients with Juvenile Paget's Disease have mutations in the OPG gene which result in undetectable serum levels of OPG and large increases in soluble RANKL levels. This disorder is a rare disease with an autosomal inheritance pattern, and it displays various deformities of long bones and vertebral column, which increase in severity during adolescence. (Whyte M P et al., 2002, N Engl J Med., 347:175-184; Cundy T et al., 2002, Hum Mol Genet., 11:2119-2127; Chong B. et al., 2003, J Bone Miner Res., 18:2095-2104).

2. Immunization

In addition to being involved in bone biology, RANKL plays a role in the immune system by regulating antigen-specific T cell responses (See Anderson et al., Nature 1997, 390(6656):175-179). RANKL is highly expressed on activated T cells while the RANK receptor is expressed at high levels on mature dendritic cells (DC). The interaction between RANKL and RANK acts as a co-stimulatory signal, which enhances DC survival and T cell proliferation by inducing DC differentiation, cytokine production and reduced apoptosis in both cell types. Immunotherapy to produce tolerance to transplanted tissues and/or organs can be achieved by blocking the co-stimulatory signal using RANK antagonists. Blocking co-stimulation prevents T cell activation by DCs, and causes alloreactive T cells to become anergic and/or undergo apoptosis (See Adler et al., Current Opinion in Immunology 2002, 14:660-665). By a similar mechanism of action, antagonizing RANK signaling could be a treatment for autoimmune disorders such as systemic lupus erythematosus, inflammatory bowel disease, diabetes, multiple sclerosis, rheumatoid arthritis, and ankylosing spondylitis (see WO 03/033664).

Compositions are provided for immunization of a subject to induce an immune response against a cross-reactive binding sequence. An immune response can be, for example, a humoral immune response including, but not limited to, an antibody response or a T helper response. These preparations include an acceptable carrier and a preparation of a cross-reactive binding sequence. The preparation is capable of eliciting an antibody response in vivo, which antibodies are capable of binding to a cross-reactive binding sequence. The preparation can be linked to, or administered with, an immunogenic moiety (e.g., an adjuvant) to increase the likelihood that an immune response will be initiated. Dosage, safety and efficacy of such preparations can be determined in vitro and tested in appropriate in vivo animal models (e.g., rodent and primate). Such compositions for vaccination can be made in any convenient manner known to one of ordinary skill in the art.

Immunotherapies involve one or more components of the immune system to trigger a cascade of biological reactions to initiate an immune response in the host. The immune system consists of a wide range of distinct cell types, one of which is the lymphocytes. Lymphocytes determine the specificity of immunity, and it is their response that orchestrates the effector limbs of the immune system. Lymphocytes differ from each other not only in the specificity of their receptors, but also in their functions. One class of lymphocytes, B cells, is a precursor of antibody-secreting cells, and function as mediators of the humoral immune response. Another class of lymphocytes, T cells, are involved in regulatory functions and function to help B cell responses (i.e., a T helper response).

Immunization (vaccination) is based on the principle of inducing the immune system to recognize a polypeptide. The initial step in immunotherapy is inducing the host immune system to first recognize a polypeptide and then inducing the system to initiate a response against that polypeptide. In healthy hosts, the immune system recognizes surface features of a polypeptide that is not a normal constituent of the host (i.e., “foreign” to the host). Once recognition occurs, the host then directs an immune response against that particular foreign polypeptide.

One of the host system's mechanisms for combating a foreign molecule is the humoral response, that is, the production of an antibody against a specific foreign molecule (called an antigen). Typically, the antibody's ability to bind the antigen is based on highly complementary structures. A particular B or T cell binds to a very specific region of the antigen, called an antigenic determinant or epitope. Thus antigens are molecules that bear one or more epitopes which can be recognized by specific receptors in an immune system.

Immunogenicity is the property of stimulating the immune system to generate a specific response. Thus, all immunogens are antigens, but not vice-versa. Although an immune system can recognize an antigen, it may not respond to the antigen unless the antigen is also immunogenic. In some cases, the immune system is not able to generate an immune response to a particular antigen if it is a self antigen, a condition that is called tolerance. Immunogenicity is promoted by several factors including foreign, non-human origin, higher molecular weight, greater molecular complexity, tertiary structures, post-translational modifications, the proper antigen dose range, the route of administration, the age of the host, and the genetic composition of the host. Tolerance can be broken by modifying a self antigen to make it foreign to the host.

As noted above, antigens can have one or more epitopes (e.g., binding sites, epitopes or cross-reactive binding sequences) that are recognized by specific receptors of the immune system. Binding sites, epitopes or cross-reactive binding sequences can be formed by the primary structure of a molecule (called a sequential epitope/cross-reactive binding sequence), or can be formed by portions of the molecule separate from the primary structure that juxtapose in the secondary or tertiary structure of the molecule (called a conformational or discontinuous epitope/cross-reactive binding sequence). Some epitopes/cross-reactive binding sequences are hidden in the three dimensional structure of the native antigen, and become immunogenic only after a conformational change in the antigen provides access to the epitope/cross-reactive binding sequence by a receptors of the immune system (called a cryptic epitope). This is a feature and function in the ability of a therapeutic reagent to initiate recognition and response to an antigen, to induce a humoral response to the antigen.

An “immunological response” to a composition or vaccine of an antigen (e.g., a cross-reactive binding sequence) is the development in the host of an antibody-mediated immune response to the composition or vaccine of interest. Such a response of the subject can include one or more of producing antibodies, B cells, and helper T cells directed specifically to an antigen or antigens included in the composition or vaccine of interest.

It is therefore expected that the immunization of an animal with a RANKL cross-reactive binding sequence composition can induce a specific and protective humoral against a RANKL cross-reactive binding sequence. Provided herein is, for example, a method of inducing a humoral immune response by administering any of the pharmaceutical compositions provided herein. In one embodiment, the immune response is a humoral immune response. In one non-limiting embodiment, induction of an immune response generates antibodies that bind to a RANKL cross-reactive binding sequence.

3. Treatment

Binding members (e.g., antibodies or antigen-binding fragments), as exemplified in one embodiment by a monoclonal antibody that can cross-react and neutralize RANKL from multiple species or in alternate embodiments as a polyclonal antibody or chimeric antibody that can cross-react and neutralize RANKL from multiple species, can demonstrate significant in vivo anti-resorptive activity and decrease bone resorption rates. In vivo assays are available to measure whether the monoclonal antibodies described herein are capable of inhibiting bone resorption: Animal disease models are available whereby bone resorption is stimulated, such as removal of ovaries to mimic loss of estrogen, or as described above. The effect of RANKL binding proteins on ovariectomy-induced bone loss has been reported and can be measured using quantitative computed tomography and measurement of biochemical markers of bone turnover, such as the N-terminal telopeptide of collagen.

Therefore, in a further aspect, there is provided a method of treatment of various diseases characterized by excessive bone loss, and any condition related to or resulting from comprising administration of a specific binding member described herein.

Provided herein is a method of preventing or treating one or more bone loss conditions, diseases or disorders comprising administering a composition comprising an antibody, antigen-binding fragment or binding protein that binds to a cross-reactive binding sequence associated with the bone loss condition, disease or disorder. The composition to be administered in the methods described herein can further include a therapeutic moiety and/or a detectable moiety.

As used herein, “prevention” refers to prophylaxis, prevention of onset of symptoms, prevention of progression of a bone loss condition, disease or disorder. As used herein, “inhibition,” “treatment” and “treating” are used interchangeably and refer to, for example, stasis of symptoms, prolongation of survival, partial or full amelioration of symptoms, and partial or full eradication of a condition, disease or disorder.

In one aspect, the one or more conditions, diseases or disorders can be associated with bone loss. In one aspect, the cross-reactive binding sequence associated with the one or more bone loss conditions or diseases is a RANKL cross-reactive binding sequence. In one embodiment, prevention or treatment of one or more conditions, diseases or disorders associated with bone loss includes administration of a composition of an antibody, antigen-binding fragment or binding protein that binds to a RANKL cross-reactive binding sequence such as, for example, those described herein. Administration of such antibodies, antigen-binding fragments or binding proteins can decrease bone loss and/or bone resorption rates, thereby preventing or treating the one or more bone loss conditions, diseases or disorders. Bone loss conditions, diseases or disorders to be prevented or treated using the subject methods include, for example, osteoporosis, rheumatoid arthritis, bone metastasis and hypercalcemia of malignancy, glucocorticoid-induced bone loss and periodontal diseases or conditions (e.g., periodontitis, dental implants, etc.), cancers such as primary tumors and metastatic malignant tumors (e.g., such as breast cancer, lung cancer, prostate cancer, and other metastatic bone tumors) and Juvenile Paget's Disease.

Radioimmunotherapy (RAIT) has entered the clinic and demonstrated efficacy using various antibody immunoconjugates. ¹³¹I labeled humanized anti-carcinoembryonic antigen (anti-CEA) antibody hMN-14 has been evaluated in colorectal cancer (Behr T M et al (2002) Cancer 94(4Suppl): 1373-81) and the same antibody with ⁹⁰Y label has been assessed in medullary thyroid carcinoma (Stein R et al (2002) Cancer 94(1):51-61). Radioimmunotherapy using monoclonal antibodies has also been assessed and reported for non-Hodgkin's lymphoma and pancreatic cancer (Goldenberg D M (2001) Crit. Rev Oncol Hematol 39(1-2):195-201; Gold D V et al (2001) Crit. Rev Oncol Hematol 39 (1-2) 147-54). Radioimmunotherapy methods with particular antibodies are also described in U.S. Pat. Nos. 6,306,393 and 6,331,175. Radioimmunoguided surgery (RIGS) has also entered the clinic and demonstrated efficacy and usefulness, including using anti-CEA antibodies and antibodies directed against tumor-associated antigens (Kim J C et al (2002) Int J Cancer 97(4):542-7; Schneebaum S et al (2001) World J Surg 25(12):1495-8; Avital S et al (2000) Cancer 89(8):1692-8; McIntosh D G et al (1997) Cancer Biother Radiopharm 12 (4):287-94).

Specific binding members as disclosed herein can be administered to a patient in need of treatment via any suitable route, usually by injection into the bloodstream or cerebro-spinal fluid (CSF). The precise dose will depend upon a number of factors, including whether the antibody is for diagnosis or for treatment, the degree of bone resorption in the patient, the precise nature of the antibody (whether whole antibody, fragment, diabody, etc), and the nature of the detectable or functional label attached to the antibody. Where a radionuclide is used for therapy, a suitable maximum single dose is about 45 mCi/m², to a maximum of about 250 mCi/m².

These formulations can, in some instances, include a second binding protein, such as the binding proteins described above and including, but not limited to, is a monoclonal antibody.

Provided herein is a method of generating an antibody, antigen-binding fragment thereof, or binding protein that binds to a RANKL cross-reactive binding sequence, where said antibody, antigen-binding fragment thereof, or biding protein is recombinantly made or synthesized using standard techniques known in the art.

In one non-limiting embodiment, induction of an immune response decreases (e.g., by at least about 5 percent, at least about 10 percent, at least about 20 percent, at least about 30 percent, at least about 40 percent, at least about 50 percent, at least about 60 percent, at least about 70 percent, at least about 80 percent, at least about 90 percent, or more) bone resorption rates. In one non-limiting embodiment, induction of an immune response decreases bone loss.

In further embodiments, the present application relates to certain therapeutic methods which would be based upon the activity of the binding member, antibody, or active fragments thereof, or upon agents or other drugs determined to possess the same activity. A first therapeutic method is associated with the prevention or treatment of bone loss associated with increased osteoclast activity such as, for example, osteoporosis, rheumatoid arthritis, bone metastasis and hypercalcemia of malignancy, glucocorticoid-induced bone loss and periodontal diseases or conditions (e.g., periodontitis, dental implants, etc.), cancers such as primary tumors and metastatic malignant tumors (e.g., such as breast cancer, lung cancer, prostate cancer, and other metastatic bone tumors) and Juvenile Paget's Disease.

In a further aspect is a method for treating bone disease in a mammal comprising administering to said mammal an effective amount of a monoclonal antibody that can cross-react and neutralize RANKL from multiple species in combination with one or more other compounds for the treatment of bone disease. An effective amount of a monoclonal antibody that can cross-react and neutralize RANKL from multiple species can be used in combination with one or more anti-resorptive or anabolic compound for treatment of bone disease, including but not limited to a bisphosphonate, a Vav3 or Vav3 pathway inhibitor, a calcitonin, a calcitriol, an estrogen, selective estrogen receptor modulators (SERM's) and a calcium source, a supplemental bone formation agent parathyroid hormone (PTH) or its derivative or fragments thereof, PTH related protein (PTHrp), a bone morphogenetic protein, osteogenin, NaF, PGE2 agonists, a statin, and a RANK ligand (RANKL), including an osteogenic form of RANKL such as GST-RANKL or other oligomerized form of RANKL.

### EXAMPLES

The invention may be better understood by reference to the following non-limiting Examples, which are provided as exemplary embodiments of the invention. The following examples are presented in order to more fully illustrate certain embodiments of the invention and should in no way be construed, however, as limiting the broad scope of the invention. While embodiments of the present invention have been shown and described herein, it will be obvious to those skilled in the art that such embodiments are provided by way of example only. Numerous variations, changes, and substitutions can now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1

### Predicted RANKL Peptide Sequences that Bind to its Receptor

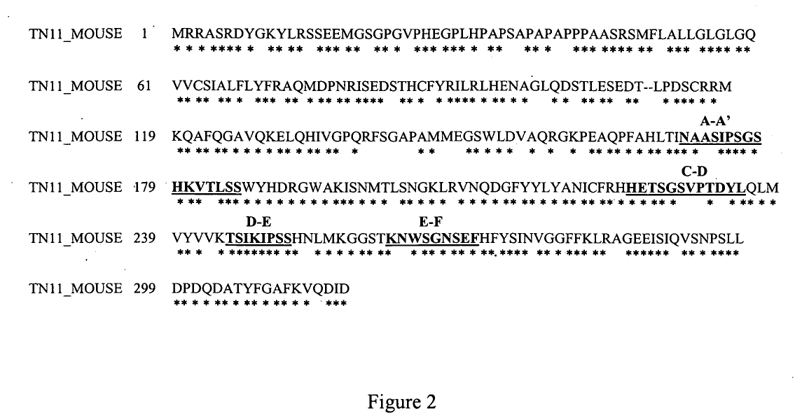

The mouse RANKL protein is a TNF-related cytokine that regulates osteoclast differentiation and maturation in vitro, and controls bone resorption and remodeling rates in vivo (see Boyle et al., Nature 423, 337-342 [2003] for review). RANKL is a type II surface protein that also exists as a soluble, cleaved form. RANKL mediates these events by the binding of its TNF-like C-terminal domain to the TNFR-related protein, RANK, thereby stimulating osteoclast specific gene expression and cellular differentiation and activation events. In addition to its role in osteoclastiogenesis, RANKL has been reported to induce human dendritic cell (DC) cluster formation, and development of mammary epithelium. Previous studies by Lam et al. (J. Clinical Investigation 108, 971-979 (2001)), and in United States Patent Application Publication Number 20030100068, May 2003, have defined subregions of the RANKL TNF-like domain that are exposed on the protein that are predicted to bind to RANK and lead to receptor activation (FIG. 1). These subregions are predicted to form loops on the protein surface that form receptor contacts. These loops are formed during the association of interdispersed b-pleated sheet structures that form the inner core of the mature RANKL protein, and are designated as described by Lam et al., supra, as the A-A′, C-D, D-E, and E-F loops respectively (FIG. 2).

Identification of these loops is useful, because as structural determinants that control osteoclastogenesis, they can provide specific coordinates on the protein that are useful in generating antibodies and binding proteins that interact with these sites. Pathological increases in bone resorption rates in man and animals result in significant bone loss, increased bone fractures, and systemic hypercalcemia. These conditions are seen in a number of human diseases, such as osteoporosis, rheumatoid arthritis, and metastatic bone disease, for example. Therefore, monoclonal antibodies and binding proteins that binding to these sites can act as potent inhibitors of RANKL activity in vivo, and can be used as therapeutic agents to treat various bone lytic diseases characterized by increased RANKL levels.

### Example 2

### Cross-Reactive RANKL Antigens

The mouse RANKL sequences, and structure/function relationships, shown in Example 1, are highly conserved throughout evolution. Mouse RANKL stimulates osteoclastogenesis from human peripheral blood mononuclear cells (Shalhoub et al., Br. J. Haematol. 111, 501-512 [2000]), as does human RANKL for mouse osteoclast precursors in the spleen and bone marrow (Lacey et al., Cell. 93, 165-76 [1998]). This suggests that the surface loop structures for mouse RANKL are highly conserved, and capable of binding to the RANK receptor from multiple species.

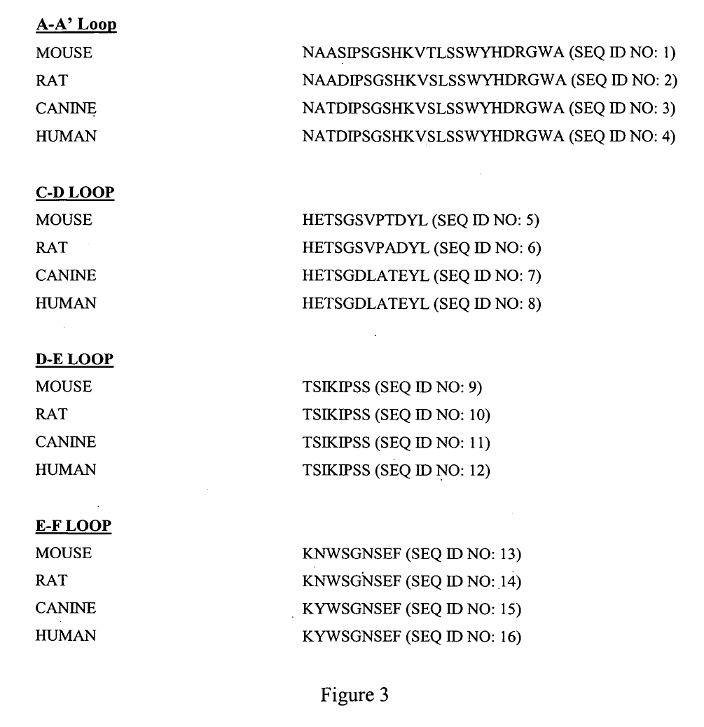

The RANKL polypeptide sequences available in public databases were aligned using the SIM method, as described by Huang and Miller, “A Time-Efficient, Linear-Space Local Similarity Algorithm”. Advances in Applied Mathematics, vol. 12 (1991), pp. 337-357, and the corresponding loop regions of RANKL were deduced. Shown in FIG. 3 are the homologous regions of the Mouse (ID: O35235), Rat (ID: Q9ESE2), Canine (ID: XP_{—}851765), and Human (ID: 014788) RANKL proteins, corresponding to the A-A′, C-D, D-E, and E-F. The high degree of similarity in these regions was used to investigate the possibility of using selected peptide regions of the loops domains as potential antigens to generate RANKL neutralizing antibodies that react equally well with RANKL derived from multiple species such as mammals.

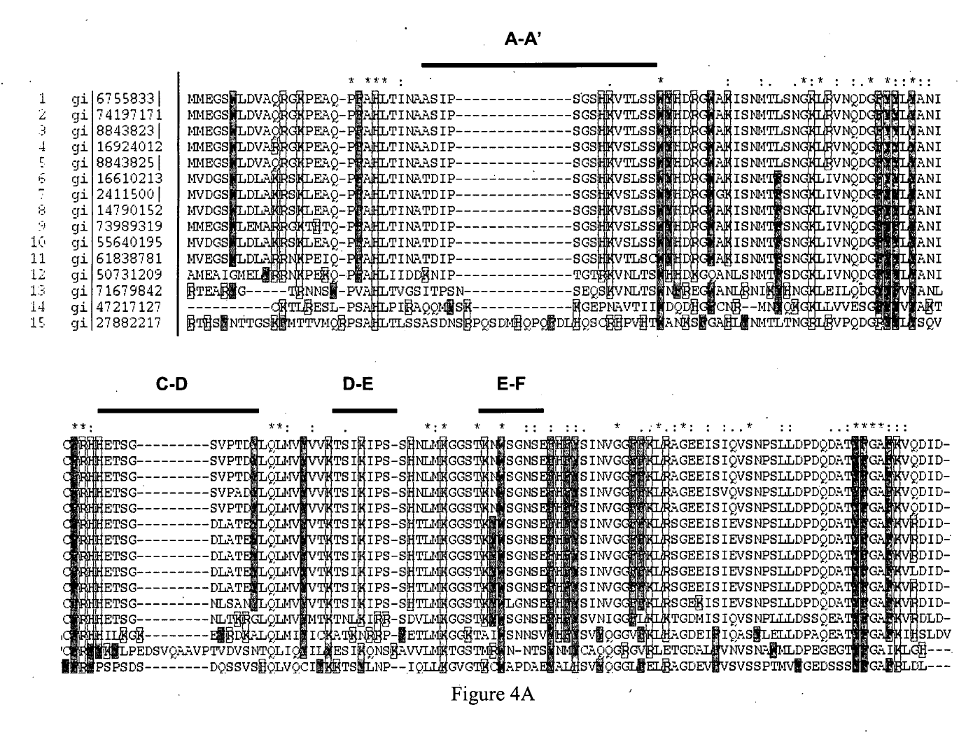

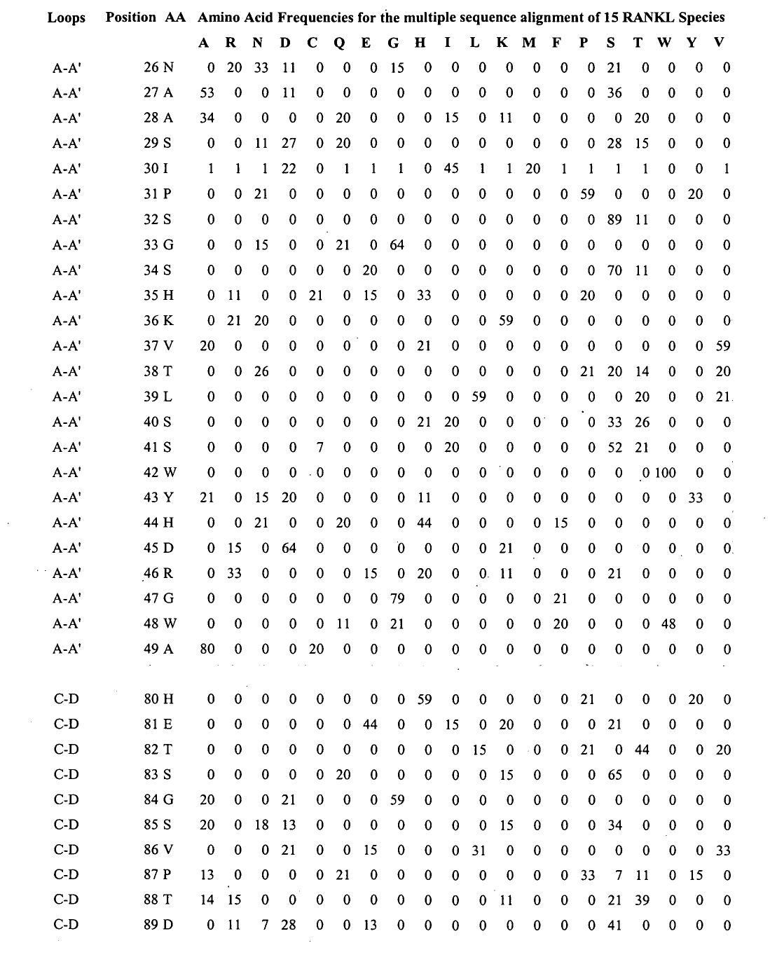

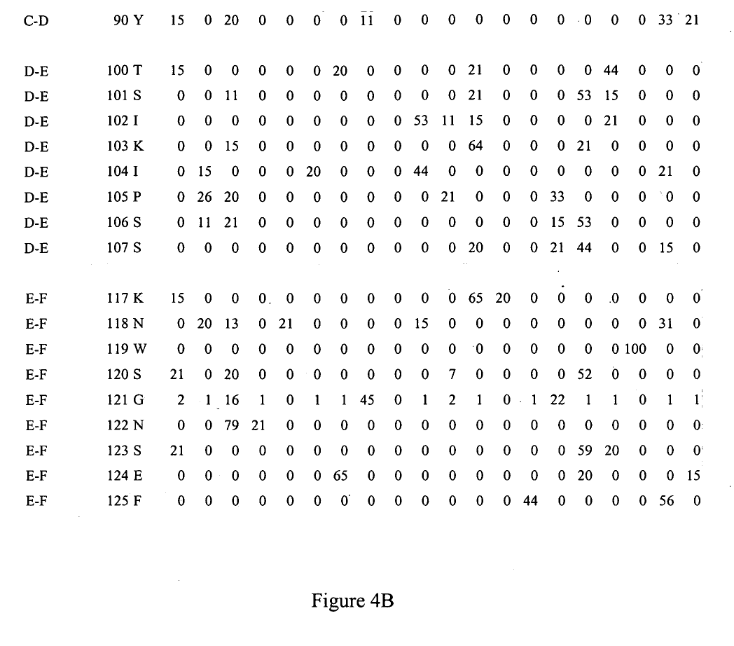

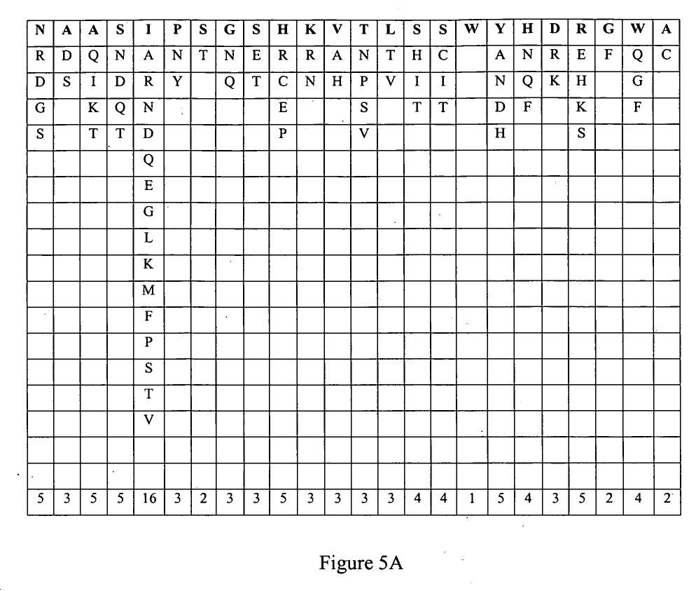

The surface loop peptide sequence alignments were used to generate cross-reactive sequences using the methods described by Luethy et al. “Improving the Suitability of sequence Profile Method the Sequence Profile Method” Protein Sci. 3, 139-146 (1994) (FIG. 4A). Sequence profiles were generated and used to search the entire non-redundant database, which includes the genome sequences from 24 eukaryotic organisms. 15 RANKL homologs were identified:

| Species | Isoforms | ID |
|---|---|---|
| | | gi\|16610213 |
| Human | 3 | gi\|2411500 |
| | | gi\|14790152 |
| | | gi\|6755833 |
| Mouse | 4 | gi\|74197171 |
| | | gi\|8843823 |
| | | gi\|8843825 |
| Rat | 1 | gi\|16924012 |
| Dog | 1 | gi\|73989319 |
| Chimp | 1 | gi\|55640195 |
| Chicken | 2 | gi\|50731209 |
| Fish | 2 | gi\|47217127 |
| | | gi\|27882217 |
| Cow | 1 | gi\|61838781 |
| Frog | 1 | gi\|55640195 |

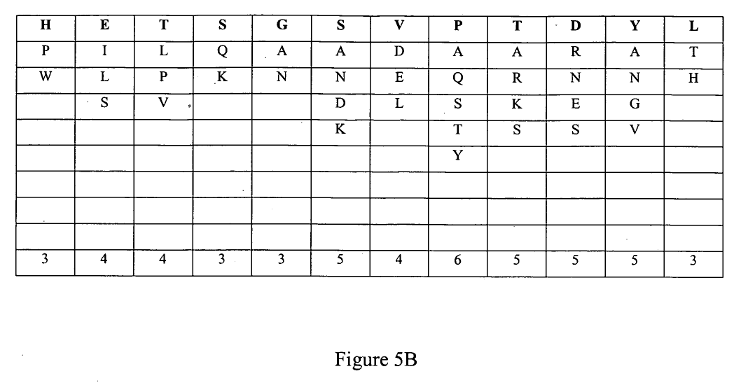

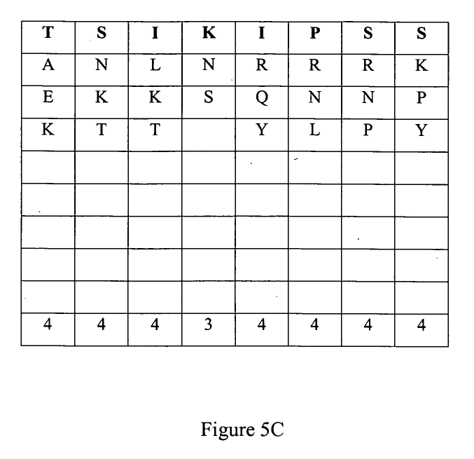

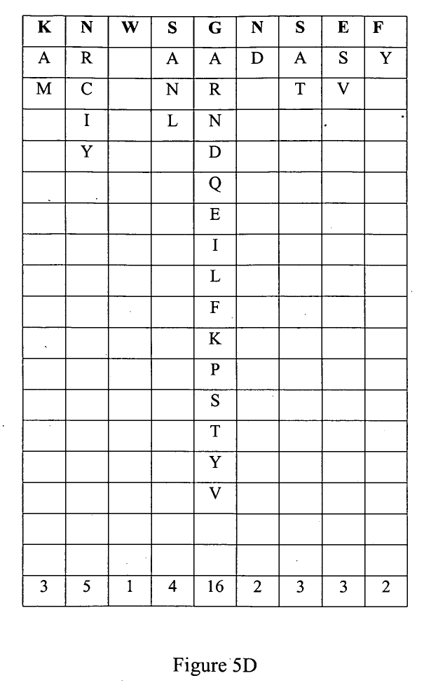

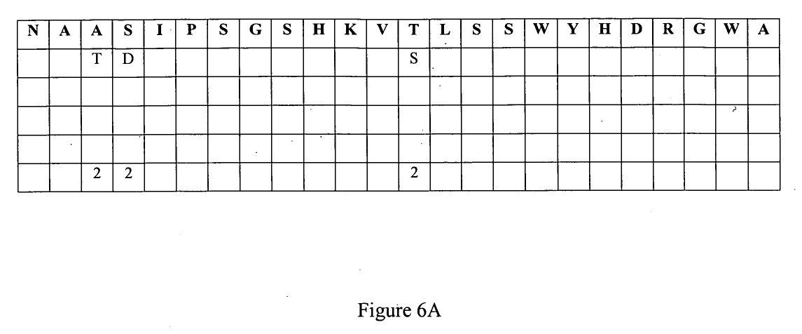

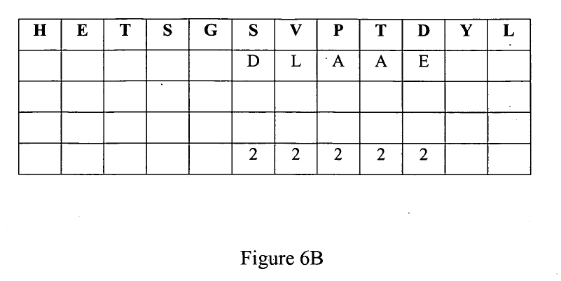

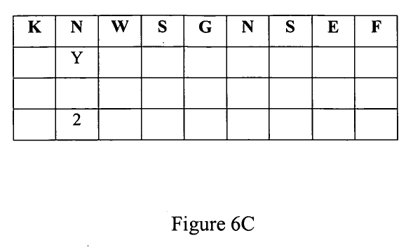

Using this compilation of 15 RANKL sequences, a multiple sequence alignment was created (FIG. 4A) and used to create a position specific scoring matrix (PSSM) file as described by Altschul S F et al., “Gapped BLAST and PSI-BLST: a new generation of protein database search programs.” Nucl. Acids Res. 25:3389-3402 (1997). The resulting matrix of substitution frequencies for each amino acid position, including the antigen loops in question, are shown in FIG. 4B. For each position of the respective surface loops (A-A′, C-D. D-E, and E-F), the amino acids found in the 15 RANKL isoforms are shown along with their frequencies. This chart can be used to predict the amino acid cross-reactive peptides that can serve as antigens to raise monoclonal antibodies, as for example, as shown in FIG. 5A-5D, respectively. Alternatively, a smaller subset of peptide antigens is described in FIG. 6A-6C, for the A-A′ C-D, and E-F, respectively, based on the multiple sequence alignment of the mouse, rat, dog, and human RANKL sequences alone.

To validate our prediction that the position specific matrix (PSSM) generated in FIG. 4 is a useful representation, we used it to screen the entire public database, and rank all of the matches by a corresponding probability, or “E” score. As shown, the top 15 matches are the input sequences used to generate the PSSM range of E=2.0e-97 to 8e-57. The next lowest score is the TNF related protein, TRAIL, which is known to be the closet homologue to the RANKL protein family (Lacey et al, 1998).

To produce antibodies that bind to the RANKL surface loops, and recognize the RANKL protein of various species, one can use protein sequences that resemble the native mouse, rat, or canine RANKL gene product, or they can be generated from cross-reactive sequences obtained from multiple RANKL protein sequence alignments. Alternatively, one can create peptides that are not identical to any known species, but are identified by the Profile method described above of having an expectation value of greater than or equal to 1.0e-55 compared against the PSSM.

To produce antigens from the RANKL surface loop regions, one can use DNA encoding the appropriate loop sequence to direct expression of a homologous peptide. The resulting peptide sequences can encode the entire loop sequences, or portions of the loop sequence that are large enough to be recognized by a mouse or human immunoglobulin.

These DNA sequences can be subcloned into plasmid vectors for expression in microorganisms such as E. coli. They can be used to direct expression of unfused gene products, or fused to heterologous protein sequences, such as fusion on to the glutathione Synthase Transferase (GST) protein, to serve as an epitope on a larger unrelated carrier protein. Alternatively, the DNA sequences can be subcloned into plasmid or episomal vectors useful for expression in eukaryotic cells, such as mammalian cells, yeast cells, or insect cells. The recombinant plasmid vectors can direct expression of the loop sequence derivatives either as un-fused proteins, or as fusions onto heterologous protein sequences, such as bovine serum albumin, or the immunoglobulin G (IgG) Fc region.

One can also produce antigens that resemble the native and cross-reactive RANKL peptide sequences described above as synthetic peptides. Peptides can be made using solid phase peptide synthesis following standard methods. The resulting peptides would be decoupled from the solid phase support, then purified using standard methods in protein purification. The purified products can then be coupled to carrier proteins to be used as antigens for the derivation of site-directed antibodies as described by Niman et al (Niman H L et al (1983) Proc Natl Acad Sci USA. 80(16):4949-53).

### Example 3

### Generation and Isolation of Monoclonal Antibodies

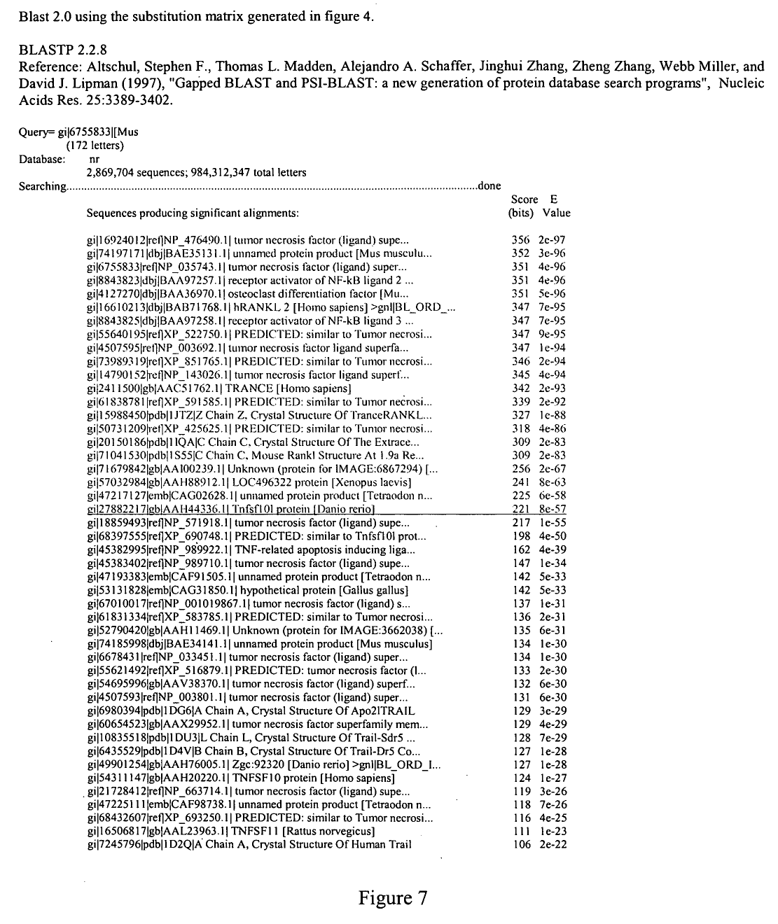

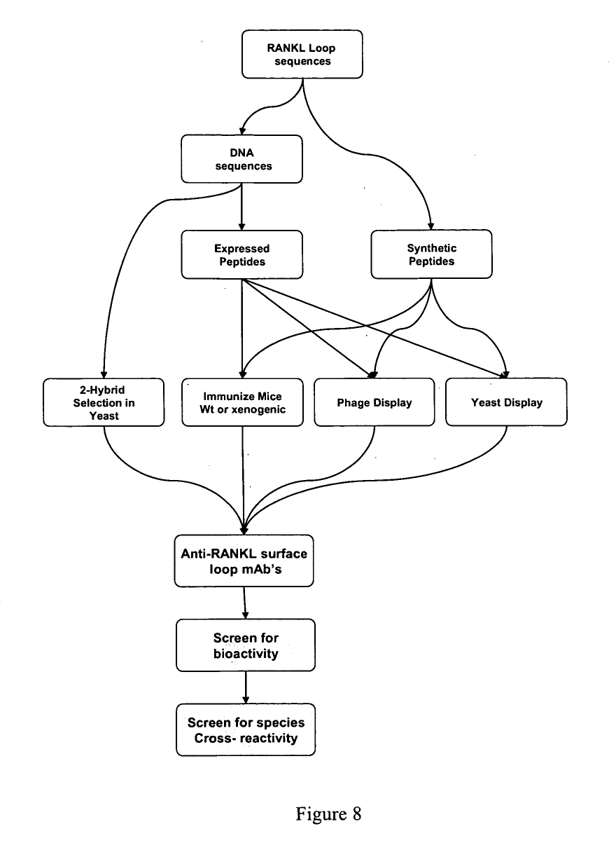

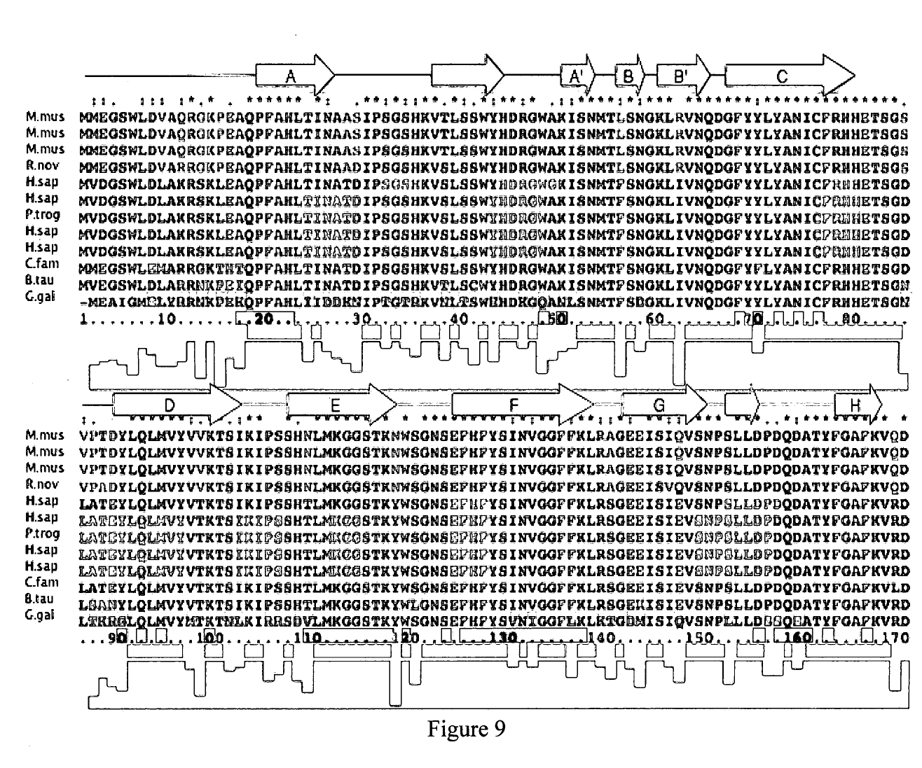

The overall strategy for producing site-directed anti-RANKL monoclonal antibodies is outlined in FIG. 8. Recombinantly expressed proteins, peptides, and synthetic peptides, either alone or as conjugates link to carrier proteins as described in Example 2 can be used to generate mouse monoclonal antibodies using standard methods known in the art, including as described by Kohler and Milstein (Kohler and Milstein (1975) Nature 256(5517):495-7) (FIG. 5), each of which is which is incorporated herein by reference in its entirety. Mice would be immunized with antigens, then boosted and their hyperimmune spleen cells used to generate hybridomas following fusion to a myeloma cell line. Upon selection, hybridoma cells would be tested for antibody expression, then cloned and expanded if secreting anti-RANKL binding IgG.

Alternatively, xenogenic mice containing human Ig locus transgenes as described by Green et al. in “Antigen-specific human monoclonal antibodies from mice engineered with human Ig heavy and light chain YACs, Nat. Genet. 1994 7(1):13-21, or by Fishwild et al. in “High avidity human IgGK monoclonal antibodies from a novel strain of minilocus transgenic mice,” Nature Biotechnology 14 (1996): 845-851, each of which is which is incorporated herein by reference in its entirety, can be used to produce hybridoma cells producing fully human, RANKL antigen-specific monoclonal antibodies.

Alternatively, anti-RANKL monoclonal antibodies can be selected from recombinant antibody libraries as described by Hoogenboom et al and Marks et al (Hoogenboom H R and Winter G. (1992) J Mol. Biol. 227(2):381-8; Marks C and Marks J D (1996) N Engl J Med 335(10):730-3; Marks J D et al (1991) J Mol Bio 222(3):581-97), and Hui et al. (Hui et al (2002) J. Mol. Biol. 315, 1063-1073), each of which is which is incorporated herein by reference in its entirety, using the aforementioned antigens. The VH and VL chain genes selected during this process would be recovered by PCR amplification, then grafted onto the CH and CL chain sequences to produce full length IgG polypeptides, as described by Hoogenboom and Charmes in “Natural and designer binding sites made by phage display technology”, Immunol. Today 21, 371-378 (2000), incorporated herein by reference in its entirety.

Alternatively, anti-RANKL monoclonal antibodies can be selected from yeast containing a cDNA library that encodes human IgG molecules on its surface using fluorescence activated cell sorting (FACS), as described by Feldhaus et al, in “Flow-Cytometric Isolation of Human Antibodies from a Nonimmune Saccharomyces cerevisiae Surface Display Library”, Nat. Biotech. 21(2):163-170 (2003), incorporated herein by reference in its entirety. Antibody secreting clones are selected by binding to fluorescently-labeled antigens by FACS analysis as described, and the VH and VL genes are rescued by PCR and grafted into an IgG backbone for further testing.

Alternatively, the DNA sequences encoding both native and cross-reactive RANKL loop sequences can be directly used to screen for antibodies and binding proteins in a yeast 2-Hybrid screening system as previously described by Hua et al., Gene 215(1):143-152, 1998) (FIG. 3), incorporated herein by reference in its entirety. Using this approach, one can insert the precise DNA sequences which encode the region to be targeted, such as the loop sequences identified form the murine RANKL crystal structure (Lam et al., United States Patent Application Publication Number 20030050223; Lam et al (2001) J Clin Invest 108(7):971-9). Cross reactive antibodies are identified by using a cDNA antibody library screened against all, or limited subfragments of, that encoded the murine RANKL surface loop domains.

The resulting monoclonal antibodies would be screened for binding to the cognate surface loop antigens that were used to select it from the all or some of the antibody generating sources described above, using standard methods for the isolation and characterization of monoclonal antibodies. Antibodies can then be tested for their ability to inhibit RANKL-induced osteoclastogenesis as previously described by Lacey et al. (1998) using mouse spleen cell precursors, or as previously described by Hsu et al., “Tumor necrosis factor receptor family member RANK mediates osteoclast differentiation and activation induced by osteoprotegerin ligand,” Proc Natl Acad Sci USA. 96, 3540-3545 (1999), using the mouse myelomonocytic cell line RAW 264.7.

Monoclonal antibodies with potent neutralizing activity can then be used to test for binding to surface loop peptides from various species. Surface loop peptides can be recombinantly expressed peptides using the DNA sequences from either or all of the 15 RANKL sequences present in the non-redundant nucleic acid database, described above. Alternatively, one can use isolated T-cells or T-cell lines from various mammalian species to provide native RANKL. RANKL expression and bioactivity is induced upon T-cell activation, as described by Kong et al., “Activated T cells regulate bone loss and joint destruction in adjuvant arthritis through osteoprotegerin ligand”. Nature 402, 304-309 (1999). Activated T-cells can stimulate osteoclastogenesis from mouse spleen cells, and this activity is blocked by using a RANKL inhibitor, osteoprotegerin. T-cells from human peripheral blood can be isolated and stimulated with immunomimetics, such as phytohemagglutinin (PHA), and used to stimulate osteoclastogenesis from either mouse spleen cells, or the RAW 264.7 cell line. The isolated anti-RANKL surface loop peptides antibodies can be tested in this assay to specifically determine their ability to bind and neutralize the human RANKL polypeptide and bioactivity.

### Example 4

This example illustrates the influence of the disruption of the interaction of RANK on CD1a+ dendritic cells (DC) by neutralizing anti-RANKL binding members. Functionally mature dendritic cells (DC) are generated in vitro from CD34+ bone marrow (BM) progenitors. Briefly, human BM cells from normal healthy volunteers are density fractionated using Ficoll medium and CD34+ cells immunoaffinity isolated using an anti-CD34 matrix column (Ceprate, CellPro). The CD34+ BM cells are then cultured in human GM-CSF (20 ng/ml), human IL-4 (20 ng/ml), human TNF-α (20 ng/ml), human CHO-derived Flt3L (FL; 100 ng/ml) in Super McCoy's medium supplemented with 10% fetal calf serum in a fully humidified 37° C. incubator (5% CO2) for 14 days. CD1a+, HLA-DR+DC are sorted using a FACStar Plus™, and used for biological evaluation of RANK and neutralizing anti-RANKL binding members.

On human CD1a+ DC derived from CD34+ bone marrow cells, only a subset (20-30%) of CD1a+ DC express RANK at the cell surface as assessed by flow cytometric analysis. However, addition of CD40L to the DC cultures results in RANK surface expression on the majority of CD1a+ DC. CD40L activates DC by enhancing in vitro cluster formation, inducing DC morphological changes and up-regulating HLA-DR, CD54, CD58, CD80 and CD86 expression

Addition of RANKL to DC cultures significantly increases the degree of DC aggregation and cluster formation above control cultures, similar to the effects seen with CD40L. Addition of neutralizing anti-RANKL binding members is expected to negate this effect. Sorted human CD1a+ DC are cultured in a cytokine cocktail (GM-CSF, IL-4, TNF-α and FL), in cocktail plus CD40L (1 μg/ml), in cocktail plus RANKL (1 μg/ml), or in cocktail plus heat inactivated RANKL (1 μg/ml) in 24-well flat bottomed culture plates in 1 ml culture media for 48-72 hours; each of the cultures can be tested in the presence of anti-RANKL binding members, and then photographed using an inversion microscope. An increase in DC aggregation and cluster formation above control cultures is not evident when heat inactivated RANKL is used, indicating that this effect is dependent on biologically active protein. Phenotypic analysis of adhesion molecule expression indicates that RANKL-induced clustering is not due to increased levels of CD2, CD11a, CD54 or CD58.

The addition of RANKL to CD1a+ DC enhances their allo-stimulatory capacity in a mixed lymphocyte reaction (MLR) by at least 3- to 10-fold, comparable to CD40L-cultured DC. Allogeneic T cells (1×105) are incubated with varying numbers of irradiated (2000 rad) DC cultured as indicated above in 96-well round bottomed culture plates in 0.2 mil culture medium for four days. The cultures are pulsed with 0.5 mCi [3H]-thymidine for eight hours and the cells are harvested onto glass fiber sheets for counting on a gas phase p counter. The addition of neutralizing anti-RANKL binding members to the assay is expected to inhibit allo-stimulatory capacity of RANKL in a MLR.

### Example 5

This example illustrates the effects of murine RANKL on cell proliferation and activation and the disruption caused by neutralizing anti-RANKL binding members.

Various cells or cell lines representative of cells that play a role in an immune response (murine spleen, thymus and lymphnode) are evaluated by culturing them under conditions promoting their viability, in the presence or absence of RANKL, and in the presence or absence of anti-RANKL binding members. One cell line, a macrophage cell line referred to as RAW 264.7 (ATCC accession number TIB 71) exhibits some signs of activation when exposed to RANKL and, therefore, a neutralizing anti-RANKL binding member, when added to the culture, will prevent activation of the cells.

RAW cells constitutively produce small amounts of TNF-α. Incubation with either human or murine RANKL enhances production of TNF-α by these cells in a dose dependent manner. Addition of neutralizing anti-RANKL binding members will prevent enhanced production of TNF-α by these cells in a dose dependent manner.

### Example 6

### Binding Assays

A number of conventional assays (e.g., Enzyme Linked Immunosorbant Assay (ELISA) or Biacore) can be used to detect interactions and binding affinity of binding members (e.g., antibodies) and a polypeptide.

Enzyme Linked Immunosorbant Assay

This example provides an exemplary ELISA human IgM that can be used to detect binding of anti-RANKL antibodies to RANKL. Wells of a 96-well microtiter plate (NeutrAvidin coated by Pierce) were coated with 0.5 μl/well of goat anti-human IgM-biotin antibody (Sigma). Antibody is added to 100 μl of PBS prior to adding to the wells for coating.

The plates are incubated overnight at 4° C., followed by washing one (1) time with phosphate buffered saline (PBS) and blocking with 3% bovine serum albumin (BSA) in PBS (200 μl/well) for 2-3 hours at room temperature to inhibit non-specific binding.

The plates are then washed 1 time with PBST (PBS-Tween 20; Sigma). PBS-1% BSA (90 μl) and 10 μl of the samples, respectively, are added to each well. A standard curve is created using IgM myeloma protein as a standard with a range starting 800 ng/ml down to 0 ng/ml (serial 1:2 dilutions).

The plates are incubated for 1 hour at room temperature and washed 3 times with PBST.

One hundred (100) μl of anti-IgM-horseradish peroxidase (HRP) (Sigma; 1:3000 dilution) in PBS-1% BSA is added to the wells and the plate is incubated for 30 minutes at room temperature, followed by washing 3 times with PBST.

One hundred (100) μl tetramethyl benzidine (TMB) substrate (Sure Blue; KPL) is added to each well and the plate is incubated for 15-20 minutes at room temperature in the dark. The reaction is stopped by adding 100 ul of hydrochloric acid (HCl) and the results are read in a plate reader at 450 nm.

In another example, a mouse and human ELISA can be conducted as follows: wells of a 96-well microtiter plate were coated with 50 μl/well of murine RANKL (17.6 ng/well or 352 ng/ml) and human RANKL (17.6 ng/well) diluted in PBS. Separate strips are used for each antigen.

The plates are incubated overnight at 4° C., followed by washing one (1) time with phosphate buffered saline (PBS) and blocking with 3% bovine serum albumin (BSA) in PBS (200 μl/well) for 2-3 hours at room temperature to inhibit non-specific binding. The plates are then washed 1 time with PBST (PB S-Tween 20; Sigma).

Fifty (50) μl of the samples and the standard, control antibody (Imgenex monoclonal antibody to RANKL, reacting to mouse and human) are added to the antigen-coated wells. A standard curve was generated in the ranges between 250 and 0 ng/ml (serial 1:2 dilutions). The plate is incubated for 1 hour at room temperature and washed 3 times with PBST.

One hundred (100) μl of HRP-conjugated antibody 1:500 dilution in PBS-1% BSA is added to the wells. For the mouse RANKL wells, goat anti-mouse (k-chain)-HRP is used and for the human RANKL wells, goat-anti-human (κ chain)-HRP is used. The plate is incubated for 30 minutes at room temperature and washed 3 times as above.

One hundred (100) μl tetramethyl benzidine (TMB) substrate (Sure Blue; KPL) is added to each well and the plate is incubated for 15-20 minutes at room temperature in the dark. The reaction is stopped by adding 100 μl of hydrochloric acid (HCl) and the results are read in a plate reader at 450 nm.

Kinetic Measurement of Anti-RANKL Antibody Binding to RANKL by Surface Plasmon Resonance (Biacore)

Mouse or human RANKL is immobilized onto a research grade CM5 sensor chip using standard amine coupling. Each of three surfaces is first activated for seven minutes using a 1:1 mixture of 0.1 mM N-hydroxysuccinimide (NHS) and 0.4 mM 1-ethyl-3-(3-dimethylaminopropyl)-carbodimide (EDC). Then, the RANKL sample is diluted 1- to 50-fold in 10 mM sodium acetate, pH 4.0, and exposed to the activated chip surface for different lengths of time (ten seconds to two minutes) to create three different density surfaces of RANKL. Each surface is then blocked with a seven-minute injection of 1 M ethanolamine, pH 8.2. Biotinylated RANKL is diluted 100-fold and injected for different amounts of time to be captured at three different surface densities (60 RU, 45 RU, 12 RU; Response Unit (RU) is termed by Biacore and relates to target molecule per surface area) onto a streptavidin-containing sensor chip. All experiments are performed on a Biacore® 2000 or T100 optical biosensor. Anti-RANKL antibody is supplied at 100 μg/mL and tested in a 3-fold dilution series in Sample Running Buffer over the three RANKL surfaces. Each of the anti-RANKL antibody concentrations (five, 3-fold dilutions of RANKL-coupled sensors) is tested three times to assess reproducibility of the assay. Each test is injected at a flow rate of 100 μL/minute for 60 seconds, followed by a three-minute dissociation phase. Bound anti-RANKL antibody is removed using a five-second pulse with sensor regeneration solution. All data are collected at a temperature-controlled 20° C. The kinetic responses for the antibody injections are analyzed using the non-linear least squares analysis program CLAMP (Myszka, D. G. and Morton, T. A. (1998) Trends Biochem. Sci., 23: 149-150). Calculations of multivalent interactions are determined using a model to fit the avidity of the bivalent interaction of anti-RANKL antibody with RANKL (Drake et al. (2004) Anal. Biochem., 328: 35-43; and Muller et al., (1998) Anal. Biochem., 261: 149-158).

### Example 7

This example describes RAW 264.7 cell in vitro osteoclastogenesis.

Preparation and Handling of Cells

Materials used in these methods include: BD Falcon 24 wells plates (VWR 62406-183); RAW264.7 cells (ATCC); DMEM-10% FBS (ATCC 30-2002); Murine MCSF (Peprotech Inc. Cat. No. 315-02); recombinant Human TRANCE/RANKL/TNSF 11 (R&D; Cat. No. 390-TN); and recombinant Human OPG/TNFRSF11B (R&D; Cat. No. 185-OS).

Raw264.7 cells are purchased from ATCC and frozen at passage three. Cells are plated in 24-well plates at a density of 5×10³ cells/well in DMEM-10% fetal bovine serum (FBS). Thawed cells are not used beyond an additional 3 passages.

Cells are incubated with murine macrophage-colony stimulating factor (M-CSF; 5 ng/ml) and with soluble human receptor activator of nuclear factor kappa B ligand (RANKL; 50 ng/ml) as appropriate to induce osteoclastogenesis. Wells grown with or without M-CSF and with or without human RANKL are used when appropriate as negative and positive controls.

Human osteoprotegerin (OPG) is used as an inhibitor of osteoclast formation both to demonstrate the inhibition of osteoclastogenesis in this assay and to provide a standard for subsequent antagonist antibody development. Respective concentrations of OPG are incubated with M-CSF and human RANKL for 30 min at 37° C. before being adding to the cells.

RAW cells are allowed to grow for 5 days in media and cytokines as indicated above, with replacement of media/cytokines every 2-3 days.

Osteoclast formation is assessed by staining cells for presence of tartrate-resistant acid phosphatase (TRAP) and by measuring the activity of the acid phosphatase using p-nitrophenyl-phosphophatase (PAP) activity colorimetric assay on day 5, each described in more detail below.

Tartrate-Resistant Acid Phosphatase Staining

Tartrate-resistant acid phosphatase (TRAP) activity was measured as a surrogate marker of in vitro osteoclastogenesis. Adherent RAW264.7 cells undergoing osteoclastogenesis (as described above, day 5) were washed with phosphate-buffered solution (PBS) and fixed with dilute citrate/acetate solution (Sigma©, St. Louis, Mo.; Catalog No. 386-1) for 30 seconds, washed with deionized water, and allowed to air dry. Cells were stained with a solution of 0.01% naphthol AS-BI phosphoric acid (Sigma©, St. Louis, Mo.; Catalog No. 386-4), tartrate (Sigma©, St. Louis, Mo.; Catalog No. 386-2), Fast Garnet GBC salt (Sigma©, Catalog No. 386-15), and dilute acetate, pH 5.2, for 5 minutes, before washing thoroughly with deionized water.

This invention may be embodied in other forms or carried out in other ways without departing from the spirit or essential characteristics thereof. The present disclosure is therefore to be considered as in all aspects illustrated and not restrictive, and all changes which come within the meaning and range of equivalency are intended to be embraced therein.

Various references are cited throughout this application, each of which is incorporated herein by reference in its entirety unless otherwise specifically noted.

What is claimed is:

1. An isolated cross-reactive RANKL polypeptide consisting essentially of an amino acid sequence selected from among SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 and SEQ ID NO: 43.

2. An antibody, or antigen-binding fragment, that binds to a cross-reactive RANKL polypeptide consisting essentially of an amino acid sequence selected from among SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 and SEQ ID NO: 43 and wherein said antibody, or antigen-binding fragment, neutralizes the activity of RANKL.

3. The antibody of claim 2, wherein said antibody is selected from among a polyclonal antibody, a monoclonal antibody, a chimeric antibody, and a humanized antibody.

4. The antigen binding fragment of claim 2, wherein said fragment is selected from among Fab, Fd, scFv, dAb, F(ab′)2, a bi-specific Fab2, a multivalent antibody fragment, and a bi-specific scFv.

5. A pharmaceutical composition comprising an antibody, or antigen-binding fragment, that binds to a cross-reactive RANKL polypeptide consisting essentially of an amino acid sequence selected from among SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 and SEQ ID NO: 43 and a pharmaceutically acceptable carrier.

6. The pharmaceutical composition of claim 5, wherein said pharmaceutically acceptable excipient is selected from the group consisting of a carrier, a buffer, a stabilizer, or any combination thereof.

7. The pharmaceutical composition of claim 5, further comprising a detectable or functional label.

8. The pharmaceutical composition of claim 7, wherein said detectable label is a radioisotope.

9. The pharmaceutical composition of claim 7, wherein said functional label is a cytotoxic drug.

10. The pharmaceutical composition of claim 5, wherein said antibody or antigen-binding fragment is conjugated to a second pharmaceutically active agent.

11. The pharmaceutical composition of claim 10, wherein said second pharmaceutically active agent is selected from among chemical ablation agents, toxins, immunomodulators, cytokines, and chemotherapeutic agents.

12. The pharmaceutical composition of claim 5, wherein said composition is for oral administration.

13. The pharmaceutical composition of claim 5, wherein said composition is for intravenous administration.

14. A method of preparing an antibody, which specifically binds a cross-reactive RANKL polypeptide of claim 1, comprising:
a) preparing a cross-reactive RANKL polypeptide of claim 1;
b) immunizing a rodent (e.g., a mouse) with a solution comprising the RANKL polypeptide of a) to generate an immune response in said mouse to said RANKL polypeptide;
c) isolating one or more spleen cells from said rodent of step b) after a sufficient amount of time for said mouse to generate said immune response;
d) fusing said isolated one or more spleen cells of step c) with a myeloma cell line to form a population of hybridoma cells;
e) culturing said hybridoma cell in a cell culture media comprising HAT;
f) selecting one or more cells within said population of hybridoma cells of e) which express a antibody that binds to said RANKL polypeptide of a); and
g) clonally expanding said one or more hybridoma cells of f).

15. The method of preparing an antibody of claim 14, further comprising:
h) identifying the antibody sequence of the antibody expressed in said one or clonal populations of cells of g).

16. The method preparing an antibody of claim 14, wherein said antibody is a monoclonal antibody.

17. A method of inhibiting bone loss associated with a condition comprising administering an antibody, or antigen-binding fragment, that binds to a cross-reactive RANKL polypeptide consisting essentially of an amino acid sequence selected from among SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 and SEQ ID NO: 43 and wherein said antibody, or antigen-binding fragment, neutralizes the activity of RANKL.

18. The method of claim 17, wherein said condition is selected from among osteoporosis, rheumatoid arthritis, bone metastasis and hypercalcemia of malignancy, glucocorticoid-induced bone loss, a periodontal disease or condition, a cancer and Juvenile Paget's Disease.

19. The method of claim 18, wherein said periodontal disease or condition is periodontitis or dental implants.

20. The method of claim 18, wherein said cancer is a primary tumor or a metastatic malignant tumor.

21. The method of claim 18, wherein said cancer is selected from among breast cancer, lung cancer, prostate cancer and bone cancer.
